SEQUENCE LISTING (32 sequences)

SEQ ID NO: 1 - protein, 1001 aa, Artificial Sequence
  Synthetic
  misc_feature (7)..(9): Xaa can be any naturally occurring amino acid
  misc_feature (11)..(11): Xaa can be any naturally occurring amino acid
  misc_feature (14)..(16): Xaa can be any naturally occurring amino acid
  misc_feature (20)..(20): Xaa can be any naturally occurring amino acid
  misc_feature (22)..(22): Xaa can be any naturally occurring amino acid
  misc_feature (29)..(29): Xaa can be any naturally occurring amino acid
  misc_feature (36)..(36): Xaa can be any naturally occurring amino acid
  misc_feature (42)..(43): Xaa can be any naturally occurring amino acid
  misc_feature (48)..(48): Xaa can be any naturally occurring amino acid
  misc_feature (52)..(53): Xaa can be any naturally occurring amino acid
  misc_feature (57)..(57): Xaa can be any naturally occurring amino acid
  misc_feature (59)..(59): Xaa can be any naturally occurring amino acid
  misc_feature (69)..(71): Xaa can be any naturally occurring amino acid
  misc_feature (73)..(73): Xaa can be any naturally occurring amino acid
  misc_feature (75)..(75): Xaa can be any naturally occurring amino acid
  misc_feature (78)..(79): Xaa can be any naturally occurring amino acid
  misc_feature (86)..(86): Xaa can be any naturally occurring amino acid
  misc_feature (89)..(89): Xaa can be any naturally occurring amino acid
  MISC_FEATURE (90)..(90): N = Isoleucine (I) or Leucine (L)
  misc_feature (101)..(101): Xaa can be any naturally occurring amino acid
  misc_feature (104)..(104): Xaa can be any naturally occurring amino acid
  misc_feature (111)..(111): Xaa can be any naturally occurring amino acid
  misc_feature (116)..(116): Xaa can be any naturally occurring amino acid
  misc_feature (121)..(121): Xaa can be any naturally occurring amino acid
  misc_feature (128)..(130): Xaa can be any naturally occurring amino acid
  misc_feature (133)..(133): Xaa can be any naturally occurring amino acid
  misc_feature (145)..(145): Xaa can be any naturally occurring amino acid
  misc_feature (148)..(148): Xaa can be any naturally occurring amino acid
  misc_feature (167)..(167): Xaa can be any naturally occurring amino acid
  misc_feature (174)..(174): Xaa can be any naturally occurring amino acid
  misc_feature (192)..(192): Xaa can be any naturally occurring amino acid
  misc_feature (194)..(195): Xaa can be any naturally occurring amino acid
  misc_feature (199)..(199): Xaa can be any naturally occurring amino acid
  misc_feature (202)..(203): Xaa can be any naturally occurring amino acid
  misc_feature (206)..(206): Xaa can be any naturally occurring amino acid
  misc_feature (217)..(219): Xaa can be any naturally occurring amino acid
  misc_feature (228)..(228): Xaa can be any naturally occurring amino acid
  misc_feature (233)..(233): Xaa can be any naturally occurring amino acid
  MISC_FEATURE (236)..(236): N = Isoleucine (I) or Leucine (L)
  misc_feature (243)..(243): Xaa can be any naturally occurring amino acid
  misc_feature (245)..(245): Xaa can be any naturally occurring amino acid
  misc_feature (262)..(262): Xaa can be any naturally occurring amino acid
  MISC_FEATURE (267)..(267): N = Isoleucine (I) or Leucine (L)
  misc_feature (277)..(278): Xaa can be any naturally occurring amino acid
  misc_feature (282)..(282): Xaa can be any naturally occurring amino acid
  misc_feature (287)..(287): Xaa can be any naturally occurring amino acid
  misc_feature (307)..(307): Xaa can be any naturally occurring amino acid
  misc_feature (314)..(314): Xaa can be any naturally occurring amino acid
  misc_feature (320)..(321): Xaa can be any naturally occurring amino acid
  misc_feature (333)..(333): Xaa can be any naturally occurring amino acid
  misc_feature (343)..(343): Xaa can be any naturally occurring amino acid
  misc_feature (357)..(357): Xaa can be any naturally occurring amino acid
  misc_feature (367)..(367): Xaa can be any naturally occurring amino acid
  misc_feature (371)..(371): Xaa can be any naturally occurring amino acid
  misc_feature (374)..(375): Xaa can be any naturally occurring amino acid
  misc_feature (378)..(378): Xaa can be any naturally occurring amino acid
  misc_feature (392)..(392): Xaa can be any naturally occurring amino acid
  misc_feature (402)..(402): Xaa can be any naturally occurring amino acid
  misc_feature (414)..(415): Xaa can be any naturally occurring amino acid
  MISC_FEATURE (418)..(418): N = Isoleucine (I) or Leucine (L)
  misc_feature (446)..(446): Xaa can be any naturally occurring amino acid
  misc_feature (448)..(449): Xaa can be any naturally occurring amino acid
  misc_feature (451)..(451): Xaa can be any naturally occurring amino acid
  misc_feature (467)..(467): Xaa can be any naturally occurring amino acid
  misc_feature (489)..(489): Xaa can be any naturally occurring amino acid
  misc_feature (500)..(500): Xaa can be any naturally occurring amino acid
  misc_feature (510)..(510): Xaa can be any naturally occurring amino acid
  misc_feature (514)..(514): Xaa can be any naturally occurring amino acid
  misc_feature (528)..(529): Xaa can be any naturally occurring amino acid
  misc_feature (532)..(532): Xaa can be any naturally occurring amino acid
  misc_feature (539)..(539): Xaa can be any naturally occurring amino acid
  misc_feature (547)..(548): Xaa can be any naturally occurring amino acid
  misc_feature (551)..(551): Xaa can be any naturally occurring amino acid
  misc_feature (553)..(554): Xaa can be any naturally occurring amino acid
  misc_feature (565)..(565): Xaa can be any naturally occurring amino acid
  misc_feature (576)..(576): Xaa can be any naturally occurring amino acid
  misc_feature (587)..(588): Xaa can be any naturally occurring amino acid
  misc_feature (600)..(600): Xaa can be any naturally occurring amino acid
  misc_feature (604)..(605): Xaa can be any naturally occurring amino acid
  misc_feature (622)..(622): Xaa can be any naturally occurring amino acid
  misc_feature (627)..(627): Xaa can be any naturally occurring amino acid
  misc_feature (629)..(629): Xaa can be any naturally occurring amino acid
  misc_feature (655)..(655): Xaa can be any naturally occurring amino acid
  misc_feature (662)..(662): Xaa can be any naturally occurring amino acid
  misc_feature (682)..(682): Xaa can be any naturally occurring amino acid
  misc_feature (686)..(686): Xaa can be any naturally occurring amino acid
  MISC_FEATURE (688)..(688): N = Isoleucine (I) or Leucine (L)
  misc_feature (691)..(691): Xaa can be any naturally occurring amino acid
  misc_feature (698)..(698): Xaa can be any naturally occurring amino acid
  misc_feature (706)..(706): Xaa can be any naturally occurring amino acid
  misc_feature (723)..(723): Xaa can be any naturally occurring amino acid
  MISC_FEATURE (737)..(737): N = Isoleucine (I) or Leucine (L)
  misc_feature (738)..(738): Xaa can be any naturally occurring amino acid
  misc_feature (760)..(761): Xaa can be any naturally occurring amino acid
  misc_feature (771)..(771): Xaa can be any naturally occurring amino acid
  misc_feature (773)..(773): Xaa can be any naturally occurring amino acid
  misc_feature (779)..(779): Xaa can be any naturally occurring amino acid
  misc_feature (786)..(786): Xaa can be any naturally occurring amino acid
  misc_feature (790)..(790): Xaa can be any naturally occurring amino acid
  misc_feature (798)..(798): Xaa can be any naturally occurring amino acid
  misc_feature (800)..(800): Xaa can be any naturally occurring amino acid
  misc_feature (834)..(834): Xaa can be any naturally occurring amino acid
  misc_feature (843)..(843): Xaa can be any naturally occurring amino acid
  misc_feature (851)..(852): Xaa can be any naturally occurring amino acid
  misc_feature (866)..(866): Xaa can be any naturally occurring amino acid
  misc_feature (870)..(870): Xaa can be any naturally occurring amino acid
  misc_feature (873)..(873): Xaa can be any naturally occurring amino acid
  misc_feature (877)..(877): Xaa can be any naturally occurring amino acid
  misc_feature (889)..(889): Xaa can be any naturally occurring amino acid
  misc_feature (896)..(896): Xaa can be any naturally occurring amino acid
  misc_feature (916)..(916): Xaa can be any naturally occurring amino acid
  misc_feature (923)..(923): Xaa can be any naturally occurring amino acid
  misc_feature (936)..(936): Xaa can be any naturally occurring amino acid
  misc_feature (962)..(962): Xaa can be any naturally occurring amino acid
  misc_feature (966)..(966): Xaa can be any naturally occurring amino acid
  misc_feature (970)..(970): Xaa can be any naturally occurring amino acid
  misc_feature (985)..(985): Xaa can be any naturally occurring amino acid
  misc_feature (996)..(996): Xaa can be any naturally occurring amino acid
MKISKVXXXVXKKXXXGKLXKXVNERNRXAKRLSNXLBKYIXXIDKIXKKEXXKKFXAXEEITLKLNQXXXBXLXKAXXDLRKDNXYSXNKKILHNEDINXEEXELLINDXLEKLXKIESXKYSYQKXXXNYXMSVQEHSKKSIXRIXESAKRNKEALDKFLKEYAXLDPRMEXLAKLRKLLELYFYFKNDXIXXEEEXNVXXHKXLKENHPDFVEXXXNKENAELNXYAIEXKKNLKYYFPXKXAKNSNDKIFEKQELKKXWIHQNENAVERILLXXGKVXYKLQXGYLAELWKIRINEIFIKYIXVGKAVAXFALRNXXKBENDILGGKIXKKLNGITSFXYEKIKAEEILQREXAVEVAFAANXLYAXDLXXIRXSILQFFGGASNWDXFLFFHFATSXISDKKWNAELIXXKKNGLVIREKLYSNNVAMFYSKDDLEKLLNXLXXFXLRASQVPSFKKVYVRXBFPQNLLKKFNDEKDDEAYSAXYYLLKEIYYNXFLPYFSANNXFFFXVKNLVLKANKDKFXXAFXDIREMNXGSPIEYLXXTQXNXXNEGRKKEEKEXDFIKFLLQIFXKGFDDYLKNNXXFILKFIPEPTEXIEIXXELQAWYIVGKFLNARKXNLLGXFXSYLKLLDDIELRALRNENIKYQSSNXEKEVLEXCLELIGLLSLDLNDYFBDEXDFAXYNGKXLDFEKKXMKDLAELXPYDQNDGENPIVNRNIXLAKKYGTLNLLEKNXDKVSEKEIKEYYELKKEIEEYXXKGEELHEEWXQXKNRVEXRDILEYXEELXGQIINYNXLXNKVLLYFQLGLHYLLLDILGRLVGYTGIWERDAXLYQIAAMYXNGLPEYIXXKKNDKYKDGQIVGXKINXFKXDKKXLYNAGLELFENXNEHKNIXIRNYIAHFNYLSKAESSLLXYSENLRXLFSYDRKLKNAVXKSLINILLRHGMVLKFKFGTDKKSVXIRSXKKIXHLKSIAKKLYYPEVXVSKEYCKLVKXLLKYK

SEQ ID NO: 2 - protein, 6 aa, Artificial Sequence
  Synthetic
  MISC_FEATURE (3)..(3): X is R, S, D, E, Q, N, G, Y, or H
  MISC_FEATURE (4)..(4): X is I, S, T, V, or L
  MISC_FEATURE (5)..(5): X is L, F, N, Y, V, I, S, D, E, or A
RNXXXH

SEQ ID NO: 3 - protein, 6 aa, Artificial Sequence
  Synthetic
  MISC_FEATURE (3)..(3): X is R, S, D, E, Q, N, G, Y, or H
  MISC_FEATURE (4)..(4): X is I, S, T, V, or L
  MISC_FEATURE (5)..(5): X is L, F, N, Y, V, I, S, D, E, or A
RHXXXH

SEQ ID NO: 4 - protein, 6 aa, Artificial Sequence
  Synthetic
  MISC_FEATURE (3)..(3): X is R, S, D, E, Q, N, G, Y, or H
  MISC_FEATURE (4)..(4): X is I, S, T, V, or L
  MISC_FEATURE (5)..(5): X is L, F, N, Y, V, I, S, D, E, or A
RKXXXH

SEQ ID NO: 5 - protein, 6 aa, Artificial Sequence
  Synthetic
  MISC_FEATURE (3)..(3): X is R, S, D, E, Q, N, G, Y, or H
  MISC_FEATURE (4)..(4): X is I, S, T, V, or L
  MISC_FEATURE (5)..(5): X is L, F, N, Y, V, I, S, D, E, or A
RNXXXH

SEQ ID NO: 6 - protein, 6 aa, Artificial Sequence
  Synthetic
  MISC_FEATURE (3)..(3): X is R, S, D, E, Q, N, G, Y, or H
  MISC_FEATURE (4)..(4): X is I, S, T, V, or L
  MISC_FEATURE (5)..(5): X is L, F, N, Y, V, I, S, D, E, or A
RHXXXH

SEQ ID NO: 7 - protein, 6 aa, Artificial Sequence
  Synthetic
  MISC_FEATURE (3)..(3): X is R, S, D, E, Q, N, G, Y, or H
  MISC_FEATURE (4)..(4): X is I, S, T, V, or L
  MISC_FEATURE (5)..(5): X is L, F, N, Y, V, I, S, D, E, or A
RNXXXH

SEQ ID NO: 8 - protein, 6 aa, Artificial Sequence
  Synthetic
  MISC_FEATURE (3)..(3): X is R, S, D, E, Q, N, G, Y, or H
  MISC_FEATURE (4)..(4): X is I, S, T, V, or L
  MISC_FEATURE (5)..(5): X is L, F, N, Y, V, I, S, D, E, or A
RKXXXH

SEQ ID NO: 9 - RNA, 25 nt, Artificial Sequence
  Synthetic
gggaacaaagcugaaguacuuaccc

SEQ ID NO: 10 - DNA, 18 bp, Artificial Sequence
  Synthetic
gggtagggcgggttggga

SEQ ID NO: 11 - RNA, 12 nt, Artificial Sequence
  Synthetic
  misc_feature (1)..(1): Biotin Tag = /5Biosg/
  misc_feature (12)..(12): 3IAbRQSp = Iowa black quencher
ucucguacguuc

SEQ ID NO: 12 - RNA, 24 nt, Artificial Sequence
  Synthetic
  misc_feature (1)..(1): Biotin Tag = /5Biosg/
  misc_feature (24)..(24): 3IAbRQSp = Iowa black quencher
ucucguacguucucucguacguuc

SEQ ID NO: 13 - DNA, 10 bp, Homo sapiens
tatatatata

SEQ ID NO: 14 - DNA, 15 bp, Homo sapiens
gtcgtcgtcgtcgtc

SEQ ID NO: 15 - DNA, 20 bp, Artificial Sequence
  Synthetic
gaccccaaaatcagcgaaat

SEQ ID NO: 16 - DNA, 24 bp, Artificial Sequence
  Synthetic
tctggttactgccagttgaatctg

SEQ ID NO: 17 - DNA, 24 bp, Artificial Sequence
  Synthetic
  misc_feature (1)..(1): FAM=6-carboxyfluorescein
  misc_feature (24)..(24): BHQ1=Black Hole Quencher-1
accccgcattacgtttggtggacc

SEQ ID NO: 18 - DNA, 25 bp, Artificial Sequence
  Synthetic
gaaattaatacgactcactataggg

SEQ ID NO: 19 - DNA, 25 bp, Artificial Sequence
  Synthetic
gaaattaatacgactcactataggg

SEQ ID NO: 20 - RNA, 28 nt, Artificial Sequence
  Synthetic
cucuucuucagguugaagagcagcagaa

SEQ ID NO: 21 - DNA, 20 bp, Artificial Sequence
  Synthetic
gaccccaaaatcagcgaaat

SEQ ID NO: 22 - DNA, 24 bp, Artificial Sequence
  Synthetic
tctggttactgccagttgaatctg

SEQ ID NO: 23 - DNA, 24 bp, Artificial Sequence
  Synthetic
accccgcattacgtttggtggacc

SEQ ID NO: 24 - DNA, 30 bp, Artificial Sequence
  Synthetic
ccaaggtaaacctttggaatttggtgccac

SEQ ID NO: 25 - DNA, 30 bp, Artificial Sequence
  Synthetic
actatcatcatctaaccaatcttcttcttg

SEQ ID NO: 26 - DNA, 475 bp, Artificial Sequence
  Synthetic
gcaccatgctattgctttaacgctgagggtgatgcatcttggtcttctactatgatcttctctcttcaccccgtcgagtgtgacgaggagtgttctgaagtagaggcttcagatttagaagaaggtgaatcagagtgcatttctgagacttcaactgaacaagttgacgtttctcatgaggtttctgacgacgagtgggctgctgcagttgatgaagcgttccccctcgatgaagcagaagatgttactgaatctgtgcaagaagaagcacaaccagtagaagtacctgttgaagatattgcgcaggttgtcatagctgacaccttacaggaaactcctgttgtgtctgatactgttgaagtcccaccgcaagtggtgaaacttccgtctgaacctcagactatccagcccgaggtaaaagaagttgcacctgtctatgaggctgataccgaacagacacagagtgttactgtta

SEQ ID NO: 27 - DNA, 475 bp, Artificial Sequence
  Synthetic
atatggaggaattttatgctgtggtgattgatgccatagaagagaaactttctccatgtaaggagcttgaaggtgtaggtgctaaagttagtgcctttttacagaaattagaggatgaatttgtaatcccctatttttatttgatgaggctggcgaagaagtttttgctcctaaattgtattgtgcctttacagctcctgaagatgatgactttcttgaggaaagtgatgttgaagaagatgatgtagaaggtgaggaaactgatttaactatcacaagtgctggacagccttgtgttgctagtgaacaggaggaatcttctgaagtcttagaggacactttggatgatggtccaagtgtggagacatctgattcacaagttgaagaagatgtagaaatgtcggattttgttgatcttgaatctgtgattcaggattatgaaaatgtttgttttgagttttatactacagagcca

SEQ ID NO: 28 - DNA, 475 bp, Artificial Sequence
  Synthetic
gaatgtgagaatcacatttgagcttgatgaacgtgttgacaaagtgcttaatgaaaagtgctctgtctacactgttgaatccggtaccgaagttactgagtttgcatgtgttgtagccagaaccagaaagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagc

SEQ ID NO: 29 - DNA, 475 bp, Artificial Sequence
  Synthetic
gtgagtttaaattggcttcacatatgtattgttctttctaccctccagatgaggatgaagaagaaggtgattgtgaagaagaagagtttgagccatcaactcaatatgagtatggtatggaggaggctgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaaca

SEQ ID NO: 30 - DNA, 25 bp, Artificial Sequence
  Synthetic
gaaattaatacgactcactataggg

SEQ ID NO: 31 - RNA, 28 nt, Artificial Sequence
  Synthetic
  misc_feature (1)..(1): /56-FAM/=isomer derivative of fluorescein attachment for oligonucleotides
  misc_feature (28)..(28): Biotin Tag = /3Bio/
rurururururururururururururu

SEQ ID NO: 32 - RNA, 14 nt, Artificial Sequence
  Synthetic
  misc_feature (1)..(1): /56-FAM/=isomer derivative of fluorescein attachment for oligonucleotides
  misc_feature (14)..(14): /3IABkFQ/=Iowa Black quencher ideal for use with fluorescein
rururururururu

(19) United States
(12) Patent Application Publication
(10) Pub. No.: US 2024/0102115 A1
(43) Pub. Date: Mar. 28, 2024

(54) CRISPR EFFECTOR SYSTEM BASED DIAGNOSTICS FOR VIRUS DETECTION

(71) Applicants: THE BROAD INSTITUTE, INC., Cambridge, MA (US); PRESIDENT AND FELLOWS OF HARVARD COLLEGE, Cambridge, MA (US)

(72) Inventors: Pardis Sabeti, Cambridge, MA (US); Cameron Myhrvold, Cambridge, MA (US); Jon Arizti Sanz, Cambridge, MA (US); Alexandra Stanton, Cambridge, MA (US); Brittany Petros, Cambridge, MA (US)

(21) Appl. No.: 18/023,538

(22) PCT Filed: Sep. 3, 2021
(86) PCT No.: PCT/US2021/049145

§ 371 Date: Feb. 27, 2023

Related U.S. Application Data
(60) Provisional application No. 63/074,307, filed on Sep. 3, 2020.

(51) Int. Cl. C12Q 1/70; C12N 9/22; C12N 15/11; C12Q 1/6806; G01N 33/543

(52) U.S. Cl. CPC C12Q 1/701; C12N 9/22; C12N 15/11; C12Q 1/6806; G01N 33/54388; C12N 2310/20

(57) ABSTRACT

Described herein are compositions and methods of CRISPR effector system mediated detection of targets, including, but not limited to viral targets. Also described herein are devices and kits for carrying out the CRISPR effector system mediated nucleic acid detection assays.

### CROSS-REFERENCE TO RELATED APPLICATIONS

This application claims the benefit of U.S. Provisional Application No. 63/074,307, filed Sep. 3, 2020. The entire contents of the above-identified applications are hereby fully incorporated herein by reference.

### STATEMENT REGARDING FEDERALLY SPONSORED RESEARCH

This invention was made with government support under Grant No. DAC00006 awarded by the Defense Advanced Research Projects Agency and GM007753 awarded by the National Institutes of Health. The government has certain rights in the invention.

### SEQUENCE LISTING

This application contains a sequence listing filed in electronic form as an ASCII.txt file entitled BROD-5175WP_ST25.txt, created on Sep. 3, 2021 and having a size of 31,590 bytes (33 KB on disk). The content of the sequence listing is incorporated herein in its entirety.

### TECHNICAL FIELD

The subject matter disclosed herein is generally directed to nucleic-acid based virus detection, and more particularly, CRISPR effector system based virus detection.

### BACKGROUND

Recent viral outbreaks, such as SARS-Cov-2, have highlighted the challenges of diagnosing viral infections, both rapidly and accurately, particularly in areas far from clinical laboratories and in large quantities. Indeed, many of the traditional nucleic-acid based virus detection techniques, although sensitive and adaptable, require significant infrastructure (e.g., specialized and expensive equipment and highly trained personnel to perform the techniques) and extensive sample manipulation. Therefore, these current techniques fail to be widely and rapidly deployed in the face of an emerging viral outbreak.

As is highlighted by the present COVID-19 pandemic, point-of-care diagnostic testing is important to the response to, control of, and prevention of infectious disease outbreaks, particularly on the global scale. Insufficient nucleic acid diagnostic testing infrastructure and the prevalence of asymptomatic transmission have accelerated the global spread of SARS-Cov-2. Response to, control of, and prevention of other exemplary viral outbreaks such as Zika virus (ZIKV), dengue virus (DENV), MERS-Cov, Avian Influenza (H1N1) and others suffered from a similar lack of insufficient diagnostic testing infrastructure, including in some cases, a lack of suitable tests sensitive enough to distinguish between closely related viruses and strains.

The paradigm for nucleic acid diagnostic testing is a centralized model where patient samples are sent to large clinical laboratories for processing and analysis. RT-qPCR, the highly specific and sensitive current gold-standard for SARS-CoV-2 diagnosis (U.S. Food and Drug Administration, Policy for CVID-19 Tests During the Public Health Emergency (revised) 202. (https://www.fda.gov/regulatory-information/search-fda-guidance-documents/policy-coronavirus-disease-2019-tests-during-public-health-emergency-revised), requires laboratory infrastructure for nucleic acid extraction, thermal cycling, and analysis of assay results. The need for thermocyclers can be eliminated through the use of isothermal (i.e., single temperature) amplification methods, such as loop-mediated isothermal amplification (LAMP) and recombinase polymerase amplification (RPA) (see e.g., Notomi et al. 2000. Nuc Acid Res. 28, E63; Park et al., J Mol Diagn. (2020), doi:10.1016/j.jmoldx.2020.03.006; Baek et al. 2020. Emerg. Microbes Infect., 1-31; Niemz et al. 2011. Trends Biotech. 29:240-250; Abott ID NOW™ COVID-19—Alere is now, (available at https://www.alere.com/en/home/product-details/id-now-covid-19.html); and Pipenburg et al. 2006. PloS Biol. 4, e204. However, isothermal amplification methods still require technological advances to increase sensitivity on unextracted RNA samples and to reduce non-specific amplification (see e.g., Zaghloul et al. 2014. World J. Hepatol. 6:916-922 and Yan et al. 2014. Mol BioSys 10:970), to allow for testing at scale outside of laboratories.

As such, an urgent need exists for compositions, devices, and techniques that can detect viruses present in view of at least the limitations of these current technique and others.

Citation or identification of any document in this application is not an admission that such a document is available as prior art to the present invention.

### SUMMARY

Described in certain example embodiments herein are compositions comprising: a nucleic acid detection system comprising: a CRISPR system comprising an effector protein and one or more guide RNAs, capable of specifically binding a virus-specific target molecule; and a viral polynucleotide preparation formulation capable of releasing virus polynucleotides from viruses, inactivating nucleases, inactivating viruses, or a combination thereof at a temperature of about 15 degrees, 25 degrees, or 37 degrees Celsius or greater.

In certain example embodiments, the composition further comprises a detection construct.

In certain example embodiments, the detection construct comprises an RNA or DNA oligonucleotide and further comprises a first molecule on a first end and a molecule on a second end.

In certain example embodiments, the composition further comprises one or more nucleic acid amplification reagents.

Described in certain example embodiments herein are methods of detecting a virus in a sample comprising: releasing virus polynucleotides from a virus in the sample; inactivating nucleases present in the sample; inactivating viruses present in the sample; amplifying virus polynucleotides in the sample; combining the sample with a nucleic acid detection system comprising: a CRISPR system comprising an effector protein and one or more guide RNAs, capable of specifically binding a virus-specific target molecule; and a detection construct; activating the effector protein such that a detectable positive signal is produced, wherein activating the effector protein occurs via specific binding of the one or more guide RNAs to one or more virus-specific target molecules and results in modification of the detection construct such that a detectable signal is produced; and detecting the detectable signal, wherein the detectable signal indicates a presence of one or more viruses in the sample, wherein amplifying and activating occur in the same reaction and wherein the method does not include a step of extracting a virus polynucleotide from the sample.

In certain example embodiments, the steps of releasing, inactivating nucleases, inactivating viruses, amplifying and activating occur in the same reaction vessel.

In certain example embodiments, the steps of releasing, inactivating nucleases, inactivating viruses, amplifying, activating, and detecting occur in the same reaction vessel.

In certain example embodiments, the step of releasing, inactivating nucleases, inactivating virus, or a combination thereof occurs in a viral polynucleotide preparation formulation capable of releasing virus polynucleotides from viruses, inactivating nucleases, inactivating viruses, or a combination thereof at a temperature of about 15 degrees Celsius or greater.

In certain example embodiments, the nucleic acid detection system is contained in the viral polynucleotide preparation formulation.

In certain example embodiments, the viral polynucleotide preparation formulation comprises one or more of the following: a buffer, wherein the buffer is optionally HEPES, an amount of sucrose, an amount mannitol, a salt, PEG-8000, and PEG-1500.

In certain example embodiments, the viral polynucleotide preparation formulation does not comprise PEG.

In certain example embodiments, the viral polynucleotide preparation formulation is lyophilized.

In certain example embodiments, inactivating nucleases is carried out at a temperature ranging from about 15 degrees C. to about 50 degrees C.

In certain example embodiments, inactivating viruses occurs at a temperature ranging from about 15 degrees C. to about 95 degrees C.

In certain example embodiments, inactivating nucleases and inactivating viruses occurs at the same temperature.

In certain example embodiments, inactivating nucleases and inactivating viruses occurs at different temperatures.

In certain example embodiments, inactivating nucleases, inactivating viruses, or both together occurs for a period of time ranging from about 5 minutes to about 60 minutes.

In certain example embodiments, the method further comprises distributing a sample or set of samples into one or more individual discrete volumes, wherein the individual discrete volumes comprise the nucleic acid detection.

In certain example embodiments, the method further comprises incubating the sample or set of samples under conditions sufficient to allow binding of the one or more guide RNAs to one or more virus-specific target molecules.

In certain example embodiments, the one or more guide RNAs comprise one or more synthetic mismatches.

In certain example embodiments, the one or more guide RNAs comprise a pan-viral guide RNA set that is capable of detecting each virus, viral strain, or both in a set of viruses.

In certain example embodiments, the guide RNAs are derived using a set cover approach.

In certain example embodiments, the amplification step occurs for a period of time ranging from about 10 minutes to 2 hours.

In certain example embodiments, the detection step is of a period of time ranging from about 10 minutes to 3 hours.

In certain example embodiments, the sample volume ranges from about 1 microliter to about 100 microliters.

In certain example embodiments, the detection construct comprises or consists of an RNA-based detection construct comprising an RNA oligonucleotide to which a detectable molecule and masking component are attached.

In certain example embodiments, the effector protein is a Cas protein having collateral polynucleotide cleavage activity.

In certain example embodiments, the Cas protein having collateral polynucleotide cleavage activity is selected from: Cas13a (C2c2), Cas13b (Group 29/30), Cas13c, Cas13d, Cas12a (Cpf1), Cas12b (C2c1), Cas12c (C2c3), Cas12d (CasY), Cas12e (CasX), Cas14, and combinations thereof.

In certain example embodiments, the sample comprises two or more viruses and wherein the method distinguishes between the two or more viruses.

In certain example embodiments, the guide RNAs are capable of detecting single nucleotide variants of one or more viruses.

In certain example embodiments, the detectable signal is an optical signal.

In certain example embodiments, the optical signal is a fluorescent signal or a colorimetric signal.

In certain example embodiments, the nucleic acid detection system is not contained in/on a substrate.

In certain example embodiments, the nucleic acid detection system is contained in/on a substrate, and wherein the substrate is exposed to the sample.]

In certain example embodiments, the same or a different nucleic acid detection system is present at multiple discrete locations on the substrate.

In certain example embodiments, the substrate is a flexible materials substrate.

In certain example embodiments, the flexible materials substrate is a paper substrate, a fabric substrate, or a flexible polymer-based substrate.

In certain example embodiments, each different nucleic acid detection system detects a different virus or viral strain at each discrete location.

In certain example embodiments, the substrate is exposed to the sample passively, by immersing the substrate in a fluid to be sampled, by applying a fluid to be tested to the substrate, or by contacting a surface to be tested with the substrate.

In certain example embodiments, the substrate is configured as a lateral flow strip.

In certain example embodiments, the detection construct comprises a first and a second molecule and wherein the method comprises detecting the first and the second molecule optionally at discrete locations on the lateral flow strip.

In certain example embodiments, the first molecule and the second molecule are detected by binding a first antibody capable of specifically binding the first molecule or the second molecule, and optionally further comprising detecting the bound first antibody, optionally with a second antibody capable of specifically binding the first antibody.

In certain example embodiments, said lateral flow strip comprises an upstream first antibody directed against the first molecule and a downstream second antibody directed against the second molecule, and wherein an uncleaved detection construct is bound by the first antibody when the target molecule is not present in said sample, and wherein a cleaved detection construct is bound both by the first antibody and the second antibody when the target nucleic acid is present in said sample.

In certain example embodiments, the sample is a biological or environmental sample

In certain example embodiments, the biological sample is obtained from a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface.

In certain example embodiments, the environmental sample is obtained from a food sample, a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof.

In certain example embodiments, the environmental sample or biological samples are crude samples and/or wherein the one or more target molecules are not purified or amplified from the sample prior to application of the method.

In certain example embodiments, the virus is a DNA virus.

In certain example embodiments, the virus is a double-stranded RNA virus, a positive sense RNA virus, a negative sense RNA virus, a retrovirus, or a combination thereof.

In certain example embodiments, the virus is a coronavirus, an Ebola virus, measles, SARS, Chikungunya virus, Marburg, MERS, Dengue, Lassa, influenza, rhabdovirus, HIV, a hepatitis virus (including hepatitis A, B, C, D, or E), an influenza virus (including an influenza A or influenza B), a human respiratory syncytial virus, Sudan ebola virus, Bundibugyo virus, Tai Forest ebola virus, Reston ebola virus, Achimota virus, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Borna disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2Y225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence, or a combination thereof.

In certain example embodiments, the virus is a coronavirus.

In certain example embodiments, the coronavirus is SARS-CoV-2.

In certain example embodiments, the method is performed in one hour or less.

In certain example embodiments, the virus polynucleotide is RNA.

In certain example embodiments, the virus polynucleotide is DNA.

Described in certain example embodiments herein are methods of monitoring viral disease outbreaks and/or evolution, comprising performing a method as in any one of preceding paragraphs or elsewhere herein.

Described in certain example embodiments herein are kits comprising one or more compositions as in any one of the preceding paragraphs or elsewhere herein.

Described in certain example embodiments herein are diagnostic devices comprising one or more individual discrete volumes, one or more of the one or more individual discrete volumes comprises: one or more nucleic acid detection systems comprising: a CRISPR system comprising an effector protein and one or more guide RNAs capable of specifically binding a virus-specific target molecule; and a viral polynucleotide preparation formulation capable of releasing virus polynucleotides from viruses, inactivating nucleases, inactivating viruses, or a combination thereof at a temperature of about 15 degrees Celsius or greater.

In certain example embodiments, one or more of the one or more individual discrete volumes further comprises a detection construction, where the detection construct is or optionally comprises an RNA-based detection construct.

In certain example embodiments, one or more of the one or more individual discrete volumes further comprises one or more nucleic amplification reagents.

In certain example embodiments, the one or more individual discrete volumes are droplets.

In certain example embodiments, the one or more individual discrete volumes are defined on a solid substrate, are spots defined on a substrate, are contained within microwells, are contained within microfluidic channels, or a combination thereof.

In certain example embodiments, the substrate is a flexible materials substrate.

In certain example embodiments, the flexible materials substrate is a paper substrate, a fabric substrate, or a flexible polymer-based substrate.

In certain example embodiments, the diagnostic device forms or comprises a lab on a chip (LOC) device.

In certain example embodiments, the LOC device is or comprises a radio frequency identification (RFID) tag system.

In certain example embodiments, the device further comprises a wireless devices configured to communicate with the RFID tag system.

In certain example embodiments, the effector protein is a Cas protein having collateral polynucleotide cleavage activity.

In certain example embodiments, the Cas protein having collateral polynucleotide cleavage activity is selected from: Cas13a (C2c2), Cas13b (Group 29/30), Cas13c, Cas13d, Cas12a (Cpf1), Cas12b (C2c1), Cas12c (C2c3), Cas12d (CasY), Cas12e (CasX), Cas14, and combinations thereof.

In certain example embodiments, the effector protein comprises or is linked to an affinity tag, wherein each individual discrete volume comprises a capture molecule capable of specifically binding the affinity tag.

In certain example embodiments, the one or more guide RNAs comprise one or more synthetic mismatches.

In certain example embodiments, the one or more guide RNAs comprise a pan-viral guide RNA set that is capable of detecting each virus, viral strain, or both in a set of viruses.

In certain example embodiments, the guide RNAs are derived using a set cover approach.

In certain example embodiments, the device further comprises one or more of the following:
i) a heating element, wherein the heating element is configured to heat the discrete volume(s) to a predetermined temperature;
ii) a mixing element;
iii) a pipetting element;
iv) one or more reservoirs configured to contain a reagent;
v) a removable cartridge configured for adding one or more samples and/or holding reagents;
vi) a sensor capable of detecting and measuring an optical signal;
vii) a controller;
viii) a transmitter configured to transmit a signal;
ix) a receiver configured to receive a signal;
x) a processor;
xi) memory; and
xii) a user interface.

In certain example embodiments, the diagnostic device is a lateral flow device.

In certain example embodiments, the diagnostic device comprises a substrate comprising a first end, wherein the first end comprises a sample loading portion and a first region loaded with a detectable ligand, the nucleic acid detection system, a detection construct, a first capture region comprising a first binding agent, and a second capture region comprising a second binding agent.

In certain example embodiments, the detection construct comprises an RNA or DNA oligonucleotide and further comprises a first molecule on a first end and a molecule on a second end.

In certain example embodiments, the sample loading portion further comprises the viral polynucleotide preparation formulation and optionally one or more amplification reagents.

In certain example embodiments, the first capture region is proximate to and on the same end of the lateral flow substrate as the sample loading portion.

In certain example embodiments, the first capture region comprises a first binding agent that is capable of specifically binding the first molecule of the detection construct.

In certain example embodiments, the first binding agent is an antibody that is fixed or otherwise immobilized to the first capture region.

In certain example embodiments, the second capture region is located towards the opposite end of the lateral flow substrate from the first binding region.

In certain example embodiments, the second capture region comprises a second binding agent that is capable of specifically binding the second molecule of the detection construct or the detectable ligand.

In certain example embodiments, the second binding agent is an antibody or an antibody-binding protein that is fixed or otherwise immobilized to the second capture region.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1D—Initial assay development for SHERLOCK-based SARS-CoV-2 detection. (FIG. 1A) Schematic of single-step SHERLOCK assays using extracted RNA with a fluorescent or colorimetric readout. RT-RPA, reverse transcriptase-recombinase polymerase amplification; C, control line; T, test line. (FIG. 1B) Schematic of the SARS-CoV-2 genome and SHERLOCK assay location. Sequence conservation across the primer and crRNA binding sites for publicly available SARS-CoV-2 genomes (see Methods in Working Examples for details). Text denotes nucleotide position with lowest percent conservation across the assay location. ORF, open reading frame; narrow rectangles, untranslated regions; T7pro, T7 polymerase promoter. (FIG. 1C) Colorimetric detection of synthetic RNA using two-step SHERLOCK after 30 min. NTC_r, non-template control introduced in RPA, NTC_d, non-template control introduced in detection; T, test line; C, control line. (FIG. 1D) Background-subtracted fluorescence of the two-step and original single-step SHERLOCK protocols using synthetic SARS-CoV-2 RNA after 3 h. The 1 h timepoint from this experiment is shown in FIG. 2E. NTC, non-template control introduced in RPA. Center=mean and error bars=s.d. for 3 technical replicates.

FIGS. 2A-2H—Optimization of the single-step SHERLOCK reaction. (FIG. 2A) Background-subtracted fluorescence of Cas13-based detection with synthetic RNA, reverse transcriptase, and RPA primers (but no RPA enzymes) after 3 h. (FIG. 2B) Single-step SHERLOCK normalized fluorescence using various buffering conditions after 3 h. (FIG. 2C) Background-subtracted fluorescence of single-step SHERLOCK with synthetic RNA and variable RPA forward and reverse primer concentrations after 3 h. (FIG. 2D) Single-step SHERLOCK normalized fluorescence over time using two different fluorescent reporters (left) and two different reverse transcriptases (right). (FIG. 2E) Background-subtracted fluorescence of the original single-step and optimized single-step SHERLOCK with synthetic RNA after 1 h. Data from the 3 h timepoint from this experiment is shown in FIG. 1D. (FIG. 2F) Colorimetric detection of synthetic RNA input using optimized single-step SHERLOCK after 3 h. Max, maximum test band intensity, 5698.4 a.u.; Min, minimum test band intensity, 104.4 a.u. (FIG. 2G) Optimized single-step SHERLOCK background-subtracted fluorescence using RNA extracted from patient samples after 1 h. (FIG. 211) Concordance between SHERLOCK and RT-qPCR for 7 patient samples and 4 controls. For (FIGS. 2C and 2E), see methods for details in the Working Examples about normalized fluorescence calculations. For (FIGS. 2B, 2D, 2F, and 2G), NTC, non-template control. For (FIGS. 2A and 2C) center=mean for 2 technical replicates. For (FIGS. 2D, 2E, and 2F), center=mean and error bars=s.d. for error for 3 technical replicates. For (FIGS. 2B and 2D) RNA input at 10⁴ cp/μL.

FIGS. 3A-311—SARS-CoV-2 detection from unextracted samples using SHINE. (FIG. 3A) Schematic of SHINE, which streamlines SARS-CoV-2 detection by using HUDSON to inactivate samples and single-step SHERLOCK to detect viral RNA with an in-tube fluorescent or colorimetric readout. Times, suggested incubation times; C, control line; T, test line. (FIG. 3B) Measurement of RNase activity using RNaseAlert after 30 min at room temperature from treated or untreated universal viral transport medium (UTM), saliva, and phosphate buffered saline (PBS). (FIG. 3C) SARS-CoV-2 RNA detection in UTM as measured by single-step SHERLOCK and the in-tube fluorescence readout after 1 h. (FIG. 3D) SARS-CoV-2 RNA detection in in saliva using SHINE with the in-tube fluorescence readout after 1 h. (FIG. 3E) Schematic of the companion smartphone application for quantitatively analyzing in-tube fluorescence and reporting binary outcomes of SARS-CoV-2 detection. (FIG. 3F) Colorimetric detection of SARS-CoV-2 RNA in unextracted patient samples using the SHINE after 1 h. (FIG. 3G) SARS-CoV-2 detection from 50 unextracted patient samples using SHINE and smartphone application quantification of in-tube fluorescence after 40 min. Threshold line plotted as mean readout value for controls plus 3 standard deviations. (FIG. 311) Concordance table between SHINE and RT-qPCR for 50 patient samples. For (FIG. 3B) center=mean for 2 technical replicates.

FIGS. 4A-4D—Additional two-step SHERLOCK testing. (FIG. 4A) Colorimetric detection of synthetic DNA using two-step SHERLOCK after 3 h. NTC, non-template control; T, test line; C, control line. (FIG. 4B) Colorimetric detection of HUDSON-treated SARS-CoV-2 viral seedstock using two-step SHERLOCK after 3 h. NTC, non-template control; T, test line; C, control line. (FIG. 4C) Ct values of RT-qPCR for extracted RNA from SARS-CoV-2 seedstock at various concentrations. Symbol indicates the result of our two-step SHERLOCK assay performed side-by-side. The vertical line demarcates 1 cp/μL. The horizontal line demarcates samples with non-quantifiable Ct values (i.e., no amplification), imputed as a Ct of 40. (FIG. 4D) Viral Ct values measured by RT-qPCR for extracted RNA from 41 RT-qPCR positive patient samples grouped by the result of the two-step SHERLOCK assay using a colorimetric, lateral flow-based readout. Inset is the concordance results of all samples tested by colorimetric two-step SHERLOCK and RT-qPCR. The association between viral Ct and two-step SHERLOCK outcome was assessed using a one-sided Wilcoxon rank sum test. * p<0.0001.

FIGS. 5A-5E—Optimization of single-step SHERLOCK for improved sensitivity. (FIG. 5A) Background-subtracted fluorescence detected after the single-step SHERLOCK reaction was incubated for 3 h with DNA as input. (FIG. 5B) Background-subtracted fluorescence of the Cas13-detection reaction (no RPA enzymes) with 3 h incubation. RNase H+, final concentration of 0.1 U/μL. (FIG. 5C) Background-subtracted fluorescence of the Cas13-detection reaction (no RPA) after 3 h incubation with varying RNase H concentrations. (FIG. 5D) Background-subtracted fluorescence detected after the single-step reaction was incubated for 3 h with varying magnesium concentrations. (FIG. 5E) Background-subtracted fluorescence detected after the single-step reaction was incubated for 3 h with varying RPA primer concentrations. For FIGS. 5A-5E, NTC, non-template controls; error bars, s.d. for 2-3 technical replicates. All listed concentrations refer to concentration within the reaction mixture before addition of the oligonucleotide template. For (a-c), Center=mean and error bars=s.d. for 3 technical replicates.

FIGS. 6A-6B—Optimization of fluorescent reporter. (FIG. 6A) Single-step SHERLOCK normalized fluorescence (see Methods in Working Examples for details) over time using quenched poly-uracil FAM reporters of varying lengths or RNaseAlert with RNA input at 10⁴ cp/μl. (FIG. 6B) Background-subtracted fluorescence of poly-uracil FAM reporters or RNaseAlert in single-step SHERLOCK after 3 h for non-template controls. For (FIGS. 6A-6B), Center=mean and error bars=s.d. for 3 (FIG. 6A) and 6 (FIG. 6B) technical replicates.

FIG. 7—Single-step SHERLOCK time course. Optimized single-step SHERLOCK assay fluorescence over time at varying RNA input concentrations. Background-subtracted fluorescence at 1 h is shown in FIG. 2E. NTC, non-template control; Center=mean and error bars=s.d. for 3 technical replicates. Note: error bars for NTC are present but small.

FIGS. 8A-8B—Single-step SHERLOCK specificity. (FIG. 8A) Normalized fluorescence of Cas13-based detection using synthetic RNA targets of four human-infecting coronaviruses as the target input after 3 h. nd, not done. n=3 replicates. (FIG. 8B) Normalized fluorescence of optimized single-step SHERLOCK using synthetic RNA targets of four human-infecting coronaviruses as target input after 3 h. NTC, non-template control; n=3 replicates. error bars, s.d. for 3-6 technical replicates. For (FIGS. 8A-8B), Center=mean and error bars=s.d. for 3 technical replicates.

FIGS. 9A-9D—HUDSON optimization experiments. (FIG. 8A) Samples were treated with 100 mM TCEP and 1 mM EDTA and subjected to a 20 min heating step at 50° C. RNase inhibitor, 4 U/μl, unless otherwise specified. (FIG. 8B) Samples were treated with 100 mM TCEP, 1 mM EDTA, and 4 U/μl RNase inhibitor. (FIG. 8C) Samples were treated with 100 mM TCEP and 1 mM EDTA and subjected to a 5 min heating step at 50° C. (FIG. 8D) Samples in UTM, VTM, or PBS were treated with 100 mM TCEP and 1 mM EDTA or nuclease-free water and subjected to a 5 min heating step at 50° C. (FIGS. 8A-8D) Positive and negative controls undergo no treatment. RNaseAlert (final concentration: 200 nM) was added immediately after the heating step and is used to measure the RNase activity.

FIG. 10—SHINE for UTM and saliva with colorimetric detection. SHINE with colorimetric readout using synthetic RNA template spiked into UTM (left) and saliva (right) after the initial HUDSON heating step.

FIGS. 11A-11C—SHINE for UTM, VTM, and saliva with in-tube fluorescent detection. (FIGS. 11A-11C) SHINE with in-tube readout using synthetic RNA template spiked into UTM (FIG. 11A), saliva (FIG. 11B) and VTM (FIG. 11C) after the initial HUDSON heating step. Transilluminator or GelDoc images were captured using a smartphone camera. NTC, non-template control.

FIGS. 12A-12B—Limit of detection of SHINE on UTM and saliva. (FIGS. 12A-12B) SHINE with in-tube readout using synthetic RNA template spiked into UTM (FIG. 12A) and saliva (FIG. 12B) after the initial HUDSON heating step. Transilluminator images were captured using a smartphone camera and analyzed by the companion smartphone application (App).

FIG. 13—SHINE on unextracted patient samples. NP swabs in UTM were used as input into the SHINE assay. Transilluminator images were captured using a smartphone camera after 40 min of single-step SHERLOCK incubation.

FIG. 14—SHINE's ability to detect viral RNA is significantly associated with the RT-qPCR threshold cycle. Viral Ct values measured by SARS-CoV-2 RT-qPCR of extracted RNA from 30 patient NP samples grouped by the result of the SHINE assay. The association between viral Ct and SHINE outcome was assessed using a one-sided Wilcoxon rank sum test. **, p=0.0017.

FIG. 15—SHINE on unextracted patient samples. NP swabs in VTM were used as input into the SHINE assay. Transilluminator images were captured using a smartphone camera after 40 min of single-step SHERLOCK incubation. Samples were tested in triplicate. Rep, replicate. App, readout with smartphone application.

FIGS. 16A-16I—Increasing the ease-of-use and deployability of SHINE. FIG. 16A RNase activity in nasal fluid mixed with universal viral transport medium (UTM) untreated or treated with FastAmp Lysis buffer supplemented with RNase inhibitor or treated with HUDSON (a heat- and chemical-treatment). Activity measured using RNaseAlert at room temperature (RT) for 30 minutes. FIG. 16B SARS-CoV-2 seedstock titer without treatment or after being incubated with lysis buffer (+5% RNase inhibitor) at RT for 5 minutes, 20 minutes or 20 minutes plus 10 minutes at 65° C. ***, infection not detected; PFU, plaque forming units. FIG. 16C SHINE fluorescence with different proportions of lysis mix (i.e., FastAmp lysis buffer, RNase inhibitor and UTM) input after a 90-minute incubation. FIG. 16D Schematic of the advantages of lyophilizing SHINE. FIG. 16E SHINE fluorescence after a 90-minute incubation on synthetic RNA target (10⁴ copies/μL) before and after lyophilization using different buffers. Fluorescence measured after 90 minutes. For buffer composition, see Example 7 herein. FIG. 16F SHINE fluorescence after a 90-minute incubation using lyophilized (LYO) reagents stored at RT, 4° C. or −20° C. over time. Target concentration: 10⁴ copies/uL. FIG. 16G Fluorescence kinetics for SHINEv1 and SHINEv2 using synthetic RNA targets; NTC, no target control. FIG. 16H Lateral-flow detection of SARS-CoV-2 RNA in lysis buffer treated viral seedstocks using SHINE. Incubated for 90 minutes. C=control band; T=test band. FIG. 16I Determination of analytical limit of detection with 20 replicates of SHINE at different concentrations of SARS-CoV-2 RNA from lysis buffer treated viral seedstocks. Incubated for 90 minutes. For (FIGS. 16A,16E, and 16G), center=mean and error bars=s.d. for 3 technical replicates. In FIG. 16C, the heatmap values represent the mean for 3 technical replicates. For FIG. 16F, center=mean and error bars=s.d. for 3 biological replicates with 3 technical replicates each.

FIGS. 17A-17D—Performance of SHINEv2 on clinical samples. FIG. 17A Schematic of side-by-side clinical sample testing using SHINEv2, BinaxNow, CareStart and RT-qPCR. FIG. 17B SHINEv2, BinaxNow and CareStart test results for a subset of clinical nasopharyngeal (NP) swab samples with different Ct values (CDC EUA N1 RT-qPCR). C=control band; T=test band. FIG. 17C Positive and negative test results for SHINEv2, BinaxNow and CareStart tests for RT-qPCR-positive clinical samples relative to viral RNA concentration and Ct value. FIG. 17D Side-by-side clinical performance of SHINEv2, BinaxNow and CareStart versus RT-qPCR. SHINEv2 reaction were incubated for 90 minutes.

FIGS. 18A-18F—Development of SHINEv2 assays for the detection of SARS-CoV-2 VOC. FIG. 18A Schematic of Cas13a-based detection of mutations in SARS-CoV-2 using a fluorescent readout. SNP, single nucleotide polymorphism; anc, ancestral; der, derived. FIG. 18B Normalized SHINE fluorescence of the anc and der crRNAs for the 69/70 deletion assay against synthetic RNA targets after 90 minutes; NTC, no-target control. FIG. 18C Normalized SHINE fluorescence of the ancestral (anc) and derived (derN/T) crRNAs for the 417 SNP detection assay against synthetic RNA targets after 90 minutes; NTC, no-target control. FIG. 18D Identification of SARS-CoV-2 variants-of-concern (VOC) using normalized SHINE fluorescence on full-genome synthetic RNA controls (full genome synthetic RNA) and RNA extracted from viral seedstock; target RNA concentration: 10⁴ copies/μL. FIG. 18E Colorimetric lateral flow-based detection of SARS-CoV-2 RNA in contrived clinical samples using the 69/70 SHINEv2 assay. SHINEv2 incubation time: 90 minutes. NTC, no-target control. T, test line; C, control line. FIG. 18F Mean fluorescence of 69/70 SHINEv2 assay on SARS-CoV-2 RNA extracted from clinical samples, after 90 minutes. For FIG. 18B and FIG. 18C, center=mean and error bars=s.d. for 3 technical replicates. In FIG. 18D and FIG. 18F, the heatmap values represent the mean for 3 technical replicates. * in FIG. 18D and FIG. 18F indicate signal above threshold.

FIGS. 19A-19C—Optimization of assay readout and processing for lateral-flow detection. FIG. 19A Lateral-flow based detection of SARS-CoV-2 RNA using SHINEv2 with different polyethylene glycol (i.e., PEG) compositions. Dilution refers to lateral flow buffer being mixed in prior to adding the paper strip. Incubated for 90 minutes. NTC, no-target control. FIG. 19B Lateral flow based SHINEv2 detection of SARS-CoV-2 RNA after a 90-minute incubation in a heat-block or using body-heat (urderarm). NTC, no-target control. FIG. 19C SHINE fluorescence on SARS-CoV-2 RNA after 90 minutes at 37° C. or 25° C.; NTC, no-target control. For FIG. 19C, center=mean and error bars=s.d. for 3 technical replicates.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris, and/or viruses or components thereof, and/or bioligc molecules (e.g., nucleic acids and/or proteins). The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures. Biological samples can also be samples obtained from an environment (e.g., air, water, soil, object surface, and/or the like) that can contain cells, cell components, cell debris, prokaryotic organisms, or components thereof, biologic molecules (e.g., nucleic acids, proteins, and/or the like), viruses or components thereof, and/or the like. Environmental samples can be obtained by any suitable methods, such as filtering, wiping, swabbing, rinsing, catching, and/or the like.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Microbial Clustered Regularly Interspaced Short Palindromic Repeats (CRISPR) and CRISPR-associated (CRISPR-Cas) adaptive immune systems contain programmable endonucleases, such as Cas9 and Cpf1 (Shmakov et al., 2017; Zetsche et al., 2015). Although both Cas9 and Cpf1 target DNA, single effector RNA-guided RNases have been recently discovered (Shmakov et al., 2015) and characterized (Abudayyeh et al., 2016; Smargon et al., 2017), including C2c2, providing a platform for specific RNA sensing. RNA-guided RNases can be easily and conveniently reprogrammed using CRISPR RNA (crRNAs) to cleave target RNAs. Unlike the DNA endonucleases Cas9 and Cpf1, which cleave only its DNA target, RNA-guided RNases, like C2c2, remains active after cleaving its RNA target, leading to “collateral” cleavage of non-targeted RNAs in proximity (Abudayyeh et al., 2016). This crRNA-programmed collateral RNA cleavage activity presents the opportunity to use RNA-guided RNases to detect the presence of a specific RNA by triggering in vivo programmed cell death or in vitro nonspecific RNA degradation that can serve as a readout (Abudayyeh et al., 2016; East-Seletsky et al., 2016).

The current SARS-CoV-2 pandemic has highlighted the great need for assays that are fast, sensitive, low-cost, user-friendly, and rapidly adaptable to detect newly-identified agents, particularly in remote and primitive or resource-limited areas. Various embodiments disclosed herein utilize polynucleotide targeting effectors to provide a robust CRISPR-based diagnostic with attomolar sensitivity. Embodiments disclosed herein can detect broth DNA and RNA with comparable levels of sensitivity and can differentiate targets from non-targets based on single base pair differences. Moreover, the embodiments disclosed herein can be prepared in formats for convenient distribution and point-of-care (POC) applications, particularly in resource-limited areas. Such embodiments are useful in multiple scenarios in human health including, for example, viral detection, bacterial strain typing, sensitive genotyping, and detection of disease-associated cell free DNA.

Embodiments disclosed herein can detect specific polynucleotides using a CRISPR system within in a single reaction that can be performed without the need for separate nucleic acid extractions from a sample. Embodiments disclosed herein can prepare a sample, which can include nucleic acid extraction, and specific polynucleotide detection in a single reaction vessel or device. Embodiments disclosed herein can prepare a sample, which can include nucleic acid extraction, and/or specific polynucleotide detection at ambient temperatures of less than about 37 degrees Celsius. Embodiments disclosed herein provide CRISPR-Cas-based detection reagents that are adapted for shipment and storage while maintaining assay integrity and fidelity.

Embodiments disclosed herein provide CRISPR-system based viral polynucleotide detection that can include nucleic acid extraction and a one-pot (e.g., single reaction) CRISPR-system based polynucleotide detection in the same reaction vessel or device. In certain exemplary embodiments are provided SARS-CoV-2 detection using the CRISPR-system based viral polynucleotide detection that includes nucleic acid extraction and a one-pot CRISPR-system based polynucleotide detection in the same reaction vessel or device. In certain exemplary embodiments, the nucleic acid extraction and/or one pot detection can occur at temperatures less than 37 degrees Celsius.

Also described in several embodiments herein are compositions that can be used to carry out one or more steps of the methods of detection described herein. Described in certain example embodiments herein are compositions comprising: a nucleic acid detection system comprising: a CRISPR-system comprising an effector protein and one or more guide RNAs, capable of specifically binding a virus-specific target molecule; and a viral polynucleotide preparation formulation capable of releasing virus polynucleotides from viruses, inactivating nucleases, inactivating viruses, or a combination thereof at a temperature of about 25 degrees Celsius or greater.

Described in certain example embodiments herein are methods of detecting a virus in a sample comprising: releasing virus polynucleotides from a virus in the sample; inactivating nucleases present in the sample; inactivating viruses present in the sample; amplifying virus polynucleotides in the sample; combining the sample with a nucleic acid detection system comprising: a CRISPR system comprising an effector protein and one or more guide RNAs, capable of specifically binding a virus-specific target molecule; and a detection construct; activating the effector protein such that a detectable positive signal is produced, wherein activating the effector protein occurs via specific binding of the one or more guide RNAs to one or more virus-specific target molecules and results in modification of the detection construct such that a detectable signal is produced; and detecting the detectable signal, wherein the detectable signal indicates a presence of one or more viruses in the sample, wherein amplifying and activating occur in the same reaction and wherein the method does not include a step of extracting a virus polynucleotide from the sample.

Also described in several embodiments herein are kits that contain one or more compositions and/or devices that can be used to carry out one or more steps of the methods of detection described herein.

Also described herein are devices, such as lateral flow devices, that can be used to carry out one or more steps of the methods of detection described herein. Described in certain example embodiments herein are diagnostic devices comprising one or more individual discrete volumes, one or more of the one or more individual discrete volumes comprises: one or more nucleic acid detection systems comprising: a CRISPR system comprising an effector protein and one or more guide RNAs capable of specifically binding a virus-specific target molecule; and a viral polynucleotide preparation formulation capable of releasing virus polynucleotides from viruses, inactivating nucleases, inactivating viruses, or a combination thereof at a temperature of about 25 degrees Celsius or greater.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Crispr System Based Viral Detection Compositions

In general, embodiments herein provide CRISPR-Cas based detection compositions that can be used to detect a nucleic acid from a target organism or virus. Certain embodiments of the compositions are optimized to facilitate processing and/or target nucleic acid detection at temperatures of less than about 65 degrees C., about 37 degrees C. or less, or about 25 degrees C. or less. Certain embodiments of the compositions are optimized to facilitate lyophilization of the compositions while maintaining assay fidelity and integrity upon reconstitution and use. Certain embodiments of the compositions are optimized to reduce processing steps to simplify assay performance and reduce user burden. Such optimization can facilitate rapid assay deployment and adaptation, particularly in remote and/or resource limited POC areas.

Described in certain example embodiments herein are compositions comprising a nucleic acid detection system comprising a CRISPR system comprising an effector protein and one or more guide RNAs, capable of specifically binding a virus-specific target molecule; and a viral polynucleotide preparation formulation capable of releasing virus polynucleotides from viruses, inactivating nucleases, inactivating viruses, or a combination thereof at a temperature of about 25 degrees Celsius or greater or about 37 degrees Celsius or greater.

In certain example embodiments, the composition further comprises a detection construct. The detection construct can be capable of producing one or more detectable signals. In certain example embodiments, the detection construct comprises an RNA or DNA oligonucleotide and further comprises a first molecule on a first end and a molecule on a second end. The detection construct can exist in an unmodified state and when modified by an activated effector of a CRISPR system, the detection construct can produce one or more detectable signals to indicate the presence of a target. In some embodiments, one or more of the detectable signals can be an assay control.

In certain example embodiments, the composition further comprises one or more nucleic acid amplification reagents. The amplification reagent(s) included can be capable of amplifying a target and/or a detectable signal. Exemplary amplification reagents are discussed in greater detail elsewhere herein.

### CRISPR Systems

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein. Some CRISPR-Cas systems have collateral activity that is triggered by target recognition.

In some embodiments, the CRISPR-Cas system of the present composition can be a Class 1 system. In some embodiments, the CRISPR-Cas system of the present composition can be a Class 2 system. In some embodiments, the CRISPR-Cas system of the present composition can be a CRISPR-Cas system having collateral activity, such as collateral nucleic acid cleavage activity.

### Class 1 CRISPR-Cas Systems

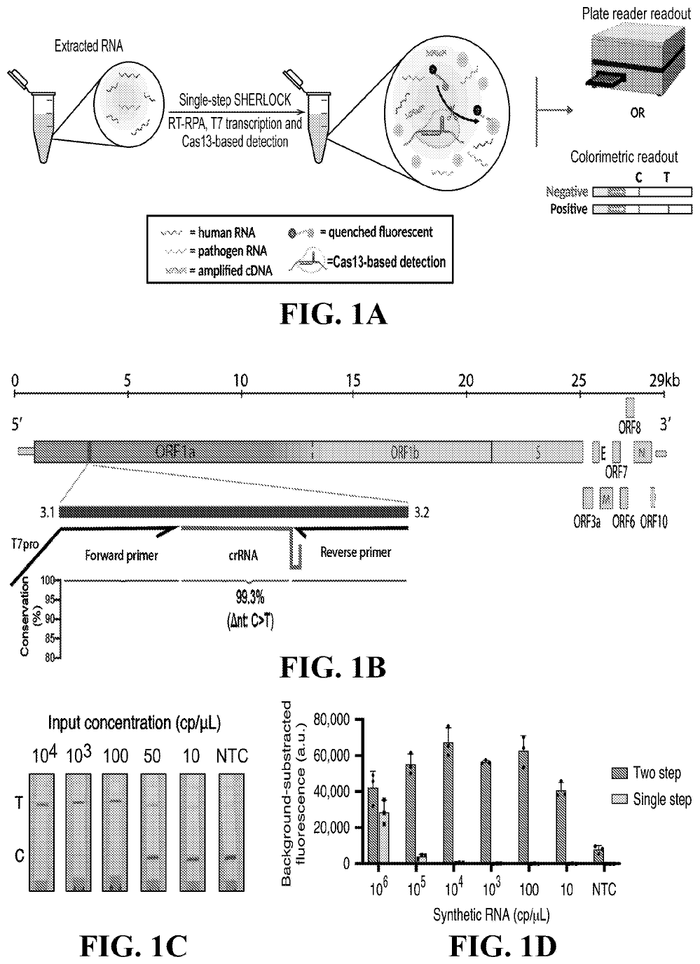

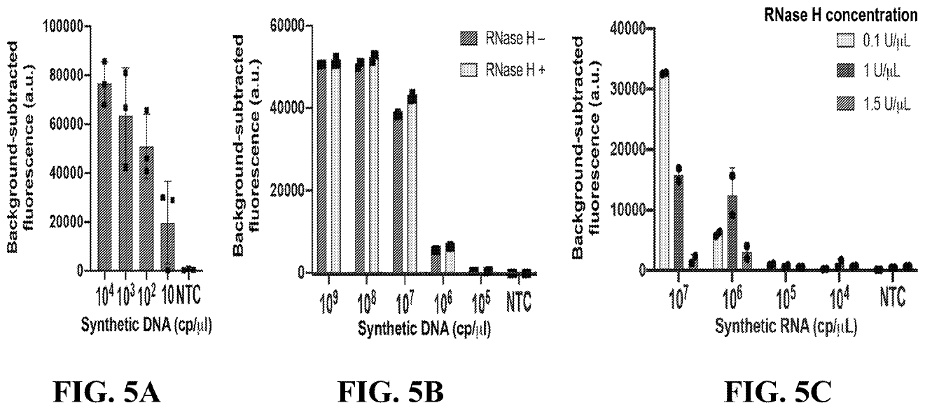

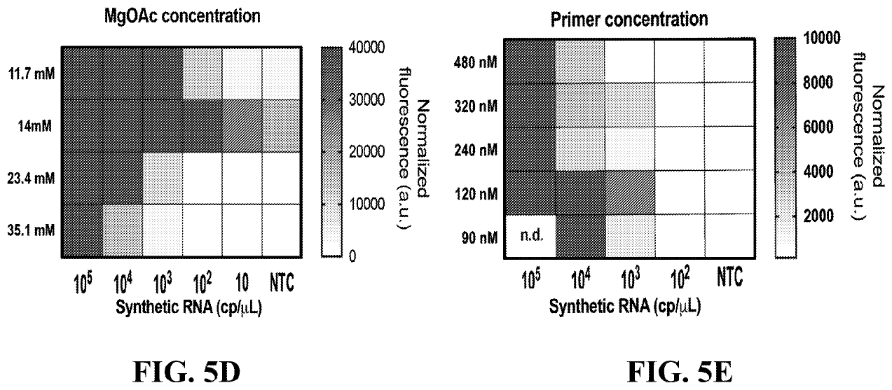

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

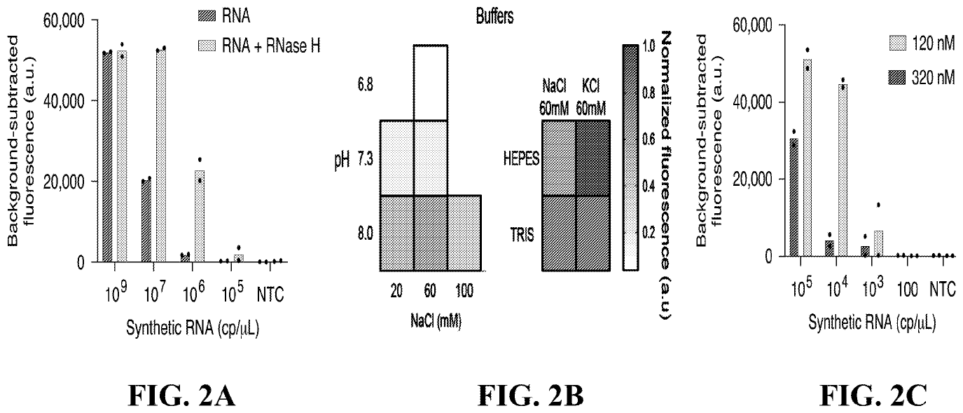

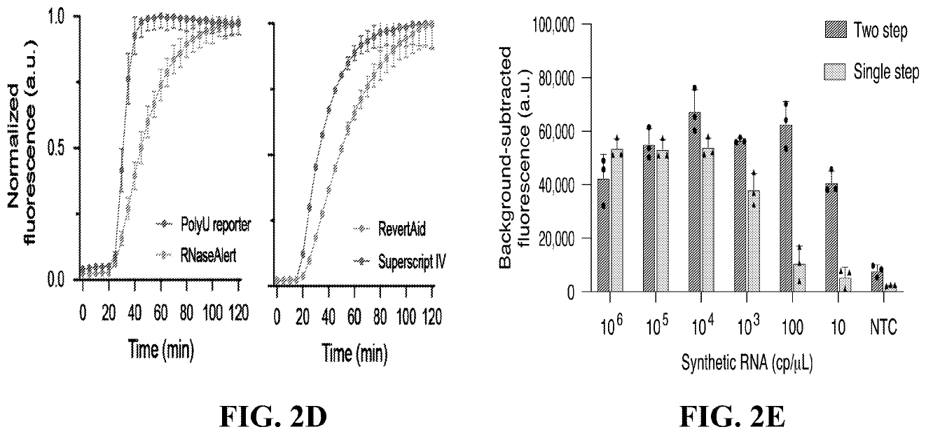

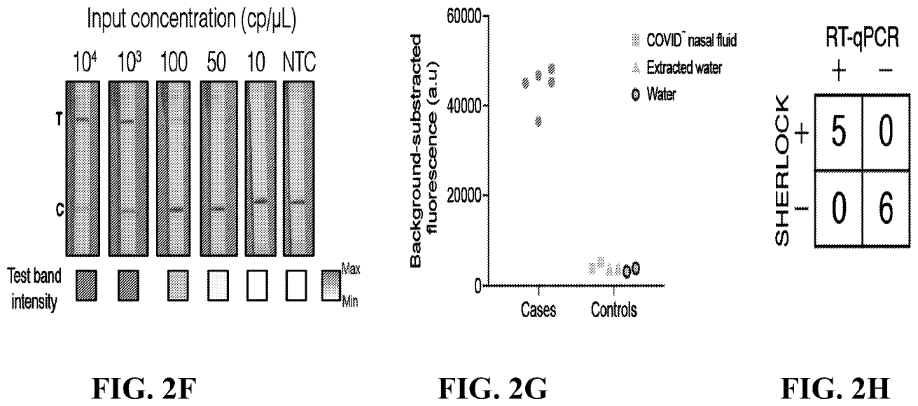

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof, homologues thereof, functional variants thereof, or modified versions thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9, homologue thereof, functional variant thereof, or modified version thereof.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasY(Cas12d), CasX (Cas12e), Cas14, and/or CasΦ, homologues thereof, functional variants thereof, or modified versions thereof.

In some embodiments, the CRISPR-Cas system includes a Cas12b. In some embodiments, the Cas12b is an Alicyclobacillus acidoterrestris Cas12b (AacCas12b) or orthologe thereof. In some embodiments, the Cas 12b is an Cas12b from Alicyclobacillus acidiphilus (AapCas12b).

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d, homologues thereof, functional variants thereof, or modified versions thereof.

Additional effectors for use according to the invention can be identified by their proximity to cas1 genes, for example, though not limited to, within the region 20 kb from the start of the cas1 gene and 20 kb from the end of the cas1 gene. In certain embodiments, the effector protein comprises at least one HEPN domain and at least 500 amino acids, and wherein the C2c2 effector protein is naturally present in a prokaryotic genome within 20 kb upstream or downstream of a Cas gene or a CRISPR array. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In certain example embodiments, the C2c2 effector protein is naturally present in a prokaryotic genome within 20 kb upstream or downstream of a Cas 1 gene. The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of. Homologous proteins may, but need not be structurally related, or are only partially structurally related. An “orthologue” of a protein as used herein is a protein of a different species which performs the same or a similar function as the protein it is an orthologue of. Orthologous proteins may, but need not be, structurally related, or are only partially structurally related.

Cas Polypeptides with Collateral Activities

Unlike the DNA endonucleases Cas9 and Cpf1, which cleave only its DNA target, RNA-guided RNases, like C2c2, remains active after cleaving its RNA target, leading to “collateral” cleavage of non-targeted RNAs in proximity (Abudayyeh et al., 2016). This crRNA-programmed collateral RNA cleavage activity presents the opportunity to use RNA-guided RNases to detect the presence of a specific RNA by triggering in vivo programmed cell death or in vitro nonspecific RNA degradation that can serve as a readout (Abudayyeh et al., 2016; East-Seletsky et al., 2016). Collateral activity of class 2 enzymes includes Cas13b and Cas12a enzymes, as described in Gootenberg et al. Science, 2018 Apr. 27; 360(6387): 439-444, incorporated herein by reference.

CRISPR Cas based systems that allow for detection down to femtomolar sensitivity can be combined with initial amplification of the target to allow for detectable attomolar concentrations, possibly lower. SHERLOCK and DETECTR employ preamplification systems with Cas enzymes, for example Cas13a or Cas 12a that target ssRNA and dsDNA respectively. See e.g., Kaminski et al. Nat. Biomed Eng. 5: 643-656 (2021); Mustafa and Makhawi. J Clin Microbiol 59(3). 2021. doi:https://doi.org/10.1128/JCM.00745-20; Srivastava et al. Front. Mol. Biosci., 23 Dec. 2020 https://doi.org/10.3389/fmolb.2020.582499; Yan et al., 2019. Cell Biol Toxicol. 35:489-492

In certain embodiments, the CRISPR-Cas system includes a Cas polypeptide that has one or more collateral activities, such as collateral nucleic acid cleavage activity. Such activities can be utilized in an assay, such as a detection assay for a target nucleic acid described elsewhere herein. In certain example embodiments, a Cas that has collateral activity (e.g., collateral nucleic acid cleavage activity) that can be included in the CRISPR-Cas system is a Cas13 (e.g. a Cas13a, 13b, Cas13c and/or Cas13d). In certain example embodiments, a Cas that has collateral activity that can be included in the CRISPR-Cas system is a Cas12 (e.g., Cas 12a, 12b, 12c, 12c1, 12c2, 12d, 12e, 12a1, 12g1, 12h1, 12i1, 12f (also known as Cas14)).

### Cas13 Polypeptides

Cas13's non-specific RNase activity can be leveraged to cleave reporters upon target recognition, allowing for the design of sensitive and specific diagnostics using Cas13, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference; WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox D B T, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6., Science. 2018 Apr. 27; 360(6387):439-444; Gootenberg J S, et al., Nucleic acid detection with CRISPR-Cas13a/C2c2., Science. 2017 Apr. 28; 356(6336):438-442; Abudayyeh 00, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimizing deaminase fusions for genome editing. Nat Commun. 2016 Nov. 2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, Zhang et al., “Two HPEN domains dictate CRISPR RNA maturation and target cleavage in Cas13d.” Nat. Comm. 10:2544 (2019), Patchsung et al., 2020. Nat. Biomed. Eng. 4:1140-1149; Aquino-Jarquin, G. Drug Discov. Today. 2021. 26(8):2025-2035; Fozouni et al., 2020. Amplification-free detection of SARS-CoV-2 with CRISPR-Cas13a and mobile phone microscopy. Cell. 184:323-333; Lotfi and Rezaei. 2020. CRISPR/Cas13: A potential therapeutic option of COVID-19 Biomedicine & Pharmacotherapy. 131:110738; Khan et al. 2020. CRISPR-Cas13 enzymology rapidly detects SARS-CoV-2 fragments in a clinical setting. medRxiv; doi: https://doi.org/10.1101/2020.12.17.20228593; Schermer et al., Rapid SARS-CoV-2 testing in primary material based on a novel multiplex RT-LAMP assay. PLoS One. https://doi.org/10.1371/journal.pone.0238612; Joung et al., “Detection of SARS-CoV-2 with SHERLOCK One-Pot TestingN Engl J Med 2020; 383:1492-1494” DOI: 10.1056/NEJMc2026172; Joung et al., “Point-of-care testing for COVID-19 using SHERLOCK diagnostics” medRxiv. Preprint. 2020 May 8. doi: 10.1101/2020.05.04.20091231; WO 2017/218573; US 20200010878; US 20200010879; US 20190177775; US 20180208977; US 20180208976; US 20190177775; U.S. Provisional Application Ser. No. 62/351,172; the disclosure of each can be adapted for use with the present invention in view of the description provided herein and each of which is incorporated herein by reference in its entirety.

In certain example embodiments, the CRISPR-Cas system includes a Cas13 (e.g. a Cas13a, 13b, Cas13c and/or Cas13d). In particular embodiments, the Type VI RNA-targeting Cas enzyme is C2c2. In other example embodiments, the Type VI RNA-targeting Cas enzyme is Cas 13b. In particular embodiments, the homologue or orthologue of a Type VI protein such as C2c2 as referred to herein has a sequence homology or identity of at least 30%, or at least 40%, or at least 50%, or at least 60%, or at least 70%, or at least 80%, more preferably at least 85%, even more preferably at least 90%, such as for instance at least 95% with a Type VI protein such as C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2). In further embodiments, the homologue or orthologue of a Type VI protein such as C2c2 as referred to herein has a sequence identity of at least 30%, or at least 40%, or at least 50%, or at least 60%, or at least 70%, or at least 80%, more preferably at least 85%, even more preferably at least 90%, such as for instance at least 95% with the wild type C2c2 (e.g., based on the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2).

In certain other example embodiments, the CRISPR system the effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. Regarding C2c2 CRISPR systems, reference is made to U.S. Provisional 62/351,662 filed on Jun. 17, 2016 and U.S. Provisional 62/376,377 filed on Aug. 17, 2016. Reference is also made to U.S. Provisional 62/351,803 filed on Jun. 17, 2016. Reference is also made to U.S. Provisional entitled “Novel Crispr Enzymes and Systems” filed Dec. 8, 2016 bearing Broad Institute No. 10035.PA4 and Attorney Docket No. 47627.03.2133. Reference is further made to East-Seletsky et al. “Two distinct RNase activities of CRISPR-C2c2 enable guide-RNA processing and RNA detection” Nature doi:10/1038/nature19802 and Abudayyeh et al. “C2c2 is a single-component programmable RNA-guided RNA targeting CRISPR effector” bioRxiv doi:10.1101/054742.

RNase function in CRISPR systems is known, for example mRNA targeting has been reported for certain type III CRISPR-Cas systems (Hale et al., 2014, Genes Dev, vol. 28, 2432-2443; Hale et al., 2009, Cell, vol. 139, 945-956; Peng et al., 2015, Nucleic acids research, vol. 43, 406-417) and provides significant advantages. In the Staphylococcus epidermis type III-A system, transcription across targets results in cleavage of the target DNA and its transcripts, mediated by independent active sites within the Cas10-Csm ribonucleoprotein effector protein complex (see, Samai et al., 2015, Cell, vol. 151, 1164-1174). A CRISPR-Cas system, composition or method targeting RNA via the present effector proteins is thus provided.

In an embodiment, the Cas protein may be a C2c2 ortholog of an organism of a genus which includes but is not limited to Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter. Species of organism of such a genus can be as otherwise herein discussed.

In an embodiment, the C2c2 or an ortholog or homolog thereof, may comprise one or more mutations (and hence nucleic acid molecule(s) coding for same may have mutation(s). The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains.

In an embodiment, the C2c2 or an ortholog or homolog thereof, may comprise one or more mutations. The mutations may be artificially introduced mutations and may include but are not limited to one or more mutations in a catalytic domain. Examples of catalytic domains with reference to a Cas enzyme may include but are not limited to HEPN domains.

In an embodiment, the C2c2 or an ortholog or homolog thereof, may be used as a generic nucleic acid binding protein with fusion to or being operably linked to a functional domain. Exemplary functional domains may include but are not limited to translational initiator, translational activator, translational repressor, nucleases, in particular ribonucleases, a spliceosome, beads, a light inducible/controllable domain or a chemically inducible/controllable domain.

In certain example embodiments, the C2c2 effector protein may be from an organism selected from the group consisting of; Leptotrichia, Listeria, Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, and Campylobacter.

In certain embodiments, the effector protein may be a Listeria sp. C2c2p, preferably Listeria seeligeria C2c2p, more preferably Listeria seeligeria serovar 1/2b str. SLCC3954 C2c2p and the crRNA sequence may be 44 to 47 nucleotides in length, with a 5′ 29-nt direct repeat (DR) and a 15-nt to 18-nt spacer.

In certain embodiments, the effector protein may be a Leptotrichia sp. C2c2p, preferably Leptotrichia shahii C2c2p, more preferably Leptotrichia shahii DSM 19757 C2c2p and the crRNA sequence may be 42 to 58 nucleotides in length, with a 5′ direct repeat of at least 24 nt, such as a 5′ 24-28-nt direct repeat (DR) and a spacer of at least 14 nt, such as a 14-nt to 28-nt spacer, or a spacer of at least 18 nt, such as 19, 20, 21, 22, or more nt, such as 18-28, 19-28, 20-28, 21-28, or 22-28 nt.

In certain example embodiments, the effector protein may be a Leptotrichia sp., Leptotrichia wadei F0279, or a Listeria sp., preferably Listeria newyorkensis FSL M6-0635.

In certain example embodiments, the C2c2 effector proteins of the invention include, without limitation, the following 21 ortholog species (including multiple CRISPR loci: Leptotrichia shahii; Leptotrichia wadei (Lw2); Listeria seeligeri; Lachnospiraceae bacterium MA2020; Lachnospiraceae bacterium NK4A179; [Clostridium] aminophilum DSM 10710; Carnobacterium gallinarum DSM 4847; Carnobacterium gallinarum DSM 4847 (second CRISPR Loci); Paludibacter propionicigenes WB4; Listeria weihenstephanensis FSL R9-0317; Listeriaceae bacterium FSL M6-0635; Leptotrichia wadei F0279; Rhodobacter capsulatus SB 1003; Rhodobacter capsulatus R121; Rhodobacter capsulatus DE442; Leptotrichia buccalis C-1013-b; Herbinix hemicellulosilytica; [Eubacterium] rectale; Eubacteriaceae bacterium CHKCI004; Blautia sp. Marseille-P2398; and Leptotrichia sp. oral taxon 879 str. F0557. Twelve (12) further non-limiting examples are: Lachnospiraceae bacterium NK4A144; Chloroflexus aggregans; Demequina aurantiaca; Thalassospira sp. TSL5-1; Pseudobutyrivibrio sp. OR37; Butyrivibrio sp. YAB3001; Blautia sp. Marseille-P2398; Leptotrichia sp. Marseille-P3007; Bacteroides ihuae; Porphyromonadaceae bacterium KH3CP3RA; Listeria riparia; and Insolitispirillum peregrinum.

In certain embodiments, the C2c2 protein according to the invention is or is derived from one of the orthologues as described in the table below, or is a chimeric protein of two or more of the orthologues as described in the table below, or is a mutant or variant of one of the orthologues as described in the table below (or a chimeric mutant or variant), including dead C2c2, split C2c2, destabilized C2c2, etc. as defined herein elsewhere, with or without fusion with a heterologous/functional domain.

In certain example embodiments, the C2c2 effector protein is selected from Table 1.

TABLE 1
Exemplary C2c2 effectors

| C2c2 orthologue | Code | Multi Letter |
|---|---|---|
| Leptotrichia shahii | C2-2 | Lsh |
| L. wadei F0279 (Lw2) | C2-3 | Lw2 |
| Listeria seeligeri | C2-4 | Lse |
| Lachnospiraceae bacterium MA2020 | C2-5 | LbM |
| Lachnospiraceae bacterium NK4A179 | C2-6 | LbNK179 |
| Clostridium aminophilum DSM 10710 | C2-7 | Ca |
| Carnobacterium gallinarum DSM | C2-8 | Cg |
| Carnobacterium gallinarum DSM 4847 | C2-9 | Cg2 |
| Paludibacter propionicigenes WB4 | C2-10 | Pp |
| Listeria weihenstephanensis FSL R9-0317 | C2-11 | Lwei |
| Listeriaceae bacterium FSL M6-0635 | C2-12 | LbFSL |
| Leptotrichia wadei F0279 | C2-13 | Lw |
| Rhodobacter capsulatus SB 1003 | C2-14 | Rc |
| Rhodobacter capsulatus R121 | C2-15 | Rc |
| Rhodobacter capsulatus DE442 | C2-16 | Rc |
| Leptotrichia buccalis C-1013-b | C2-17 | LbuC2c2 |
| Herbinix hemicellulosilytics | C2-18 | HheC2c2 |
| Eubacterium rectale | C2-19 | EreC2c2 |
| Eubacteriaceae bacterium CHKC1004 | C2-20 | EbaC2c2 |
| Blautia sp. Marseille-P2398 | C2-21 | BsmC2c2 |
| Leptotrichia sp. oral taxon 879 str. F0557 | C2-22 | LspC2c2 |
| Lachnospiraceae bacterium NK4a144 | | |
| Chloroflexus aggregans | | |
| Demequina aurantiaca | | |
| Thalassospira sp. TSL5-1 | | |
| Pseudobutyrivibrio sp. OR37 | | |
| Butyrivibrio sp. YAB3001 | | |
| Blautia sp. Marseille-P2398 | | |
| Leptotrichia sp. Marseille-P300 | | |
| Bacteroides ihuae | | |
| Porphyromonadaceae bacterium KH3CP3RA | | |
| Listeria riparia | | |
| Insolitispirillum peregrinum | | |

The wild-type protein sequences of the above species are listed in Table 2.

TABLE 2
Wild-Type C2c2 Effectors

| | | |
|---|---|---|
| C2c2-2 | | L. shahii (Lsh) |
| c2c2-3 | | L. wadei (Lw2) |
| c2c2-4 | | Listeria seeligeri |
| c2c2-5 | 1 | Lachnospiraceae bacterium MA2020 |
| c2c2-6 | 2 | Lachnospiraceae bacterium NK4A179 |
| c2c2-7 | 3 | Clostridium aminophilum DSM 10710 |
| c2c2-8 | 5 | Carnobacterium gallinarum DSM 4847 |
| c2c2-9 | 6 | Carnobacterium gallinarum DSM 4847 |
| c2c2-10 | 7 | Paludibacter propionicigenes WB4 |
| c2c2-11 | 9 | Listeria weihenstephanensis FSL R9-0317 |
| c2c2-12 | 10 | Listeriaceae bacterium FSL M6-0635 = Listeria newyorkensis FSL |
| | | M6-0635 |
| c2c2-13 | 12 | Leptotrichia wadei F0279 |
| c2c2-14 | 15 | Rhodobacter capsulatus SB 1003 |
| c2c2-15 | 16 | Rhodobacter capsulatus R121 |
| c2c2-16 | 17 | Rhodobacter capsulatus DE442 |
| LbuC2c2 | | Leptorichia buccalis C-1013-b |
| HheC2c2 | | Herbinix hemicellulosilytica |
| EreC2c2 | | Eubacterium rectale |
| EbaC2C2 | | Eubacteriaceae bacterium CHKCI004 |
| C2c2 NK4A144 | | Lachnospiraceae bacterium NK4A144 |
| C2c2 Chloro_agg | | RNA-binding protein S1 Chloroflexus aggregans |
| C2c2 Dem_Aur | | Demequina aurantiaca |
| C2c2 Thal_Sp_TSL5 | | Thalassospira sp. TSL5-1 |
| C2c2 Pseudo_sp | | Pseudobutyrivibrio sp. OR37 |
| C2c2_Buty_sp | | Butyrivibrio sp. YAB3001 |
| C2c2_Blautia_sp | | Blautia sp. Marseille-P2398 |
| C2c2_Lepto_sp_Marseille | | Leptotrichia sp. Marseille-P3007 |
| C2c2_Bacteroides_ihuae | | Bacteroides ihuae |
| C2c2_Porph_bacterium | | Porphyromonadaceae bacterium KH3CP3RA |
| C2c2_Listeria_riparia | | Listeria riparia |
| C2c2_insolitis_peregrinum | | Insolitispirillum peregrinum |

In an embodiment of the invention, there is provided effector protein which comprises an amino acid sequence having at least 80% sequence homology to the wild-type sequence of any of Leptotrichia shahii C2c2, Lachnospiraceae bacterium MA2020 C2c2, Lachnospiraceae bacterium NK4A179 C2c2, Clostridium aminophilum (DSM 10710) C2c2, Carnobacterium gallinarum (DSM 4847) C2c2, Paludibacter propionicigenes (WB4) C2c2, Listeria weihenstephanensis (FSL R9-0317) C2c2, Listeriaceae bacterium (FSL M6-0635) C2c2, Listeria newyorkensis (FSL M6-0635) C2c2, Leptotrichia wadei (F0279) C2c2, Rhodobacter capsulatus (SB 1003) C2c2, Rhodobacter capsulatus (R121) C2c2, Rhodobacter capsulatus (DE442) C2c2, Leptotrichia wadei (Lw2) C2c2, or Listeria seeligeri C2c2.

In an embodiment of the invention, the effector protein comprises an amino acid sequence having at least 80% sequence homology to a Type VI effector protein consensus sequence including but not limited to a consensus sequence described herein

According to the invention, a consensus sequence can be generated from multiple C2c2 orthologs, which can assist in locating conserved amino acid residues, and motifs, including but not limited to catalytic residues and HEPN motifs in C2c2 orthologs that mediate C2c2 function. One such consensus sequence, generated from the 33 orthologs mentioned above using Geneious alignment is SEQ ID NO: 1.

(SEQ ID NO: 1)
MKISKVXXXVXKKXXXGKLXKXVNERNRXAKRLSNXLBKYIXXIDKIXK
KEXXKKFXAXEEITLKLNQXXXBXLXKAXXDLRKDNXYSXJKKILHNED
INXEEXELLINDXLEKLXKIESXKYSYQKXXXNYXMSVQEHSKKSIXRI
XESAKRNKEALDKFLKEYAXLDPRMEXLAKLRKLLELYFYFKNDXIXXE
EEXNVXXHKXLKENHPDFVEXXXNKENAELNXYAIEXKKJLKYYFPXKX
AKNSNDKIFEKQELKKXWIHQJENAVERILLXXGKVXYKLQXGYLAELW
KIRINEIFIKYIXVGKAVAXFALRNXXKBENDILGGKIXKKLNGITSFX
YEKIKAEEILQREXAVEVAFAANXLYAXDLXXIRXSILQFFGGASNWDX
FLFFHFATSXISDKKWNAELIXXKKJGLVIREKLYSNNVAMFYSKDDLE
KLLNXLXXFXLRASQVPSFKKVYVRXBFPQNLLKKENDEKDDEAYSAXY
YLLKEIYYNXFLPYFSANNXFFFXVKNLVLKANKDKFXXAFXDIREMNX
GSPIEYLXXTQXNXXNEGRKKEEKEXDFIKFLLQIFXKGFDDYLKNNXX
FILKFIPEPTEXIEIXXELQAWYIVGKFLNARKXNLLGXFXSYLKLLDD
IELRALRNENIKYQSSNXEKEVLEXCLELIGLLSLDLNDYFBDEXDFAX
YJGKXLDFEKKXMKDLAELXPYDQNDGENPIVNRNIXLAKKYGTLNLLE
KJXDKVSEKEIKEYYELKKEIEEYXXKGEELHEEWXQXKNRVEXRDILE
YXEELXGQIINYNXLXNKVLLYFQLGLHYLLLDILGRLVGYTGIWERDA
XLYQIAAMYXNGLPEYIXXKKNDKYKDGQIVGXKINXFKXDKKXLYNAG
LELFENXNEHKNIXIRNYIAHFNYLSKAESSLLXYSENLRXLFSYDRKL
KNAVXKSLINILLRHGMVLKFKFGTDKKSVXIRSXKKIXHLKSIAKKLY
YPEVXVSKEYCKLVKXLLKYK

In another non-limiting example, a sequence alignment tool to assist generation of a consensus sequence and identification of conserved residues is the MUSCLE alignment tool (www.ebi.ac.uk/Tools/msa/muscle/). For example, using MUSCLE, the following amino acid locations conserved among C2c2 orthologs can be identified in Leptotrichia wadei C2c2:K2; K5; V6; E301; L331; I335; N341; G351; K352; E375; L392; L396; D403; F446; I466; I470; R474 (HEPN); H475; H479 (HEPN), E508; P556; L561; I595; Y596; F600; Y669; I673; F681; L685; Y761; L676; L779; Y782; L836; D847; Y863; L869; I872; K879; I933; L954; I958; R961; Y965; E970; R971; D972; R1046 (HEPN), H1051 (HEPN), Y1075; D1076; K1078; K1080; I1083; I1090.

In certain example embodiments, the RNA-targeting effector protein is a Type VI-B effector protein, such as Cas13b and Group 29 or Group 30 proteins. In certain example embodiments, the RNA-targeting effector protein comprises one or more HTEPN domains. In certain example embodiments, the RNA-targeting effector protein comprises a C-terminal vEPN domain, a N-terminal 1EPN domain, or both. Regarding example Type V-B effector proteins that may be used in the context of this invention, reference is made to U.S. application Ser. No. 15/331,792 entitled “Novel CRISPR Enzymes and Systems” and filed Oct. 21, 2016, International Patent Application No. PCT/U2016/058302 entitled “Novel CRISPR Enzymes and Systems”, and filed Oct. 21, 2016, and Smargon et a. “Cas13b is a Type VI-B CRISPR-associated RNA-Guided RNase differentially regulated by accessory proteins Csx27 and Csx28” Molecular Cell, 65, 1-13 (2017); dx.doi.org/10.1016/j.molcel.2016.12.023, and U.S. Provisional Application No. to be assigned, entitled “Novel Cas13b Orthologues CRISPR Enzymes and System” filed Mar. 15, 2017. In some embodiments, the Cas13b enzyme is derived from Bergeyella zoohelcum. In certain other example embodiments, the effector protein is, or comprises an amino acid sequence having at least 80 sequence homology to a Cas set forth and/or from an organism listed in Table 3.

TABLE 3
Exemplary Cas13 proteins

| | |
|---|---|
| Bergeyella zoohelcum | 1 |
| Prevotella intermedia | 2 |
| Prevotella buccae | 3 |
| Porphyromonas gingivalis | 4 |
| Bacteroides pyogenes | 5 |
| Alistipes sp. ZOR0009 | 6 |
| Prevotella sp. MA2016 | 7a |
| Prevotella sp. MA2016 | 7b |
| Riemerella anatipestifer | 8 |
| Prevotella aurantiaca | 9 |
| Prevotella saccharolytica | 10 |
| HMPREF9712_03108 [Myroides odoratimimus | 11 |
| CCUG 10230] | |
| Prevotella intermedia | 12 |
| Capnocytophaga canimorsus | 13 |
| Porphyromonas gulae | 14 |
| Prevotella sp. P5-125 | 15 |
| Flavobacterium branchiophilum | 16 |
| Myroides odoratimimus | 17 |
| Flavobacterium columnare | 18 |
| Porphyromonas gingivalis | 19 |
| Porphyromonas sp. COT-052 OH4946 | 20 |
| Prevotella intermedia | 21 |
| PIN17_0200 [Prevotella intermedia 17] | AFJ07523 |
| Prevotella intermedia | BAU18623 |
| HMPREF6485_0083 [Prevotella buccae ATCC | EFU31981 |
| 33574] | |
| HMPREF9144_1146 [Prevotella pallens ATCC | EGQ18444 |
| 700821] | |
| HMPREF9714_02132 [Myroides odoratimimus | EHO08761 |
| CCUG 12901] | |
| HMPREF9711_00870 [Myroides odoratimimus | EKB06014 |
| CCUG 3837] | |
| HMPREF9699_02005 [Bergeyella zoohelcum | EKB54193 |
| ATCC 43767] | |
| HMPREF9151_01387 [Prevotella | EKY00089 |
| saccharolytica F0055] | |
| A343_1752 [Porphyromonas gingivalis JCVI | EOA10535 |
| SC001] | |
| HMPREF1981_03090 [Bacteroides pyogenes | ERI81700 |
| F0041] | |
| HMPREF1553_02065 [Porphyromonas | ERJ65637 |
| gingivalis F0568] | |
| HMPREF1988_01768 [Porphyromonas | ERJ81987 |
| gingivalis F0185] | |
| HMPREF1990_01800 [Porphyromonas | ERJ87335 |
| gingivalis W4087] | |
| M573_117042 [Prevotella intermedia ZT] | KJJ86756 |
| A2033_10205 [Bacteroidetes bacterium | OFX18020.1 |
| GWA2_31_91 | |
| SAMN05421542_0666 [Chryseobacterium | SDI27289.1 |
| jejuense] | |
| SAMN05444360_11366 [Chryseobacterium | SHM52812.1 |
| carnipullorum] | |
| SAMN05421786_1011119 [Chryseobacterium | SIS70481.1 |
| ureilyticum] | |
| Prevotella buccae | WP_004343581 |
| Porphyromonas gingivalis | WP_005873511 |
| Porphyromonas gingivalis | WP_005874195 |
| Prevotella pallens | WP_006044833 |
| Myroides odoratimimus | WP_006261414 |
| Myroides odoratimimus | WP_006265509 |
| Prevotella sp. MSX73 | WP_007412163 |
| Porphyromonas gingivalis | WP_012458414 |
| Paludibacter propionicigenes | WP_013446107 |
| Porphyromonas gingivalis | WP_013816155 |
| Flavobacterium columnare | WP_014165541 |
| Psychroflexus torquis | WP_015024765 |
| Riemerella anatipestifer | WP_015345620 |
| Prevotella pleuritidis | WP_021584635 |
| Porphyromonas gingivalis | WP_021663197 |
| Porphyromonas gingivalis | WP_021665475 |
| Porphyromonas gingivalis | WP_021677657 |
| Porphyromonas gingivalis | WP_021680012 |
| Porphyromonas gingivalis | WP_023846767 |
| Prevotella falsenii | WP_036884929 |
| Prevotella pleuritidis | WP_036931485 |
| [Porphyromonas gingivalis | WP_039417390 |
| Porphyromonas gulae | WP_039418912 |
| Porphyromonas gulae | WP_039419792 |
| Porphyromonas gulae | WP_039426176 |
| Porphyromonas gulae | WP_039431778 |
| Porphyromonas gulae | WP_039437199 |
| Porphyromonas gulae | WP_039442171 |
| Porphyromonas gulae | WP_039445055 |
| Capnocytophaga cynodegmi | WP_041989581 |
| Prevotella sp. P5-119 | WP_042518169 |
| Prevotella sp. P4-76 | WP_044072147 |
| Prevotella sp. P5-60 | WP_044074780 |
| Phaeodactylibacter xiamenensis | WP_044218239 |
| Flavobacterium sp. 316 | WP_045968377 |
| Porphyromonas gulae | WP_046201018 |
| Chryseobacterium sp. YR477 | WP_047431796 |
| Riemerella anatipestifer | WP_049354263 |
| Porphyromonas gingivalis | WP_052912312 |
| Porphyromonas gingivalis | WP_058019250 |
| Flavobacterium columnare | WP_060381855 |
| Porphyromonas gingivalis | WP_061156470 |
| Porphyromonas gingivalis | WP_061156637 |
| Riemerella anatipestifer | WP_061710138 |
| Flavobacterium columnare | WP_063744070 |
| Riemerella anatipestifer | WP_064970887 |
| Sinomicrobium oceani | WP_072319476.1 |
| Reichenbachiella agariperforans | WP_073124441.1 |

In certain example embodiments, the Class 2 type VI CRISPR system is a Cas13c system. In certain example embodiments, the Cas13c orthologue is selected from Table 4, which includes Cas13c orthologues for expression in mammalian cells.

TABLE 4

Fusobacterium necrophorum subsp.
funduliforme ATCC 51357 contig00003
Fusobacterium necrophorum DJ-2 contig0065,
whole genome shotgun sequence
Fusobacterium necrophorum BFTR-1 contig0068
Fusobacterium necrophorum subsp.
funduliforme 1_1 36S cont1.14
Fusobacterium perfoetens ATCC 29250
T364DRAFT_scaffold00009.9_C
Fusobacterium ulcerans ATCC 49185
cont2.38
Anaerosalibacter sp. ND1 genome
assembly Anaerosalibacter massiliensis ND1

In certain example embodiments, the RNA-targeting effector protein is a Cas13c effector protein as disclosed in U.S. Provisional Patent Application No. 62/525,165 filed Jun. 26, 2017, and PCT Application No. US 2017/047193 filed Aug. 16, 2017. Example wildtype orthologue sequences of Cas13c are provided in Table 5.

TABLE 5
Name

EHO19081
WP_094899336
WP_040490876
WP_047396607
WP_035935671
WP_035906563
WP_042678931
WP_062627846
WP_005959231
WP_027128616
WP_062624740
WP_096402050

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual 1-EPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas 13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

In certain example embodiments, the Cas 13d is as in U.S. Pat. No. 10,666,592, which is incorporated by reference as if expressed in its entirety herein.

In some embodiments, one or more elements of a nucleic acid-targeting system is derived from a particular organism comprising an endogenous CRISPR RNA-targeting system. In certain example embodiments, the effector protein CRISPR RNA-targeting system comprises at least one HEPN domain, including but not limited to the HEPN domains described herein, HEPN domains known in the art, and domains recognized to be HEPN domains by comparison to consensus sequence motifs. Several such domains are provided herein. In one non-limiting example, a consensus sequence can be derived from the sequences of C2c2 or Cas13b orthologs provided herein. In certain example embodiments, the effector protein comprises a single HEPN domain. In certain other example embodiments, the effector protein comprises two HEPN domains.

In one example embodiment, the effector protein comprise one or more HEPN domains comprising a RxxxxH motif sequence. The RxxxxH motif sequence can be, without limitation, from a HEPN domain described herein or a HEPN domain known in the art. RxxxxH motif sequences further include motif sequences created by combining portions of two or more HEPN domains. As noted, consensus sequences can be derived from the sequences of the orthologs disclosed in U.S. Provisional Patent Application 62/432,240 entitled “Novel CRISPR Enzymes and Systems,” U.S. Provisional Patent Application 62/471,710 entitled “Novel Type VI CRISPR Orthologs and Systems” filed on Mar. 15, 2017, and U.S. Provisional Patent application entitled “Novel Type VI CRISPR Orthologs and Systems,” labeled as attorney docket number 47627-05-2133 and filed on Apr. 12, 2017.

In an embodiment of the invention, a HEPN domain comprises at least one RxxxxH motif comprising the sequence of R{N/H/K}X1X2X3H (SEQ ID NO: 2-4). In an embodiment of the invention, a HEPN domain comprises a RxxxxH motif comprising the sequence of R{N/H}X1X2X3H (SEQ ID NO: 5-6). In an embodiment of the invention, a HEPN domain comprises the sequence of R{N/K}X1X2X3H (SEQ ID NO: 7-8). In certain embodiments, X1 is R, S, D, E, Q, N, G, Y, or H. In certain embodiments, X2 is I, S, T, V, or L. In certain embodiments, X3 is L, F, N, Y, V, I, S, D, E, or A.

### Cas12 Polypeptides

In certain example embodiments, a Cas that has collateral activity that can be included in the CRISPR-Cas system is or includes one or more Cas12 polypeptides (e.g., Cas 12a (also known as Cpf1), 12b (also known as C2c1), 12c, 12c1, 12c2, 12d, 12e, 12a1, 12g1, 12h1, 12i1, 12f (also known as Cas14) See e.g., Kaminski et al., Nat. Biomed. Eng. 5:643-656 (2021)). In some embodiments, the Cas12 protein can have trans-cleavage activity (also referred to as collateral cleavage), which cleaves ssDNA indiscriminately. In some embodiments, the Cas12 has multiple-turnover nuclease activity, which can be harnessed in the context of an assay described herein for amplified detection of targets.

Cas12's non-specific cleavage can be leveraged to cleave reporters upon target recognition, allowing for the design of sensitive and specific diagnostics using Cas12, including single nucleotide variants, detection based on rRNA sequences, screening for drug resistance, monitoring microbe outbreaks, genetic perturbations, and screening of environmental samples, as described, for example, in Broughton et al. 2020. CRISPR-Cas12-based detection of SARS-CoV-2. Nat. Biotech. 38:870-874, https://doi.org/10.1038/s41587-020-0513-4; Leung et al. 2021. CRISPR-Cas12-based nucleic acids detection systems. Methods.; S1046-2023(21)00063-3.doi: 10.1016/j.ymeth.2021.02.018; Mahas et al., Viruses. 2021. 13:466, https://doi.org/10.3390/v13030466; Ali et al., 2020. iSCAN: An RT-LAMP-coupled CRISPR-Cas12 module for rapid, sensitive detection of SARS-CoV-2Vir. Res. 288:198129. https://doi.org/10.1016/j.virusres.2020.198129; Ramachandran et al., 2020. Electric field-driven microfluidics for rapid CRISPR-based diagnostics and its application to detection of SARS-CoV-2. PNAS Nov. 24, 2020 117 (47) 29518-29525; Mukama et al., An ultrasensitive and specific point-of care CRISPR-Cas12 based lateral flow biosensor for the rapid detection of nucleic acids. Biosens Bioelectron. 2020 Jul. 1; 159:112143. doi: 10.1016/j.bios.2020.112143; Chen et al., 2018. CRISPR-Cas12a target binding unleashes indiscriminate single-stranded DNase activity. Science. Apr. 27; 360(6387):436-439. doi: 10.1126/science.aar6245; Kellner et al., 2019. Nat Protoc. 2019 October; 14(10):2986-3012. doi: 10.1038/s41596-019-0210-2; Broughton et al., 2020. Rapid Detection of 2019 Novel Coronavirus SARS-CoV-2 Using a CRISPR-based DETECTR Lateral Flow Assay. 2020. medRxiv. March 27; 2020.03.06.20032334. doi: 10.1101/2020.03.06.20032334; Wu et al. 2021. CRISPR-Cas12-Based Rapid Authentication of Halal Food. J Agric Food Chem. 2021 Aug. 26. doi: 10.1021/acs.jafc.1c03078; Long et al. 2021. CRISPR/Cas12-Based Ultra-Sensitive and Specific Point-of-Care Detection of HBV. Int J Mol Sci. 2021 May 3; 22(9):4842. doi: 10.3390/ijms22094842; Curti et al., Viruses. 2021 Mar. 5; 13(3):420. doi: 10.3390/v13030420; Li et al., Cell Discovery (2018)4:20. DOI 10.1038/s41421-018-0028-z; Lucia et al. 2020. An ultrasensitive, rapid, and portable coronavirus SARS-Cov-2 sequence detection method based on CRISPR-Cas12. bioRxiv preprint doi: https/doi.org/10.1101/2020.02.29.971127; MammothBiosciences. 2020. Broughton et al., available at https://mammoth.bio/wp-content/uploads/2020/04/200423-A-protocol-for-rapid-detection-of-SARS-CoV-2-using-CRISPR-diagnostics_3.pdf; East-Seletsky et al., Nat. 538:270, doi:10.1038/nature19802; International Pat. Pub. WO2019/233358; WO2019/011022; U.S. Pat. Nos. 10,337,051; 10,449,4664, 10,253,365; US 2020/0299768; US 2020/0399697; US 2019/0241954; the disclosure of each can be adapted for use with the present invention in view of the description provided herein and each of which is incorporated herein by reference in its entirety.

### Cpf1 Orthologs

The present invention encompasses the use of a Cpf1 effector protein, derived from a Cpf1 locus denoted as subtype V-A. Herein such effector proteins are also referred to as “Cpf1p”, e.g., a Cpf1 protein (and such effector protein or Cpf1 protein or protein derived from a Cpf1 locus is also called “CRISPR enzyme”). Presently, the subtype V-A loci encompasses cas1, cas2, a distinct gene denoted cpf1 and a CRISPR array. Cpf1 (CRISPR-associated protein Cpf1, subtype PREFRAN) is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in all Cas9 proteins, and the RuvC-like domain is contiguous in the Cpf1 sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided Cpf1 also make it an ideal switchable nuclease for non-specific cleavage of nucleic acids. In one embodiment, a Cpf1 system is engineered to provide and take advantage of collateral non-specific cleavage of RNA. In another embodiment, a Cpf1 system is engineered to provide and take advantage of collateral non-specific cleavage of ssDNA. Accordingly, engineered Cpf1 systems provide platforms for nucleic acid detection and transcriptome manipulation. Cpf1 is developed for use as a mammalian transcript knockdown and binding tool. Cpf1 is capable of robust collateral cleavage of RNA and ssDNA when activated by sequence-specific targeted DNA binding.

Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April; 22(4):359-66. doi: 10.1002/pro.2225.). See also Shmakov et al. (2015) for application in the field of CRISPR-Cas loci. Homologous proteins may but need not be structurally related, or are only partially structurally related. The Cpf1 gene is found in several diverse bacterial genomes, typically in the same locus with cas1, cas2, and cas4 genes and a CRISPR cassette (for example, FNFX1_1431-FNFX1_1428 of Francisella cf. novicida Fx1). In particular embodiments, the effector protein is a Cpf1 effector protein from an organism from a genus comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus.

In further particular embodiments, the Cpf1 effector protein is from an organism selected from S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii.

The effector protein may comprise a chimeric effector protein comprising a first fragment from a first effector protein (e.g., a Cpf1) ortholog and a second fragment from a second effector (e.g., a Cpf1) protein ortholog, and wherein the first and second effector protein orthologs are different. At least one of the first and second effector protein (e.g., a Cpf1) orthologs may comprise an effector protein (e.g., a Cpf1) from an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus; e.g., a chimeric effector protein comprising a first fragment and a second fragment wherein each of the first and second fragments is selected from a Cpf1 of an organism comprising Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium or Acidaminococcus wherein the first and second fragments are not from the same bacteria; for instance a chimeric effector protein comprising a first fragment and a second fragment wherein each of the first and second fragments is selected from a Cpf1 of S. mutans, S. agalactiae, S. equisimilis, S. sanguinis, S. pneumonia; C. jejuni, C. coli; N. salsuginis, N. tergarcus; S. auricularis, S. carnosus; N. meningitides, N. gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae, wherein the first and second fragments are not from the same bacteria. In a more preferred embodiment, the Cpf1p is derived from a bacterial species selected from Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens and Porphyromonas macacae. In certain embodiments, the Cpf1p is derived from a bacterial species selected from Acidaminococcus sp. BV3L6, Lachnospiraceae bacterium MA2020. In certain embodiments, the effector protein is derived from a subspecies of Francisella tularensis 1, including but not limited to Francisella tularensis subsp. Novicida.

In some embodiments, the Cpf1p is derived from an organism from the genus of Eubacterium. In some embodiments, the CRISPR effector protein is a Cpf1 protein derived from an organism from the bacterial species of Eubacterium rectale. In some embodiments, the amino acid sequence of the Cpf1 effector protein corresponds to NCBI Reference Sequence WP_055225123.1, NCBI Reference Sequence WP_055237260.1, NCBI Reference Sequence WP_055272206.1, or GenBank ID OLA16049.1. In some embodiments, the Cpf1 effector protein has a sequence homology or sequence identity of at least 60%, more particularly at least 70, such as at least 80%, more preferably at least 85%, even more preferably at least 90%, such as for instance at least 95%, with NCBI Reference Sequence WP_055225123.1, NCBI Reference Sequence WP_055237260.1, NCBI Reference Sequence WP_055272206.1, or GenBank ID OLA16049.1. The skilled person will understand that this includes truncated forms of the Cpf1 protein whereby the sequence identity is determined over the length of the truncated form. In some embodiments, the Cpf1 effector recognizes the PAM sequence of TTTN or CTTN.

In particular embodiments, the homologue or orthologue of Cpf1 as referred to herein has a sequence homology or identity of at least 80%, more preferably at least 85%, even more preferably at least 90%, such as for instance at least 95% with Cpf1. In further embodiments, the homologue or orthologue of Cpf1 as referred to herein has a sequence identity of at least 80%, more preferably at least 85%, even more preferably at least 90%, such as for instance at least 95% with the wild type Cpf1. Where the Cpf1 has one or more mutations (mutated), the homologue or orthologue of said Cpf1 as referred to herein has a sequence identity of at least 80%, more preferably at least 85%, even more preferably at least 90%, such as for instance at least 95% with the mutated Cpf1.

In an embodiment, the Cpf1 protein may be an ortholog of an organism of a genus which includes, but is not limited to Acidaminococcus sp, Lachnospiraceae bacterium or Moraxella bovoculi; in particular embodiments, the type V Cas protein may be an ortholog of an organism of a species which includes, but is not limited to Acidaminococcus sp. BV3L6; Lachnospiraceae bacterium ND2006 (LbCpf1) or Moraxella bovoculi 237. In particular embodiments, the homologue or orthologue of Cpf1 as referred to herein has a sequence homology or identity of at least 80%, more preferably at least 85%, even more preferably at least 90%, such as for instance at least 95% with one or more of the Cpf1 sequences disclosed herein. In further embodiments, the homologue or orthologue of Cpf as referred to herein has a sequence identity of at least 80%, more preferably at least 85%, even more preferably at least 90%, such as for instance at least 95% with the wild type FnCpf1, AsCpf1 or LbCpf1. The skilled person will understand that this includes truncated forms of the Cpf1 protein whereby the sequence identity is determined over the length of the truncated form. In certain of the following, Cpf1 amino acids are followed by nuclear localization signals (NLS) (italics), a glycine-serine (GS) linker, and 3×HA tag. Further Cpf1 orthologs include NCBI WP_055225123.1, NCBI WP_055237260.1, NCBI WP_055272206.1, and GenBank OLA16049.1.

### C2c1 Orthologs

The present invention encompasses the use of a C2c1 effector proteins, derived from a C2c1 locus denoted as subtype V-B. Herein such effector proteins are also referred to as “C2c1p”, e.g., a C2c1 protein (and such effector protein or C2c1 protein or protein derived from a C2c1 locus is also called “CRISPR enzyme”). Presently, the subtype V-B loci encompasses cas1-Cas4 fusion, cas2, a distinct gene denoted C2c1 and a CRISPR array. C2c1 (CRISPR-associated protein C2c1) is a large protein (about 1100-1300 amino acids) that contains a RuvC-like nuclease domain homologous to the corresponding domain of Cas9 along with a counterpart to the characteristic arginine-rich cluster of Cas9. However, C2c1 lacks the HNH nuclease domain that is present in all Cas9 proteins, and the RuvC-like domain is contiguous in the C2c1 sequence, in contrast to Cas9 where it contains long inserts including the HNH domain. Accordingly, in particular embodiments, the CRISPR-Cas enzyme comprises only a RuvC-like nuclease domain.

The programmability, specificity, and collateral activity of the RNA-guided C2c1 also make it an ideal switchable nuclease for non-specific cleavage of nucleic acids. In one embodiment, a C2c1 system is engineered to provide and take advantage of collateral non-specific cleavage of RNA. In another embodiment, a C2c1 system is engineered to provide and take advantage of collateral non-specific cleavage of ssDNA. Accordingly, engineered C2c1 systems provide platforms for nucleic acid detection and transcriptome manipulation, and inducing cell death. C2c1 is developed for use as a mammalian transcript knockdown and binding tool. C2c1 is capable of robust collateral cleavage of RNA and ssDNA when activated by sequence-specific targeted DNA binding.

In certain embodiments, C2c1 is provided or expressed in an in vitro system or in a cell, transiently or stably, and targeted or triggered to non-specifically cleave cellular nucleic acids. In one embodiment, C2c1 is engineered to knock down ssDNA, for example viral ssDNA. In another embodiment, C2c1 is engineered to knock down RNA. The system can be devised such that the knockdown is dependent on a target DNA present in the cell or in vitro system or triggered by the addition of a target nucleic acid to the system or cell.

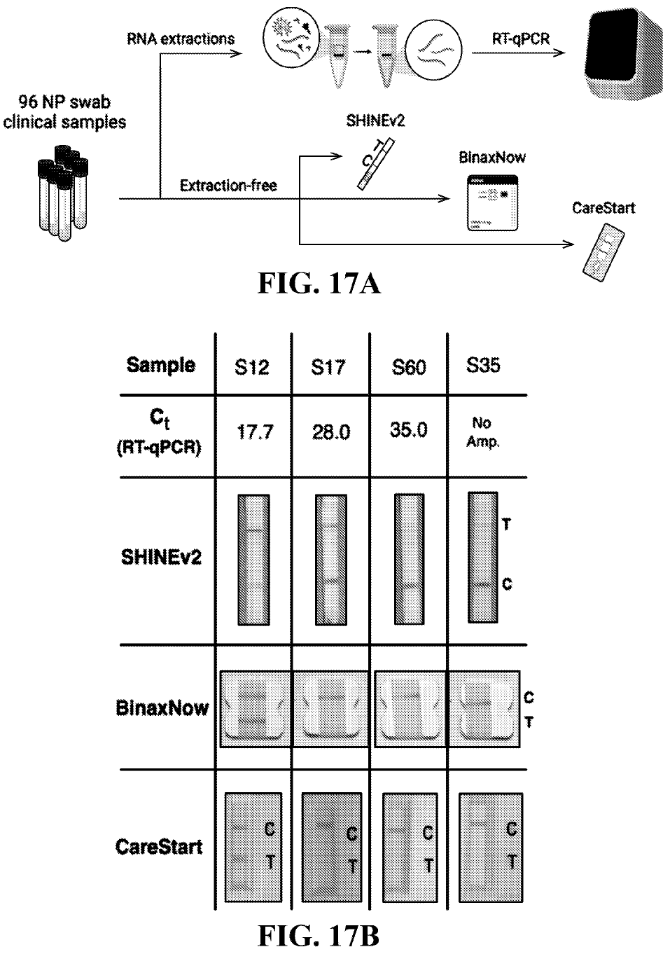

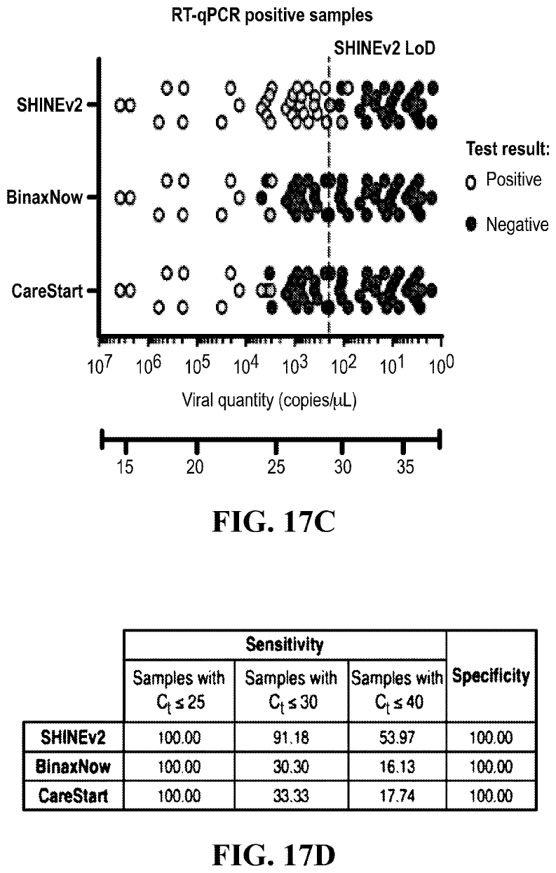

C2c1 (also known as Cas12b) proteins are RNA guided nucleases. In certain embodiments, the Cas protein may comprise at least 80% sequence identity to a polypeptide as described in International Patent Publication WO 2016/205749 at FIG. 17-21, FIG. 41A-41M, 44A-44E, incorporated herein by reference. Its cleavage relies on a tracr RNA to recruit a guide RNA comprising a guide sequence and a direct repeat, where the guide sequence hybridizes with the target nucleotide sequence to form a DNA/RNA heteroduplex. Based on current studies, C2c1 nuclease activity also requires relies on recognition of PAM sequence. C2c1 PAM sequences are T-rich sequences. In some embodiments, the PAM sequence is 5′ TTN 3′ or 5′ ATTN 3′, wherein N is any nucleotide. In a particular embodiment, the PAM sequence is 5′ TTC 3′. In a particular embodiment, the PAM is in the sequence of Plasmodium falciparum.

In particular embodiments, the effector protein is a C2c1 effector protein from an organism from a genus comprising Alicyclobacillus, Desulfovibrio, Desulfonatronum, Opitutaceae, Tuberibacillus, Bacillus, Brevibacillus, Candidatus, Desulfatirhabdium, Citrobacter, Elusimicrobia, Methylobacterium, Omnitrophica, Phycisphaerae, Planctomycetes, Spirochaetes, and Verrucomicrobiaceae.

In further particular embodiments, the C2c1 effector protein is from a species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF 112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060).

The effector protein may comprise a chimeric effector protein comprising a first fragment from a first effector protein (e.g., a C2c1) ortholog and a second fragment from a second effector (e.g., a C2c1) protein ortholog, and wherein the first and second effector protein orthologs are different. At least one of the first and second effector protein (e.g., a C2c1) orthologs may comprise an effector protein (e.g., a C2c1) from an organism comprising Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; e.g., a chimeric effector protein comprising a first fragment and a second fragment wherein each of the first and second fragments is selected from a C2c1 of an organism comprising Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae wherein the first and second fragments are not from the same bacteria; for instance a chimeric effector protein comprising a first fragment and a second fragment wherein each of the first and second fragments is selected from a C2c1 of Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF 112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060), wherein the first and second fragments are not from the same bacteria.

In a more preferred embodiment, the C2c1p is derived from a bacterial species selected from Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF 112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In certain embodiments, the C2c1p is derived from a bacterial species selected from Alicyclobacillus acidoterrestris (e.g., ATCC 49025), Alicyclobacillus contaminans (e.g., DSM 17975).

In particular embodiments, the homologue or orthologue of C2c1 as referred to herein has a sequence homology or identity of at least 80%, more preferably at least 85%, even more preferably at least 90%, such as for instance at least 95% with C2c1. In further embodiments, the homologue or orthologue of C2c1 as referred to herein has a sequence identity of at least 80%, more preferably at least 85%, even more preferably at least 90%, such as for instance at least 95% with the wild type C2c1. Where the C2c1 has one or more mutations (mutated), the homologue or orthologue of said C2c1 as referred to herein has a sequence identity of at least 80%, more preferably at least 85%, even more preferably at least 90%, such as for instance at least 95% with the mutated C2c1.

In an embodiment, the C2c1 protein may be an ortholog of an organism of a genus which includes, but is not limited to Alicyclobacillus, Desulfovibrio, Desulfonatronum, Opitutaceae, Tuberibacillus, Bacillus, Brevibacillus, Candidatus, Desulfatirhabdium, Elusimicrobia, Citrobacter, Methylobacterium, Omnitrophicai, Phycisphaerae, Planctomycetes, Spirochaetes, and Verrucomicrobiaceae; in particular embodiments, the type V Cas protein may be an ortholog of an organism of a species which includes, but is not limited to Alicyclobacillus acidoterrestris (e.g., ATCC 49025), Alicyclobacillus contaminans (e.g., DSM 17975), Alicyclobacillus macrosporangiidus (e.g. DSM 17980), Bacillus hisashii strain C4, Candidatus Lindowbacteria bacterium RIFCSPLOWO2, Desulfovibrio inopinatus (e.g., DSM 10711), Desulfonatronum thiodismutans (e.g., strain MLF-1), Elusimicrobia bacterium RIFOXYA12, Omnitrophica WOR_2 bacterium RIFCSPHIGHO2, Opitutaceae bacterium TAV5, Phycisphaerae bacterium ST-NAGAB-D1, Planctomycetes bacterium RBG_13_46_10, Spirochaetes bacterium GWB1_27_13, Verrucomicrobiaceae bacterium UBA2429, Tuberibacillus calidus (e.g., DSM 17572), Bacillus thermoamylovorans (e.g., strain B4166), Brevibacillus sp. CF 112, Bacillus sp. NSP2.1, Desulfatirhabdium butyrativorans (e.g., DSM 18734), Alicyclobacillus herbarius (e.g., DSM 13609), Citrobacter freundii (e.g., ATCC 8090), Brevibacillus agri (e.g., BAB-2500), Methylobacterium nodulans (e.g., ORS 2060). In particular embodiments, the homologue or orthologue of C2c1 as referred to herein has a sequence homology or identity of at least 80%, more preferably at least 85%, even more preferably at least 90%, such as for instance at least 95% with one or more of the C2c1 sequences disclosed herein. In further embodiments, the homologue or orthologue of C2c1 as referred to herein has a sequence identity of at least 80%, more preferably at least 85%, even more preferably at least 90%, such as for instance at least 95% with the wild type AacC2c1 or BthC2c1.

In particular embodiments, the C2c1 protein of the invention has a sequence homology or identity of at least 60%, more particularly at least 70, such as at least 80%, more preferably at least 85%, even more preferably at least 90%, such as for instance at least 95% with AacC2c1 or BthC2c1. In further embodiments, the C2c1 protein as referred to herein has a sequence identity of at least 60%, such as at least 70%, more particularly at least 80%, more preferably at least 85%, even more preferably at least 90%, such as for instance at least 95% with the wild type AacC2c1. In particular embodiments, the C2c1 protein of the present invention has less than 60% sequence identity with AacC2c1. The skilled person will understand that this includes truncated forms of the C2c1 protein whereby the sequence identity is determined over the length of the truncated form.

In certain methods according to the present invention, the CRISPR-Cas protein is preferably mutated with respect to a corresponding wild-type enzyme such that the mutated CRISPR-Cas protein lacks the ability to cleave one or both DNA strands of a target locus containing a target sequence. In particular embodiments, one or more catalytic domains of the C2c1 protein are mutated to produce a mutated Cas protein which cleaves only one DNA strand of a target sequence.

In particular embodiments, the CRISPR-Cas protein may be mutated with respect to a corresponding wild-type enzyme such that the mutated CRISPR-Cas protein lacks substantially all DNA cleavage activity. In some embodiments, a CRISPR-Cas protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form.

In certain embodiments of the methods provided herein the CRISPR-Cas protein is a mutated CRISPR-Cas protein which cleaves only one DNA strand, i.e. a nickase. More particularly, in the context of the present invention, the nickase ensures cleavage within the non-target sequence, i.e., the sequence which is on the opposite DNA strand of the target sequence and which is 3′ of the PAM sequence. By means of further guidance, and without limitation, an arginine-to-alanine substitution (R911A) in the Nuc domain of C2c1 from Alicyclobacillus acidoterrestris converts C2c1 from a nuclease that cleaves both strands to a nickase (cleaves a single strand). It will be understood by the skilled person that where the enzyme is not AacC2c1, a mutation may be made at a residue in a corresponding position.

### Cas 12c Orthologs

In certain embodiments, the effector protein, particularly a Type V loci effector protein, more particularly a Type V-C loci effector protein, even more particularly a C2c3p, may originate, may be isolated or may be derived from a bacterial metagenome selected from the group consisting of the bacterial metagenomes listed in the Table in FIG. 43A-43B of PCT/US2016/038238, specifically incorporated by reference, which presents analysis of the Type-V-C Cas12c loci.

In certain embodiments, the effector protein, particularly a Type V loci effector protein, more particularly a Type V-C loci effector protein, even more particularly a C2c3p, may comprise, consist essentially of or consist of an amino acid sequence selected from the group consisting of amino acid sequences shown in the multiple sequence alignment in FIG. 13I of PCT/US2016/038238, specifically incorporated by reference.

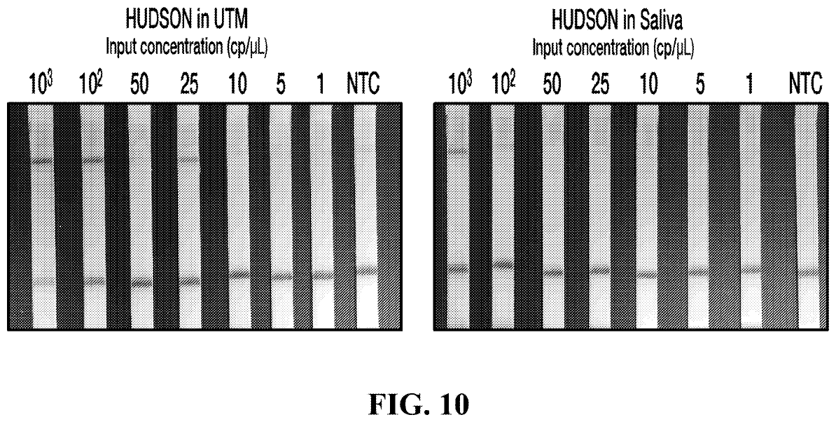

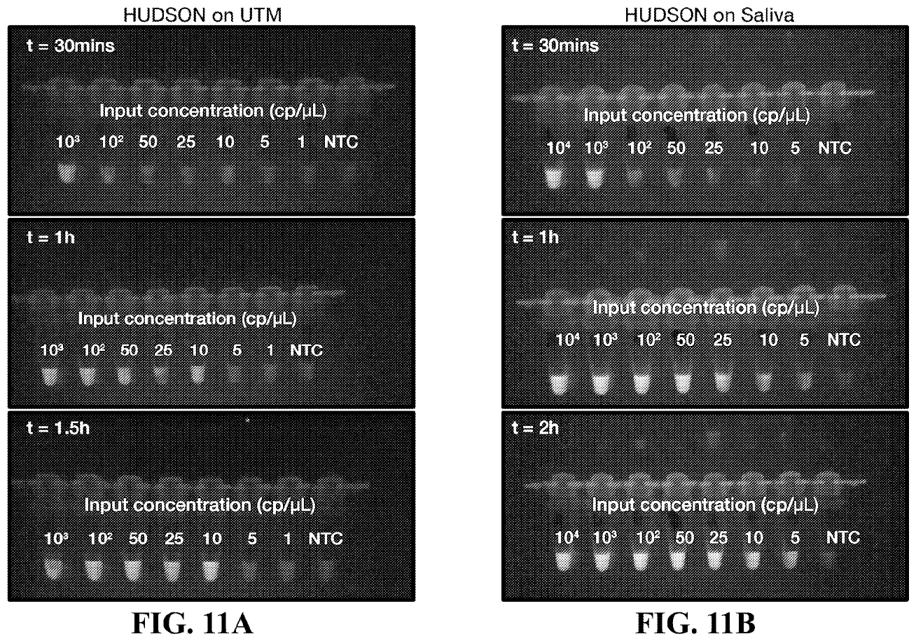

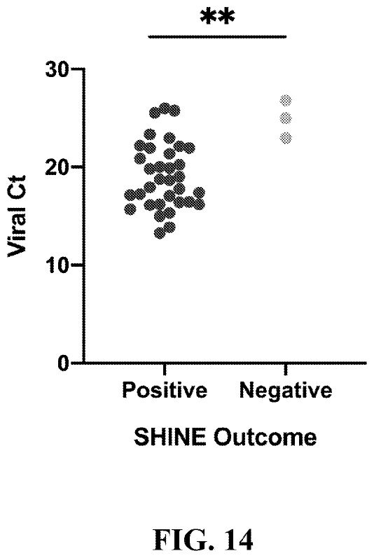

In certain embodiments, a Type V-C locus as intended herein may encode Cas1 and the C2c3p effector protein. See FIG. 14 of PCT/US2016/038238, specifically incorporated by reference, depicting the genomic architecture of the Cas12c CRISPR-Cas loci. In certain embodiments, a Cas1 protein encoded by a Type V-C locus as intended herein may cluster with Type I-B system. See FIG. 10A and 10B and FIG. 10C-V of PCT/US2016/038238, specifically incorporated by reference, illustrating a Cas1 tree including Cas1 encoded by representative Type V-C loci.

In certain embodiments, the effector protein, particularly a Type V loci effector protein, more particularly a Type V-C loci effector protein, even more particularly a C2c3p, such as a native C2c3p, may be about 1100 to about 1500 amino acids long, e.g., about 1100 to about 1200 amino acids long, or about 1200 to about 1300 amino acids long, or about 1300 to about 1400 amino acids long, or about 1400 to about 1500 amino acids long, e.g., about 1100, about 1200, about 1300, about 1400 or about 1500 amino acids long, or at least about 1100, at least about 1200, at least about 1300, at least about 1400 or at least about 1500 amino acids long.

In certain embodiments, the effector protein, particularly a Type V loci effector protein, more particularly a Type V-C loci effector protein, even more particularly a C2c3p, and preferably the C-terminal portion of said effector protein, comprises the three catalytic motifs of the RuvC-like nuclease (i.e., RuvCI, RuvCII and RuvCIII). In certain embodiments, said effector protein, and preferably the C-terminal portion of said effector protein, may further comprise a region corresponding to the bridge helix (also known as arginine-rich cluster) that in Cas9 protein is involved in crRNA-binding. In certain embodiments, said effector protein, and preferably the C-terminal portion of said effector protein, may further comprise a Zn finger region. Preferably, the Zn-binding cysteine residue(s) may be conserved in C2c3p. In certain embodiments, said effector protein, and preferably the C-terminal portion of said effector protein, may comprise the three catalytic motifs of the RuvC-like nuclease (i.e., RuvCI, RuvCII and RuvCIII), the region corresponding to the bridge helix, and the Zn finger region, preferably in the following order, from N to C terminus: RuvCI-bridge helix-RuvCII-Zinc finger-RuvCIII. See FIGS. 13A and 13C of PCT/US2016/038238, specifically incorporated by reference, for illustration of representative Type V-C effector proteins domain architecture.

In certain embodiments, Type V-C loci as intended herein may comprise CRISPR repeats between 20 and 30 bp long, more typically between 22 and 27 bp long, yet more typically 25 bp long, e.g., 20, 21, 22, 23, 24, 25, 26, 27, 28, 29, or 30 bp long.

Orthologous proteins may but need not be structurally related, or are only partially structurally related. In particular embodiments, the homologue or orthologue of a Type V protein such as Cas12c as referred to herein has a sequence homology or identity of at least 80%, more preferably at least 85%, even more preferably at least 90%, such as for instance at least 95% with a Cas12c. In further embodiments, the homologue or orthologue of a Type V Cas12c as referred to herein has a sequence identity of at least 80%, more preferably at least 85%, even more preferably at least 90%, such as for instance at least 95% with the wild type Cas12c.

In an embodiment, the Type V RNA-targeting Cas protein may be a Cas12c ortholog of an organism of a genus which includes but is not limited to Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma and Campylobacter.

In an embodiment, the Cas12c or an ortholog or homolog thereof, may comprise one or more mutations (and hence nucleic acid molecule(s) coding for same may have mutation(s). The mutations may be artificially introduced mutations and may include but are not limited to one or more mutations in a catalytic domain. Examples of catalytic domains with reference to a Cas9 enzyme may include but are not limited to RuvC I, RuvC II, RuvC III and HNH domains. In an embodiment, the Cas12c or an ortholog or homolog thereof, may comprise one or more mutations. The mutations may be artificially introduced mutations and may include but are not limited to one or more mutations in a catalytic domain. Examples of catalytic domains with reference to a Cas enzyme may include but are not limited to RuvC I, RuvC II, RuvC III, HNH domains, and HEPN domains.

### Cas12f Orthologs

In some embodiments, the Cas12 is Cas12f (also known as Cas14). In general, Cas12f is smaller in size than other Cas12 proteins or Cas9, which can be advantageous for in-cell detection assays. Cas12f can also have increased specificity towards ssDNA than Cas12a, making it advantageously suitable for use in assays that are configured to detect single nucleotide differences at certain protospacer sites (see e.g., Harrington et al. Science. 2018. 362:839-842).

In some embodiments, the Cas12f is any one set forth and described in Karvelis et al., 2020. Nucleic Acids Res. 48(9):5016-5023; Harrington et al. Science 2018. 362(6416):839-842; Savag, D. F. 2019. Biochemistry. 58(8):1024-1025. Aquino-Jarquin G. Nanomedicine. 2019.18:428-431; and/or Takeda et al., Mol. Cell. 2021. 81(3):558-570. In some embodiments, the Cas12f proteins are about 400 to about 700 amino acids in size.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

As used herein, the term “crRNA” or “guide RNA” or “single guide RNA,” “gRNA” refers to a polynucleotide comprising any polynucleotide sequence having sufficient complementarity with a target nucleic acid sequence to hybridize with the target nucleic acid sequence and to direct sequence-specific binding of a RNA-targeting complex comprising the gRNA and a CRISPR effector protein to the target nucleic acid sequence. In general, a gRNA may be any polynucleotide sequence (i) being able to form a complex with a CRISPR effector protein and (ii) comprising a sequence having sufficient complementarity with a target polynucleotide sequence to hybridize with the target sequence and direct sequence-specific binding of a CRISPR complex to the target sequence. As used herein the term “capable of forming a complex with the CRISPR effector protein” refers to the gRNA having a structure that allows specific binding by the CRISPR effector protein to the gRNA such that a complex is formed that is capable of binding to a target RNA in a sequence specific manner and that can exert a function on said target RNA. Structural components of the gRNA may include direct repeats and a guide sequence (or spacer). The sequence specific binding to the target RNA is mediated by a part of the gRNA, the “guide sequence”, being complementary to the target RNA. In embodiments of the invention the term guide RNA, i.e. RNA capable of guiding Cas to a target locus, is used as in foregoing cited documents such as WO 2014/093622 (PCT/US2013/074667). As used herein the term “wherein the guide sequence is capable of hybridizing” refers to a subsection of the gRNA having sufficient complementarity to the target sequence to hybridize thereto and to mediate binding of a CRISPR complex to the target RNA. In general, a guide sequence is any polynucleotide sequence having sufficient complementarity with a target polynucleotide sequence to hybridize with the target sequence and direct sequence-specific binding of a CRISPR complex to the target sequence.

In certain embodiments, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

In certain embodiments, use is made of chemically modified guide RNAs. Examples of guide RNA chemical modifications include, without limitation, incorporation of 2′-O-methyl (M), 2′-O-methyl 3′phosphorothioate (MS), or 2′-O-methyl 3′thioPACE (MSP) at one or more terminal nucleotides. Such chemically modified guide RNAs can comprise increased stability and increased activity as compared to unmodified guide RNAs, though on-target vs. off-target specificity is not predictable. (See, Hendel, 2015, Nat Biotechnol. 33(9):985-9, doi: 10.1038/nbt.3290, published online 29 Jun. 2015). Chemically modified guide RNAs further include, without limitation, RNAs with phosphorothioate linkages and locked nucleic acid (LNA) nucleotides comprising a methylene bridge between the 2′ and 4′ carbons of the ribose ring.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap. genomics. org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity withand to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide RNA is selected to reduce the degree of secondary structure within the RNA-targeting guide RNA. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In a classic CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In certain embodiments, modulations of cleavage efficiency can be exploited by introduction of mismatches, e.g., 1 or more mismatches, such as 1 or 2 mismatches between spacer sequence and target sequence, including the position of the mismatch along the spacer/target. The more central (i.e., not 3′ or 5′) for instance a double mismatch is, the more cleavage efficiency is affected. Accordingly, by choosing mismatch position along the spacer, cleavage efficiency can be modulated. By means of example, if less than 100% cleavage of targets is desired (e.g., in a cell population), 1 or more, such as preferably 2 mismatches between spacer and target sequence may be introduced in the spacer sequences. The more central along the spacer of the mismatch position, the lower the cleavage percentage.

In certain example embodiments, the cleavage efficiency may be exploited to design single guides that can distinguish two or more targets that vary by a single nucleotide, such as a single nucleotide polymorphism (SNP), variation, or (point) mutation. The CRISPR effector may have reduced sensitivity to SNPs (or other single nucleotide variations) and continue to cleave SNP targets with a certain level of efficiency. Thus, for two targets, or a set of targets, a guide RNA may be designed with a nucleotide sequence that is complementary to one of the targets i.e., the on-target SNP. The guide RNA is further designed to have a synthetic mismatch. As used herein a “synthetic mismatch” refers to a non-naturally occurring mismatch that is introduced upstream or downstream of the naturally occurring SNP, such as at most 5 nucleotides upstream or downstream, for instance 4, 3, 2, or 1 nucleotide upstream or downstream, preferably at most 3 nucleotides upstream or downstream, more preferably at most 2 nucleotides upstream or downstream, most preferably 1 nucleotide upstream or downstream (i.e. adjacent the SNP). When the CRISPR effector binds to the on-target SNP, only a single mismatch will be formed with the synthetic mismatch and the CRISPR effector will continue to be activated and a detectable signal produced. When the guide RNA hybridizes to an off-target SNP, two mismatches will be formed, the mismatch from the SNP and the synthetic mismatch, and no detectable signal generated. Thus, the systems disclosed herein may be designed to distinguish SNPs within a population. For, example the systems may be used to distinguish pathogenic strains that differ by a single SNP or detect certain disease specific SNPs, such as but not limited to, disease associated SNPs, such as without limitation cancer associated SNPs, viral associated SNPs.

In certain embodiments, the guide RNA is designed such that the SNP is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the SNP is located on position 1, 2, 3, 4, 5, 6, 7, 8, or 9 of the spacer sequence (starting at the 5′ end). In certain embodiments, the guide RNA is designed such that the SNP is located on position 2, 3, 4, 5, 6, or 7 of the spacer sequence (starting at the 5′ end). In certain embodiments, the guide RNA is designed such that the SNP is located on position 3, 4, 5, or 6 of the spacer sequence (starting at the 5′ end). In certain embodiments, the guide RNA is designed such that the SNP is located on position 3 of the spacer sequence (starting at the 5′ end).

In certain embodiments, the guide RNA is designed such that the mismatch (e.g.the synthetic mismatch, i.e. an additional mutation besides a SNP) is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the mismatch is located on position 1, 2, 3, 4, 5, 6, 7, 8, or 9 of the spacer sequence (starting at the 5′ end). In certain embodiments, the guide RNA is designed such that the mismatch is located on position 4, 5, 6, or 7 of the spacer sequence (starting at the 5′ end. In certain embodiments, the guide RNA is designed such that the mismatch is located on position 5 of the spacer sequence (starting at the 5′ end).

In certain embodiments, the guide RNA is designed such that the mismatch is located 2 nucleotides upstream of the SNP (i.e., one intervening nucleotide).

In certain embodiments, the guide RNA is designed such that the mismatch is located 2 nucleotides downstream of the SNP (i.e., one intervening nucleotide).

In certain embodiments, the guide RNA is designed such that the mismatch is located on position 5 of the spacer sequence (starting at the 5′ end) and the SNP is located on position 3 of the spacer sequence (starting at the 5′ end).

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 6 shows several Cas polypeptides and the PAM sequence they recognize.

TABLE 6
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Viral Polynucleotide Preparation Formulation

In some embodiments, the composition, device, methods and the like described herein can include a sample preparation formulation. The sample preparation formulation is also referred to as a viral polynucleotide preparation formulation in the context of methods for detection of viral polynucleotides. The sample preparation formulation can include one or more reagents, active agents, buffers, etc., that can facilitate sample preparation and/or one or more other downstream reactions (such as target amplification, detection, and/or signal amplification). The sample preparation formulation can be formulated to facilitate storage of the one or more reagents and/or samples until one or more downstream reactions are employed (such as target amplification, detection, and/or signal amplification) In some embodiments, the sample preparation formulation is formulated for lyophilization, freeze drying, or other desiccation or other preservation technique. In some embodiments, the sample preparation formulation is lyophilized. The lyophilized sample preparation formulation can be reconstituted during use. In some embodiments, when the sample is a fluid, the fluid is sufficient to reconstitute the sample preparation formulation. In some embodiments, additional fluid can be added to the sample and the lyophilized sample preparation formulation to fully reconstitute the sample preparation formulation. In some embodiments, the sample preparation formulation is formulated to stabilize one or more of the compositions therein and/or sample therein. In some embodiments, the sample preparation formulation can be formulated such that it can be stored for a period of time. In some embodiments a lyophilized, freeze dried or otherwise de

In some embodiments, the sample preparation formulation is formulated such that the sample preparation formulation can be stored for 1, 2, 3, 4, 5, 6, 7, 8, 9, 10, 11, 12 or more days, weeks, months, or years prior to introduction of a sample or use in a downstream reaction, such as (such as target amplification, detection, and/or signal amplification). In some embodiments, the sample preparation formulation is formulated such that the sample preparation formulation can be stored for 1, 2, 3, 4, 5, 6, 7, 8, 9, 10, 11, 12 or more days, weeks, months, or years prior to introduction of a sample or use in a downstream reaction, such as (such as target amplification, detection, and/or signal amplification), while maintaining assay fidelity and integrity. The sample preparation formulation can be formulated to lyse a cell and/or virus of sample.

The sample preparation formulation can include, without limitation, water, solvents, enzymes (e.g., DNase, RNAse, DNase inhibitors, RNAse inhibitors, protein inhibitors, etc.), pH buffers, salts, other active agents, stabilizers (e.g., DNA and/or RNA stabilizes), and combinations thereof.

The sample preparation formulation can be liquid. The sample preparation formulation can be solid (such as lyophilized, freeze dried, or otherwise desiccated or dehydrated) that can be rehydrated upon introduction of a sample or other hydration liquid. The sample preparation formulation can be contained in a reaction vessel, reaction area or location, and/or device, such as any described elsewhere herein.

In some embodiments, the sample preparation formulation is formulated to carry out one or more sample preparation steps at a temperature ranging from about 15-95 degrees C. In some embodiments, the sample preparation formulation is configured to carry out one or more sample preparation steps at a temperature ranging about 15-50 degrees C., 15-37 degrees C., 15-30 degrees C., 15-25 degrees C., or about 22-25 degrees C. In some embodiments, the sample preparation formulation is formulated to carry out one or more sample preparation steps at a temperature of less than 65 degrees C., less than 55 degrees C., less than 50 degrees C., less than 45 degrees C., less than 40 degrees C., less than 37 degrees C., less than 35 degrees C., less than 30 degrees C., less than 25 degrees C. or less than 20 degrees C. Other features of the sample preparation formulation are described elsewhere herein.

### Detection Constructs

The systems and methods described herein comprise a detection construct. As used herein, a “detection construct” refers to a molecule that can be cleaved or otherwise deactivated by an activated CRISPR system effector protein described herein. Depending on the nuclease activity of the CRISPR effector protein, the detection construct may be an RNA-based detection construct or a DNA-based detection construct. The Nucleic Acid-based detection construct comprises a nucleic acid element that is cleavable by a CRISPR effector protein. Cleavage of the nucleic acid element releases agents or produces conformational changes that allow a detectable signal to be produced. Example constructs demonstrating how the nucleic acid element may be used to prevent or mask generation of detectable signal are described below and embodiments of the invention comprise variants of the same. Prior to cleavage, or when the detection construct is in an ‘active’ state, the detection construct blocks the generation or detection of a positive detectable signal. In certain embodiments, detection constructs are designed for cutting motifs of particular Cas proteins. See, International Publication WO 2019/126577, incorporated herein by reference in its entirety, and specifically paragraphs [00314]-[00356], Table 25, and Examples 8-10, for teaching of design of detection constructs for Cas proteins with preferred cutting motifs. For example, when AapCas12b is used, a reporter designed with A and T bases can be utilized because of preferred cleavage specificity. In an aspect, a reporter comprising sequence TTTTTTT is utilized with AapCas12b systems. In embodiments, the reporter comprises a AAAAA sequence or a TTTTT sequence. In an aspect, the reporter is selected from WCV328, WCV329, WCV333. The reporter can be selected from WCV0333/5HEX/TTTTTTT/3IABkFQ/homopolymer hex probe, WCV0328/5HEX/AAAAA/3IABkFQ/homopolymer hex probe, and WCV0329/5HEX/TTTTT/3IABkFQ/homopolymer hex probe.

It will be understood that in certain example embodiments a minimal background signal may be produced in the presence of an active detection construct. A positive detectable signal may be any signal that can be detected using optical, fluorescent, chemiluminescent, electrochemical or other detection methods known in the art. The term “positive detectable signal” is used to differentiate from other detectable signals that may be detectable in the presence of the detection construct. For example, in certain embodiments a first signal may be detected when the masking agent is present or when a CRISPR system has not been activated (i.e. a negative detectable signal), which then converts to a second signal (e.g. the positive detectable signal) upon detection of the target molecules and cleavage or deactivation of the masking agent, or upon activation of the CRISPR effector protein. The positive detectable signal, then, is a signal detected upon activation of the CRISPR effector protein, and may be, in a colorimetric or fluorescent assay, a decrease in fluorescence or color relative to a control or an increase in fluorescence or color relative to a control, depending on the configuration of the lateral flow substrate, and as described further herein.

In certain example embodiments, the detection construct may comprise a HCR initiator sequence and a cutting motif, or a cleavable structural element, such as a loop or hairpin, that prevents the initiator from initiating the HCR reaction. The cutting motif may be preferentially cut by one of the activated CRISPR effector proteins. Upon cleavage of the cutting motif or structure element by an activated CRISPR effector protein, the initiator is then released to trigger the HCR reaction, detection thereof indicating the presence of one or more targets in the sample. In certain example embodiments, the detection construct comprises a hairpin with a RNA loop. When an activated CRISPR effector protein cuts the RNA loop, the initiator can be released to trigger the HCR reaction.

In certain example embodiments, the detection construct may suppress generation of a gene product. The gene product may be encoded by a reporter construct that is added to the sample. The detection construct may be an interfering RNA involved in a RNA interference pathway, such as a short hairpin RNA (shRNA) or small interfering RNA (siRNA). The detection construct may also comprise microRNA (miRNA). While present, the detection construct suppresses expression of the gene product. The gene product may be a fluorescent protein or other RNA transcript or proteins that would otherwise be detectable by a labeled probe, aptamer, or antibody but for the presence of the detection construct. Upon activation of the effector protein the detection construct is cleaved or otherwise silenced allowing for expression and detection of the gene product as the positive detectable signal. In preferred embodiments, the detection construct comprise two or more detectable signals, for example, fluorescent signals, that can be read on different channels of a fluorimeter.

In specific embodiments, the detection construct comprises a silencing RNA that suppresses generation of a gene product encoded by a reporting construct, wherein the gene product generates the detectable positive signal when expressed.

In certain example embodiments, the detection construct may sequester one or more reagents needed to generate a detectable positive signal such that release of the one or more reagents from the detection construct results in generation of the detectable positive signal. The one or more reagents may combine to produce a colorimetric signal, a chemiluminescent signal, a fluorescent signal, or any other detectable signal and may comprise any reagents known to be suitable for such purposes. In certain example embodiments, the one or more reagents are sequestered by RNA aptamers that bind the one or more reagents. The one or more reagents are released when the effector protein is activated upon detection of a target molecule and the RNA or DNA aptamers are degraded.

In certain example embodiments, the detection construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the detection construct are able to generate a detectable signal, for example by aggregation or simple increase in solution concentration. In certain example embodiments, the immobilized masking agent is a RNA- or DNA-based aptamer that can be cleaved by the activated effector protein upon detection of a target molecule.

In certain other example embodiments, the detection construct binds to an immobilized reagent in solution thereby blocking the ability of the reagent to bind to a separate labeled binding partner that is free in solution. Thus, upon application of a washing step to a sample, the labeled binding partner can be washed out of the sample in the absence of a target molecule. However, if the effector protein is activated, the detection construct is cleaved to a degree sufficient to interfere with the ability of the detection construct to bind the reagent thereby allowing the labeled binding partner to bind to the immobilized reagent. Thus, the labeled binding partner remains after the wash step indicating the presence of the target molecule in the sample. In certain aspects, the detection construct that binds the immobilized reagent is a DNA or RNA aptamer. The immobilized reagent may be a protein and the labeled binding partner may be a labeled antibody. Alternatively, the immobilized reagent may be streptavidin and the labeled binding partner may be labeled biotin. The label on the binding partner used in the above embodiments may be any detectable label known in the art. In addition, other known binding partners may be used in accordance with the overall design described herein.

In certain example embodiments, the detection construct may comprise a ribozyme. Ribozymes are RNA molecules having catalytic properties. Ribozymes, both naturally and engineered, comprise or consist of RNA that may be targeted by the effector proteins disclosed herein. The ribozyme may be selected or engineered to catalyze a reaction that either generates a negative detectable signal or prevents generation of a positive control signal. Upon deactivation of the ribozyme by the activated effector protein the reaction generating a negative control signal, or preventing generation of a positive detectable signal, is removed thereby allowing a positive detectable signal to be generated. In one example embodiment, the ribozyme may catalyze a colorimetric reaction causing a solution to appear as a first color. When the ribozyme is deactivated the solution then turns to a second color, the second color being the detectable positive signal. An example of how ribozymes can be used to catalyze a colorimetric reaction are described in Zhao et al. “Signal amplification of glucosamine-6-phosphate based on ribozyme glmS,” Biosens Bioelectron. 2014; 16:337-42, and provide an example of how such a system could be modified to work in the context of the embodiments disclosed herein. Alternatively, ribozymes, when present can generate cleavage products of, for example, RNA transcripts. Thus, detection of a positive detectable signal may comprise detection of non-cleaved RNA transcripts that are only generated in the absence of the ribozyme.

In some embodiments, the detection construct may be a ribozyme that generates a negative detectable signal, and wherein a positive detectable signal is generated when the ribozyme is deactivated.

In certain example embodiments, the one or more reagents is a protein, such as an enzyme, capable of facilitating generation of a detectable signal, such as a colorimetric, chemiluminescent, or fluorescent signal, that is inhibited or sequestered such that the protein cannot generate the detectable signal by the binding of one or more DNA or RNA aptamers to the protein. Upon activation of the effector proteins disclosed herein, the DNA or RNA aptamers are cleaved or degraded to an extent that they no longer inhibit the protein's ability to generate the detectable signal. In certain example embodiments, the aptamer is a thrombin inhibitor aptamer. In certain example embodiments the thrombin inhibitor aptamer has a sequence of GGGAACAAAGCUGAAGUACUUACCC (SEQ ID NO: 9). When this aptamer is cleaved, thrombin will become active and will cleave a peptide colorimetric or fluorescent substrate. In certain example embodiments, the colorimetric substrate is para-nitroanilide (pNA) covalently linked to the peptide substrate for thrombin. Upon cleavage by thrombin, pNA is released and becomes yellow in color and easily visible to the eye. In certain example embodiments, the fluorescent substrate is 7-amino-4-methylcoumarin a blue fluorophore that can be detected using a fluorescence detector. Inhibitory aptamers may also be used for horseradish peroxidase (HRP), beta-galactosidase, or calf alkaline phosphatase (CAP) and within the general principals laid out above.

In certain embodiments, RNAse or DNAse activity is detected colorimetrically via cleavage of enzyme-inhibiting aptamers. One potential mode of converting DNAse or RNAse activity into a colorimetric signal is to couple the cleavage of a DNA or RNA aptamer with the re-activation of an enzyme that is capable of producing a colorimetric output. In the absence of RNA or DNA cleavage, the intact aptamer will bind to the enzyme target and inhibit its activity. The advantage of this readout system is that the enzyme provides an additional amplification step: once liberated from an aptamer via collateral activity (e.g. Cpf1 collateral activity), the colorimetric enzyme will continue to produce colorimetric product, leading to a multiplication of signal.

In certain embodiments, an existing aptamer that inhibits an enzyme with a colorimetric readout is used. Several aptamer/enzyme pairs with colorimetric readouts exist, such as thrombin, protein C, neutrophil elastase, and subtilisin. These proteases have colorimetric substrates based upon pNA and are commercially available. In certain embodiments, a novel aptamer targeting a common colorimetric enzyme is used. Common and robust enzymes, such as beta-galactosidase, horseradish peroxidase, or calf intestinal alkaline phosphatase, could be targeted by engineered aptamers designed by selection strategies such as SELEX. Such strategies allow for quick selection of aptamers with nanomolar binding efficiencies and could be used for the development of additional enzyme/aptamer pairs for colorimetric readout.

In certain embodiments, the detection construct may be a DNA or RNA aptamer and/or may comprise a DNA or RNA-tethered inhibitor.

In certain embodiments, the detection construct may comprise a DNA or RNA oligonucleotide to which a detectable ligand and a masking component are attached.

In certain embodiments, RNAse or DNase activity is detected colorimetrically via cleavage of RNA-tethered inhibitors. Many common colorimetric enzymes have competitive, reversible inhibitors: for example, beta-galactosidase can be inhibited by galactose. Many of these inhibitors are weak, but their effect can be increased by increases in local concentration. By linking local concentration of inhibitors to DNase RNAse activity, colorimetric enzyme and inhibitor pairs can be engineered into DNase and RNAse sensors. The colorimetric DNase or RNAse sensor based upon small-molecule inhibitors involves three components: the colorimetric enzyme, the inhibitor, and a bridging RNA or DNA that is covalently linked to both the inhibitor and enzyme, tethering the inhibitor to the enzyme. In the uncleaved configuration, the enzyme is inhibited by the increased local concentration of the small molecule; when the DNA or RNA is cleaved (e.g., by Cas13 or Cas12 collateral cleavage), the inhibitor will be released and the colorimetric enzyme will be activated.

In certain embodiments, the aptamer or DNA- or RNA-tethered inhibitor may sequester an enzyme, wherein the enzyme generates a detectable signal upon release from the aptamer or DNA or RNA tethered inhibitor by acting upon a substrate. In some embodiments, the aptamer may be an inhibitor aptamer that inhibits an enzyme and prevents the enzyme from catalyzing generation of a detectable signal from a substance. In some embodiments, the DNA- or RNA-tethered inhibitor may inhibit an enzyme and may prevent the enzyme from catalyzing generation of a detectable signal from a substrate.

In certain embodiments, RNAse activity is detected colorimetrically via formation and/or activation of G-quadruplexes. G quadruplexes in DNA can complex with heme (iron (III)-protoporphyrin IX) to form a DNAzyme with peroxidase activity. When supplied with a peroxidase substrate (e.g., ABTS: (2,2′-Azinobis [3-ethylbenzothiazoline-6-sulfonic acid]-diammonium salt)), the G-quadruplex-heme complex in the presence of hydrogen peroxide causes oxidation of the substrate, which then forms a green color in solution. An example G-quadruplex forming DNA sequence is: GGGTAGGGCGGGTTGGGA (SEQ ID NO: 10). By hybridizing an additional DNA or RNA sequence, referred to herein as a “staple,” to this DNA aptamer, formation of the G-quadraplex structure will be limited. Upon collateral activation, the staple will be cleaved allowing the G quadraplex to form and heme to bind. This strategy is particularly appealing because color formation is enzymatic, meaning there is additional amplification beyond collateral activation.

In certain embodiments, the detection construct may comprise an RNA oligonucleotide designed to bind a G-quadruplex forming sequence, wherein a G-quadruplex structure is formed by the G-quadruplex forming sequence upon cleavage of the detection construct, and wherein the G-quadruplex structure generates a detectable positive signal.

In certain example embodiments, the detection construct may be immobilized on a solid substrate in an individual discrete volume (defined further below) and sequesters a single reagent. For example, the reagent may be a bead comprising a dye. When sequestered by the immobilized reagent, the individual beads are too diffuse to generate a detectable signal, but upon release from the detection construct are able to generate a detectable signal, for example by aggregation or simple increase in solution concentration. In certain example embodiments, the immobilized masking agent is a DNA- or RNA-based aptamer that can be cleaved by the activated effector protein upon detection of a target molecule.

In one example embodiment, the detection construct comprises a detection agent that changes color depending on whether the detection agent is aggregated or dispersed in solution. For example, certain nanoparticles, such as colloidal gold, undergo a visible purple to red color shift as they move from aggregates to dispersed particles. Accordingly, in certain example embodiments, such detection agents may be held in aggregate by one or more bridge molecules. At least a portion of the bridge molecule comprises RNA or DNA. Upon activation of the effector proteins disclosed herein, the RNA or DNA portion of the bridge molecule is cleaved allowing the detection agent to disperse and resulting in the corresponding change in color. In certain example embodiments, the detection agent is a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A13+, Ru3+, Zn2+, Fe3+, Ni2+ and Ca2+ ions.

When the RNA or DNA bridge is cut by the activated CRISPR effector, the aforementioned color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate. In certain example embodiments the nanoparticles are modified to include DNA linkers extending from the surface of the nanoparticle. Individual particles are linked together by single-stranded RNA (ssRNA) or single-stranded DNA bridges that hybridize on each end to at least a portion of the DNA linkers. Thus, the nanoparticles will form a web of linked particles and aggregate, appearing as a dark precipitate. Upon activation of the CRISPR effectors disclosed herein, the ssRNA or ssDNA bridge will be cleaved, releasing the AU NPS from the linked mesh and producing a visible red color. Example DNA linkers and bridge sequences are listed below. Thiol linkers on the end of the DNA linkers may be used for surface conjugation to the AuNPS. Other forms of conjugation may be used. In certain example embodiments, two populations of AuNPs may be generated, one for each DNA linker. This will help facilitate proper binding of the ssRNA bridge with proper orientation. In certain example embodiments, a first DNA linker is conjugated by the 3′ end while a second DNA linker is conjugated by the 5′ end.

In certain other example embodiments, the detection construct may comprise an RNA or DNA oligonucleotide to which are attached a detectable label and a masking agent of that detectable label. An example of such a detectable label/masking agent pair is a fluorophore and a quencher of the fluorophore. Quenching of the fluorophore can occur as a result of the formation of a non-fluorescent complex between the fluorophore and another fluorophore or non-fluorescent molecule. This mechanism is known as ground-state complex formation, static quenching, or contact quenching. Accordingly, the RNA or DNA oligonucleotide may be designed so that the fluorophore and quencher are in sufficient proximity for contact quenching to occur. Fluorophores and their cognate quenchers are known in the art and can be selected for this purpose by one having ordinary skill in the art. The particular fluorophore/quencher pair is not critical in the context of this invention, only that selection of the fluorophore/quencher pairs ensures masking of the fluorophore. Upon activation of the effector proteins disclosed herein, the RNA or DNA oligonucleotide is cleaved thereby severing the proximity between the fluorophore and quencher needed to maintain the contact quenching effect. Accordingly, detection of the fluorophore may be used to determine the presence of a target molecule in a sample.

In certain other example embodiments, the detection construct may comprise one or more RNA oligonucleotides to which are attached one or more metal nanoparticles, such as gold nanoparticles. In some embodiments, the detection construct comprises a plurality of metal nanoparticles crosslinked by a plurality of RNA or DNA oligonucleotides forming a closed loop. In one embodiment, the v comprises three gold nanoparticles crosslinked by three RNA or DNA oligonucleotides forming a closed loop. In some embodiments, the cleavage of the RNA or DNA oligonucleotides by the CRISPR effector protein leads to a detectable signal produced by the metal nanoparticles.

In certain other example embodiments, the detection construct may comprise one or more RNA or DNA oligonucleotides to which are attached one or more quantum dots. In some embodiments, the cleavage of the RNA or DNA oligonucleotides by the CRISPR effector protein leads to a detectable signal produced by the quantum dots.

In one example embodiment, the detection construct may comprise a quantum dot. The quantum dot may have multiple linker molecules attached to the surface. At least a portion of the linker molecule comprises RNA or DNA. The linker molecule is attached to the quantum dot at one end and to one or more quenchers along the length or at terminal ends of the linker such that the quenchers are maintained in sufficient proximity for quenching of the quantum dot to occur. The linker may be branched. As above, the quantum dot/quencher pair is not critical, only that selection of the quantum dot/quencher pair ensures masking of the fluorophore. Quantum dots and their cognate quenchers are known in the art and can be selected for this purpose by one having ordinary skill in the art. Upon activation of the effector proteins disclosed herein, the RNA or DNA portion of the linker molecule is cleaved thereby eliminating the proximity between the quantum dot and one or more quenchers needed to maintain the quenching effect. In certain example embodiments the quantum dot is streptavidin conjugated. RNA or DNA are attached via biotin linkers and recruit quenching molecules with the sequences/5Biosg/UCUCGUACGUUC/3IAbRQSp/(SEQ ID NO: 11) or /5Biosg/UCUCGUACGUUCUCUCGUACGUUC/3IAbRQSp/(SEQ ID NO: 12) where /5Biosg/is a biotin tag and/31AbRQSp/is an Iowa black quencher (Iowa Black FQ). Upon cleavage, by the activated effectors disclosed herein the quantum dot will fluoresce visibly.

In specific embodiments, the detectable ligand may be a fluorophore and the masking component may be a quencher molecule.

In a similar fashion, fluorescence energy transfer (FRET) may be used to generate a detectable positive signal. FRET is a non-radiative process by which a photon from an energetically excited fluorophore (i.e. “donor fluorophore”) raises the energy state of an electron in another molecule (i.e. “the acceptor”) to higher vibrational levels of the excited singlet state. The donor fluorophore returns to the ground state without emitting a fluoresce characteristic of that fluorophore. The acceptor can be another fluorophore or non-fluorescent molecule. If the acceptor is a fluorophore, the transferred energy is emitted as fluorescence characteristic of that fluorophore. If the acceptor is a non-fluorescent molecule the absorbed energy is loss as heat. Thus, in the context of the embodiments disclosed herein, the fluorophore/quencher pair is replaced with a donor fluorophore/acceptor pair attached to the oligonucleotide molecule. When intact, the detection construct generates a first signal (negative detectable signal) as detected by the fluorescence or heat emitted from the acceptor. Upon activation of the effector proteins disclosed herein the RNA oligonucleotide is cleaved and FRET is disrupted such that fluorescence of the donor fluorophore is now detected (positive detectable signal).

In certain example embodiments, the detection construct comprises the use of intercalating dyes which change their absorbance in response to cleavage of long RNAs or DNAs to short nucleotides. Several such dyes exist. For example, pyronine-Y will complex with RNA and form a complex that has an absorbance at 572 nm. Cleavage of the RNA results in loss of absorbance and a color change. Methylene blue may be used in a similar fashion, with changes in absorbance at 688 nm upon RNA cleavage. Accordingly, in certain example embodiments the detection construct comprises a RNA and intercalating dye complex that changes absorbance upon the cleavage of RNA by the effector proteins disclosed herein.

In certain example embodiments, the detection construct may comprise an initiator for an HCR reaction. See e.g., Dirks and Pierce. PNAS 101, 15275-15728 (2004). HCR reactions utilize the potential energy in two hairpin species. When a single-stranded initiator having a portion of complementary to a corresponding region on one of the hairpins is released into the previously stable mixture, it opens a hairpin of one speces. This process, in turn, exposes a single-stranded region that opens a hairpin of the other species. This process, in turn, exposes a single stranded region identical to the original initiator. The resulting chain reaction may lead to the formation of a nicked double helix that grows until the hairpin supply is exhausted. Detection of the resulting products may be done on a gel or colorimetrically. Example colorimetric detection methods include, for example, those disclosed in Lu et al. “Ultra-sensitive colorimetric assay system based on the hybridization chain reaction-triggered enzyme cascade amplification ACS Appl Mater Interfaces, 2017, 9(1):167-175, Wang et al. “An enzyme-free colorimetric assay using hybridization chain reaction amplification and split aptamers” Analyst 2015, 150, 7657-7662, and Song et al. “Non-covalent fluorescent labeling of hairpin DNA probe coupled with hybridization chain reaction for sensitive DNA detection.” Applied Spectroscopy, 70(4): 686-694 (2016).

In certain example embodiments, the detection construct suppresses generation of a detectable positive signal until cleaved or modified by an activated CRISPR effector protein. In some embodiments, the detection construct may suppress generation of a detectable positive signal by masking the detectable positive signal or generating a detectable negative signal instead.

### Encoding Polynucleotides and Vectors and Delivery

Any of the polypeptides described here and elsewhere herein, including but not limited to any one or more of those of the CRISPR systems or component thereof (e.g., a Cas polypeptide or guide molecule) described herein, can be encoded by one or more polynucleotides. In some embodiments, the polynucleotide encodes a Cas12 or a Cas13 polypeptide. In some embodiments, the encoding polynucleotide is codon optimized for expression in a host cell. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59. In some embodiments, a polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

The encoding polynucleotides and other polynucleotides (e.g., a guide polynucleotide) can be included in one or more vectors of a vector system. The vector system can be used to express one or more of the polypeptides that are described herein which can then be subsequently harvested and used as desired and/or produce delivery particles (e.g. viral or other particles) to facilitate delivery of the polynucleotide to a cell.

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties. Delivery can include a physical delivery method (e.g., microinjection, electroporation, hydrodynamic delivery, transfection, transduction, biolistics, implantable deivices, and/or the like), delivery by a delivery vehicle (e.g., nanoparticles (e.g., polymeric, metal, lipid, self-assembling, inorganic, and/or the like), exosomes, liposomes, micelles, viral and non-viral vector systems, viral particles, virus like particles, cell penetrating peptides, nanoclews, sugar particles, lipid particles, ribonucleoprotein complexs, iTOP, streptolysin 0, spherical nucleic acids, supercharged proteins, and the like). Delivery can be targeted, such as to aspecific cell type. Targeing moietiys and targeting strategies are generally known in the art and can be applied to the delivery systems described herein. Delivery can be responsive or otherwise controllable. Strategies for responsive and/or controllable delivery are generally known in the the art and can be applied to the delivery systems described herein.

In some embodiments, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9(1):11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M R et al., Plant Mol Biol. 1989 September; 13(3):273-85, which are incorporated by reference herein in their entireties.

The delivery systems may comprise one or more cargos. The cargos may comprise one or more components of the CRISPR-Cas systems and compositions herein. A cargo may comprise one or more of the following: i) a vector or vector system (viral or non-viral) encoding one or more Cas proteins; ii) a vector or vector system (viral or non-viral) encoding one or more guide RNAs described herein, iii) mRNA of one or more Cas proteins; iv) one or more guide RNAs; v) one or more Cas proteins; vi) one or more polynucleotides encoding one or more Cas proteins; vii) one or more polynucleotides encoding one or more guide RNAs, or viii) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNA.

In some embodiments, a cargo may comprise one or more Cas proteins described herein and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November; 33(11):1162-4.

In some embodiments, the cargo(s) can be any of the polynucleotide(s), e.g., CRISPR-Cas System polynucleotides described herein.

### Devices

The assays or component thereof can be carried out on a device, such as tube, capillary, lateral flow strip, chip, cartridge or another device. The systems and/or assays described herein can be embodied on diagnostic devices. Devices can include very simple devices such as tubes for containing a single sample that contains all the reagents necessary to carry out a CRISPR-Cas collateral acivity reaction described herein and provide a result (such as a colometric, turbidity shift, or fluorescent signal) all within the single tube. Other devices can be complex fully automated devices that are capable of handling tens to thousands of samples at time. As is described in greater detail elsewhere herein, one or more compositions (e.g., sample preparation, target amplification reaction, and/or CRISPR-Cas collateral activity detection reagents) can be included in the device. In some embodiments, they are included in one or more compartments and/or locations within the device in a free-dried, lyophilized or some other form. Devices can contain or be configured for optical-based readouts, lateral flow readouts, electrical readouts or others that are described herein and will be appreciated in view of the description provided herein.

### Discrete Volumes

In some embodiments the devices can include individual discrete volumes. In certain embodiments, the CRISPR effector protein is bound to each discrete volume in the device. Each discrete volume may comprise a different guide RNA specific for a different target molecule. In certain embodiments, a sample is exposed to a solid substrate comprising more than one discrete volume each comprising a guide RNA specific for a target molecule. Not being bound by a theory, each guide RNA will capture its target molecule from the sample and the sample does not need to be divided into separate assays. Thus, a valuable sample may be preserved. The effector protein may be a fusion protein comprising an affinity tag. Affinity tags are well known in the art (e.g., HA tag, Myc tag, Flag tag, His tag, biotin). The effector protein may be linked to a biotin molecule and the discrete volumes may comprise streptavidin. In other embodiments, the CRISPR effector protein is bound by an antibody specific for the effector protein. Methods of binding a CRISPR enzyme has been described previously (see, e.g., US20140356867A1).

Several substrates and configurations of devices capable of defining multiple individual discrete volumes within the device may be used. As used herein “individual discrete volume” refers to a discrete space, such as a container, receptacle, or other arbitrary defined volume or space that can be defined by properties that prevent and/or inhibit migration of target molecules, for example a volume or space defined by physical properties such as walls, for example the walls of a well, tube, or a surface of a droplet, which may be impermeable or semipermeable, or as defined by other means such as chemical, diffusion rate limited, electro-magnetic, or light illumination, or any combination thereof that can contain a target molecule and a indexable nucleic acid identifier (for example nucleic acid barcode). By “diffusion rate limited” (for example diffusion defined volumes) is meant spaces that are only accessible to certain molecules or reactions because diffusion constraints effectively defining a space or volume as would be the case for two parallel laminar streams where diffusion will limit the migration of a target molecule from one stream to the other. By “chemical” defined volume or space is meant spaces where only certain target molecules can exist because of their chemical or molecular properties, such as size, where for example gel beads may exclude certain species from entering the beads but not others, such as by surface charge, matrix size or other physical property of the bead that can allow selection of species that may enter the interior of the bead. By “electro-magnetically” defined volume or space is meant spaces where the electro-magnetic properties of the target molecules or their supports such as charge or magnetic properties can be used to define certain regions in a space such as capturing magnetic particles within a magnetic field or directly on magnets. By “optically” defined volume is meant any region of space that may be defined by illuminating it with visible, ultraviolet, infrared, or other wavelengths of light such that only target molecules within the defined space or volume may be labeled. One advantage to the use of non-walled, or semipermeable discrete volumes is that some reagents, such as buffers, chemical activators, or other agents may be passed through the discrete volume, while other materials, such as target molecules, may be maintained in the discrete volume or space. Typically, a discrete volume will include a fluid medium, (for example, an aqueous solution, an oil, a buffer, and/or a media capable of supporting cell growth) suitable for labeling of the target molecule with the indexable nucleic acid identifier under conditions that permit labeling. Exemplary discrete volumes or spaces useful in the disclosed methods include droplets (for example, microfluidic droplets and/or emulsion droplets), hydrogel beads or other polymer structures (for example poly-ethylene glycol di-acrylate beads or agarose beads), tissue slides (for example, fixed formalin paraffin embedded tissue slides with particular regions, volumes, or spaces defined by chemical, optical, or physical means), microscope slides with regions defined by depositing reagents in ordered arrays or random patterns, tubes (such as, centrifuge tubes, microcentrifuge tubes, test tubes, cuvettes, conical tubes, and the like), bottles (such as glass bottles, plastic bottles, ceramic bottles, Erlenmeyer flasks, scintillation vials and the like), wells (such as wells in a plate), plates, pipettes, or pipette tips among others. In certain embodiments, the compartment is an aqueous droplet in a water-in-oil emulsion. In specific embodiments, any of the applications, methods, or systems described herein requiring exact or uniform volumes may employ the use of an acoustic liquid dispenser.

### Samples

The device can be configured to hold, store, collect, receive, process and/or otherwise manipulate a sample and/or detect a component thereof. In some embodiments, the sample is a solid, semisolid, or liquid. In some embodiments, the sample is a biological sample. In some embodiments, the sample is obtained from a subject. In some embodiments, the sample is a bodily fluid. In some embodiments, the bodily fluid is saliva or nasal secretions. In some embodiments, the sample is not a bodily fluid but contains one or more cells from the subject, such as hair cells, skin cells, solid tissue or tumor cells. In some embodiments, the sample is obtained from a plant. In some embodiments, the sample is an environmental sample, such as air, soil, water, or a sample of molecules, organisms, viruses, and other particles present on an object surface. In some embodiments, the sample is a feedstuff or foodstuff or component thereof. Other exemplary samples that may be analyzed using the systems and devices described herein include biological samples of a subject or environmental samples. Environmental samples may include surfaces or fluids. The biological samples may include, but are not limited to, saliva, blood, plasma, sera, stool, urine, sputum, mucous, lymph, synovial fluid, spinal fluid, cerebrospinal fluid, a swab from skin or a mucosal membrane, or combination thereof. In an example embodiment, the environmental sample is taken from a solid surface, such as a surface used in the preparation of food or other sensitive compositions and materials.

A sample for use with the invention may be a biological or environmental sample, such as a surface sample, a fluid sample, or a food sample (fresh fruits or vegetables, meats). Food samples may include a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, spinal fluid, cerebrospinal fluid, ascites, pleural effusion, seroma, pus, bile, aqueous or vitreous humor, transudate, exudate, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In particular embodiments, the methods and systems can be utilized for direct detection from patient samples. In an aspect, the methods and systems can further allow for direct detection from patient samples with a visual readout to further facilitate field-deployability. In an aspect, a field depoloyable version can include, for example the lateral flow devices and systems as described herein, and/or colorimetric detection. The methods and systems can be utilized to distinguish multipe viral species and strains and identify clinically relevant mutations, important with viral outbreaks such as the coronavirus outbreak in Wuhan (2019-nCoV). In an aspect, the sample is from a nasophyringeal swab or a saliva sample. See., e.g. FIG. 40, see also, Wyllie et al., “Saliva is more sensitive for SARS-CoV-2 detection in COVID-19 patients than nasopharyngeal swabs,” DOI: 10.1101/2020.04.16.20067835.

### Flexible Substrates

In certain example embodiments, the device comprises a flexible material substrate on which a number of spots or discrete volumes may be defined. Flexible substrate materials suitable for use in diagnostics and biosensing are known within the art. The flexible substrate materials may be made of plant derived fibers, such as cellulosic fibers, or may be made from flexible polymers such as flexible polyester films and other polymer types. Within each defined spot, reagents of the system described herein are applied to the individual spots. Each spot may contain the same reagents except for a different guide RNA or set of guide RNAs, or where applicable, a different detection aptamer to screen for multiple targets at once. Thus, the systems and devices herein may be able to screen samples from multiple sources (e.g. multiple clinical samples from different individuals) for the presence of the same target, or a limited number of target, or aliquots of a single sample (or multiple samples from the same source) for the presence of multiple different targets in the sample. In certain example embodiments, the elements of the systems described herein are freeze dried onto the paper or cloth substrate. Example flexible material based substrates that may be used in certain example devices are disclosed in Pardee et al. Cell. 2016, 165(5):1255-66 and Pardee et al. Cell. 2014, 159(4):950-54. Suitable flexible material-based substrates for use with biological fluids, including blood are disclosed in International Patent Application Publication No. WO/2013/071301 entitled “Paper based diagnostic test” to Shevkoplyas et al. U.S. Patent Application Publication No. 2011/0111517 entitled “Paper-based microfluidic systems” to Siegel et al. and Shafiee et al. “Paper and Flexible Substrates as Materials for Biosensing Platforms to Detect Multiple Biotargets” Scientific Reports 5:8719 (2015). Further flexible based materials, including those suitable for use in wearable diagnostic devices are disclosed in Wang et al. “Flexible Substrate-Based Devices for Point-of-Care Diagnostics” Cell 34(11):909-21 (2016). Further flexible based materials may include nitrocellulose, polycarbonate, methylethyl cellulose, polyvinylidene fluoride (PVDF), polystyrene, or glass (see e.g., US20120238008). In certain embodiments, discrete volumes are separated by a hydrophobic surface, such as but not limited to wax, photoresist, or solid ink.

In some embodiments, the substrate, such as a flexible substrate, is a single use substrate, such as swab, strip, or cloth that is used to swab a surface or sample fluid or is placed in a prepared sample for detection by an assay described herein. For example, the system could be used to test for the presence of a pathogen on a food by swabbing the surface of a food product, such as a fruit or vegetable. Similarly, the single use substrate may be used to swab other surfaces for detection of certain microbes or agents, such as for use in security screening. Single use substrates may also have applications in forensics, where the CRISPR systems are designed to detect, for example identifying DNA SNPs that may be used to identify a suspect, or certain tissue or cell markers to determine the type of biological matter present in a sample. Likewise, the single use substrate could be used to collect a sample from a patient—such as a saliva sample from the mouth—or a swab of the skin. In other embodiments, a sample or swab may be taken of a meat product on order to detect the presence of absence of contaminants on or within the meat product.

### Microfluidic Devices

In certain example embodiments, the device is configured as a microfluidic device. It will be appreciated that the microfluidic device can incorporate a chip, cartridge, flexible substrate, lateral flow strip, and/or other components described elsewhere herien. In some embodimetns the microfluidic device can be configured to drive a sample through the device such that it contacts one or more CRISPR-Cas collateral cleavage detection reaction reagetns (such as those that may be present on a flexible substrate within the device) and thus carries out a CRISPR-Cas collateral cleavage detection reaction. In some emobdiments, the microfluidic device is configured to generate and/or merge different droplets (i.e., individual discrete volumes). For example, a first set of droplets may be formed containing samples to be screened and a second set of droplets formed containing the elements of the systems described herein. The first and second set of droplets are then merged and then diagnostic methods as described herein are carried out on the merged droplet set. Microfluidic devices disclosed herein may be silicone-based chips and may be fabricated using a variety of techniques, including, but not limited to, hot embossing, molding of elastomers, injection molding, LIGA, soft lithography, silicon fabrication and related thin film processing techniques. Suitable materials for fabricating the microfluidic devices include, but are not limited to, cyclic olefin copolymer (COC), polycarbonate, poly(dimethylsiloxane) (PDMS), and poly(methylacrylate) (PMMA). In one embodiment, soft lithography in PDMS may be used to prepare the microfluidic devices. For example, a mold may be made using photolithography which defines the location of flow channels, valves, and filters within a substrate. The substrate material is poured into a mold and allowed to set to create a stamp. The stamp is then sealed to a solid support, such as but not limited to, glass. Due to the hydrophobic nature of some polymers, such as PDMS, which absorbs some proteins and may inhibit certain biological processes, a passivating agent may be necessary (Schoffner et al. Nucleic Acids Research, 1996, 24:375-379). Suitable passivating agents are known in the art and include, but are not limited to, silanes, parylene, n-Dodecyl-b-D-matoside (DDM), pluronic, Tween-20, other similar surfactants, polyethylene glycol (PEG), albumin, collagen, and other similar proteins and peptides.

In certain example embodiments, the system and/or device may be adapted for conversion to a flow-cytometry readout in or allow to sensitive and quantitative measurements of millions of cells in a single experiment and improve upon existing flow-based methods, such as the PrimeFlow assay. In certain example embodiments, cells may be cast in droplets containing unpolymerized gel monomer, which can then be cast into single-cell droplets suitable for analysis by flow cytometry. A detection construct comprising a fluorescent detectable label may be cast into the droplet comprising unpolymerized gel monomer. Upon polymerization of the gel monomer to form a bead within a droplet. Because gel polymerization is through free-radical formation, the fluorescent reporter becomes covalently bound to the gel. The detection construct may be further modified to comprise a linker, such as an amine. A quencher may be added post-gel formation and will bind via the linker to the reporter construct. Thus, the quencher is not bound to the gel and is free to diffuse away when the reporter is cleaved by the CRISPR effector protein. Amplification of signal in droplet may be achieved by coupling the detection construct to a hybridization chain reaction (HCR initiators) amplification. DNA/RNA hybrid hairpins may be incorporated into the gel which may comprise a hairpin loop that has a RNase sensitive domain. By protecting a strand displacement toehold within a hairpin loop that has a RNase sensitive domain, HCR initiators may be selectively deprotected following cleavage of the hairpin loop by the CRISPR effector protein. Following deprotection of HCR initiators via toehold mediated strand displacement, fluorescent HCR monomers may be washed into the gel to enable signal amplification where the initiators are deprotected.

An example of microfluidic device that may be used in the context of the invention is described in Hou et al. “Direct Detection and drug-resistance profiling of bacteremias using inertial microfluidics” Lap Chip. 15(10):2297-2307 (2016). Further LOC embodiments are described elsewhere herein.

In one aspect, the embodiments disclosed herein are directed to a nucleic acid detection system comprising a CRISPR system, one or more guide RNAs designed to bind to corresponding target molecules, a reporter construct (also referred to herein as a detection construct in this context), and optional amplification reagents (discussed in greater detail elsewhere herein) to amplify target nucleic acid molecules and/or detectable signals in a sample. The reporter construct is a molecule that comprises an oligonucleotide component (DNA or RNA) that can be cleaved by an activated CRISPR effector protein. The composition of the oligonucleotide component may be generic i.e., not the same as a target molecule. The reporter construct is configured so that it prevents or masks generation of a detectable positive signal when in the uncleaved configuration but allows or facilitates generation of a positive detectable signal when cleaved. In the context of the present invention, reporting constructs comprising a first molecule and a second molecule connected by an RNA or DNA nucleic acid linker. Use of an RNA or DNA linker will depend on whether the CRISPR effector protein(s) used have RNA or DNA collateral activity. The first and second molecule are generally part of a binding pair, where the other binding partner is affixed to the lateral flow substrate as described in further detail below. The systems further comprise a detection agent that specifically binds the second molecule and further comprises a detectable label. In general, these reactions are referred to herein as CRISPR-Cas collateral cleavage detection reactions. If a target molecule is present in a sample, the corresponding guide molecule will guide the CRSIPR Cas/guide complex to the target molecule by hybridizing with the target molecule, thereby triggering the CRISPR effector protein's nuclease activity. This activated CRISPR effector protein will cleave both the target molecule and then non-specifically cleave the linker portion of the RNA construct.

### Lateral Flow Devices

In certain embodiments, the detection assay can be provided on a lateral flow device, as described in International Publication WO 2019/071051, incorporated herein by reference. The lateral flow device can be adapted to detect one or more coronaviruses and/or other viruses in combination of the coronavirus. The lateral flow device may comprise a flexible substrate, such as a paper substrate or a flexible polymer-based substrate, which can include freeze-dried reagents for detection assays with a visual readout of the assay results. See, WO 2019/071051 at [0145]-[0151] and Example 2, specifically incorporated herein by reference. In an aspect, lyophilized reagents can include preferred excipients that aid in rate of reaction, specificity, or other variables. The excipients may comprise trehalose, histidine, and/or glycine. In certain embodiments, the coronavirus assay can be utilized with isothermal amplification reagents, allowing amplification without complex instrumentation that may be unavailable in the field, as described in WO 2019/071051. Accordingly, the assay can be adapted for field diagnostics, including use of visual readout on a lateral flow device, rapid, sensitive detection and can be deployed for early and direct detection. Colorimetric detection can be utilized and may be particularly suited for field deployable applications, as described in International Application PCT/US2019/015726, published as WO2019/148206. In particular, colorimetric detection can be as described in WO2019/148206 at FIGS. 102, 105, 107-111 and [00306]-[00324], incorporated herein by reference.

In one embodiment, the invention provides a lateral flow device comprising a substrate comprising a first end and a second end. The first end may comprise a sample loading portion, a first region comprising a detectable ligand, two or more CRISPR effector systems, two or more detection constructs, and one or more first capture regions, each comprising a first binding agent. The substrate may also comprise two or more second capture regions between the first region of the first end and the second end, each second capture region comprising a different binding agent. Each of the two or more CRISPR effector systems may comprise a CRISPR effector protein and one or more guide sequences, each guide sequence configured to bind one or more target molecules.

The embodiments disclosed herein are directed to lateral flow detection devices that comprise a CRISPR-Cas detection system described herein. Examples of such systems include, but are not limited to SERLOCK and DETECTR systems.

The device may comprise a lateral flow substrate for detecting a CRISPR-Cas collateral cleavage detection reaction. Substrates suitable for use in lateral flow assays are known in the art. These may include but are not necessarily limited to membranes or pads made of cellulose and/or glass fiber, polyesters, nitrocellulose, or absorbent pads (J Saudi Chem Soc 19(6):689-705; 2015), and other embodiments further described herein. The CRISPR-Cas detection system, i.e., one or more CRISPR systems and corresponding reporter constructs are added to the lateral flow substrate at a defined reagent portion of the lateral flow substrate, typically on one end of the lateral flow substrate. Reporting constructs used within the context of the present invention can comprise a first molecule and a second molecule linked by an RNA or DNA linker. The lateral flow substrate further comprises a sample portion. The sample portion may be equivalent to, continuous with, or adjacent to the reagent portion. In an aspect, the lateral flow substrate can be utilized for visual readout of a detectable signal in one-pot reactions, e.g. wherein steps of extracting, amplifying and detecting are performed in an individual discrete volume.

### Lateral Flow Substrate

In some embodiments, the device is a lateral flow device. In some embodiments, the lateral flow device can be composed of a CRISPR system and detection construct described elsewhere herein and a lateral flow substrate for carrying out the detection reaction and/or nucleic acid release from the sample.

In certain example embodiments, a lateral flow device comprises a lateral flow substrate on which detection can be performed. Substrates suitable for use in lateral flow assays are known in the art. These may include, but are not necessarily limited to, membranes or pads made of cellulose and/or glass fiber, polyesters, nitrocellulose, or absorbent pads (J Saudi Chem Soc 19(6):689-705; 2015).

Lateral support substrates comprise a first and second end, and one or more capture regions that each comprise binding agents. The first end may comprise a sample loading portion, a first region comprising a detectable ligand, two or more CRISPR effector systems, two or more detection constructs, and one or more first capture regions, each comprising a first binding agent. The substrate may also comprise two or more second capture regions between the first region of the first end and the second end, each second capture region comprising a different binding agent. Each of the two or more CRISPR effector systems may comprise a CRISPR effector protein and one or more guide sequences, each guide sequence configured to bind one or more target molecules. The lateral flow substrates may be configured to detect a CRISPR-Cas collateral activity detection reaction.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

The embodiments disclosed herein can be prepared in freeze-dried format for convenient distribution and point-of-care (POC) applications. Such embodiments are useful in multiple scenarios in human health including, for example, viral detection, bacterial strain typing, sensitive genotyping, and detection of disease-associated cell free DNA. Accordingly, the lateral substrate comprising one or more of the elements of the system, including detectable ligands, CRISPR effector systems, detection constructs and binding agents may be freeze-dried to the lateral flow substrate and packaged as a ready to use device. Alternatively, all or a portion of the elements of the system may be added to the reagent portion of the lateral flow substrate at the time of using the device.

### First End and Second End of the Substrate

The substrate of the lateral flow device comprises a first and second end. The CRISPR-Cas detection system described herein, i.e., one or more CRISPR systems and corresponding reporter constructs are added to the lateral flow substrate at a defined reagent portion of the lateral flow substrate, typically on a first end of the lateral flow substrate. Reporting constructs used within the context of the present invention comprise a first molecule and a second molecule linked by an RNA or DNA linker. The lateral flow substrate further comprises a sample portion. The sample portion may be equivalent to, continuous with, or adjacent to the reagent portion.

In certain example embodiments, the first end comprises a first region. The first region comprises a detectable ligand, two or more CRISPR effector systems, two or more detection constructs, and one or more first capture regions, each comprising a first binding agent.

### Capture Regions

The lateral flow substrate can comprise one or more capture regions. In embodiments the first end of the lateral flow substrate comprises one or more first capture regions, with two or more second capture regions between the first region of the first end of the substrate and the second end of the substrate. The capture regions may be provided as a capture line, typically a horizontal line running across the device, but other configurations are possible. The first capture region is proximate to and on the same end of the lateral flow substrate as the sample loading portion.

### Binding Agents

Specific binding-integrating molecules comprise any members of binding pairs that can be used in the present invention. Such binding pairs are known to those skilled in the art and include, but are not limited to, antibody-antigen pairs, enzyme-substrate pairs, receptor-ligand pairs, and streptavidin-biotin. In addition to such known binding pairs, novel binding pairs may be specifically designed. A characteristic of binding pairs is the binding between the two members of the binding pair.

A first binding agent that specifically binds the first molecule of the reporter construct is fixed or otherwise immobilized to the first capture region. The second capture region is located towards the opposite end of the lateral flow substrate from the first capture region. A second binding agent is fixed or otherwise immobilized at the second capture region. The second binding agent specifically binds the second molecule of the reporter construct, or the second binding agent may bind a detectable ligand. For example, the detectable ligand may be a particle, such as a colloidal particle, that when it aggregates can be detected visually, and generates a detectable positive signal. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding region comprises a second binding agent capable of specifically or non-specifically binding the detectable ligand on the antibody of the detectable ligand. Binding agents can be, for example, antibodies, that recognize a particular affinity tag. Such binding agents can further contain, for example, detectable labels, such as isotope labels and/or nucleic acid barcodes. A barcode is a short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, guide RNAs of the CRISPR effector systems described herein may be used to detect the barcode.

### Detectable Ligands

The first region is loaded with a detectable ligand, such as those disclosed herein, for example a gold nanoparticle. The detectable ligand may be a particle, such as a colloidal particle, that when it aggregates can be detected visually. The particle may be modified with an antibody that specifically binds the second molecule on the reporter construct. If the reporter construct is not cleaved it will facilitate accumulation of the detectable ligand at the first binding region. If the reporter construct is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G. In some examples, the detectable ligand is a gold nanoparticle, which may be modified with a first antibody, such as an anti-FITC antibody.

### Lateral Flow Detection Constructs

The first region also comprises a detection construct. In one example embodiment, a RNA detection construct and a CRISPR effector system (a CRISPR effector protein and one or more guide sequences configured to bind to one or more target sequences) as disclosed herein. In one example embodiment, and for purposes of further illustration, the RNA construct may comprise a FAM molecule on a first end of the detection construction and a biotin on a second end of the detection construct. Upstream of the flow of solution from the first end of the lateral flow substrate is a first test band. The test band may comprise a biotin ligand. Accordingly, when the RNA detection construct is present it its initial state, i.e., in the absence of target, the FAM molecule on the first end will bind the anti-FITC antibody on the gold nanoparticle, and the biotin on the second end of the RNA construct will bind the biotin ligand allowing for the detectable ligand to accumulate at the first test, generating a detectable signal. Generation of a detectable signal at the first band indicates the absence of the target ligand. In the presence of target, the CRISPR effector complex forms and the CRISPR effector protein is activated resulting in cleavage of the RND detection construct. In the absence of intact RNA detection construct the colloidal gold will flow past the second strip. The lateral flow device may comprise a second band, upstream of the first band. The second band may comprise a molecule capable of binding the antibody-labeled colloidal gold molecule, for example an anti-rabbit antibody capable of binding a rabbit anti-FITC antibody on the colloidal gold. Therefore, in the presence of one or more targets, the detectable ligand will accumulate at the second band, indicating the presence of the one or more targets in the sample.

In some embodiments, the first end of the lateral flow device comprises two detection constructs and each of the two detection constructs comprises an RNA or DNA oligonucleotide, comprising a first molecule on a first end and a second molecule on a second end. The first molecule and the second molecule may be linked by an RNA or DNA linker.

In some embodiments, the first molecule on the first end of the first detection construct may be FAM and the second molecule on the second end of the first detection construct may be biotin, or vice versa. In some embodiments, the first molecule on the first end of the second detection construct may be FAM and the second molecule on the second end of the second detection construct may be Digoxigenin (DIG), or vice versa.

In some embodiments, the first end may comprise three detection constructs, wherein each of the three detection constructs comprises an RNA or DNA oligonucleotide, comprising a first molecule on a first end and a second molecule on a second end. In specific embodiments, the first and second molecules on the detection constructs comprise Tye 665 and Alexa 488; Tye 665 and FAM, and Tye 665 and Digoxigenin (DIG), respectively.

In some embodiments, the first end of the lateral flow device comprises two or more CRISPR effector systems, also referred to as a CRISPR-Cas or CRISPR system. In some embodiments, such a CRISPR effector system may include a CRISPR effector protein and one or more guide sequences configured to bind to one or more target sequences.

### Samples

When utilizing the detection systems with a lateral flow substrate, samples to be screened are loaded at the sample loading portion of the lateral flow substrate. The samples must be liquid samples or samples dissolved in an appropriate solvent, usually aqueous. The liquid sample reconstitutes the CRISPR-Cas collateral activity detection reagents such that a CRISPR-Cas collateral activity detection reaction can occur. The liquid sample begins to flow from the sample portion of the substrate towards the first and second capture regions. Exemplary samples are described in greater detail elsewhere herein.

See also WO 2019/071051, which is incorporated by reference herein.

### Cartridges and Chips

The cartridge, also referred to herein as a chip, according to the present invention comprises a series of components of ampoules and chambers that are communicatively coupled with one or more other components on the cartridge. The coupling is typically a fluidic communication, for example, via channels. The cartridge may comprise a membrane that seals one or more of the chambers and/or ampoules. In an aspect, the membrane allows for storage of reagents, buffers and other solid or fluid components which cover and seal the cartridge. The membrane can be configured to be punctured, pierced or otherwise released from sealing or covering one or more components of the cartridge by a means for releasing reagents. In some embodiments, the cartridge contains one or more wells, substrates (e.g., a flexible substrate), or other discrete volumes.

In some embodiments, the device is configured as lab-on-chip (LOC) diagnostic system. In some embodiments, the LOC is configured as a wireless lab-on-chip (LOC) diagnostic sensor system (see e.g., U.S. Pat. No. 9,470,699). In certain embodiments, CRISPR-Cas collateral activity detection assay is performed in a LOC controlled and/or read by a wireless device (e.g., a cell phone, a personal digital assistant (PDA), a tablet) and results and/or reaction are reported to and/or measured by said device. In some embodiments, the LOC may be a microfluidic device. The LOC may be a passive chip, wherein the chip is powered and controlled through a wireless device. In certain embodiments, the LOC includes a microfluidic channel for holding reagents and a channel for introducing a sample. In certain embodiments, a signal from the wireless device delivers power to the LOC and activates mixing of the sample and assay reagents. Specifically, in the case of the present invention, the system may include a masking agent, CRISPR effector protein, and guide RNAs specific for a target molecule. Upon activation of the LOC, the microfluidic device may mix the sample and assay reagents. Upon mixing, a sensor detects a signal and transmits the results to the wireless device. In certain embodiments, the unmasking agent is a conductive RNA molecule. The conductive RNA molecule may be attached to the conductive material. Conductive molecules can be conductive nanoparticles, conductive proteins, metal particles that are attached to the protein or latex or other beads that are conductive. In certain embodiments, if DNA or RNA is used then the conductive molecules can be attached directly to the matching DNA or RNA strands. The release of the conductive molecules may be detected across a sensor. The assay may be a one step process. Lab-on-the chip technology is well described in the scientific literature and consists of multiple microfluidic channels, input or chemical wells. Reactions in wells can be measured using radio frequency identification (RFID) tag technology since conductive leads from RFID electronic chip can be linked directly to each of the test wells. An antenna can be printed or mounted in another layer of the electronic chip or directly on the back of the device. Furthermore, the leads, the antenna and the electronic chip can be embedded into the LOC chip, thereby preventing shorting of the electrodes or electronics. Since LOC allows complex sample separation and analyses, this technology allows LOC tests to be done independently of a complex or expensive reader. Rather a simple wireless device such as a cell phone or a PDA can be used. In one embodiment, the wireless device also controls the separation and control of the microfluidics channels for more complex LOC analyses. In one embodiment, a LED and other electronic measuring or sensing devices are included in the LOC-RFID chip. Not being bound by a theory, this technology is disposable and allows complex tests that require separation and mixing to be performed outside of a laboratory.

As noted above, certain embodiments enable the use of nucleic acid binding beads to concentrate target nucleic acid but that do not require elution of the isolated nucleic acid. Thus, in certain example embodiments, the cartridge may further comprise an activatable magnet, such as an electro-magnet. A means for activating the magnet may be located on the device, or the means for supplying the magnet or activating the magnet on the cartridge may be provided by a second device, such as those disclosed in further detail below.

The overall size of the device may be between 10, 15, 20, 25, 30, 35, 40, 45, 55, 60, 65, 70, 75, 80, 85, 90, 95, or 100 mm in width, and 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mm. The sizing of ampoules, chambers, and channels can be selected to be in line with the reaction volumes discussed herein and to fit within the general size parameters of the overall cartridge.

### Ampoules

The ampoules, also referred to as blisters, allow for storage and release of reagents throughout the cartridge. Ampoules can include liquid or solid reagents, for example, lysis reagents in one ampoule and reaction reagents in another ampoule. The reagents can be as described elsewhere herein and can be adapted for the use in the cartridge. The ampoule may be sealed by a film that allows for the bursting, puncture or other release of the contents of the ampoules. See, e.g., Becker, H. & Gartner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002. Considerations for ampoules can include as discussed in, for example, Smith, S., et al., Blister pouches for effective reagent storage on microfluidic chips for blood cell counting. Microfluid Nanofluid 20, 163 (2016). DOI:10.1007/s10404-016-1830-2. In an aspect, the seal is a frangible seal formed of a composite-layer film that is assembled to the cartridge main body or other part of the device. While referred to herein as an ampoule, the ampoule may comprise a cavity on a chip which comprises a sealed film that is opened by the release means.

### Chambers

The chambers on the chip may located and sized for fluidic communication via channels or other communication means with ampoules and/or other chambers on the chip. A chamber for receiving a sample can be provided. The sample can be injected, placed in a receptacle into the chamber for receiving a sample, or otherwise transferred to the chamber. A lysis chamber may comprise, for example, capture beads, that may be used for concentration and/or extraction of the desired target material from the sample. Alternatively, the beads may be comprised in an ampoule comprising lysis reagents that are in fluidic communication with the lysis chamber. An amplification chamber may also be provided with, for example, one or more lyophilized components of the system in the amplification chamber and/or communicatively connected to an ampoule comprising one or more components of the amplification reaction.

When the cartridge comprises a magnet, it may be configured near one or more of the chambers. In an aspect, the magnet is near the lysis well, and may be configured such that the device has a means for activating the magnet. Embodiments comprising a magnet in the cartridge may be utilized with methodologies using magnetic beads for extraction of particular target molecules.

### System for Detection Assays

A system configured for use with the cartridge and to perform an assay, also referred to as a sample analysis apparatus, detection system or detection device, is configured system to receive the cartridge and conduct an assay comprising isothermal amplification of nucleic acids and detection of target nucleic acids on the cartridge. The system may comprise: a body; a door housing which may be provided in an opened state or a closed state and configured to be coupled to the body of the sample analysis apparatus by a hinge or other closure means; a cartridge accommodating unit included in the detection system and configured to accommodate the cartridge. The system may further comprise one or more means for releasing reagents for extractions, amplification and/or detection; one or more heating means for extractions, amplification and/or detection, a means for mixing reagents for extraction, amplification, and/or detections, and/or a means for reading the results of the assay. The device may further comprise a user interface for programming the device and/or readout of the results of the assay.

### Means for Release of Reagents

The system may comprise means for releasing reagents for extraction, amplification and/or detection. Release of reagents can be performed by a crushing, puncturing, applying heat or pressure until burst, cutting, or other means for the opening of the ampoule and release of contents. e.g., Becker, H. & Gartner, C. Microfluidics-enabled diagnostic systems: markets, challenges, and examples. In Microchip Diagnostics: Methods and Protocols (eds Taly, V. et al.) (Springer, New York, 2017); Czurratis et al., doi: 10.1088/0960-1317/25/4/045002.

### Mechanical Actuators

### Heating Means

The heating means or heating element can be provided, for example, by electrical or chemical elements. One or more heating means can be utilized, or circuits providing regulation of temperature to one or more locations within the detection device can be utilized. In an embodiment, the device is configured to comprise a heating means for heating the lysis (extraction) chamber and at the amplification chamber of the cartridge, sample vessel or other part of the device. In an aspect, the heating element is disposed under the extraction well. The system can be designed with one or more heating means for extraction, amplification and/or detection. In some embodiments, the device does not include a power source. In some embodiments, the heating element provides heat to a of about 65, 60, 55, 50, 45, 40, 39, 38, 37, 36, 35, 34, 33, 32, 31, 30, 29, 28, 27, 26, 25 degrees C. or less. In some embodiments, the device does not contain any heating element.

### Power Sources

In some embodiments, the device can include a power source. The power source can be coupled to one or more of the components of the device. In some embodiments, the power source is electrically coupled to one or more components of the device so as to provide electrical energy to the cone or more components. Suitable power sources that can be incorporated with the device are batteries (single use and rechargeable), solar powered power sources and batteries. In some embodiments, the power source can be coupled to an outside power source (e.g., an electric power grid) so as to recharge the on-board power source. In some embodiments, the device does not include a power source.

### Mixing Means

A means for mixing reagents for extraction, amplification and/or detection can be provided. A means for mixing reagents may comprise a means for mixing one or more fluids, or a fluid with a solid or lyophilized reaction mixture can also be provided. Means for mixing that disturb the laminar flow can be provided. In an aspect, the mixing means is a passive mixer, in another aspect, the mixing means is an active mixer. See, e.g. Nam-Trung Nguyen and Zhigang Wu 2005 J. Micromech. Microeng. 15 R1, doi: 10.1088/0960-1317/15/2/R01 for discussion of mixing approaches. In an aspect, the active mixer can be based on external sources such as pressure, temperature, hydrodynamics (with electrical or magnetic forces), dielectrophoresis, electrokinetics, or acoustics. Examples of passive mixing means can be provided by use of geometric approaches, such as a curved path or channel, see, e.g., U.S. Pat. No. 7,160,025, or an expansion/contraction of a channel cross section or diameter. When the cartridge is utilized with beads, channels and wells are configured and sized for the flow of beads.

### Means for Reading the Results of the Assay

A means for reading the results of the assay can be provided in the system. The means for reading the results of the assay will depend in part on the type of detectable signal generated by the assay. In particular embodiments, the assay generates a detectable fluorescent or color readaout. In these instances, the means for reading the results of the assay will be an optic means, for example a single channel or multi-channel optical means such as a fluorimeter, colorimeter or other spectroscopic sensor.

A combination of means for reading the results of the assay can be utilized, and may include readings such as turbidity, temperature, magnetic, radio, or electrical properties and or optical properties, including scattering, polarization effects, etc.

The system may further comprise a user interface for programming the device and/or readout of the results of the assay. The user interface may comprise an LED screen. The system can be further configured for a USB port that can allow for docking of four or more devices.

In an aspect, the system comprises a means for activating a magnet that is disposed within or on the cartridge.

### Wearable Devices

The systems described herein, may further be incorporated into wearable medical devices that assess biological samples, such as biological fluids or an environmental sample, of a subject or in a subject's environment outside the clinic setting and report the outcome of the assay remotely to a central server accessible by a medical care professional. In some embodiments the device may include the ability to self-sample blood, saliva, sweat, such as the devices disclosed in U.S. Patent Application Publication No. 2015/0342509 entitled “Needle-free Blood Draw to Peeters et al., U.S. Patent Application Publication No. 2015/0065821 entitled “Nanoparticle Phoresies” to Andrew Conrad.

In some embodiments, the device is configured as a dosimeter or badge that serves as a sensor or indicator such that the wearer is notified of exposure to certain microbes or other agents. For example, the systems described herein may be used to detect a particular pathogen. Likewise, aptamer-based embodiments disclosed above may be used to detect both polypeptide as well as other agents, such as chemical agents, to which a specific aptamer may bind. Such a device may be useful for surveillance of soldiers or other military personnel, as well as clinicians, researchers, hospital staff, and the like, in order to provide information relating to exposure to potentially dangerous microbes as quickly as possible, for example for biological or chemical warfare agent detection. In other embodiments, such a surveillance badge may be used for preventing exposure to dangerous microbes or pathogens in immunocompromised patients, burn patients, patients undergoing chemotherapy, children, or elderly individuals.

### Other Device Features

In certain example embodiments, the device may comprise individual wells, such as microplate wells. The size of the microplate wells may be the size of standard 6, 24, 96, 384, 1536, 3456, or 9600 sized wells. In certain example embodiments, the elements of the systems described herein may be freeze dried and applied to the surface of the well prior to distribution and use.

The devices disclosed herein may further comprise inlet and outlet ports, or openings, which in turn may be connected to valves, tubes, channels, chambers, and syringes and/or pumps for the introduction and extraction of fluids into and from the device. The devices may be connected to fluid flow actuators that allow directional movement of fluids within the microfluidic device. Example actuators include, but are not limited to, syringe pumps, mechanically actuated recirculating pumps, electroosmotic pumps, bulbs, bellows, diaphragms, or bubbles intended to force movement of fluids. In certain example embodiments, the devices are connected to controllers with programmable valves that work together to move fluids through the device. In certain example embodiments, the devices are connected to the controllers discussed in further detail below. The devices may be connected to flow actuators, controllers, and sample loading devices by tubing that terminates in metal pins for insertion into inlet ports on the device.

As shown herein the elements of the system are stable when freeze dried or lyophilized, therefore embodiments that do not require a supporting device are also contemplated, i.e., the system may be applied to any surface or fluid that will support the reactions disclosed herein and allow for detection of a positive detectable signal from that surface or solution. In addition to freeze-drying, the systems may also be stably stored and utilized in a pelletized form. Polymers useful in forming suitable pelletized forms are known in the art.

The devices disclosed herein may also include elements of point of care (POC) devices known in the art for analyzing samples by other methods. See, for example St John and Price, “Existing and Emerging Technologies for Point-of-Care Testing” (Clin Biochem Rev. 2014 August; 35(3): 155-167).

Radio frequency identification (RFID) tag systems include an RFID tag that transmits data for reception by an RFID reader (also referred to as an interrogator). In a typical RFID system, individual objects (e.g., store merchandise) are equipped with a relatively small tag that contains a transponder. The transponder has a memory chip that is given a unique electronic product code. The RFID reader emits a signal activating the transponder within the tag through the use of a communication protocol. Accordingly, the RFID reader is capable of reading and writing data to the tag. Additionally, the RFID tag reader processes the data according to the RFID tag system application. Currently, there are passive and active type RFID tags. The passive type RFID tag does not contain an internal power source, but is powered by radio frequency signals received from the RFID reader. Alternatively, the active type RFID tag contains an internal power source that enables the active type RFID tag to possess greater transmission ranges and memory capacity. The use of a passive versus an active tag is dependent upon the particular application.

Since the electrical conductivity of the surface area can be measured precisely quantitative results are possible on the disposable wireless RFID electro-assays. Furthermore, the test area can be very small allowing for more tests to be done in a given area and therefore resulting in cost savings. In certain embodiments, separate sensors each associated with a different CRISPR effector protein and guide RNA immobilized to a sensor are used to detect multiple target molecules. Not being bound by a theory, activation of different sensors may be distinguished by the wireless device.

In addition to the conductive methods described herein, other methods may be used that rely on RFID or Bluetooth as the basic low-cost communication and power platform for a disposable RFID assay. For example, optical means may be used to assess the presence and level of a given target molecule. In certain embodiments, an optical sensor detects unmasking of a fluorescent masking agent.

In certain embodiments, the device of the present invention may include handheld portable devices for diagnostic reading of an assay (see e.g., Vashist et al., Commercial Smartphone-Based Devices and Smart Applications for Personalized Healthcare Monitoring and Management, Diagnostics 2014, 4(3), 104-128; mReader from Mobile Assay; and Holomic Rapid Diagnostic Test Reader).

As noted herein, certain embodiments allow detection via colorimetric change which has certain attendant benefits when embodiments are utilized in POC situations and or in resource poor environments where access to more complex detection equipment to readout the signal may be limited. However, portable embodiments disclosed herein may also be coupled with hand-held spectrophotometers that enable detection of signals outside the visible range. An example of a hand-held spectrophotometer device that may be used in combination with the present invention is described in Das et al. “Ultra-portable, wireless smartphone spectrophotometer for rapid, non-destructive testing of fruit ripeness.” Nature Scientific Reports. 2016, 6:32504, DOI: 10.1038/srep32504. Finally, in certain embodiments utilizing quantum dot-based detection constructs, use of a handheld UV light, or other suitable device, may be successfully used to detect a signal owing to the near complete quantum yield provided by quantum dots.

### Kits

Any of the compounds, compositions, formulations, particles, cells, devices, and combinations thereof, described herein or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, dipsticks, substrates, bottles, and the like. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, devices, described herein or a combination thereof contained therein, safety information regarding the content of the compounds, compositions, formulations, particles, devices, and cells described herein or a combination thereof contained therein, information regarding the dosages, working amounts, indications for use, and/or recommended treatment regimen(s) for the compound(s) formulations, devices, and combinations thereof contained therein. In some embodiments, the instructions can provide directions for sample collection, sample preparation, and/or use of the compounds, compositions, formulations, particles, devices and cells described herein or a combination thereof. In some embodiments, the instructions can be specific to the target(s) being detected by a CRISPR effector detection system. In some embodiments, the instructions are specific to detecting a viral target, such as a viral polynucleotide. Exemplary virus that can be detected by the kits described herein are described elsewhere herein. In some embodiments, the viral target is SARS-CoV-2.

### Methods and Assays

Also described and provided herein are methods for detecting target nucleic acids in a sample. Such methods employ one or more of the CRISPR-Cas nucleic acid detection systems described herein, compositions described herein, and/or devices described herein. In general the method includes amplification of one or more target sequences in a sample followed by detection of one or more amplified target sequences by a CRISPR-Cas collateral activity nucleic acid detection system and assay described herein. The target sequences can be present in a sample. In some embodiments, the sample is processed prior to amplification. Such processing can include lysis of one or more cells or virus or viral like particles present in the sample to release target nucleic acids. In some embodiments, the method does not require or include extraction of the nucleic acids from the sample prior to amplification and/or target detection. In some embodiments, the sample preparation (e.g., lysis) and amplification occur in the same reaction vessel or location. In some embodiments, the sample preparation (e.g., lysis), target amplification, and CRISPR-Cas based nucleic acid detection occur in the same reaction vessel or location. In some embodiments, the reaction vessel or location contains the sample preparation, amplification, and/or CRISPR-Cas detection compositions and/or systems. In these embodiments, the sample can be added to the vessel and processing, amplification and detection can occur in the same vessel with no requirement to remove or add reagents to the vessel prior to obtaining a result. In some embodiments, the reagents, compositions, and systems are included in a vessel in a dehydrated (e.g., freeze dried, lyophilized, etc.) form and can be reconstituted when ready to use. In some embodiments, the processing (e.g., lysis, amplification, and/or CRISPR-Cas nucleic acid detection) can be performed at ambient or at about body temperature.

In some embodiments, the method can employ a Cas13 or Cas12 CRISPR-Cas system for target nucleic acid detection. See e.g., Jong et al. N Engl J Med. 2020. 383(15):1492-1494; Broughton, et al. CRISPR-Cas12-based detection of SARS-CoV-2. Nat Biotechnol (2020), doi:10.1038/s41587-020-0513-4 (DETECTR detection); Gootenberg et al., Science. 2018 Apr. 27; 360(6387):439-444. doi: 10.1126/science.aaq0179 (multiplexing lateral flow platform for point-of-care diagnostics); and Chen, et al., Science. 2018 Apr. 27; 360(6387):436-439. doi: 10.1126/science.aar6245 (Cas12 detection), each of which is incorporated by reference. Similarly, data from field deployable technologies can be utilized in accordance with the present invention. See, Myrhvold et al., Science 27 Apr. 2018: 360:6387, pp. 444-448; doi:10.1126/science.aas8836 (field deployable viral diagnostics), which are incorporated herien by herein by reference. Point-of-care testing is a preferred data source and may include population-scale diagnostics. See, e.g. Joung et al., Point-of-care testing for COVID-19 using SHERLOCK diagnostics” doi: 10.1101/2020.05.04.20091231; Schmid-Burgk, et al., “LAMP-Seq: Population-Scale COVID-19 Diagnostics Using Combinatorial Barcoding,” doi: 10.1101/2020.04.06.025635, each of which is incorporated herein by reference.

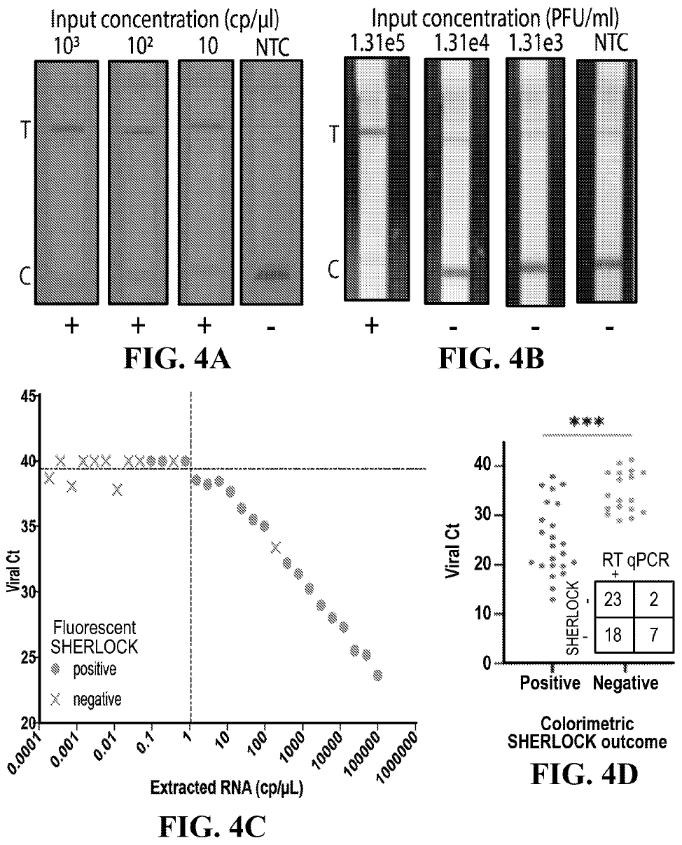

Certain example embodiments disclosed herein provide are based on low-cost CRISPR-based diagnostic that enables single-molecule detection of DNA or RNA with single-nucleotide specificity (Gootenberg, 2018; Gootenberg, et al, Science. 2017 Apr. 28; 356(6336):438-442 (2017); Myhrvold, et al., Science 360, 444-448 (2018)). Nucleic acid detection with SHERLOCK relies on the collateral activity of Type VI and Type V Cas proteins, such as Cas13 and Cas12, which unleashes promiscuous cleavage of reporters upon target detection (Gooteneberg et al., 2018)(Abudayyeh, et al., Science. 353(6299)(2016); East-Seletsky et al. Nature 538:270-273 (2016); Smargon et al. Mol Cell 65(4):618-630 (2017)). Certain embodiments disclosed herein, are capable of single-molecule detection in less than an hour and can be used for multiplexed target detection when using CRISPR enzymes with orthogonal cleavage preference, such as Cas13a from Leptotrichia wadei (LwaCas13a), Cas13b from Capnocytophaga canimorsus Cc5 (CcaCas13b), and Cas12a from Acidaminococcus sp. BV3L6 (AsCas12a); Alicyclobacillus acidiphilus (Aap) Cas 12b and Brevibacillus sp. SYSU G02855 (BrCas12b); (Gootenberg, 2018; Myhrvold et al. Science 360(6387):444-448 (2018); Gootenberg, 2017; Chen et al. Science 360(6387):436-439 (2018); Li et al. Cell Rep 25(12):3262-3272 (2018); Li et al. Nat Protoc 13(5):899-914 (2018)). Guide molecules used herein are designed using a model for high activity-based Cas guide selection for coronavirus would facilitate design of optimal diagnostic assays, especially in applications requiring high-activity guides like lateral flow detection, and enable guide RNA design for in vivo RNA targeting applications with Cas13 has also been detailed in U.S. Provisional Applications 62/818,702 filed Mar. 14, 2019, now PCT/US20/22795 and 62/890,555, filed Aug. 22, 2019, now PCT/US20/22795, both entitled CRISPR Effector System Based Multiplex Diagnostics, incorporated herein by reference in their entirety, and, in particular, Examples 1-4, Tables 1-8 and FIG. 4A of U.S. Provisional Application 62/890,555.

Embodiments disclosed herein utilize Cas proteins possessing non-specific nuclease collateral activity to cleave detectable reporters upon target recognition, providing sensitive and specific diagnostics, including single nucleotide variants, detection based on rRNA sequences, screening for drug resistance, monitoring microbe outbreaks, genetic perturbations, and screening of environmental samples, as described, for example, in PCT/US18/054472 filed Oct. 22, 2018 at [0183]-[0327], incorporated herein by reference. Reference is made to WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and U.S. Pat. No. 62,767,077 filed Novemebr 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox D B T, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6., Science. 2018 Apr. 27; 360(6387):439-444; Gootenberg J S, et al., Nucleic acid detection with CRISPR-Cas13a/C2c2., Science. 2017 Apr. 28; 356(6336):438-442; Abudayyeh 00, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimising deaminase fusions for genome editing. Nat Commun. 2016 Nov. 2; 7:13330, Myhrvold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, each of which is incorporated herein by reference in its entirety. Exemplary Cas proteins that can be included in the CRISPR-Cas nucleic acid detection systems are also described in elsewhere herein.

The low cost and adaptability of the assay platform described herein lends itself to a number of applications including (i) general viral RNA/DNA quantitation, (ii) rapid, multiplexed RNA/DNA expression detection, and (iii) sensitive detection of target nucleic acids in both clinical and environmental samples. Additionally, the systems disclosed herein may be adapted for detection of transcripts within biological settings, such as cells. Given the highly specific nature of the CRISPR effectors described herein, it may possible to track allelic specific expression of transcripts or disease-associated mutations and/or the presence of microorganisms in live cells.

In certain example embodiments, a single guide RNA specific to a single target is placed in separate volumes. Each volume may then receive a different sample or aliquot of the same sample. In certain example embodiments, multiple guide RNA each to separate target may be placed in a single well such that multiple targets may be screened in a different well. In order to detect multiple guide RNAs in a single volume, in certain example embodiments, multiple effector proteins with different specificities may be used. For example, different orthologs with different sequence specificities may be used. For example, one orthologue may preferentially cut A, while others preferentially cut C, U, or T. Accordingly, guide RNAs that are all, or comprise a substantial portion, of a single nucleotide may be generated, each with a different fluorophore. In this way up to four different targets may be screened in a single individual discrete volume.

Generally, the CRISPR effector system detection method can be composed of two parts: 1) sample preparation and 2) CRISPR effector system detection of one or more targets present in the sample. The CRISPR effector system detection portion of the method can include a transcription step followed by CRISPR-effector system mediated detection of a target. In some embodiments, the CRISPR effector system detection portion of the method can also include target amplification and/or signal amplification/enrichment. These steps are described in greater detail below and elsewhere herein. In some embodiments, one or more of the steps within each of the portions of the method are performed in the same reaction vessel, reaction area/location, and/or device. In some embodiments all of the steps of the method are performed in the same reaction vessel, same reaction vessel, reaction area/location, and/or device.

In some embodiments, the CRISPR effector systems and methods herein are capable of detecting down to at least attomolar concentrations of target molecules, such as viral polynucleotides. In some embodiments, the CRISPR effector systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of viral DNA or RNA per microliter (cp/μL). In some embodiments, the CRISPR effector systems and methods herein are capable of detecting down to about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 copies of viral DNA or RNA per microliter (cp/μL) using a fluorescent or colorimetric readout.

In some embodiments, the polynucleotides are released from cells in the sample and the CRISRP-effector system detection can occur on the released polynucleotides without extracting the sample polynucleotides from other components in the sample. This can allow for the sample preparation and CRISRP-effector detection reaction to be performed in the same reaction vessel.

In some embodiments, one or more or all of the steps included in the CRISPR-effector system detection reaction can occur at about 22-55 degrees C. (including any target and/or signal amplification). In some embodiments, one or more or all of the steps included in the CRISPR-effector system detection reaction can occur at about 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, to/or 55 degrees C., 22, 23, 24, 25, 26, 27, 28, 29, 30, 31, 32, 33, 34, 35, 36, to/or 37 degrees C., about 22, 23, 24, 25, 26, 27, 28, 29, 30, 31, 32, or/to 33 degrees C., about 22, 23, 24, 25, 26, or/to 27 degrees C., or about 22, 23, 24, to/or 25 degrees C. In some embodiments, one or more or all of the steps included in the CRISPR-effector system detection reaction can occur at about room temperature (about 22-25 degrees C.).

In some embodiments, the CRISPR-effector system detection reaction can occur as a two-step reaction in which amplification of target(s) and target detection via the CRISPR effector system occur in separate reactions. In some embodiments, The CRISPR-effector system detection reaction (including any target and/or signal amplification) can occur as a single, one-pot reaction. In some embodiments where the CRISPR-effector system detection reaction is a one-pot reaction, target amplification is achieved using LAMP or RPA. In some embodiments where the CRISRP-effector system detection reaction is a one-pot reaction the CRISPR-effector system includes a Cas 12 (such as a Cas12b) or a Cas13 (such as a cas13a). In some embodiments where the CRISRP-effector system detection reaction is a one-pot reaction and target amplification is achieved using LAMP, the CRISPR-effector system includes a Cas12, such as a Cas12b. In some embodiments where the CRISRP-effector system detection reaction is a one-pot reaction and target amplification is achieved using RPA, the CRISPR-effector system includes a Cas13, such as a Cas13a. In some embodiments, sample preparation and a single, one-pot CRISPR effector system can occur in the same reaction vessel, thus eliminating the need to move potentially hazardous samples from one reaction vessel to another.

In some embodiments, the total time to perform the CRISPR-effector system detection method (from sample preparation to detection) can be greater than 0 hours but less than about 4, 3.5, 3, 2.5, 2, 1.5, 1, or 0.5 hours. In some embodiments, the total time to perform the CRISPR-effector system detection method (from sample preparation to detection) can occur within about 20 to 120 minutes, such as within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, to/or 120 minutes. In some embodiments, the total time to perform the CRISPR-effector system detection method (from sample preparation to detection) can occur within about 20 to about 60 minutes, e.g. within about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or/to 60 minutes. In some embodiments, the total time to perform the CRISPR-effector system detection method (from sample preparation to detection) can occur within about 20 to about 45 minutes, e.g. within about 20, 21, 22, 23, 24, 25, 26, 27, 28, 29, 30, 31, 32, 33, 34, 35, 36, 37, 38, 39, 40, 41, 42, 43, 44, and/or 45 minutes. In some embodiments, the total time to perform the CRISPR-effector system detection method (from sample preparation to detection) can occur within about 20 to about 30 minutes, e.g., within about 20, 21, 22, 23, 24, 25, 26, 27, 28, 29, and/or 30 minutes.

In some embodiments, the CRISPR-effector system detection reaction can occur within about 1 to about 60 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In some embodiments, the CRISPR-effector system detection reaction can occur within about 1 to about 45 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In some embodiments, the CRISPR-effector system detection reaction can occur within about 1 to about 30 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In some embodiments, the CRISPR-effector system detection reaction can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In some embodiments, the CRISPR-effector system detection reaction can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In some embodiments, the CRISPR-effector system detection reaction can occur within about 1 to about 15 minutes, e.g., within about 1, 2, 3, 4, 5, 6, 7, 8, 9, 10, 11, 12, 13, to/or about 15 minutes. In some embodiments, the CRISPR-effector system detection reaction can occur within about 1 to about 10 minutes, e.g., within about 1, 2, 3, 4, 5, 6, 7, 8, 9, to/or about 10 minutes. In some embodiments, the CRISPR-effector system detection reaction can occur within about 1 to about 5 minutes, e.g., within about 1, 2, 3, 4, to/or about 5 minutes.

In some embodiments, the method includes preparation of the reagents for one or more steps, such as sample preparation, amplification, and/or CRISPR/Cas detection, for storage. Such storage preparation can include, but is not limited to lyophilizing, freeze drying, or otherwise dehydrating them. They can be prepared for storage inside of individual reaction vessels or locations within a device or other vessel. In some of these embodiments, the reagents, compositions, systems or combinations thereof are e.g., lyophilized or freeze dried inside of the reaction vessel or at the specific discreet locations on a substrate or otherwise in a device. They can be stored at a temperature ranging from ambient temperature (e.g., about 25-32 degrees C.) to about −20 or −80 degrees Celsius. In some embodiments, they are stored for 1, 2, 3, 4, 5, 6, 7, 8, 9, 10, 11, or 12 days, weeks, months or years. In some embodiments, the reagents, compositions, systems or combinations thereof are prepared and stored at about 4 degrees C. for about, 2, 3, 4, 5, 6, 7, 8, 9, 10, 11, or 12 days, weeks, months or years or more.

Due to the sensitivity of said systems, a number of applications that require from the rapid and sensitive detection may benefit from the embodiments disclosed herein and are contemplated to be within the scope of the invention.

Example assays and applications are described in further detail below.

### Sample Preparation

In some embodiments, the sample preparation can include extraction-free release of polynucleotides (e.g., DNA and/or RNA) from cells and/or microorganisms, such as viruses, present in the sample. In some embodiments, the sample preparation can include virus inactivation and/or nuclease inactivation. In some embodiments sample preparation is composed of inactivating nucleases present in a sample followed by virus inactivation. The step of sample preparation can occur prior to any target amplification and/or CRISPR-effector system detection. In some embodiments, sample preparation can include nuclease inactivation and/or viral inactivation by 1, 2, 3, 4 or more thermal (heat or cold) inactivation steps, chemical inactivation steps, biologic inactivation, physiologic inactivation, physical inactivation steps, or any combination thereof. Viral inactivation can, in some embodiments, result in lysis of the viral particles. In some embodiments, the same methods and reagents can be applied to other microbes (e.g., bacteria and eukaryotic cells).

In some embodiments, sample preparation includes one or more thermal steps. In some embodiments, nuclease inactivation can include one or more thermal steps. In some embodiments, viral inactivation can include one or more thermal steps. Thermal steps can be heating, cooling, cycles of heating and cooling at one or more rates of temperature change. Without being bound by theory, in some embodiments, heating and/or cooling, and/or one or more heating/cooling cycles as described herein can disrupt the integrity, function, and/or activity of biological molecules and structures (such as enzymes, membranes, viral capsids, and the like). In some embodiments, the sample presentation can be composed of or include 1, 2, 3, 4, or more heating steps at one or more different temperatures. In some embodiments, the sample presentation can be composed of or include 1, 2, 3, 4, or more cooling steps at one or more different temperatures. The duration of thermal each step can be independently selected from about 0.5 to about 60 minutes or more, such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, 60 minutes or more.

In some embodiments, one or more or all of the sample preparation steps can occur at about 15-95 degrees C. In some embodiments, one or more or all of the sample preparation steps can occur at about 15-30 degrees C., about 20-25 degrees C., or about 22-25 degrees C. In some embodiments, one or more or all of the sample preparations steps can occur at about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to/or 95 degrees C. In some embodiments, the one or more or all of the sample preparations steps can occur at about 15, 16, 17, 18, 19, 20, 21, 22, 23, 24, 25, 26, 27, 28, 29, 30, 31, 32, 33, 34, 35, 36, or/to 37 degrees C., about 15, 16, 17, 18, 19, 20, 21, 22, 23, 24, 25, 26, 27, 28, 29, 30, 31, 32, or/to 33 degrees C., about 22, 23, 24, 25, 26, or/to 27 degrees C., about 15, 16, 17, 18, 19, 20, 21, 22, 23, 24, 25, 26, 27, 28, 29, to/or 30 degrees C., about 15, 16, 17, 18, 19, 20, 21, 22, 23, 24, to/or 25 degrees C., about 20, 21, 22, 23, 24, 25, 26, 27, 28, 29, to/or 30 degrees C., about 20, 21, 22, 23, 24, or/to 25 degrees C., or about 22, 23, 24, or/to 25 degrees C. In some embodiments, one or more or all of the sample preparation steps reaction can be performed at about room temperature (about 15-30 degrees C.). In some embodiments, one or more or all of the sample preparations steps can be carried out at 37° C. to 50° C., such as about 37, 38, 39, 40, 41, 42, 43, 44, 45, 46, 47, 48, 49, to/or about 50 degrees C. In some embodiments, one or more or all of the sample preparation steps can be carried out at about 64-95 degrees C., such as 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to/or 95 degrees C.

In some embodiments, nuclease inactivation can occur at about 15-50 degrees C. In some embodiments, one or more or all of the sample preparations steps can occur at about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, to/or 50 degrees C. In some embodiments, nuclease inactivation can occur at about 15, 16, 17, 18, 19, 20, 21, 22, 23, 24, 25, 26, 27, 28, 29, 30, 31, 32, 33, 34, 35, 36, or/to 37 degrees C., about 15, 16, 17, 18, 19, 20, 21, 22, 23, 24, 25, 26, 27, 28, 29, 30, 31, 32, or/to 33 degrees C., about 15, 16, 17, 18, 19, 20, 21, 22, 23, 24, 25, 26, or/to 27 degrees C., about 15, 16, 17, 18, 19, 20, 21, 22, 23, 24, to/or 25 degrees C., about 15, 16, 17, 18, 19, 20, 21, 22, 23, 24, 25, 26, 27, 28, 29, to/or 30 degrees C., about 15, 16, 17, 18, 19, 20, 21, 22, 23, 24, to/or 25 degrees C., about 20, 21, 22, 23, 24, 25, 26, 27, 28, 29, to/or 30 degrees C., about 20, 21, 22, 23, 24, or/to 25 degrees C., or about 22, 23, 24, or/to 25 degrees C. In some embodiments, nuclease inactivation can occur at about room temperature (about 15-30 degrees C.). In some embodiments, nuclease inactivation can occur at about 37° C. to 50° C., such as about 37, 38, 39, 40, 41, 42, 43, 44, 45, 46, 47, 48, 49, to/or about 50 degrees C.

In some embodiments, viral inactivation can occur at about 15 to about 95 degrees C., such as about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to/or 95 degrees C. In some embodiments, viral inactivation can occur at about 15-37 degrees C., e.g., 15, 16, 17, 18, 19, 20, 21, 22, 23, 24, 25, 26, 27, 28, 29, 30, 31, 32, 33, 34, 35, 36, and/or 37 degrees C. In some embodiments, viral inactivation can occur at about 15 to about 33 degrees C., such as, 15, 16, 17, 18, 19, 20, 21, 22, 23, 24, 25, 26, 27, 28, 29, 30, 31, 32, /or 33 degrees C. In some embodiments, viral inactivation can occur at about 15 to about 30 degrees C., such as, 15, 16, 17, 18, 19, 20, 21, 22, 23, 24, 25, 26, 27, 28, 29, to/or 30 degrees C. In some embodiments, viral inactivation can occur at about 15 to about 25 degrees C., such as, 15, 16, 17, 18, 19, 20, 21, 22, 23, 24, to/or 25 degrees C. In some embodiments, viral inactivation can occur at about 22 to about 25 degrees C., such as, 22, 23, 24, to/or 25 degrees C. In some embodiments, the viral inactivation step is carried out at a temperature ranging from 64° C. to 95° C., such as 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to/or 95 degrees C.

In some embodiments, the sample preparation can occur within about 1 to about 60 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In some embodiments, the sample preparation can occur within about 1 to about 45 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In some embodiments, the sample preparation can occur within about 1 to about 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In some embodiments, the sample preparation can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In some embodiments, the sample preparation can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In some embodiments, the sample preparation can occur within about 1 to about 15 minutes, e.g., within about 1, 2, 3, 4, 5, 6, 7, 8, 9, 10, 11, 12, 13, to/or about 15 minutes. In some embodiments, the sample preparation can occur within about 1 to about 10 minutes, e.g., within about 1, 2, 3, 4, 5, 6, 7, 8, 9, to/or about 10 minutes. In some embodiments, the sample preparation can occur within about 1 to about 5 minutes, e.g., within about 1, 2, 3, 4, to/or about 5 minutes.

In some embodiments, the nuclease and/or viral inactivation can occur within about 1 to about 60 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, to/or about 60 minutes. In some embodiments, the nuclease and/or viral inactivation can occur within about 1 to about 45 minutes, e.g. within about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, to/or about 45 minutes. In some embodiments, the nuclease and/or viral inactivation can occur within about 1 to about 60 minutes, e.g., within about 1, 2, 3, 4, 5, 6, 7, 8, 9, 10, 11, 12, 13, 14, 15, 16, 17, 18, 19, 20, 21, 22, 23, 24, 25, 26, 27, 28, 29, to/or about 30 minutes. In some embodiments, the nuclease and/or viral inactivation can occur within about 1 to about 25 minutes, e.g., within about 1, 2, 3, 4, 5, 6, 7, 8, 9, 10, 11, 12, 13, 14, 15, 16, 17, 18, 19, 20, 21, 22, 23, 24, to/or about 25 minutes. In some embodiments, the nuclease and/or viral inactivation can occur within about 1 to about 20 minutes, e.g., within about 1, 2, 3, 4, 5, 6, 7, 8, 9, 10, 11, 12, 13, 14, 15, 16, 17, 18, 19, to/or about 20 minutes. In some embodiments, the nuclease and/or viral inactivation can occur within about 1 to about 15 minutes, e.g., within about 1, 2, 3, 4, 5, 6, 7, 8, 9, 10, 11, 12, 13, to/or about 15 minutes. In some embodiments, the nuclease and/or viral inactivation can occur within about 1 to about 10 minutes, e.g., within about 1, 2, 3, 4, 5, 6, 7, 8, 9, to/or about 10 minutes. In some embodiments, the nuclease and/or viral inactivation can occur within about 1 to about 5 minutes, e.g., within about 1, 2, 3, 4, to/or about 5 minutes. In some embodiments, the nuclease inactivation step is of a duration selected from 5 minutes, 10 minutes, 15 minutes, and 20 minutes.

In some embodiments, one or more sample preparation steps can include one or more steps incubating the sample for a period of time at a temperature ranging from about 15-95 degrees C., 15-64 degrees C., 15-37 degrees C., 15-30 degrees C., 15-27 degrees C., 15-25 degrees C., 20-30 degrees C., 22-25 degrees C., −80 degrees C. to about 0 degrees C., −60 degrees C. to about 0 degrees C., −40 degrees C. to about 0 degrees C., −20 degrees C. to about 0 degrees C., −10 degrees C. to about 0 degrees C., −5 degrees C. to about 0 degrees C., or a combination thereof. In some embodiments, the period of time for each incubation can range from 0.5 min to about 60 minutes, such as about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, 50, 50.5, 51, 51.5, 52, 52.5, 53, 53.5, 54, 54.5, 55, 55.5, 56, 56.5, 57, 57.5, 58, 58.5, 59, 59.5, to/or 60 minutes. In some embodiments, the period of time for each incubation can range from about 1 hour to about 24 hours, such as about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, to/or about 24 hours.

In some embodiments, sample preparation can include one or more chemical inactivation steps. In some embodiments, nuclease inactivation can include one or more chemical inactivation steps. In some embodiments, viral inactivation can include one or more chemical inactivation steps. Chemical inactivation steps can include, but are not limited to, treatment with DEPC, 2-Mercaptoethanol, EDTA, EGTA, DTT, TCEP 2-nitro-5-thiocyanobenzoic acid, Ca²⁺, Sodium dodecyl sulfate, Carbodiimide and cholesterol sulfate, Iodoacetate, DNase inactivation reagent (Ambion Life Sciences), RNaseZap (Qiagen), SecurRIN advanced RNase inhibitor (e.g., cat no. RNI0301 from HghiQu GmbH), RNAse alert (Ambion), and combinations thereof. Any of these compositions or combinations thereof can be included in the sample preparation formulation.

The sample preparation formulation can contain one or more buffers such as HEPES, HBSS, HEPPS, EPPS, MES, Bis-Tris, ADA, ACES, PIPES, MOPSO Bis-6Tris Propane, BES, MOPS, TES, DIPSO, MOBS, TAPSO, HEPPSO, POPSO, Tricine, Gly-Gly, Bicinie, HEPBS, TAPS, AMPD, TABS, AMPSO, CHES, CAPSO, AMP, CAPS, and/or the like. In some embodiments, the pH of the buffer can be greater than 7.0, such as about 8, 8.5, 9, 9.5, 10, or about 10.5. In some embodiments, the buffer included is HEPES, pH about 8.0. In some embodiments, the HEPES is about 20 mM to about 100 mM

In some embodiments, the sample preparation formulation includes a salt. Exemplary salts include but are not limited to, NaCl, KCl, K₂SO₄, NaF, NaBr, NaI, Na₂SO₄, and NaHCO₃. In some embodiments, the sample preparation formulation includes at least KCl. In some embodiments, the salt is included in the sample preparation formulation at about 100 to 500 mM. In some embodiments, the salt is included in the sample preparation formulation at about 300 mM.

In some embodiments, the sample preparation formulation contains an amount of glycerol. In some embodiments, the glycerol is included at about 0.1, 0.5, 1, 1.5, 2, 3, 4, 5, 6, 7, 8, 9 to/or about 10% w/v or v/v.

In some embodiments, the sample preparation formulation contains an amount of sucrose. In some embodiments, the amount of sucrose is about 0.1, 0.5, 1, 1.5, 2, 3, 4, 5, 6, 7, 8, 9 to/or about 10% w/v or v/v. In some embodiments, the sucrose is about 5% w/v.

In some embodiments, the sample preparation formulation contains an amount of mannitol. In some embodiments, the amount of mannitol is about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, to/or 500 mM, such as about 100, 120, 125, 130, 135, 140, 145, 150, 155, or about 160 mM. In some embodiments, the amount of mannitol is about 150 mM.

In some embodiments, the sample preparation formulation contains an amount

In some embodiments, the sample preparation formulation includes a reducing agent, such as DTT or beta mercaptoethanol.

In some embodiments, the sample preparation formulation includes one or more polyethylene glycols (PEG). In some embodiments, the PEG can have a molecular weight ranging from about 1000 to 10,000, such as about 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, to/or 10000. In some embodiments, the sample preparation formulation contains a PEG with a molecular weight of about 8000. In some embodiments, the sample preparation formulation contains two PEGs each with a different molecular weight. In some of these embodiments, the two PEGs contained in the sample preparation formulation is PEG-1500 and PEG-8000. In some embodiments, the PEG can be included in the sample preparation formulation at about 0.01 to about 10 percent w/v or more., such as 0, 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.05, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, 3, 3.05, 3.1, 3.15, 3.2, 3.25, 3.3, 3.35, 3.4, 3.45, 3.5, 3.55, 3.6, 3.65, 3.7, 3.75, 3.8, 3.85, 3.9, 3.95, 4, 4.05, 4.1, 4.15, 4.2, 4.25, 4.3, 4.35, 4.4, 4.45, 4.5, 4.55, 4.6, 4.65, 4.7, 4.75, 4.8, 4.85, 4.9, 4.95, 5, 5.05, 5.1, 5.15, 5.2, 5.25, 5.3, 5.35, 5.4, 5.45, 5.5, 5.55, 5.6, 5.65, 5.7, 5.75, 5.8, 5.85, 5.9, 5.95, 6, 6.05, 6.1, 6.15, 6.2, 6.25, 6.3, 6.35, 6.4, 6.45, 6.5, 6.55, 6.6, 6.65, 6.7, 6.75, 6.8, 6.85, 6.9, 6.95, 7, 7.05, 7.1, 7.15, 7.2, 7.25, 7.3, 7.35, 7.4, 7.45, 7.5, 7.55, 7.6, 7.65, 7.7, 7.75, 7.8, 7.85, 7.9, 7.95, 8, 8.05, 8.1, 8.15, 8.2, 8.25, 8.3, 8.35, 8.4, 8.45, 8.5, 8.55, 8.6, 8.65, 8.7, 8.75, 8.8, 8.85, 8.9, 8.95, 9, 9.05, 9.1, 9.15, 9.2, 9.25, 9.3, 9.35, 9.4, 9.45, 9.5, 9.55, 9.6, 9.65, 9.7, 9.75, 9.8, 9.85, 9.9, 9.95, 10, 11, 12, 13, 14, 15, 16, 17, 18, 19, 20, 21, 22, 23, 24, to/or about 25% w/v. In some embodiments, the sample preparation formulation contains PEG-8000 at about 0.35% to about 3.5% w/v. In some embodiments, the sample preparation formulation contains PEG-8000 at about 3.5% w/v. In some embodiments, the sample preparation formulation contains PEG-8000 at about 03.5% w/v. In some embodiments, the sample preparation formulation contains PEG-1500 at about 3.5% w/v. In some embodiments, the sample preparation formulation contains PEG-1500 at about 3.5% w/v and PEG-8000 at about 0.35%. In some embodiments, the sample preparation formulation does not contain PEG.

In some embodiments, each of the compounds or compositions used in a step of the sample preparation reaction and/or contained in the sample preparation formulation can be included in the sample preparation formulation or reaction at a concentration of 1 to 1000, e.g. 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 pM, nM, M, mM, or M. In some embodiments, each of the chemicals a step of sample preparation reaction and/or solution can be included at 0.01 to about 100 w/v, v/v, or w/w percent of the reaction solution and/or sample preparation formulation, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10, 10.1, 10.2, 10.3, 10.4, 10.5, 10.6, 10.7, 10.8, 10.9, 11, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9, 13, 13.1, 13.2, 13.3, 13.4, 13.5, 13.6, 13.7, 13.8, 13.9, 14, 14.1, 14.2, 14.3, 14.4, 14.5, 14.6, 14.7, 14.8, 14.9, 15, 15.1, 15.2, 15.3, 15.4, 15.5, 15.6, 15.7, 15.8, 15.9, 16, 16.1, 16.2, 16.3, 16.4, 16.5, 16.6, 16.7, 16.8, 16.9, 17, 17.1, 17.2, 17.3, 17.4, 17.5, 17.6, 17.7, 17.8, 17.9, 18, 18.1, 18.2, 18.3, 18.4, 18.5, 18.6, 18.7, 18.8, 18.9, 19, 19.1, 19.2, 19.3, 19.4, 19.5, 19.6, 19.7, 19.8, 19.9, 20, 20.1, 20.2, 20.3, 20.4, 20.5, 20.6, 20.7, 20.8, 20.9, 21, 21.1, 21.2, 21.3, 21.4, 21.5, 21.6, 21.7, 21.8, 21.9, 22, 22.1, 22.2, 22.3, 22.4, 22.5, 22.6, 22.7, 22.8, 22.9, 23, 23.1, 23.2, 23.3, 23.4, 23.5, 23.6, 23.7, 23.8, 23.9, 24, 24.1, 24.2, 24.3, 24.4, 24.5, 24.6, 24.7, 24.8, 24.9, 25, 25.1, 25.2, 25.3, 25.4, 25.5, 25.6, 25.7, 25.8, 25.9, 26, 26.1, 26.2, 26.3, 26.4, 26.5, 26.6, 26.7, 26.8, 26.9, 27, 27.1, 27.2, 27.3, 27.4, 27.5, 27.6, 27.7, 27.8, 27.9, 28, 28.1, 28.2, 28.3, 28.4, 28.5, 28.6, 28.7, 28.8, 28.9, 29, 29.1, 29.2, 29.3, 29.4, 29.5, 29.6, 29.7, 29.8, 29.9, 30, 30.1, 30.2, 30.3, 30.4, 30.5, 30.6, 30.7, 30.8, 30.9, 31, 31.1, 31.2, 31.3, 31.4, 31.5, 31.6, 31.7, 31.8, 31.9, 32, 32.1, 32.2, 32.3, 32.4, 32.5, 32.6, 32.7, 32.8, 32.9, 33, 33.1, 33.2, 33.3, 33.4, 33.5, 33.6, 33.7, 33.8, 33.9, 34, 34.1, 34.2, 34.3, 34.4, 34.5, 34.6, 34.7, 34.8, 34.9, 35, 35.1, 35.2, 35.3, 35.4, 35.5, 35.6, 35.7, 35.8, 35.9, 36, 36.1, 36.2, 36.3, 36.4, 36.5, 36.6, 36.7, 36.8, 36.9, 37, 37.1, 37.2, 37.3, 37.4, 37.5, 37.6, 37.7, 37.8, 37.9, 38, 38.1, 38.2, 38.3, 38.4, 38.5, 38.6, 38.7, 38.8, 38.9, 39, 39.1, 39.2, 39.3, 39.4, 39.5, 39.6, 39.7, 39.8, 39.9, 40, 40.1, 40.2, 40.3, 40.4, 40.5, 40.6, 40.7, 40.8, 40.9, 41, 41.1, 41.2, 41.3, 41.4, 41.5, 41.6, 41.7, 41.8, 41.9, 42, 42.1, 42.2, 42.3, 42.4, 42.5, 42.6, 42.7, 42.8, 42.9, 43, 43.1, 43.2, 43.3, 43.4, 43.5, 43.6, 43.7, 43.8, 43.9, 44, 44.1, 44.2, 44.3, 44.4, 44.5, 44.6, 44.7, 44.8, 44.9, 45, 45.1, 45.2, 45.3, 45.4, 45.5, 45.6, 45.7, 45.8, 45.9, 46, 46.1, 46.2, 46.3, 46.4, 46.5, 46.6, 46.7, 46.8, 46.9, 47, 47.1, 47.2, 47.3, 47.4, 47.5, 47.6, 47.7, 47.8, 47.9, 48, 48.1, 48.2, 48.3, 48.4, 48.5, 48.6, 48.7, 48.8, 48.9, 49, 49.1, 49.2, 49.3, 49.4, 49.5, 49.6, 49.7, 49.8, 49.9, 50, 50.1, 50.2, 50.3, 50.4, 50.5, 50.6, 50.7, 50.8, 50.9, 51, 51.1, 51.2, 51.3, 51.4, 51.5, 51.6, 51.7, 51.8, 51.9, 52, 52.1, 52.2, 52.3, 52.4, 52.5, 52.6, 52.7, 52.8, 52.9, 53, 53.1, 53.2, 53.3, 53.4, 53.5, 53.6, 53.7, 53.8, 53.9, 54, 54.1, 54.2, 54.3, 54.4, 54.5, 54.6, 54.7, 54.8, 54.9, 55, 55.1, 55.2, 55.3, 55.4, 55.5, 55.6, 55.7, 55.8, 55.9, 56, 56.1, 56.2, 56.3, 56.4, 56.5, 56.6, 56.7, 56.8, 56.9, 57, 57.1, 57.2, 57.3, 57.4, 57.5, 57.6, 57.7, 57.8, 57.9, 58, 58.1, 58.2, 58.3, 58.4, 58.5, 58.6, 58.7, 58.8, 58.9, 59, 59.1, 59.2, 59.3, 59.4, 59.5, 59.6, 59.7, 59.8, 59.9, 60, 60.1, 60.2, 60.3, 60.4, 60.5, 60.6, 60.7, 60.8, 60.9, 61, 61.1, 61.2, 61.3, 61.4, 61.5, 61.6, 61.7, 61.8, 61.9, 62, 62.1, 62.2, 62.3, 62.4, 62.5, 62.6, 62.7, 62.8, 62.9, 63, 63.1, 63.2, 63.3, 63.4, 63.5, 63.6, 63.7, 63.8, 63.9, 64, 64.1, 64.2, 64.3, 64.4, 64.5, 64.6, 64.7, 64.8, 64.9, 65, 65.1, 65.2, 65.3, 65.4, 65.5, 65.6, 65.7, 65.8, 65.9, 66, 66.1, 66.2, 66.3, 66.4, 66.5, 66.6, 66.7, 66.8, 66.9, 67, 67.1, 67.2, 67.3, 67.4, 67.5, 67.6, 67.7, 67.8, 67.9, 68, 68.1, 68.2, 68.3, 68.4, 68.5, 68.6, 68.7, 68.8, 68.9, 69, 69.1, 69.2, 69.3, 69.4, 69.5, 69.6, 69.7, 69.8, 69.9, 70, 70.1, 70.2, 70.3, 70.4, 70.5, 70.6, 70.7, 70.8, 70.9, 71, 71.1, 71.2, 71.3, 71.4, 71.5, 71.6, 71.7, 71.8, 71.9, 72, 72.1, 72.2, 72.3, 72.4, 72.5, 72.6, 72.7, 72.8, 72.9, 73, 73.1, 73.2, 73.3, 73.4, 73.5, 73.6, 73.7, 73.8, 73.9, 74, 74.1, 74.2, 74.3, 74.4, 74.5, 74.6, 74.7, 74.8, 74.9, 75, 75.1, 75.2, 75.3, 75.4, 75.5, 75.6, 75.7, 75.8, 75.9, 76, 76.1, 76.2, 76.3, 76.4, 76.5, 76.6, 76.7, 76.8, 76.9, 77, 77.1, 77.2, 77.3, 77.4, 77.5, 77.6, 77.7, 77.8, 77.9, 78, 78.1, 78.2, 78.3, 78.4, 78.5, 78.6, 78.7, 78.8, 78.9, 79, 79.1, 79.2, 79.3, 79.4, 79.5, 79.6, 79.7, 79.8, 79.9, 80, 80.1, 80.2, 80.3, 80.4, 80.5, 80.6, 80.7, 80.8, 80.9, 81, 81.1, 81.2, 81.3, 81.4, 81.5, 81.6, 81.7, 81.8, 81.9, 82, 82.1, 82.2, 82.3, 82.4, 82.5, 82.6, 82.7, 82.8, 82.9, 83, 83.1, 83.2, 83.3, 83.4, 83.5, 83.6, 83.7, 83.8, 83.9, 84, 84.1, 84.2, 84.3, 84.4, 84.5, 84.6, 84.7, 84.8, 84.9, 85, 85.1, 85.2, 85.3, 85.4, 85.5, 85.6, 85.7, 85.8, 85.9, 86, 86.1, 86.2, 86.3, 86.4, 86.5, 86.6, 86.7, 86.8, 86.9, 87, 87.1, 87.2, 87.3, 87.4, 87.5, 87.6, 87.7, 87.8, 87.9, 88, 88.1, 88.2, 88.3, 88.4, 88.5, 88.6, 88.7, 88.8, 88.9, 89, 89.1, 89.2, 89.3, 89.4, 89.5, 89.6, 89.7, 89.8, 89.9, 90, 90.1, 90.2, 90.3, 90.4, 90.5, 90.6, 90.7, 90.8, 90.9, 91, 91.1, 91.2, 91.3, 91.4, 91.5, 91.6, 91.7, 91.8, 91.9, 92, 92.1, 92.2, 92.3, 92.4, 92.5, 92.6, 92.7, 92.8, 92.9, 93, 93.1, 93.2, 93.3, 93.4, 93.5, 93.6, 93.7, 93.8, 93.9, 94, 94.1, 94.2, 94.3, 94.4, 94.5, 94.6, 94.7, 94.8, 94.9, 95, 95.1, 95.2, 95.3, 95.4, 95.5, 95.6, 95.7, 95.8, 95.9, 96, 96.1, 96.2, 96.3, 96.4, 96.5, 96.6, 96.7, 96.8, 96.9, 97, 97.1, 97.2, 97.3, 97.4, 97.5, 97.6, 97.7, 97.8, 97.9, 98, 98.1, 98.2, 98.3, 98.4, 98.5, 98.6, 98.7, 98.8, 98.9, 99, 99.1, 99.2, 99.3, 99.4, 99.5, 99.6, 99.7, 99.8, 99.9, 100 w/v, v/v, or w/w percent of the reaction and/or sample preparation formulation.

In some embodiments, sample preparation can include one or more biological inactivation steps. In some embodiments, nuclease inactivation can include one or more biological activation steps. In some embodiments, viral inactivation can include one or more biological inactivation steps. In some embodiments, the biological inactivation step can include exposing the sample to an enzyme or other biological molecule. In some embodiments, the enzyme or biological molecule can inactivate one or more enzymes or other molecules in the sample, such as but not limited to, one or more nucleases. In some embodiments, the enzyme or other biological molecule can bind one or more components the sample (such as a binding protein like albumin etc.) such that the bound components are inactive. In some embodiments, the enzyme or other biological molecule included in a biologic inactivation step can include, but not limited to, a DNAse inhibitor enzyme (see e.g. Eur J Med Chem. 2014 Dec. 17; 88:101-11.doi: 10.1016/j.ejmech.2014.07.040.Epub 2014 Jul. 15.), an RNAse inhibitor enzyme (e.g. QIAGEN RNase Inhibitor (Cat. No. 129916 QIAGEN, human placental RNAse inhibitor), proteinase K, and combinations thereof. Any of these compositions or combinations thereof can be included in the sample preparation formulation.

The biological molecule can be included in the sample preparation formulation or reaction at a concentration of 1 to 1000, e.g. 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 pM, nM, M, mM, or M. The biological molecule can be included in the sample preparation formulation or reaction at a concentration of 1 to 1000, e.g. 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000 units per pL, nL, μL, mL, or L.

In some embodiments, sample preparation can include one or more physiological inactivation steps. In some embodiments, nuclease inactivation can include one or more physiological inactivation steps. In some embodiments, viral inactivation can include one or more physiological inactivation steps. The phrase “physiological inactivation” refers to conditions that deviate from the normal working physiological conditions (e.g. pH, osmolarity, temperature, salinity, etc.) necessary for causing or maintaining the activation of a component (e.g. an enzyme) present in a sample that result in the inactivation or inhibition of the function or activity of the component. In some embodiments, the pH of the sample can be altered by 1, 2, 3, 4, 5, 6, 7, 8, 9, 10, 11, 12, 13, or 14 pH units away from normal physiological conditions for the sample and/or a component thereof within it. In some embodiments, the pH of the sample is adjusted to less than about 7, such as pH about 1, 2, 3, 4, 5, or about 6. In some embodiments, the pH of the sample is adjusted to greater than about 7, such as pH about 8, 9, 10, 11, 12, 13, or 14. In some embodiments, the pH is adjusted to about 7. It will be appreciated that some enzymes are active in an acidic or basic environment, and thus even a neutral pH (about 7) can serve, in some embodiments, to inactivate or inhibit such an enzyme or component of the sample. In some embodiments, the osmolarity and/or salinity of the sample can be altered outside of a normal physiological state with any suitable buffers or reagents.

In some embodiments, sample preparation can include one or more physical inactivation steps. In some embodiments, nuclease inactivation can include one or more physical inactivation steps. In some embodiments, viral inactivation can include one or more physical inactivation steps. In some embodiments, physical inactivation can include, without limitation, mechanical methods (shaking, vibrations (including resonant vibrations, acoustic vibrations, mechanical vibrations), centrifugation, electromagnetic waves, sounds waves, light waves, magnetic fields, thermal shifts (heat-cold transitions and cycles), physical bombardment, and combinations thereof.

Where the sample preparation step includes one or more reagents, active agents, buffers, and the like, these can be contained in a sample preparation formulation or viral polynucleotide preparation formulation, in the context of viral detection. In some embodiments, the reagents of the sample preparation formulation can be contained in a reaction vessel, reaction location, and/or device in solid or liquid form and the sample can be added to the reagents. In some embodiments, one or more reactions involved in sample preparation can begin once the sample is contacted and/or mixed with the sample preparation formulation. In some embodiments, the sample preparation formulation and/or viral polynucleotide preparation formulation is shelf-stable. In some embodiments, the sample preparation formulation and/or viral polynucleotide preparation formulation is shelf-stable at ambient temperature. In some embodiments, the sample preparation formulation and/or viral polynucleotide preparation formulation is shelf-stable at a temperature ranging from about 15 to about 30 degrees C. the sample preparation formulation and/or viral polynucleotide preparation formulation is stable at a temperature of about 0 to about 15 degrees C., such as 0, 1, 2, 3, 4, 5, 6, 7, 8, 9, 10, 11, 12, 13, 14 or about 15 degrees C. In some embodiments, a lyophilized, freeze dried, or otherwise dehydrated or desiccated sample preparation formulation is stable, such as shelf-stable, at a temperature ranging from about 0 degrees C. or about 4 degrees C. to about 15 or about 25 degrees C., such as about 0, 1, 2, 3, or 4 to about 5, 6, 7, 8. 9, 10, 11, 12, 13, 14, 15, 16, 17, 18, 19, 20, 21, 22, 23, 24, or about 25 degrees C. In some embodiments, the sample preparation formulation, particularly in a lyophilized, freeze dried, dehydrated or otherwise desiccated form is able to be stored for 1, 2, 3, 4, 5, 6, 7, 8, 9, 10, 11, or 12 days, weeks, months, or years at a temperature ranging from about −80 degrees C., −20 degrees C., 0 degrees C., about 4 degrees C., to about 5, 6, 7, 8. 9, 10, 11, 12, 13, 14, 15, 16, 17, 18, 19, 20, 21, 22, 23, 24, or about 25 degrees or more and still maintain downstream reaction (e.g., amplification, CRISPR-Cas based nucleic acid detection) fidelity and integrity.

In some embodiments, the sample preparation formulation and/or viral polynucleotide preparation formulation or one or more components thereof are lyophilized.

In some embodiments, the sample preparation formulation can be part of or combined with reagents for amplification and/or enrichment of a target polynucleotide, and/or compositions and systems for CRISPR-Cas based nucleic acid detection described elsewhere herein.

In one example embodiments, the sample preparation formulation includes 0.1 M HEPES (pH 8.0), 300 mM KCl, and 25% PEG-8000 w/v. In some these embodiments, this formulation can be diluted 1×, 2×, 3×, 4×, 5×, 6× or more to a working sample preparation formulation.

In one example embodiments, the sample preparation formulation includes about 20 mM HEPES (pH 8.0), about 60 mM KCl, about 3.5-5%% PEG-8000 w/v.

In one example embodiments, the sample preparation formulation, such as one formulated for lyophilization, includes 20 mM HEPES (pH 8.0), 5% w/v sucrose, and 150 mM mannitol. In some embodiments, the

In one example embodiments, the sample preparation formulation includes about 20 mM HEPES (pH 8.0), about 3.5% w/v PEG-1500 and about 0.35% w/v PEG-8000.

In one example embodiments, the sample preparation formulation includes about 20 mM HEPES (pH 8.0), about 3.5% w/v PEG-1500 and about 0.35% w/v PEG-8000, and an amount of KCl.

Amplification and Enrichment of Target and/or Signal

### Target Amplification

In certain example embodiments, target RNAs and/or DNAs may be amplified prior to activating the CRISPR effector protein. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). In certain embodiments, the amplification can utilize a transposase-based isothermal amplification method (see e.g. WO 2020/006049, which is incorporated by reference herein as if expressed in its entirety), nickase-based isothermal amplification method (see e.g. WO 2020/006067, which is incorporated by reference herein as if expressed in its entirety), or a helicase-based amplification method (see e.g. WO 2020/006036, which is incorporated by reference herein as if expressed in its entirety). In some embodiments, amplification is via LAMP. In some embodiments, amplification is via RPA.

In certain example embodiments, the RNA or DNA amplification is nucleic acid sequence-based amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by the guide RNAs thus further enhancing the sensitivity of the assay. Binding of the target RNAs by the guide RNAs then leads to activation of the CRISPR effector protein and the methods proceed as outlined above. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the CRISPR effector system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or apatamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification reagents can include one or more primers and/or probes optimized for amplification of a target sequence by one or more of the amplification methods previously described. Primer and probe design for the methods described herein will be within the purview of one of ordinary skill in the art in view of the context and disclosure only provided herein.

Amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

In some embodiments, the amplification reagent or component thereof is shelf-stable. In some embodiments, the amplification reagent or component thereof is shelf-stable at ambient temperature. In some embodiments, the amplification reagent or component thereof is shelf-stable at 15-30 degrees C.

### Target Polynucleotide Enrichment

In certain example embodiments, target RNA or DNA may first be enriched prior to detection or amplification of the target RNA or DNA. In certain example embodiments, this enrichment may be achieved by binding of the target nucleic acids by a CRISPR effector system.

Current target-specific enrichment protocols require single-stranded nucleic acid prior to hybridization with probes. Among various advantages, the present embodiments can skip this step and enable direct targeting to double-stranded DNA (either partly or completely double-stranded). In addition, the embodiments disclosed herein are enzyme-driven targeting methods that offer faster kinetics and easier workflow allowing for isothermal enrichment. In certain example embodiments enrichment may take place between 20-37° C. In certain example embodiments, a set of guide RNAs to different target nucleic acids are used in a single assay, allowing for detection of multiple targets an/or multiple variants of a single target.

In certain example embodiments, a dead CRISPR effector protein may bind the target nucleic acid in solution and then subsequently be isolated from said solution. For example, the dead CRISPR effector protein bound to the target nucleic acid, may be isolated from the solution using an antibody or other molecule, such as an aptamer, that specifically binds the dead CRISPR effector protein.

In other example embodiments, the dead CRISPR effector protein may bound to a solid substrate. A fixed substrate may refer to any material that is appropriate for or can be modified to be appropriate for the attachment of a polypeptide or a polynucleotide. Possible substrates include, but are not limited to, glass and modified functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundes, and a variety of other polymers. In some embodiments, the solid support comprises a patterned surface suitable for immobilization of molecules in an ordered pattern. In certain embodiments a patterned surface refers to an arrangement of different regions in or on an exposed layer of a solid support. In some embodiments, the solid support comprises an array of wells or depressions in a surface. The composition and geometry of the solid support can vary with its use. In some embodiments, the solids support is a planar structure such as a slide, chip, microchip and/or array. As such, the surface of the substrate can be in the form of a planar layer. In some embodiments, the solid support comprises one or more surfaces of a flowcell. The term “flowcell” as used herein referes to a chamber comprising a solid surface across which one or more fluid reagent can be flowed. Example flowcells and related fluidic systems and detection platforms that can be readily used in the methods of the present disclosure are described, for example, in Bentley et al. Nature 456:53-59 (2008), WO 04/0918497, U.S. Pat. No. 7,057,026; WO 91/06678; WO 07/123744; U.S. Pat. Nos. 7,329,492; 7,211,414; 7,315,019; 7,405,281, and US 2008/0108082. In some embodiments, the solid support or its surface is non-planar, such as the inner or outer surface of a tube or vessel. In some embodiments, the solid support comprise microspheres or beads. “Microspheres,” “bead,” “particles,” are intended to mean within the context of a solid substrate to mean small discrete particles made of various material including, but not limited to, plastics, ceramics, glass, and plystyrene. In certain embodiments, the microspheres are magnetic microsphers or eads. Alternatively or additionally, the beads may be porous. The bead sizes range from nanometers, e.g., 100 nm, to millimeters, e.g. 1 mm.

A sample containing, or suspected of containing, the target nucleic acids may then be exposed to the substrate to allow binding of the target nucleic acids to the bound dead CRISPR effector protein. Non-target molecules may then be washed away. In certain example embodiments, the target nucleic acids may then be released from the CRISPR effector protein/guide RNA complex for further detection using the methods disclosed herein. In certain example embodiments, the target nucleic acids may first be amplified as described herein.

In certain example embodiments, the CRISPR effector may be labeled with a binding tag. In certain example embodiments the CRISPR effector may be chemically tagged. For example, the CRISPR effector may be chemically biotinylated. In another example embodiment, a fusion may be created by adding additional sequence encoding a fusion to the CRISPR effector. One example of such a fusion is an AviTag™, which employs a highly targeted enzymatic conjugation of a single biotin on a unique 15 amino acide peptide tag. In certain embodiments, the CRISPR effector may be labeled with a capture tag such as, but not limited to, GST, Myc, hemagglutinin (HA), green fluorescent protein (GFP), flag, His tag, TAP tag, and Fc tag. The binding tag, whether a fusion, chemical tag, or capture tag, may be used to either pull down the CRISPR effector system once it has bound a target nucleic acid or to fix the CRISPR effector system on the solid substrate.

In certain example embodiments, the guide RNA may be labeled with a binding tag. In certain example embodiments, the entire guide RNA may be labeled using in vitro transcription (IVT) incorporating one or more biotinylated nucleotides, such as, biotinylated uracil. In some embodiments, biotin can be chemically or enzymatically added to the guide RNA, such as, the addition of one or more biotin groups to the 3′ end of the guide RNA. The binding tag may be used to pull down the guide RNA/target nucleic acid complex after binding has occurred, for example, by exposing the guide RNA/target nucleic acid to a streptavidin coated solid substrate.

Accordingly, in certain example embodiments, an engineered or non-naturally-occurring CRISPR effector may be used for enrichment purposes. In an embodiment, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of the RNA strand at the target locus of interest. In a preferred embodiment, the one or more mutations may comprise two mutations. In a preferred embodiment the one or more amino acid residues are modified in a C2c2 effector protein, e.g., an engineered or non-naturally-occurring effector protein or C2c2. In particular embodiments, the one or more modified of mutated amino acid residues are one or more of those in C2c2 corresponding to R597, H602, R1278 and H1283 (referenced to Lsh C2c2 amino acids), such as mutations R597A, H602A, R1278A and H1283A, or the corresponding amino acid residues in Lsh C2c2 orthologues.

In particular embodiments, the one or more modified of mutated amino acid residues are one or more of those in C2c2 corresponding to K2, K39, V40, E479, L514, V518, N524, G534, K535, E580, L597, V602, D630, F676, L709, 1713, R717 (HEPN), N718, H722 (HEPN), E773, P823, V828, 1879, Y880, F884, Y997, L1001, F1009, L1013, Y1093, L1099, L1111, Y1114, L1203, D1222, Y1244, L1250, L1253, K1261, 11334, L1355, L1359, R1362, Y1366, E1371, R1372, D1373, R1509 (HEPN), H1514 (HEPN), Y1543, D1544, K1546, K1548, V1551, 11558, according to C2c2 consensus numbering. In certain embodiments, the one or more modified of mutated amino acid residues are one or more of those in C2c2 corresponding to R717 and R1509. In certain embodiments, the one or more modified of mutated amino acid residues are one or more of those in C2c2 corresponding to K2, K39, K535, K1261, R1362, R1372, K1546 and K1548. In certain embodiments, said mutations result in a protein having an altered or modified activity. In certain embodiments, said mutations result in a protein having a reduced activity, such as reduced specificity. In certain embodiments, said mutations result in a protein having no catalytic activity (i.e. “dead” C2c2). In an embodiment, said amino acid residues correspond to Lsh C2c2 amino acid residues, or the corresponding amino acid residues of a C2c2 protein from a different species.

The above enrichment systems may also be used to deplete a sample of certain nucleic acids. For example, guide RNAs may be designed to bind non-target RNAs to remove the non-target RNAs from the sample. In one example embodiment, the guide RNAs may be designed to bind nucleic acids that do carry a particular nucleic acid variation. For example, in a given sample a higher copy number of non-variant nucleic acids may be expected. Accordingly, the embodiments disclosed herein may be used to remove the non-variant nucleic acids from a sample, to increase the efficiency with which the detection CRISPR effector system can detect the target variant sequences in a given sample.

Amplification and/or Enhancement of Detectable Signal

In certain example embodiments, further modification may be introduced that further amplify the detectable positive signal. For example, activated CRISPR effector protein collateral activation may be use to generate a secondary target or additional guide sequence, or both. In one example embodiment, the reaction solution would contain a secondary target that is spiked in at high concentration. The secondary target may be distinct from the primary target (i.e. the target for which the assay is designed to detect) and in certain instances may be common across all reaction volumes. A secondary guide sequence for the secondary target may be protected, e.g. by a secondary structural feature such as a hairpin with a RNA loop, and unable to bind the second target or the CRISPR effector protein. Cleavage of the protecting group by an activated CRISPR effector protein (i.e. after activation by formation of complex with the primary target(s) in solution) and formation of a complex with free CRISPR effector protein in solution and activation from the spiked in secondary target. In certain other example embodiments, a similar concept is used with a second guide sequence to a secondary target sequence. The secondary target sequence may be protected a structural feature or protecting group on the secondary target. Cleavage of a protecting group off the secondary target then allows additional CRISPR effector protein/second guide sequence/secondary target complex to form. In yet another example embodiment, activation of CRISPR effector protein by the primary target(s) may be used to cleave a protected or circularized primer, which is then released to perform an isothermal amplification reaction, such as those disclosed herein, on a template that encodes a secondary guide sequence, secondary target sequence, or both. Subsequent transcription of this amplified template would produce more secondary guide sequence and/or secondary target sequence, followed by additional CRISPR effector protein collateral activation.

In some embodiments another CRISPR system can be used to enrich or amplify the detectable signal. In some embodiments the first CRISPR system(s) that is/are activated upon target binding can produce, such as via collateral activity, species that can activate (or be targets of) a second CRISPR system thus amplifying the signal for detection. In some embodiments a CRISPR type-III effector can be used as the signal amplifying system. In some embodiments, the type III effector is Csm6, which is which is activated by cyclic adenylate molecules or linear adenine homopolymers terminated with a 2′,3′-cyclic phosphate. In some embodiments, the first CRISPR system includes a Cas13 (e.g. Cas 13a, 13b, 13c, or 13d) and/or a Cas 12a effector(s) and the amplification system or molecule is or includes Csm6. See also Gootenberg et al. 2018. Science. 360:439-44 and WO 2019/051318, which are incorporated by reference herein as if expressed in their entireties.

### Microbe Detection and Applications

In certain example embodiments, the systems, devices, and methods, disclosed herein are directed to detecting the presence of one or more microbial agents in a sample, such as a biological sample obtained from a subject. In certain example embodiments, the microbe may be a bacterium, a fungus, a yeast, a protozoa, a parasite, or a virus. Accordingly, the methods disclosed herein can be adapted for use in other methods (or in combination) with other methods that require quick identification of microbe species, monitoring the presence of microbial proteins (antigens), antibodies, antibody genes, detection of certain phenotypes (e.g. bacterial resistance), monitoring of disease progression and/or outbreak, and antibiotic screening. Because of the rapid and sensitive diagnostic capabilities of the embodiments disclosed here, detection of microbe species type, down to a single nucleotide difference, and the ability to be deployed as a POC device, the embodiments disclosed herein may be used guide therapeutic regimens, such as selection of the appropriate antibiotic or antiviral. The embodiments disclosed herein may also be used to screen environmental samples (air, water, surfaces, food etc.) for the presence of microbial contamination.

Disclosed is a method to identify microbial species, such as bacterial, viral, fungal, yeast, or parasitic species, or the like. Particular embodiments disclosed herein describe methods and systems that will identify and distinguish microbial species within a single sample, or across multiple samples, allowing for recognition of many different microbes. The present methods allow the detection of pathogens and distinguishing between two or more species of one or more organisms, e.g., bacteria, viruses, yeast, protozoa, and fungi or a combination thereof, in a biological or environmental sample, by detecting the presence of a target nucleic acid sequence in the sample. A positive signal obtained from the sample indicates the presence of the microbe. Multiple microbes can be identified simultaneously using the methods and systems of the invention, by employing the use of more than one effector protein, wherein each effector protein targets a specific microbial target sequence. In this way, a multi-level analysis can be performed for a particular subject in which any number of microbes can be detected at once. In some embodiments, simultaneous detection of multiple microbes may be performed using a set of probes that can identify one or more microbial species.

Multiplex analysis of samples enables large-scale detection of samples, reducing the time and cost of analyses. However, multiplex analyses are often limited by the availability of a biological sample. In accordance with the invention, however, alternatives to multiplex analysis may be performed such that multiple effector proteins can be added to a single sample and each detection construct may be combined with a separate quencher dye. In this case, positive signals may be obtained from each quencher dye separately for multiple detection in a single sample.

Disclosed herein are methods for distinguishing between two or more species of one or more organisms in a sample. The methods are also amenable to detecting one or more species of one or more organisms in a sample.

### Microbe Detection

In some embodiments, a method for detecting microbes in samples is provided comprising distributing a sample or set of samples into one or more individual discrete volumes, the individual discrete volumes comprising a CRISPR system as described herein; incubating the sample or set of samples under conditions sufficient to allow binding of the one or more guide RNAs to one or more microbe-specific targets; activating the CRISPR effector protein via binding of the one or more guide RNAs to the one or more target molecules, wherein activating the CRISPR effector protein results in modification of the RNA-based detection construct such that a detectable positive signal is generated; and detecting the detectable positive signal, wherein detection of the detectable positive signal indicates a presence of one or more target molecules in the sample. The one or more target molecules may be mRNA, gDNA (coding or non-coding), trRNA, or RNA comprising a target nucleotide tide sequence that may be used to distinguish two or more microbial species/strains from one another. The guide RNAs may be designed to detect target sequences. The embodiments disclosed herein may also utilize certain steps to improve hybridization between guide RNA and target RNA sequences. Methods for enhancing ribonucleic acid hybridization are disclosed in WO 2015/085194, entitled “Enhanced Methods of Ribonucleic Acid Hybridization” which is incorporated herein by reference. The microbe-specific target may be RNA or DNA or a protein. If DNA method may further comprise the use of DNA primers that introduce a RNA polymerase promoter as described herein. If the target is a protein then aptamers can be utilized and the method includes one or more specific to protein detection described herein.

### Detection of Single Nucleotide Variants

In some embodiments, one or more identified target sequences may be detected using guide RNAs that are specific for and bind to the target sequence as described herein. The systems and methods of the present invention can distinguish even between single nucleotide polymorphisms present among different microbial species and therefore, use of multiple guide RNAs in accordance with the invention may further expand on or improve the number of target sequences that may be used to distinguish between species. For example, in some embodiments, the one or more guide RNAs may distinguish between microbes at the species, genus, family, order, class, phylum, kingdom, or phenotype, or a combination thereof.

Detection Based on rRNA Sequences

In certain example embodiments, the devices, systems, and methods disclosed herein may be used to distinguish multiple microbial species in a sample. In certain example embodiments, identification may be based on ribosomal RNA sequences, including the 16S, 23S, and 5S subunits. Methods for identifying relevant rRNA sequences are disclosed in U.S. Patent Application Publication No. 2017/0029872. In certain example embodiments, a set of guide RNA may designed to distinguish each species by a variable region that is unique to each species or strain. Guide RNAs may also be designed to target RNA genes that distinguish microbes at the genus, family, order, class, phylum, kingdom levels, or a combination thereof. In certain example embodiments where amplification is used, a set of amplification primers may be designed to flanking constant regions of the ribosomal RNA sequence and a guide RNA designed to distinguish each species by a variable internal region. In certain example embodiments, the primers and guide RNAs may be designed to conserved and variable regions in the 16S subunit respectfully. Other genes or genomic regions that uniquely variable across species or a subset of species such as the RecA gene family, RNA polymerase β subunit, may be used as well. Other suitable phylogenetic markers, and methods for identifying the same, are discussed for example in Wu et al. arXiv:1307.8690 [q-bio.GN].

In certain example embodiments, a method or diagnostic is designed to screen microbes across multiple phylogenetic and/or phenotypic levels at the same time. For example, the method or diagnostic may comprise the use of multiple CRISPR systems with different guide RNAs. A first set of guide RNAs may distinguish, for example, between mycobacteria, gram positive, and gram-negative bacteria. These general classes can be even further subdivided. For example, guide RNAs could be designed and used in the method or diagnostic that distinguish enteric and non-enteric within gram negative bacteria. A second set of guide RNA can be designed to distinguish microbes at the genus or species level. Thus, a matrix may be produced identifying all mycobacteria, gram positive, gram negative (further divided into enteric and non-enteric) with each genus of species of bacteria identified in a given sample that fall within one of those classes. The foregoing is for example purposes only. Other means for classifying other microbe types are also contemplated and would follow the general structure described above.

### Screening for Drug Resistance

In certain example embodiments, the devices, systems and methods disclosed herein may be used to screen for microbial genes of interest, for example antibiotic and/or antiviral resistance genes. Guide RNAs may be designed to distinguish between known genes of interest. Samples, including clinical samples, may then be screened using the embodiments disclosed herein for detection of such genes. The ability to screen for drug resistance at POC would have tremendous benefit in selecting an appropriate treatment regime. In certain example embodiments, the antibiotic resistance genes are carbapenemases including KPC, NDM1, CTX-M15, OXA-48. Other antibiotic resistance genes are known and may be found for example in the Comprehensive Antibiotic Resistance Database (Jia et al. “CARD 2017: expansion and model-centric curation of the Comprehensive Antibiotic Resistance Database.” Nucleic Acids Research, 45, D566-573).

Ribavirin is an effective antiviral that hits a number of RNA viruses. Several clinically important virues have evolved ribavirin resitance including Foot and Mouth Disease Virus doi:10.1128/JVI.03594-13; polio virus (Pfeifer and Kirkegaard. PNAS, 100(12):7289-7294, 2003); and hepatitis C virus (Pfeiffer and Kirkegaard, J. Virol. 79(4):2346-2355, 2005). A number of other persistant RNA viruses, such as hepatitis and HIV, have evolved resitance to existing antiviral drugs: hepatitis B virus (lamivudine, tenofovir, entecavir) doi:10/1002/hep22900; hepatits C virus (telaprevir, BILN2061, ITMN-191, SCh6, boceprevir, AG-021541, ACH-806) doi:10.1002/hep.22549; and HIV (many drug resistance mutations) hivb.standford.edu. The embodiments disclosed herein may be used to detect such variants among others.

Aside from drug resistance, there are a number of clinically relevant mutations that could be detected with the embodiments disclosed herein, such as persistent versus acute infection in LCMV (doi:10.1073/pnas.1019304108), and increased infectivity of Ebola (Diehl et al. Cell. 2016, 167(4):1088-1098.

As described herein elsewhere, closely related microbial species (e.g. having only a single nucleotide difference in a given target sequence) may be distinguished by introduction of a synthetic mismatch in the gRNA.

### Set Cover Approaches

In particular embodiments, a set of guide RNAs is designed that can identify, for example, all microbial species within a defined set of microbes. In certain example embodiments, the methods for generating guide RNAs as described herein may be compared to methods disclosed in WO 2017/040316, incorporated herein by reference. As described in WO 2017040316, a set cover solution may identify the minimal number of target sequences probes or guide RNAs needed to cover an entire target sequence or set of target sequences, e.g. a set of genomic sequences. Set cover approaches have been used previously to identify primers and/or microarray probes, typically in the 20 to 50 base pair range. See, e.g. Pearson et al., cs.virginia.edu/-robins/papers/primers_dam11_final.pdf., Jabado et al. Nucleic Acids Res. 2006 34(22):6605-11, Jabado et al. Nucleic Acids Res. 2008, 36(1):e3 doi10.1093/nar/gkm1106, Duitama et al. Nucleic Acids Res. 2009, 37(8):2483-2492, Phillippy et al. BMC Bioinformatics. 2009, 10:293 doi:10.1186/1471-2105-10-293. However, such approaches generally involved treating each primer/probe as k-mers and searching for exact matches or allowing for inexact matches using suffix arrays. In addition, the methods generally take a binary approach to detecting hybridization by selecting primers or probes such that each input sequence only needs to be bound by one primer or probe and the position of this binding along the sequence is irrelevant. Alternative methods may divide a target genome into pre-defined windows and effectively treat each window as a separate input sequence under the binary approach—i.e. they determine whether a given probe or guide RNA binds within each window and require that all of the windows be bound by the state of some probe or guide RNA. Effectively, these approaches treat each element of the “universe” in the set cover problem as being either an entire input sequence or a pre-defined window of an input sequence, and each element is considered “covered” if the start of a probe or guide RNA binds within the element. These approaches limit the fluidity to which different probe or guide RNA designs are allowed to cover a given target sequence.

In contrast, the embodiments disclosed herein are directed to detecting longer probe or guide RNA lengths, for example, in the range of 70 bp to 200 bp that are suitable for hybrid selection sequencing. In addition, the methods disclosed WO 2017/040316 herein may be applied to take a pan-target sequence approach capable of defining a probe or guide RNA sets that can identify and facilitate the detection sequencing of all species and/or strains sequences in a large and/or variable target sequence set. For example, the methods disclosed herein may be used to identify all variants of a given virus, or multiple different viruses in a single assay. Further, the method disclosed herein treat each element of the “universe” in the set cover problem as being a nucleotide of a target sequence, and each element is considered “covered” as long as a probe or guide RNA binds to some segment of a target genome that includes the element. Theses type of set cover methods may used instead of the binary approach of previous methods, the methods disclosed in herein better model how a probe or guide RNA may hybridize to a target sequence. Rather than only asking if a given guide RNA sequence does or does not bind to a given window, such approaches may be used to detect a hybridization pattern—i.e. where a given probe or guide RNA binds to a target sequence or target sequences—and then determines from those hybridization patterns the minimum number of probes or guide RNAs needed to cover the set of target sequences to a degree sufficient to enable both enrichment from a sample and sequencing of any and all target sequences. These hybridization patterns may be determined by defining certain parameters that minimize a loss function, thereby enabling identification of minimal probe or guide RNA sets in a way that allows parameters to vary for each species, e.g. to reflect the diversity of each species, as well as in a computationally efficient manner that cannot be achieved using a straightforward application of a set cover solution, such as those previously applied in the probe or guide RNA design context.

The ability to detect multiple transcript abundances may allow for the generation of unique microbial signatures indicative of a particular phenotype. Various machine learning techniques may be used to derive the gene signatures. Accordingly, the guide RNAs of the CRISPR systems may be used to identify and/or quantitate relative levels of biomarkers defined by the gene signature in order to detect certain phenotypes. In certain example embodiments, the gene signature indicates susceptibility to an antibiotic, resistance to an antibiotic, or a combination thereof.

In one aspect of the invention, a method comprises detecting one or more pathogens. In this manner, differentiation between infection of a subject by individual microbes may be obtained. In some embodiments, such differentiation may enable detection or diagnosis by a clinician of specific diseases, for example, different variants of a disease. Preferably the pathogen sequence is a genome of the pathogen or a fragment thereof. The method further may comprise determining the evolution of the pathogen. Determining the evolution of the pathogen may comprise identification of pathogen mutations, e.g. nucleotide deletion, nucleotide insertion, nucleotide substitution. Amongst the latter, there are non-synonymous, synonymous, and noncoding substitutions. Mutations are more frequently non-synonymous during an outbreak. The method may further comprise determining the substitution rate between two pathogen sequences analyzed as described above. Whether the mutations are deleterious or even adaptive would require functional analysis, however, the rate of non-synonymous mutations suggests that continued progression of this epidemic could afford an opportunity for pathogen adaptation, underscoring the need for rapid containment. Thus, the method may further comprise assessing the risk of viral adaptation, wherein the number non-synonymous mutations is determined. (Gire, et al., Science 345, 1369, 2014).

### Monitoring Microbe Outbreaks

In some embodiments, a CRISPR system or methods of use thereof as described herein may be used to determine the evolution of a pathogen outbreak. The method may comprise detecting one or more target sequences from a plurality of samples from one or more subjects, wherein the target sequence is a sequence from a microbe causing the outbreaks. Such a method may further comprise determining a pattern of pathogen transmission, or a mechanism involved in a disease outbreak caused by a pathogen.

The pattern of pathogen transmission may comprise continued new transmissions from the natural reservoir of the pathogen or subject-to-subject transmissions (e.g. human-to-human transmission) following a single transmission from the natural reservoir or a mixture of both. In one embodiment, the pathogen transmission may be bacterial or viral transmission, in such case, the target sequence is preferably a microbial genome or fragments thereof. In one embodiment, the pattern of the pathogen transmission is the early pattern of the pathogen transmission, i.e. at the beginning of the pathogen outbreak. Determining the pattern of the pathogen transmission at the beginning of the outbreak increases likelihood of stopping the outbreak at the earliest possible time thereby reducing the possibility of local and international dissemination.

Determining the pattern of the pathogen transmission may comprise detecting a pathogen sequence according to the methods described herein. Determining the pattern of the pathogen transmission may further comprise detecting shared intra-host variations of the pathogen sequence between the subjects and determining whether the shared intra-host variations show temporal patterns. Patterns in observed intrahost and interhost variation provide important insight about transmission and epidemiology (Gire, et al., 2014).

Detection of shared intra-host variations between the subjects that show temporal patterns is an indication of transmission links between subject (in particular between humans) because it can be explained by subject infection from multiple sources (superinfection), sample contamination recurring mutations (with or without balancing selection to reinforce mutations), or co-transmission of slightly divergent viruses that arose by mutation earlier in the transmission chain (Park, et al., Cell 161(7):1516-1526, 2015). Detection of shared intra-host variations between subjects may comprise detection of intra-host variants located at common single nucleotide polymorphism (SNP) positions. Positive detection of intra-host variants located at common (SNP) positions is indicative of superinfection and contamination as primary explanations for the intra-host variants. Superinfection and contamination can be parted on the basis of SNP frequency appearing as inter-host variants (Park, et al., 2015). Otherwise superinfection and contamination can be ruled out. In this latter case, detection of shared intra-host variations between subjects may further comprise assessing the frequencies of synonymous and nonsynonymous variants and comparing the frequency of synonymous and nonsynonymous variants to one another. A nonsynonymous mutation is a mutation that alters the amino acid of the protein, likely resulting in a biological change in the microbe that is subject to natural selection. Synonymous substitution does not alter an amino acid sequence. Equal frequency of synonymous and nonsynonymous variants is indicative of the intra-host variants evolving neutrally. If frequencies of synonymous and nonsynonymous variants are divergent, the intra-host variants are likely to be maintained by balancing selection. If frequencies of synonymous and nonsynonymous variants are low, this is indicative of recurrent mutation. If frequencies of synonymous and nonsynonymous variants are high, this is indicative of co-transmission (Park, et al., 2015).

Like Ebola virus, Lassa virus (LASV) can cause hemorrhagic fever with high case fatality rates. Andersen et al. generated a genomic catalog of almost 200 LASV sequences from clinical and rodent reservoir samples (Andersen, et al., Cell Volume 162, Issue 4, p 738-750, 13 Aug. 2015). Andersen et al. show that whereas the 2013-2015 EVD epidemic is fueled by human-to-human transmissions, LASV infections mainly result from reservoir-to-human infections. Andersen et al. elucidated the spread of LASV across West Africa and show that this migration was accompanied by changes in LASV genome abundance, fatality rates, codon adaptation, and translational efficiency. The method may further comprise phylogenetically comparing a first pathogen sequence to a second pathogen sequence, and determining whether there is a phylogenetic link between the first and second pathogen sequences. The second pathogen sequence may be an earlier reference sequence. If there is a phylogenetic link, the method may further comprise rooting the phylogeny of the first pathogen sequence to the second pathogen sequence. Thus, it is possible to construct the lineage of the first pathogen sequence. (Park, et al., 2015).

The method may further comprise determining whether the mutations are deleterious or adaptive. Deleterious mutations are indicative of transmission-impaired viruses and dead-end infections, thus normally only present in an individual subject. Mutations unique to one individual subject are those that occur on the external branches of the phylogenetic tree, whereas internal branch mutations are those present in multiple samples (i.e. in multiple subjects). Higher rate of nonsynonymous substitution is a characteristic of external branches of the phylogenetic tree (Park, et al., 2015).

In internal branches of the phylogenetic tree, selection has had more opportunity to filter out deleterious mutants. Internal branches, by definition, have produced multiple descendent lineages and are thus less likely to include mutations with fitness costs. Thus, lower rate of nonsynonymous substitution is indicative of internal branches (Park, et al., 2015).

Synonymous mutations, which likely have less impact on fitness, occurred at more comparable frequencies on internal and external branches (Park, et al., 2015).

By analyzing the sequenced target sequence, such as viral genomes, it is possible to discover the mechanisms responsible for the severity of the epidemic episode such as during the 2014 Ebola outbreak. For example, Gire et al. made a phylogenetic comparison of the genomes of the 2014 outbreak to all 20 genomes from earlier outbreaks suggests that the 2014 West African virus likely spread from central Africa within the past decade. Rooting the phylogeny using divergence from other ebolavirus genomes was problematic (6, 13). However, rooting the tree on the oldest outbreak revealed a strong correlation between sample date and root-to-tip distance, with a substitution rate of 8×10-4 per site per year (13). This suggests that the lineages of the three most recent outbreaks all diverged from a common ancestor at roughly the same time, around 2004, which supports the hypothesis that each outbreak represents an independent zoonotic event from the same genetically diverse viral population in its natural reservoir. They also found out that the 2014 EBOV outbreak might be caused by a single transmission from the natural reservoir, followed by human-to-human transmission during the outbreak. Their results also suggested that the epidemic episode in Sierra Leon might stem from the introduction of two genetically distinct viruses from Guinea around the same time (Gire, et al., 2014).

It has been also possible to determine how the Lassa virus spread out from its origin point, in particular thanks to human-to-human transmission and even retrace the history of this spread 400 years back (Andersen, et al., Cell 162(4):738-50, 2015).

In relation to the work needed during the 2013-2015 EBOV outbreak and the difficulties encountered by the medical staff at the site of the outbreak, and more generally, the method of the invention makes it possible to carry out sequencing using fewer selected probes such that sequencing can be accelerated, thus shortening the time needed from sample taking to results procurement. Further, kits and systems can be designed to be usable on the field so that diagnostics of a patient can be readily performed without need to send or ship samples to another part of the country or the world.

In any method described above, sequencing the target sequence or fragment thereof may used any of the sequencing processes described above. Further, sequencing the target sequence or fragment thereof may be a near-real-time sequencing. Sequencing the target sequence or fragment thereof may be carried out according to previously described methods (Experimental Procedures: Matranga et al., 2014; and Gire, et al., 2014). Sequencing the target sequence or fragment thereof may comprise parallel sequencing of a plurality of target sequences. Sequencing the target sequence or fragment thereof may comprise Illumina sequencing.

Analyzing the target sequence or fragment thereof that hybridizes to one or more of the selected probes may be an identifying analysis, wherein hybridization of a selected probe to the target sequence or a fragment thereof indicates the presence of the target sequence within the sample.

Currently, primary diagnostics are based on the symptoms a patient has. However, various diseases may share identical symptoms so that diagnostics rely much on statistics. For example, malaria triggers flu-like symptoms: headache, fever, shivering, joint pain, vomiting, hemolytic anemia, jaundice, hemoglobin in the urine, retinal damage, and convulsions. These symptoms are also common for septicemia, gastroenteritis, and viral diseases. Amongst the latter, Ebola hemorrhagic fever has the following symptoms fever, sore throat, muscular pain, headaches, vomiting, diarrhea, rash, decreased function of the liver and kidneys, internal and external hemorrhage.

When a patient is presented to a medical unit, for example in tropical Africa, basic diagnostics will conclude to malaria because statistically, malaria is the most probable disease within that region of Africa. The patient is consequently treated for malaria although the patient might not actually have contracted the disease and the patient ends up not being correctly treated. This lack of correct treatment can be life-threatening especially when the disease the patient contracted presents a rapid evolution. It might be too late before the medical staff realizes that the treatment given to the patient is ineffective and comes to the correct diagnostics and administers the adequate treatment to the patient.

The method of the invention provides a solution to this situation. Indeed, because the number of guide RNAs can be dramatically reduced, this makes it possible to provide on a single chip selected probes divided into groups, each group being specific to one disease, such that a plurality of diseases, e.g. viral infection, can be diagnosed at the same time. Thanks to the invention, more than 3 diseases can be diagnosed on a single chip, preferably more than 4, 5, 6, 7, 8, 9, 10, 11, 12, 13, 14, 15, 16, 17, 18, 19, 20 diseases at the same time, preferably the diseases that most commonly occur within the population of a given geographical area. Since each group of selected probes is specific to one of the diagnosed diseases, a more accurate diagnostics can be performed, thus diminishing the risk of administering the wrong treatment to the patient.

In other cases, a disease such as a viral infection may occur without any symptoms, or had caused symptoms but they faded out before the patient is presented to the medical staff. In such cases, either the patient does not seek any medical assistance or the diagnostics is complicated due to the absence of symptoms on the day of the presentation.

The present invention may also be used in concert with other methods of diagnosing disease, identifying pathogens and optimizing treatment based upon detection of nucleic acids, such as mRNA in crude, non-purified samples.

The method of the invention also provides a powerful tool to address this situation. Indeed, since a plurality of groups of selected guide RNAs, each group being specific to one of the most common diseases that occur within the population of the given area, are comprised within a single diagnostic, the medical staff only need to contact a biological sample taken from the patient with the chip. Reading the chip reveals the diseases the patient has contracted.

In some cases, the patient is presented to the medical staff for diagnostics of particular symptoms. The method of the invention makes it possible not only to identify which disease causes these symptoms but at the same time determine whether the patient suffers from another disease he was not aware of.

This information might be of utmost importance when searching for the mechanisms of an outbreak. Indeed, groups of patients with identical viruses also show temporal patterns suggesting a subject-to-subject transmission links.

In some embodiments, a CRISPR system or methods of use thereof as described herein may be used to predict disease outcome in patients suffering from viral diseases. In specific embodiments, such viral diseases may include, but are not necessarily limited to, Lassa fever. Specific factors related to Lassa fever disease outcome may include but are not necessarily limited to, age, extent of kidney injury, and/or CNS injury.

### Screening Microbial Genetic Perturbations

In certain example embodiments, the CRISPR systems disclosed herein may be used to screen microbial genetic perturbations. Such methods may be useful, for example to map out microbial pathways and functional networks. Microbial cells may be genetically modified and then screened under different experimental conditions. As described above, the embodiments disclosed herein can screen for multiple target molecules in a single sample, or a single target in a single individual discrete volume in a multiplex fashion. Genetically modified microbes may be modified to include a nucleic acid barcode sequence that identifies the particular genetic modification carried by a particular microbial cell or population of microbial cells. A barcode is s short sequence of nucleotides (for example, DNA, RNA, or combinations thereof) that is used as an identifier. A nucleic acid barcode may have a length of 4-100 nucleotides and be either single or double-stranded. Methods for identifying cells with barcodes are known in the art. Accordingly, guide RNAs of the CRISPR effector systems described herein may be used to detect the barcode. Detection of the positive detectable signal indicates the presence of a particular genetic modification in the sample. The methods disclosed herein may be combined with other methods for detecting complimentary genotype or phenotypic readouts indicating the effect of the genetic modification under the experimental conditions tested. Genetic modifications to be screened may include, but are not limited to, a gene knock-in, a gene knock-out, inversions, translocations, transpositions, or one or more nucleotide insertions, deletions, substitutions, mutations, or addition of nucleic acids encoding an epitope with a functional consequence such as altering protein stability or detection. In a similar fashion, the methods described herein may be used in synthetic biology application to screen the functionality of specific arrangements of gene regulatory elements and gene expression modules.

In certain example embodiments, the methods may be used to screen hypomorphs. Generation of hypomorphs and their use in identifying key bacterial functional genes and identification of new antibiotic therapeutics as disclosed in PCT/US2016/060730 entitled “Multiplex High-Resolution Detection of Micro-organism Strains, Related Kits, Diagnostic Methods and Screening Assays” filed Nov. 4, 2016, which is incorporated herein by reference.

The different experimental conditions may comprise exposure of the microbial cells to different chemical agents, combinations of chemical agents, different concentrations of chemical agents or combinations of chemical agents, different durations of exposure to chemical agents or combinations of chemical agents, different physical parameters, or both. In certain example embodiments the chemical agent is an antibiotic or antiviral. Different physical parameters to be screened may include different temperatures, atmospheric pressures, different atmospheric and non-atmospheric gas concentrations, different pH levels, different culture media compositions, or a combination thereof.

### Screening Environmental Samples

The methods disclosed herein may also be used to screen environmental samples for contaminants by detecting the presence of target nucleic acids. For example, in some embodiments, the invention provides a method of detecting microbes, comprising: exposing a CRISPR system as described herein to a sample; activating an RNA effector protein via binding of one or more guide RNAs to one or more microbe-specific target RNAs or one or more trigger RNAs such that a detectable positive signal is produced. The positive signal can be detected and is indicative of the presence of one or more microbes in the sample. In some embodiments, the CRISPR system may be on a substrate as described herein, and the substrate may be exposed to the sample. In other embodiments, the same CRISPR system, and/or a different CRISPR system may be applied to multiple discrete locations on the substrate. In further embodiments, the different CRISPR system may detect a different microbe at each location. As described in further detail above, a substrate may be a flexible materials substrate, for example, including, but not limited to, a paper substrate, a fabric substrate, or a flexible polymer-based substrate.

In accordance with the invention, the substrate may be exposed to the sample passively, by temporarily immersing the substrate in a fluid to be sampled, by applying a fluid to be tested to the substrate, or by contacting a surface to be tested with the substrate. Any means of introducing the sample to the substrate may be used as appropriate.

As described herein, a sample for use with the invention may be a biological or environmental sample, such as a food sample (fresh fruits or vegetables, meats), a beverage sample, a paper surface, a fabric surface, a metal surface, a wood surface, a plastic surface, a soil sample, a freshwater sample, a wastewater sample, a saline water sample, exposure to atmospheric air or other gas sample, or a combination thereof. For example, household/commercial/industrial surfaces made of any materials including, but not limited to, metal, wood, plastic, rubber, or the like, may be swabbed and tested for contaminants. Soil samples may be tested for the presence of pathogenic bacteria or parasites, or other microbes, both for environmental purposes and/or for human, animal, or plant disease testing. Water samples such as freshwater samples, wastewater samples, or saline water samples can be evaluated for cleanliness and safety, and/or potability, to detect the presence of, for example, Cryptosporidium parvum, Giardia lamblia, or other microbial contamination. In further embodiments, a biological sample may be obtained from a source including, but not limited to, a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface. In some particular embodiments, an environmental sample or biological samples may be crude samples and/or the one or more target molecules may not be purified or amplified from the sample prior to application of the method. Identification of microbes may be useful and/or needed for any number of applications, and thus any type of sample from any source deemed appropriate by one of skill in the art may be used in accordance with the invention.

In some embodiments, Checking for food contamination by bacteria, such as E. coli, in restaurants or other food providers; food surfaces; Testing water for pathogens like Salmonella, Campylobacter, or E. coli; also checking food quality for manufacturers and regulators to determine the purity of meat sources; identifying air contamination with pathogens such as Legionella; Checking whether beer is contaminated or spoiled by pathogens like Pediococcus and Lactobacillus; contamination of pasteurized or un-pasteurized cheese by bacteria or fungi during manufacture.

A microbe in accordance with the invention may be a pathogenic microbe or a microbe that results in food or consumable product spoilage. A pathogenic microbe may be pathogenic or otherwise undesirable to humans, animals, or plants. For human or animal purposes, a microbe may cause a disease or result in illness. Animal or veterinary applications of the present invention may identify animals infected with a microbe. For example, the methods and systems of the invention may identify companion animals with pathogens including, but not limited to, kennel cough, rabies virus, and heartworms. In other embodiments, the methods and systems of the invention may be used for parentage testing for breeding purposes. A plant microbe may result in harm or disease to a plant, reduction in yield, or alter traits such as color, taste, consistency, odor, For food or consumable contamination purposes, a microbe may adversely affect the taste, odor, color, consistency or other commercial properties of the food or consumable product. In certain example embodiments, the microbe is a bacterial species. The bacteria may be a psychrotroph, a coliform, a lactic acid bacteria, or a spore-forming bacteria. In certain example embodiments, the bacteria may be any bacterial species that causes disease or illness, or otherwise results in an unwanted product or trait. Bacteria in accordance with the invention may be pathogenic to humans, animals, or plants.

### Example Microbes

The embodiment disclosed herein may be used to detect a number of different microbes. The term microbe as used herein includes bacteria, fungus, protozoa, parasites and viruses.

### Bacteria

The following provides an example list of the types of microbes that might be detected using the embodiments disclosed herein. In certain example embodiments, the microbe is a bacterium. Examples of bacteria that can be detected in accordance with the disclosed methods include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

Near-real-time microbial diagnostics are needed for food, clinical, industrial, and other environmental settings (see e.g., Lu T K, Bowers J, and Koeris M S., Trends Biotechnol. 2013 June; 31(6):325-7). In certain embodiments, the assay described herein is configured for detection of foodborne pathogens using guide RNAs specific to a pathogen (e.g., Campylobacter jejuni, Clostridium perfringens, Salmonella spp., Escherichia coli, Bacillus cereus, Listeria monocytogenes, Shigella spp., Staphylococcus aureus, Staphylococcal enteritis, Streptococcus, Vibrio cholerae, Vibrio parahaemolyticus, Vibrio vulrificus, Yersinia enterocolitica and Yersinia pseudotuberculosis, Brucella spp., Corynebacterium ulcerans, Coxiella burnetii, or Plesiomonas shigelloides).

### Fungi

In certain example embodiments, the microbe is a fungus or a fungal species. Examples of fungi that can be detected in accordance with the disclosed methods include without limitation any one or more of (or any combination of), Aspergillus, Blastomyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium.

In certain example embodiments, the fungus is a yeast. Examples of yeast that can be detected in accordance with disclosed methods include without limitation one or more of (or any combination of), Aspergillus species (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), a Geotrichum species, a Saccharomyces species, a Hansenula species, a Candida species (such as Candida albicans), a Kluyveromyces species, a Debaryomyces species, a Pichia species, or combination thereof. In certain example embodiments, the fungi is a mold. Example molds include, but are not limited to, a Penicillium species, a Cladosporium species, a Byssochlamys species, or a combination thereof.

### Protozoa

In certain example embodiments, the microbe is a protozoan. Examples of protozoa that can be detected in accordance with the disclosed methods and devices include without limitation any one or more of (or any combination of), Euglenozoa, Heterolobosea, Diplomonadida, Amoebozoa, Blastocystic, and Apicomplexa. Example Euglenoza include, but are not limited to, Trypanosoma cruzi (Chagas disease), T. brucei gambiense, T. brucei rhodesiense, Leishmania braziliensis, L. infantum, L. mexicana, L. major, L. tropica, and L. donovani. Example Heterolobosea include, but are not limited to, Naegleria fowleri. Example Diplomonadid include, but are not limited to, Giardia intestinalis (G. lamblia, G. duodenalis). Example Amoebozoa include, but are not limited to, Acanthamoeba castellanii, Balamuthia madrillaris, Entamoeba histolytica. Example Blastocystis include, but are not limited to, Blastocystic hominis. Example Apicomplexa include, but are not limited to, Babesia microti, Cryptosporidium parvum, Cyclospora cayetanensis, Plasmodium falciparum, P. vivax, P. ovale, P. malariae, and Toxoplasma gondii. Babesia microti, Cryptosporidium parvum, Cyclospora cayetanensis, Plasmodium falciparum, P. vivax, P. ovale, P. malariae, and Toxoplasma gondii.

### Parasites

In certain example embodiments, the microbe is a parasite. Examples of parasites that can be detected in accordance with disclosed methods include without limitation one or more of (or any combination of), an Onchocerca species and a Plasmodium species.

### Viruses

In certain example embodiments, the systems, devices, and methods, disclosed herein are directed to detecting viruses in a sample. The embodiments disclosed herein may be used to detect viral infection (e.g. of a subject or plant), or determination of a viral strain, including viral strains that differ by a single nucleotide polymorphism. The virus may be a DNA virus, a RNA virus, or a retrovirus. Non-limiting example of viruses useful with the present invention include, but are not limited to Ebola, measles, SARS, Chikungunya, hepatitis, Marburg, yellow fever, MERS, Dengue, Lassa, influenza, rhabdovirus or HIV. A hepatitis virus may include hepatitis A, hepatitis B, or hepatitis C. An influenza virus may include, for example, influenza A or influenza B. An HIV may include HIV 1 or HIV 2. In certain example embodiments, the viral sequence may be a human respiratory syncytial virus, Sudan ebola virus, Bundibugyo virus, Tai Forest ebola virus, Reston ebola virus, Achimota, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyoxivirus, Australian bat lyssavirus, Avian bornavirus, Avian metapneumovirus, Avian paramyoxviruses, penguin or Falkland Islandsvirus, BK polyomavirus, Bagaza virus, Banna virus, Bat hepevirus, Bat sapovirus, Bear Canon mammarenavirus, Beilong virus, Betacoronoavirus, Betapapillomavirus 1-6, Bhanja virus, Bokeloh bat lyssavirus, Boma disease virus, Bourbon virus, Bovine hepacivirus, Bovine parainfluenza virus 3, Bovine respiratory syncytial virus, Brazoran virus, Bunyamwere virus, Caliciviridae virus. California encephalitis virus, Candiru virus, Canine distemper virus, Canaine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyoxiviurs SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human gential-associated circular DNA virus-1, Human herpesvirus 1-8, Human immunodeficiency virus 1/2, Huan mastadenovirus A-G, Human papillomavirus, Human parainfluenza virus 1-4, Human paraechovirus, Human picobirnavirus, Human smacovirus, Ikoma lyssavirus, Ilheus virus, Influenza A-C, Ippy mammarenavirus, Irkut virus, J-virus, JC polyomavirus, Japanses encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2\.225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Procine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan equine encephalitis virus, Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence. Examples of RNA viruses that may be detected include one or more of (or any combination of) Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus. In certain example embodiments, the virus is Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

In certain example embodiments, the virus may be a plant virus selected from the group comprising Tobacco mosaic virus (TMV), Tomato spotted wilt virus (TSWV), Cucumber mosaic virus (CMV), Potato virus Y (PVY), the RT virus Cauliflower mosaic virus (CaMV), Plum pox virus (PPV), Brome mosaic virus (BMV), Potato virus X (PVX), Citrus tristeza virus (CTV), Barley yellow dwarf virus (BYDV), Potato leafroll virus (PLRV), Tomato bushy stunt virus (TBSV), rice tungro spherical virus (RTSV), rice yellow mottle virus (RYMV), rice hoja blanca virus (RHBV), maize rayado fino virus (MRFV), maize dwarf mosaic virus (MDMV), sugarcane mosaic virus (SCMV), Sweet potato feathery mottle virus (SPFMV), sweet potato sunken vein closterovirus (SPSVV), Grapevine fanleaf virus (GFLV), Grapevine virus A (GVA), Grapevine virus B (GVB), Grapevine fleck virus (GFkV), Grapevine leafroll-associated virus-1, -2, and -3, (GLRaV-1, -2, and -3), Arabis mosaic virus (ArMV), or Rupestris stem pitting-associated virus (RSPaV). In a preferred embodiment, the target RNA molecule is part of said pathogen or transcribed from a DNA molecule of said pathogen. For example, the target sequence may be comprised in the genome of an RNA virus. It is further preferred that CRISPR effector protein hydrolyzes said target RNA molecule of said pathogen in said plant if said pathogen infects or has infected said plant. It is thus preferred that the CRISPR system is capable of cleaving the target RNA molecule from the plant pathogen both when the CRISPR system (or parts needed for its completion) is applied therapeutically, i.e. after infection has occurred or prophylactically, i.e. before infection has occurred.

In certain example embodiments, the virus may be a retrovirus. Example retroviruses that may be detected using the embodiments disclosed herein include one or more of or any combination of viruses of the Genus Alpharetrovirus, Betaretrovirus, Gammaretrovirus, Deltaretrovirus, Epsilonretrovirus, Lentivirus, Spumavirus, or the Family Metaviridae, Pseudoviridae, and Retroviridae (including HIV), Hepadnaviridae (including Hepatitis B virus), and Caulimoviridae (including Cauliflower mosaic virus).

In certain example embodiments, the virus is a DNA virus. Example DNA viruses that may be detected using the embodiments disclosed herein include one or more of (or any combination of) viruses from the Family Myoviridae, Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, Rhizidovirus, among oIn some embodiments, a method of diagnosing a species-specific bacterial infection in a subject suspected of having a bacterial infection is described as obtaining a sample comprising bacterial ribosomal ribonucleic acid from the subject; contacting the sample with one or more of the probes described, and detecting hybridization between the bacterial ribosomal ribonucleic acid sequence present in the sample and the probe, wherein the detection of hybridization indicates that the subject is infected with Escherichia coli, Klebsiella pneumoniae, Pseudomonas aeruginosa, Staphylococcus aureus, Acinetobacter baumannii, Candida albicans, Enterobacter cloacae, Enterococcus faecalis, Enterococcus faecium, Proteus mirabilis, Staphylococcus agalactiae, or Staphylococcus maltophilia or a combination thereof.

### SARS-CoV-2

In some embodiments, the systems and assay described herien is configured to detect a target nucleic acid of SARS-CoV-2 and/or SARS-CoV-2 variant.

As used herein, the term “variant” refers to any virus having one or more mutations as compared to a known virus. A strain is a genetic variant or subtype of a virus. The terms ‘strain’, ‘variant’, and ‘isolate’ may be used interchangeably. In certain embodiments, a variant has developed a “specific group of mutations” that causes the variant to behave differently than that of the strain it originated from. While there are many thousands of variants of SARS-CoV-2, (Koyama, Takahiko Koyama; Platt, Daniela; Parida, Laxmi (June 2020). “Variant analysis of SARS-CoV-2 genomes”. Bulletin of the World Health Organization. 98: 495-504) there are also much larger groupings called clades. Several different clade nomenclatures for SARS-CoV-2 have been proposed. As of December 2020, GISAID, referring to SARS-CoV-2 as hCoV-19 identified seven clades (O, S, L, V, G, GH, and GR) (Alm E, Broberg E K, Connor T, et al. Geographical and temporal distribution of SARS-CoV-2 clades in the WHO European Region, January to June 2020 [published correction appears in Euro Surveill. 2020 August; 25(33):]. Euro Surveill. 2020; 25(32):2001410). Also as of December 2020, Nextstrain identified five (19A, 19B, 20A, 20B, and 20C) (Cited in Alm et al. 2020). Guan et al. identified five global clades (G614, S84, V251, 1378 and D392) (Guan Q, Sadykov M, Mfarrej S, et al. A genetic barcode of SARS-CoV-2 for monitoring global distribution of different clades during the COVID-19 pandemic. Int J Infect Dis. 2020; 100:216-223). Rambaut et al. proposed the term “lineage” in a 2020 article in Nature Microbiology; as of December 2020, there have been five major lineages (A, B, B.1, B.1.1, and B.1.777) identified (Rambaut, A.; Holmes, E. C.; O'Toole, Á.; et al. “A dynamic nomenclature proposal for SARS-CoV-2 lineages to assist genomic epidemiology”. 5: 1403-1407).

Genetic variants of SARS-CoV-2 have been emerging and circulating around the world throughout the COVID-19 pandemic (see, e.g., The US Centers for Disease Control and Prevention; www.cdc.gov/coronavirus/2019-ncov/variants/variant-info.html). Exemplary, non-limiting variants applicable to the present disclosure include variants of SARS-CoV-2, particularly those having substitutions of therapeutic concern. Table 7 shows exemplary, non-limiting genetic substitutions in SARS-CoV-2 variants.

TABLE 7

| | Common Pango Lineages with Spike |
|---|---|
| Spike Protein Substitution | Protein Substitutions |
| L452R | A.2.5, B.1, B.1.429, B.1.427, B.1.617.1, |
| | B.1.526.1, B.1.617.2, C.36.3 |
| E484K | B.1.1.318, B.1.1.7, B.1.351, B.1.525, |
| | B.1.526, B.1.621, B.1.623, P.1, |
| | P.1.1, P.1.2, R.1 |
| K417N, E484K, N501Y | B.1.351, B.1.351.3 |
| K417T, E484K, N501Y | P.1, P.1.1, P.1.2 |

Phylogenetic Assignment of Named Global Outbreak (PANGO) Lineages is software tool developed by members of the Rambaut Lab. The associated web application was developed by the Centre for Genomic Pathogen Surveillance in South Cambridgeshire and is intended to implement the dynamic nomenclature of SARS-CoV-2 lineages, known as the PANGO nomenclature. It is available at cov-lineages.org.

In some embodiments, the SARS-CoV-2 variant is and/or includes: B.1.1.7, also known as Alpha (WHO) or UK variant, having the following spike protein substitutions: 69del, 70del, 144del, (E484K*), (S494P*), N501Y, A570D, D614G, P681H, T7161, S982A, and D1118H (K1191N*); B.1.351, also known as Beta (WHO) or South Africa variant, having the following spike protein substitutions: D80A, D215G, 241del, 242del, 243del, K417N, E484K, N501Y, D614G, and A701V; B.1.427, also known as Epsilon (WHO) or US California variant, having the following spike protein substitutions: L452R, and D614G; B.1.429, also known as Epsilon (WHO) or US California variant, having the following spike protein substitutions: S13I, W152C, L452R, and D614G; B.1.617.2, also known as Delta (WHO) or India variant, having the following spike protein substitutions: T19R, (G142D), 156del, 157del, R158G, L452R, T478K, D614G, P681R, and D950N; and P.1, also known as Gamma (WHO) or Japan/Brazil variant, having the following spike protein substitutions: L18F, T20N, P26S, D138Y, R190S, K417T, E484K, N501Y, D614G, H655Y, and T1027I, or any combination thereof.

In some embodiments, the SARS-CoV-2 variant is classified and/or otherwise identified as a Variant of Concern (VOC) by the World Health Organization and/or the U.S. Centers for Disease Control. A VOC is a variant for which there is evidence of an increase in transmissibility, more severe disease (e.g., increased hospitalizations or deaths), significant reduction in neutralization by antibodies generated during previous infection or vaccination, reduced effectiveness of treatments or vaccines, or diagnostic detection failures.

In some embodiments, the SARS-Cov-2 variant is classified and/or otherwise identified as a Variant of High Consequence (VHC) by the World Health Organization and/or the U.S. Centers for Disease Control. A variant of high consequence has clear evidence that prevention measures or medical countermeasures (MCMs) have significantly reduced effectiveness relative to previously circulating variants.

In some embodiments, the SARS-Cov-2 variant is classified and/or otherwise identified as a Variant of Interest (VOI) by the World Health Organization and/or the U.S. Centers for Disease Control. A VOI is a variant with specific genetic markers that have been associated with changes to receptor binding, reduced neutralization by antibodies generated against previous infection or vaccination, reduced efficacy of treatments, potential diagnostic impact, or predicted increase in transmissibility or disease severity.

In some embodiments, the SARS-Cov-2 variant is classified and/or is otherwise identified as a Variant of Note (VON). As used herein, VON refers to both “variants of concern” and “variants of note” as the two phrases are used and defined by Pangolin (cov-lineages.org) and provided in their available “VOC reports” available at cov-lineages.org.

In some embodiments the SARS-Cov-2 variant is a VOC. In some embodiments, the SARS-CoV-2 variant is or includes an Alpha variant (e.g., Pango lineage B.1.1.7), a Beta variant (e.g., Pango lineage B.1.351, B.1.351.1, B.1.351.2, and/or B.1.351.3), a Delta variant (e.g., Pango lineage B.1.617.2, AY.1, AY.2, AY.3 and/or AY.3.1); a Gamma variant (e.g., Pango lineage P.1, P.1.1, P.1.2, P.1.4, P.1.6, and/or P.1.7), or any combination thereof.

In some embodiments the SARS-Cov-2 variant is a VOL. In some embodiments, the SARS-CoV-2 variant is or includes an Eta variant (e.g., Pango lineage B.1.525 (Spike protein substitutions A67V, 69del, 70del, 144del, E484K, D614G, Q677H, F888L)); an Iota variant (e.g., Pango lineage B.1.526 (Spike protein substitutions L5F, (D80G*), T95I, (Y144-*), (F157S*), D253G, (L452R*), (S477N*), E484K, D614G, A701V, (T859N*), (D950H*), (Q957R*))); a Kappa variant (e.g., Pango lineage B.1.617.1 (Spike protein substitutions (T95I), G142D, E154K, L452R, E484Q, D614G, P681R, Q1071H)); Pango lineage variant B.1.617.2 (Spike protein substitutions T19R, G142D, L452R, E484Q, D614G, P681R, D950N)), Lambda (e.g., Pango lineage C.37); or any combination thereof.

In some embodiments SARS-Cov-2 variant is a VON. In some embodiments, the SARS-Cov-2 variant is or includes Pango lineage variant P.1 (alias, B.1.1.28.1.) as described in Rambaut et al. 2020. Nat. Microbiol. 5:1403-1407) (spike protein substitutions: T20N, P26S, D138Y, R190S, K417T, E484K, N501Y, H655Y, TI027I)); an Alpha variant (e.g., Pango lineage B.1.1.7); a Beta variant (e.g., Pango lineage B.1.351, B.1.351.1, B.1.351.2, and/or B.1.351.3); Pango lineage variant B.1.617.2 (Spike protein substitutions T19R, G142D, L452R, E484Q, D614G, P681R, D950N)); an Eta variant (e.g., Pango lineage B.1.525); Pango lineage variant A.23.1 (as described in Bugembe et al. medRxiv. 2021. doi: https://doi.org/10.1101/2021.02.08.21251393) (spike protein substitutions: F157L, V367F, Q613H, P681R); or any combination thereof.

In additional example embodiments, the virus is a virus listed in Table 8 below, or a virus of the indicated genus/family.

TABLE 8
Exemplary Viral Targets

| Virus | Genus, Family | Host | Transmission | Disease |
|---|---|---|---|---|
| Adeno-associated | Dependovirus | Human, vertebrates | Respiratory | None |
| virus | | | | |
| Aichi virus | Kobuvirus, Picornaviridae | Human | Fecal-oral | Gastroenteritis |
| Australian bat | Lyssavirus, | Human, bats | Zoonosis, animal | Fatal encephalitis |
| lyssavirus | Rhabdoviridae | | bite | |
| BK polyomavirus | Polyomavirus, | Human | Respiratory | None |
| | Polyomaviridae | | fluids or urine | |
| Banna virus | Seadornavirus, Reoviridae | Human, cattle, pig, | Zoonosis, | Encephalitis |
| | | mosquitoes | arthropod bite | |
| Barmah forest | Alphavirus, Togaviridae | Human, marsupials, | Zoonosis, | Fever, joint pain |
| virus | | mosquitoes | arthropod bite | |
| Bunyamwera virus | Orthobunyavirus, | Human, mosquitoes | Zoonosis, | Encephalitis |
| | Bunyaviridae | | arthropod bite | |
| Bunyavirus La | Orthobunyavirus, | Human, deer, | Zoonosis, | Encephalitis |
| Crosse | Bunyaviridae | mosquitoes, tamias | arthropod bite | |
| Bunyavirus | Orthobunyavirus, | Human, rodents, | Zoonosis, | Encephalitis |
| snowshoe hare | Bunyaviridae | mosquitoes | arthropod bite | |
| Cercopithecine | Lymphocryptovirus, | Human, monkeys | Zoonosis, animal | Encephalitis |
| herpesvirus | Herpesviridae | | bite | |
| Chandipura virus | Vesiculovirus, | Human, sandflies | Zoonosis, | Encephalitis |
| | Rhabdoviridae | | athropod bite | |
| Chikungunya virus | Alphavirus, Togaviridae | Human, monkeys, | Zoonosis, | Fever, joint pain |
| | | mosquitoes | arthropod bite | |
| Cosavirus A | Cosavirus, Picornaviridae | Human | Fecal-oral | — |
| | | | (probable) | |
| Cowpox virus | Orthopoxvirus, Poxviridae | Human, mammals | Zoonosis, | None |
| | | | contact | |
| Coxsackievirus | Enterovirus, | Human | Fecal-oral | Meningitis, |
| | Picornaviridae | | | myocarditis, |
| | | | | paralysis |
| Crimean-Congo | Nairovirus, Bunyaviridae | Human, vertebrates, | Zoonosis, | Hemorrhagic |
| hemorrhagic fever | | ticks | arthropod bite | fever |
| virus | | | | |
| Dengue virus | Flavivirus, | Human, mosquitoes | Zoonosis, | Hemorrhagic |
| | Flaviviridae | | arthropod bite | fever |
| Dhori virus | Thogotovirus, | Human, ticks | Zoonosis, | Fever, encephalitis |
| | Orthomyxoviridae | | arthropod bite | |
| Dugbe virus | Nairovirus, Bunyaviridae | Human, ticks | Zoonosis, | Thrombocytopaenia |
| | | | arthropod bite | |
| Duvenhage virus | Lyssavirus, | Human, mammals | Zoonosis, animal | Fatal encephalitis |
| | Rhabdoviridae | | bite | |
| Eastern equine | Alphavirus, Togaviridae | Human, birds, | Zoonosis, | Encephalitis |
| encephalitis virus | | mosquitoes | arthropod bite | |
| Ebolavirus | Ebolavirus, Filoviridae | Human, | Zoonosis, | Hemorrhagic |
| | | monkeys, bats | contact | fever |
| Echovirus | Enterovirus, | Human | Fecal-oral | Common cold |
| | Picornaviridae | | | |
| Encephalomyocarditis | Cardiovirus, | Human, mouse, rat, pig | Zoonosis | Encephalitis |
| virus | Picornaviridae | | | |
| Epstein-Barr virus | Lymphocryptovirus, | Human | Contact, saliva | Mononucleosis |
| | Herpesviridae | | | |
| European bat | Lyssavirus, | Human, bats | Zoonosis, animal | Fatal encephalitis |
| lyssavirus | Rhabdovirus | | bite | |
| GB virus | Pegivirus, Flaviviridae | Human | Blood, | None |
| C/Hepatitis G | | | occasionally | |
| virus | | | sexual | |
| Hantaan virus | Hantavirus, Bunyaviridae | Human, rodents | Zoonosis, urine, | Renal or |
| | | | saliva | respiratory |
| | | | | syndrome |
| Hendra virus | Henipavirus, | Human, | Zoonosis, animal | Encephalitis |
| | paramyxoviridae | horse, bats | bite | |
| Hepatitis A virus | Hepatovirus, | Human | Fecal-oral | Hepatitis |
| | picornaviridae | | | |
| Hepatitis B virus | Orthohepadnavirus, | Human, Chimpanzees | Sexual contact, | Hepatitis |
| | Hepadnaviridae | | blood | |
| Hepatitis C virus | Hepacivirus, | Human | Sexual, blood | Hepatitis |
| | Flaviviridae | | | |
| Hepatitis E virus | Hepevirus, | Human, pig, monkeys, | Zoonosis, food | Hepatitis |
| | Unassigned | some rodents, chicken | | |
| Hepatitis delta | Deltavirus, Unassigned | Human | Sexual contact, | Hepatitis |
| virus | | | blood | |
| Horsepox virus | Orthopoxvirus, Poxviridae | Human, horses | Zoonosis, | None |
| | | | contact | |
| Human adenovirus | Mastadenovirus, | Human | Respiratory, | Respiratory |
| | Adenoviridae | | fecal-oral | |
| Human astrovirus | Mamastrovirus, | Human | Fecal-oral | Gastroenteritis |
| | Astroviridae | | | |
| Human | Alphacoronavirus, | Human | Respiratory | Respiratory |
| coronavirus | Coronaviridae | | | |
| Human | Cytomegalovirus, | Human | Contact, urine, | Mononucleosis, |
| cytomegalovirus | Herpesviridae | | saliva | pneumonia |
| Human | Enterovirus, | Human | Fecal-oral | Diarrhea, |
| enterovirus 68, 70 | Picornaviridae | | | neurological |
| | | | | disorder |
| Human | Simplexvirus, | Human | Sexual contact, | Skin lesions |
| herpesvirus 1 | Herpesviridae | | saliva | |
| Human | Simplexvirus, | Human | Sexual contact, | Skin lesions |
| herpesvirus 2 | Herpesviridae | | saliva | |
| Human | Roseolovirus, | Human | Respiratory, | Skin lesions |
| herpesvirus 6 | Herpesviridae | | contact | |
| Human | Roseolovirus, | Human | Respiratory, | Skin lesions |
| herpesvirus 7 | Herpesviridae | | contact | |
| Human | Rhadinovirus, | Human | Sexual contact, | Skin lymphoma |
| herpesvirus 8 | Herpesviridae | | saliva | |
| Human | Lentivirus, Retroviridae | Human | Sexual contact, | AIDS |
| immunodeficiency | | | blood | |
| virus | | | | |
| Human | Mupapillomavirus, | Human | Contact | Skin warts |
| papillomavirus 1 | Papillomaviridae | | | |
| Human | Alphapapillomavirus, | Human | Contact | Skin warts |
| papillomavirus 2 | Papillomaviridae | | | |
| Human | Alphapapillomavirus, | Human | Sexual | Genital warts, |
| papillomavirus | Papillomaviridae | | | cervical cancer |
| 16, 18 | | | | |
| Human | Respirovirus, | Human | Respiratory | Respiratory |
| parainfluenza | Paramyxoviridae | | | |
| Human parvovirus | Erythrovirus, Parvoviridae | Human | Respiratory | Skin lesion |
| B19 | | | | |
| Human respiratory | Pneumovirus, | Human | Respiratory | Respiratory |
| syncytial virus | Paramyxoviridae | | | |
| Human rhinovirus | Enterovirus | Human | Respiratory | Respiratory |
| Human SARS | Betacoronavirus, | Human, palm civet | Zoonosis | Respiratory |
| coronavirus | Coronaviridae | | | |
| Human | Spumavirus, | Human | Contact, saliva | None |
| spumaretrovirus | Retroviridae | | | |
| Human T- | Deltaretrovirus, | Human | Sexual contact, | Leukemia |
| lymphotropic virus | Retroviridae | | maternal- | |
| | | | neonatal | |
| Human torovirus | Torovirus, Coronaviridae | Human | Fecal-oral | Gastroenteritis |
| Influenza A virus | Influenzavirus A, | Human, birds, pigs | Respiratory or | Flu |
| | Orthomyxoviridae | | Zoonosis, animal | |
| | | | contact | |
| Influenza B virus | Influenzavirus B, | Human | Respiratory | Flu |
| | Orthomyxoviridae | | | |
| Influenza C virus | Influenzavirus C, | Human | Respiratory | Flu |
| | Orthomyxoviridae | | | |
| Isfahan virus | Vesiculovirus, | Human, sandflies, | Zoonosis, | Undocumented, |
| | Rhabdoviridae | gerbils | arthropod bite | encephalitis? |
| JC polyomavirus | Polyomavirus, | Human | Fecal-oral or | Encephalitis |
| | Polyomaviridae | | urine | |
| Japanese | Flavivirus, | Human, horses, birds, | Zoonosis, | Encephalitis |
| encephalitis virus | Flaviviridae | mosquitoes | arthropod borne | |
| Junin arenavirus | Arenavirus, | Human, rodents | Zoonosis, fomite | Hemorrhagic |
| | Arenaviridae | | | fever |
| KI Polyomavirus | Polyomavirus, | Human | Fecal-oral or | Encephalitis |
| | Polyomaviridae | | urine | |
| Kunjin virus | Flavivirus, | Human, horses, birds, | Zoonosis, | Encephalitis |
| | Flaviviridae | mosquitoes | arthropod borne | |
| Lagos bat virus | Lyssavirus, | Human, mammals | Zoonosis, animal | Fatal encephalitis |
| | Rhabdoviridae | | bite | |
| Lake Victoria | Marburgvirus, Filoviridae | Human, monkeys, bats | Zoonosis, fomite | Hemorrhagic |
| marburgvirus | | | | fever |
| Langat virus | Flavivirus, | Human, ticks | Zoonosis, | Encephalitis |
| | Flaviviridae | | arthropod borne | |
| Lassa virus | Arenavirus, | Human, rats | Zoonosis, | Hemorrhagic |
| | Arenaviridae | | fomites | fever |
| Lordsdale virus | Norovirus, Caliciviridae | Human | Fecal-oral | Gastroenteritis |
| Louping ill virus | Flavivirus, | Human, mammals, | Zoonosis, | Encephalitis |
| | Flaviviridae | ticks | arthropod bite | |
| Lymphocytic | Arenavirus, | Human, rodents | Zoonosis, fomite | Encephalitis |
| choriomeningitis | Arenaviridae | | | |
| virus | | | | |
| Machupo virus | Arenavirus, | Human, monkeys, | Zoonosis, fomite | Encephalitis |
| | Arenaviridae | mouse | | |
| Mayaro virus | Alphavirus, Togaviridae | Human, mosquitoes | Zoonosis, | Fever, joint pain |
| | | | arthropod bite | |
| MERS | Betacoronavirus, | Human, Tomb bat | Zoonosis | Respiratory |
| coronavirus | Coronaviridae | | | |
| Measles virus | Morbilivirus, | Human | Respiratory | Fever, rash |
| | Paramyxoviridae | | | |
| Mengo | Cardiovirus, | Human, mouse, rabbit | Zoonosis | Encephalitis |
| encephalomyocarditis | Picornaviridae | | | |
| virus | | | | |
| Merkel cell | Polyomavirus, | Human | — | Merkel cell |
| polyomavirus | Polyomaviridae | | | carcinoma |
| Mokola virus | Lyssavirus, | Human, rodents, cat, | Zoonosis, animal | Encephalitis |
| | Rhabdoviridae | dog shrew | bite | |
| Molluscum | Molluscipoxvirus, | Human | Contact | Skin lesions |
| contagiosum virus | Poxviridae | | | |
| Monkeypox virus | Orthopoxvirus | Human, mouse, prairie | Zoonosis, | Skin lesions |
| | | dog | contact | |
| Mumps virus | Rubulavirus, | Human | Respiratory, | Mumps |
| | Paramyxoviridae | | saliva | |
| Murray valley | Flavivirus | Human, mosquitoes | Zoonosis, | Encephalitis |
| encephalitis virus | | | arthropod bite | |
| New York virus | Hantavirus, Bunyavirus | Human, mouse | Zoonosis, urine, | Hemorrhagic |
| | | | saliva | fever |
| Nipah virus | Henipavirus, | Human, bats | Zoonosis, animal | Encephalitis |
| | Paramyxoviridae | | bite | |
| Norwalk virus | Norovirus, Caliciviridae | Human | Fecal-oral | Gastroenteritis |
| O'nyong-nyong | Alphavirus, Togaviridae | Human, mosquitoes | Zoonosis, | Fever, joint pain |
| virus | | | arthropod bite | |
| Orf virus | Parapoxvirus, Poxviridae | Human, mammals | Zoonosis, | Skin lesions |
| | | | contact | |
| Oropouche virus | Orthobunyavirus | Human, wild | Zoonosis, | Fever, joint pain |
| | | animals(sloths) | arthropod bite | |
| Pichinde virus | Arenavirus, | Human, rat, guinea pig | Zoonosis, fomite | Hemorrhagic |
| | Arenaviridae | | | fever |
| Poliovirus | Enterovirus, | Human, mammals | Fecal-oral | Poliomyelitis |
| | Picornaviridae | | | |
| Punta toro | Phlebovirus, Bunyaviridae | Human, sandflies | Zoonosis, | Hemorrhagic |
| phlebovirus | | | arthropod bite | fever |
| Puumala virus | Hantavirus, Bunyavirus | Human, bank vole | Zoonosis, urine, | Hemorrhagic |
| | | | saliva | fever |
| Rabies virus | Lyssavirus, | Human, mammals | Zoonosis, animal | Fatal encephalitis |
| | Rhabdoviridae | | bite | |
| Rift valley fever | Phlebovirus, Bunyaviridae | Human, mammals, | Zoonosis, | Hemorrhagic |
| virus | | mosquitoes, sandflies | arthropod bite | fever |
| Rosavirus A | Rosavirus, Picornaviridae | Human | | |
| Ross river virus | Alphavirus, Togaviridae | Human, mosquitoes, | Zoonosis, | Fever, joint pain |
| | | marsupials | arthropod bite | |
| Rotavirus A | Rotavirus, Reoviridae | Human | Fecal-oral | Gastroenteritis |
| Rotavirus B | Rotavirus, Reoviridae | Human | Fecal-oral | Gastroenteritis |
| Rotavirus C | Rotavirus, Reoviridae | Human | Fecal-oral | Gastroenteritis |
| Rubella virus | Rubivirus, Togaviridae | Human | Respiratory | Rubella |
| Sagiyama virus | Alphavirus, Togaviridae | Human, horse, pig, | Zoonosis, | Fever, joint pain |
| | | mosquitoes | arthropod bite | |
| Salivirus A | Salivirus, Picornaviridae | Human | | Gastroenteritis |
| Sandfly fever | Phlebovirus, Bunyaviridae | Human, sandflies | Zoonosis, | Hemorrhagic |
| sicilian virus | | | arthropod bite | fever |
| Sapporo virus | Sapovirus, Caliciviridae | Human | Fecal-oral | Gastroenteritis |
| Semliki forest | Alphavirus, Togaviridae | Human, birds, | Zoonosis, | Fever, joint pain |
| virus | | hedgehog, mosquitoes | arthropod bite | |
| Seoul virus | Hantavirus, Bunyavirus | Human, rats | Zoonosis, urine, | Hemorrhagic |
| | | | saliva | fever |
| Simian foamy | Spumavirus, | Human, monkeys | Zoonosis, | None |
| virus | Retroviridae | | contact | |
| Simian virus 5 | Rubulavirus, | Human, dog | Zoonosis, | Undocumented |
| | Paramyxoviridae | | contact | |
| Sindbis virus | Alphavirus, Togaviridae | Human, birds, | Zoonosis, | Pogosta_disease |
| | | mosquitoes | arthropod bite | Fever, joint pain |
| Southampton virus | Norovirus, Caliciviridae | Human | Fecal-oral | Gastroenteritis |
| St. louis | Flavivirus, | Human, birds, | Zoonosis, | Encephalitis |
| encephalitis virus | Flaviviridae | mosquitoes | arthropod bite | |
| Tick-borne | Flavivirus, | Human, ticks | Zoonosis, | Encephalitis |
| powassan virus | Flaviviridae | | arthropod bite | |
| Torque teno virus | Alphatorquevirus | Human | Sexual, blood | None |
| Toscana virus | Phlebovirus, Bunyaviridae | Human, mosquitoes | Zoonosis, | Hemorrhagic |
| | | | arthropod bite | fever |
| Uukuniemi virus | Phlebovirus, Bunyaviridae | Human, ticks | Zoonosis, | Hemorrhagic |
| | | | arthropod bite | fever |
| Vaccinia virus | Orthopoxvirus, Poxviridae | Human, mammals | Contact | None |
| Varicella-zoster | Varicellovirus, | Human | Respiratory, | Varicella |
| virus | Herpesviridae | | contact | |
| Variola virus | Orthopoxvirus, Poxviridae | Human | Respiratory | Variola |
| Venezuelan equine | Alphavirus, Togaviridae | Human, rodents, | Zoonosis, | Fever, joint pain |
| encephalitis virus | | mosquitoes | arthropod bite | |
| Vesicular | Vesiculovirus, | Human, cattle, horse, | Zoonosis, | Encephalitis |
| stomatitis virus | Rhabdoviridae | pig, flies | athropod bite | |
| Western equine | Alphavirus, Togaviridae | Human, vertebrates, | Zoonosis, | Fever, joint pain |
| encephalitis virus | | mosquitoes | arthropod bite | |
| WU polyomavirus | Polyomavirus, | Human | Respiratory | None |
| | Polyomaviridae | | fluids or urine | |
| West Nile virus | Flavivirus, | Human, birds, ticks, | Zoonosis, | Hemorrhagic |
| | Flaviviridae | mosquitoes | arthropod bite | fever |
| Yaba monkey | Orthopoxvirus, Poxviridae | Human, monkeys | Zoonosis, | None |
| tumor virus | | | contact | |
| Yaba-like disease | Orthopoxvirus, Poxviridae | Human, monkeys | Zoonosis, | None |
| virus | | | contact | |
| Yellow fever virus | Flavivirus, | Human, monkeys, | Zoonosis, | Hemorrhagic |
| | Flaviviridae | mosquitoes | arthropod bite | fever |
| Zika virus | Flavivirus, Flaviviridae | Human, monkeys, | Zoonosis, | Fever, joint pain, |
| | | mosquitoes | arthropod bite | rash |

In certain embodiments, the virus is the virus is a virus listed in Table 9.

TABLE 9
Exemplary Virus Targets

| Virus | Tissue/cell type | Citation |
|---|---|---|
| Lassa virus | DCs, vascular endothelial cells | Kunz, S. et. al. 2005. Journal of |
| | | Virology |
| Ebola virus | Numerous (DCs, macrophages, | Martines, R. B. et. al. 2015. Journal of |
| | hepatocytes, etc.) | Pathology. |
| SARS-CoV | Lung | To, K F. et. al. 2004. Journal of |
| | | Pathology. |
| Zika | Numerous (bodily fluids, placenta, | Miner, J. J. & Diamond, M. S. 2017. |
| | brain, etc.) | Cell Host & Microbe. |
| Dengue | Numerous (DCs, macrophages, | Flipse, J. et. al. 2016. Journal of |
| | liver, etc.) | General Virology. |
| Chikungunya | Numerous (immune cells, liver, | Schwartz, O. & Albert, M. L. 2010. |
| | central nervous system, etc.) | Nature Reviews. |
| Influenza | Lung epithelial cells or | Medina, R. A. & Garcia-Sastre A. |
| | macrophages | 2011 Nature Reviews. |
| HIV | T cells, macrophages | Weiss, R. A. 2002. IUBMB Life. |
| Rotavirus | Intestine | Lopez, S & Arias, C. F. 2006. CTMI |
| Herpes Simplex (HSV-1) | Epithelial cells, neuronal cells | Schelhaas, M. et. al. 2003. Journal of |
| | | General Virology. |
| HCV | Liver | Ding, Q, et. al. 2014. Cell Host & |
| | | Microbe. |
| HBV | Liver | Schieck, A. et. al. 2013. Hepatology. |

In certain embodiments, the virus is a virus listed in Table 10.

TABLE 10
List of Viruses with FDA-Approved Vaccines (15-16):

| | |
|---|---|
| 1. | Adenovirus |
| 2. | Hepatitis A |
| 3. | Hepatitis B |
| 4. | Human Papillomavirus (HPV) |
| 5. | Influenza |
| 6. | Japanese Encephalitis Virus |
| 7. | Measles |
| 8. | Mumps |
| 9. | Polio |
| 10. | Rabies |
| 11. | Rotavirus |
| 12. | Rubella |
| 13. | Shingles/Zoster (HSV) |
| 14. | Smallpox* |
| 15. | Varicella (Chicken Pox) |
| 16. | Yellow Fever |

List of Viruses with FDA-Approved Antiviral Drugs (9):

| | |
|---|---|
| 1. | Cytomegalovirus |
| 2. | Human Immunodeficiency Virus (HIV) |
| 3. | Hepatitis B |
| 4. | Hepatitis C |
| 5. | Influenza |
| 6. | Respiratory Syncytial Virus |
| 7. | Human Papillomavirus (HPV) |
| 8. | Herpes Simplex Virus (Shingles) |
| 9. | Varicella Zoster Virus (Chicken pox) |

List of Viruses with FDA-Approved Nucleic Acid Diagnostics (11):

| | |
|---|---|
| 1. | Adenovirus |
| 2. | Cytomegalovirus |
| 3. | Dengue |
| 4. | Enterovirus |
| 5. | Herpes Simplex Virus |
| 6. | Hepatitis B |
| 7. | Hepatitis C |
| 8. | Human Metapneumovirus |
| 9. | Human Papillomavirus |
| 10. | Influenza |
| 11. | Respiratory Syncytial Virus |

In certain embodiments, the virus is a drug resistant virus. By means of example, and without limitation, the virus may be a ribavirin resistant virus. Ribavirin is a very effective antiviral that hits a number of RNA viruses. Below are a few important viruses that have evolved ribavirin resistance. Foot and Mouth Disease Virus: doi:10.1128/JVL.03594-13. Polio virus: www.pnas.org/content/100/12/7289.full.pdf. Hepatitis C Virus: jvi.asm.org/content/79/4/2346.full. A number of other persistent RNA viruses, such as hepatitis and HIV, have evolved resistance to existing antiviral drugs. Hepatitis B Virus (lamivudine, tenofovir, entecavir): doi:10.1002/hep.22900. Hepatitis C Virus (Telaprevir, BILN2061, ITMN-191, SCH6, Boceprevir, AG-021541, ACH-806): doi:10.1002/hep.22549. HIV has many drug resistant mutations, see hivdb.stanford.edu/ for more information. Aside from drug resistance, there are a number of clinically relevant mutations that could be targeted with the CRISPR systems according to the invention as described herein. For instance, persistent versus acute infection in LCMV: doi:10.1073/pnas.1019304108; or increased infectivity of Ebola: http://doi.org/10.1016/j.cell.2016.10.014 and http://doi.org/10.1016/j.cell.2016.10.013.

### Malaria Detection and Monitoring

Malaria is a mosquito-borne pathology caused by Plasmodium parasites. The parasites are spread to people through the bites of infected female Anopheles mosquitoes. Five Plasmodium species cause malaria in humans: Plasmodium falciparum, Plasmodium vivax, Plasmodium ovale, Plasmodium malariae, and Plasmodium knowlesi. Among them, according to the World Health Organization (WHO), Plasmodium falciparum: and Plasmodium vivax are responsible for the greatest threat. P. falciparum is the most prevalent malaria parasite on the African continent and is responsible for most malaria-related deaths globally. P. vivax is the dominant malaria parasite in most countries outside of sub-Saharan Africa.

Treatment against Plasmodium sp. include aryl-amino alcohols such as quinine or quinine derivatives such as chloroquine, amodiaquine, mefloquine, piperaquine, lumefantrine, primaquine; lipophilic hydroxynaphthoquinone analog, such as atovaquone; antifolate drugs, such as the sulfa drugs sulfadoxine, dapsone and pyrimethamine; proguanil; the combination of atovaquone/proguanil; atemisins drugs; and combinations thereof. In some embodiments. The method includes screening for resistance against one or more of these compounds.

Target sequences for the assays described herein include those that are diagnostic for the presence of a mosquito-borne pathogen include a sequence that diagnostic for the presence of Plasmodium, notably Plasmodia species affecting humans such as Plasmodium falciparum, Plasmodium vivax, Plasmodium ovale, Plasmodium malariae, and Plasmodium knowlesi, including sequences from the genomes thereof.

Target sequences for the assays described herein include those that are diagnostic for monitoring drug resistance to treatment against Plasmodium, including but not limited to, Plasmodia species affecting humans such as Plasmodium falciparum, Plasmodium vivax, Plasmodium ovale, Plasmodium malariae, and Plasmodium knowlesi.

Further target sequences include sequences include target molecules/nucleic acid molecules coding for proteins involved in essential biological process for the Plasmodium parasite and notably transporter proteins, such as protein from drug/metabolite transporter family, the ATP-binding cassette (ABC) protein involved in substrate translocation, such as the ABC transporter C subfamily or the Na+/H+ exchanger, membrane glutathione S-transferase; proteins involved in the folate pathway, such as the dihydropteroate synthase, the dihydrofolate reductase activity or the dihydrofolate reductase-thymidylate synthase; and proteins involved in the translocation of protons across the inner mitochondrial membrane and notably the cytochrome b complex. Additional target may also include the gene(s) coding for the heme polymerase.

Further target sequences include target molecules/nucleic acid molecules coding for proteins involved in essential biological process may be selected from the P. falciparum chloroquine resistance transporter gene (pfcrt), the P. falciparum multidrug resistance transporter 1 (pfmdr1), the P. falciparum multidrug resistance-associated protein gene (Pfmrp), the P. falciparum Na+/H+ exchanger gene (pfnhe), the gene coding for the P. falciparum exported protein 1, the P. falciparum Ca2+ transporting ATPase 6 (pfatp6); the P. falciparum dihydropteroate synthase (pfdhps), dihydrofolate reductase activity (pfdhpr) and dihydrofolate reductase-thymidylate synthase (pfdhfr) genes, the cytochrome b gene, gtp cyclohydrolase and the Kelch13 (K13) gene as well as their functional heterologous genes in other Plasmodium species.

A number of mutations, notably single point mutations, have been identified in the proteins which are the targets of the current malaria treatments and associated with specific resistance phenotypes. Accordingly, the invention allows for the detection of various resistance phenotypes of mosquito-borne parasites, such as plasmodium by detection of those targets that are associated with the specific resistance phenotypes.

In some embodiments, the method detects one or more mutation(s) and/or one or more single nucleotide polymorphisms in target nucleic acids/molecules. In some embodiments, any one of the mutations below, or their combination thereof, can be used as drug resistance marker and can be detected using the methods, assays, devices, compositions, and/or devices described herein.

Single point mutations in P. falciparum K13 that can be detected by an assay described herein include the following single point mutations in positions 252, 441, 446, 449, 458, 493, 539, 543, 553, 561, 568, 574, 578, 580, 675, 476, 469, 481, 522, 537, 538, 579, 584 and 719 and notably mutations E252Q, P441L, F446I, G449A, N458Y, Y493H, R539T, I543T, P553L, R561H, V568G, P574L, A578S, C580Y, A675V, M4761; C469Y; A481V; S522C; N537I; N537D; G538V; M579I; D584V; and H719N. These mutations are generally associated with artemisins drugs resistance phenotypes (Artemisinin and artemisinin-based combination therapy resistance, April 2016 WHO/HTM/GMP/2016.5).

Mutations in the P. falciparum dihydrofolate reductase (DHFR) (PfDHFR-TS, PFD0830w) that can be detected by the assays described herein include mutations in positions 108, 51, 59 and 164, notably 108 D, 164L, 511 and 59R which modulate resistance to pyrimethamine. Other polymorphisms that can be detected by the methods described herein include 437G, 581G, 540E, 436A and 613S, which are associated with resistance to sulfadoxine. Additional mutations that can be detected by the assays described herein include Ser108Asn, Asn51Ile, Cys59Arg, Ile164Leu, Cys50Arg, Ile164Leu, Asn188Lys, Ser189Arg and Val213Ala, Ser108Thr and Ala16Val. Mutations Ser108Asn, Asn51Ile, Cys59Arg, Ile164Leu, Cys50Arg, Ile164Leu are notably associated with pyrimethamine based therapy and/or chloroguanine-dapsone combination therapy resistances and can be detected by the assays described herein. Cycloguanil resistance appears to be associated with the double mutations Ser108Thr and Ala16Val, which can be detected by the assays described herein. Amplification of dhfr may also be of high relevance for therapy resistance notably pyrimethamine resistance and can be detected ny the assays described herein.

Mutations in the P. falciparum dihydropteroate synthase (DHPS) (PfDHPS, PF08_0095) can be detected by the assays described herein, and include, without limitation, mutations in positions 436, 437, 581 and 613 Ser436Ala/Phe, Ala437Gly, Lys540Glu, Ala581Gly and Ala613Thr/Ser. Polymorphism in position 581 and/or 613 have also been associated with resistance to sulfadoxine-pyrimethamine base therapies and can be detected by an assay described herein.

Mutations in the P. falciparum chloroquine-resistance transporter (PfCRT) can be detected by the assays described herein. In some embodiments, the polymorphism in position 76, notably the mutation Lys76Thr, is associated with resistance to chloroquine and can be detected by an assay described herein. Further polymorphisms include Cys72Ser, Met74Ile, Asn75Glu, Ala220Ser, Gln271Glu, Asn326Ser, Ile356Thr and Arg371Ile which may be associated with chloroquine resistance can be detected by an assay described herein. PfCRT is also phosphorylated at the residues S33, S411 and T416, which may regulate the transport activity or specificity of the protein, which can be detected by an assay described herein.

Mutations in the P. falciparum multidrug-resistance transporter 1 (PfMDR1) (PFE1150w) can be detected by an assay described herein. For example, polymorphisms in positions 86, 184, 1034, 1042, notably Asn86Tyr, Tyr184-Phe, Ser1034Cys, Asn1042Asp and Asp1246Tyr have been identified and reported to influence have been reported to influence susceptibilities to lumefantrine, artemisinin, quinine, mefloquine, halofantrine and chloroquine and can be detected by an assay described herein. Additionally, amplification of PfMDR1 is associated with reduced susceptibility to lumefantrine, artemisinin, quinine, mefloquine, and halofantrine and can be detected by an assay described herein. Deamplification of PfMDR1 leads to an increase in chloroquine resistance and can be detected by an assay described herein. Amplification of pfmdr1 may also be detected. The phosphorylation status of PfMDR1 is also of high relevance and can be detected by an assay described herein.

Mutations in the P. falciparum multidrug-resistance associated protein (PfMRP) (gene reference PFA0590w) can be detected by an assay described herein. For example polymorphisms in positions 191 and/or 437, such as Y191H and A437S have been identified and associated with chloroquine resistance phenotypes and can be detected by an assay described herein.

Mutations in the P. falciparum NA+/H+ enchanger (PfNHE) (ref PF13_0019) can be detected by an assay described herein. For example, increased repetition of the DNNND in microsatellite ms4670 may be a marker for quinine resistance and can be detected by an assay described herein.

Mutations altering the ubiquinol binding site of the cytochrome b protein encoded by the cytochrome be gene (cytb, mal_mito_3) are associated with atovaquone resistance and can be detected by an assay described herein. Mutations in positions 26, 268, 276, 133 and 280 and notably Tyr26Asn, Tyr268Ser, M1331 and G280D may be associated with atovaquone resistance and can be detected by an assay described herein.

In P Vivax, mutations in PvMDR1, the homolog of Pf MDR1 have been associated with chloroquine resistance, notably polymorphism in position 976 such as the mutation Y976F and can be detected by an assay described herein.

The above mutations are defined in terms of protein sequences. However, the skilled person is able to determine the corresponding mutations, including SNPs, to be identified as a nucleic acid target sequence.

Other identified drug-resistance markers are known in the art, for example as described in “Susceptibility of Plasmodium falciparum to antimalarial drugs (1996-2004)”; WHO; Artemisinin and artemisinin-based combination therapy resistance (April 2016 WHO/HTM/GMP/2016.5); “Drug-resistant malaria: molecular mechanisms and implications for public health” FEBS Lett. 2011 Jun. 6; 585(11):1551-62. doi:10.1016/j.febslet.2011.04.042. Epub 2011 Apr. 23. Review. PubMed PMID: 21530510; the contents of which are herewith incorporated by reference and can be detected by an assay described herein.

As to polypeptides that may be detected in accordance with the present invention, gene products of all genes mentioned herein may be used as targets. Correspondingly, it is contemplated that such polypeptides could be used for species identification, typing and/or detection of drug resistance.

In certain example embodiments, the systems, devices, and methods, disclosed herein are directed to detecting the presence of one or more mosquito-borne parasite in a sample, such as a biological sample obtained from a subject. In certain example embodiments, the parasite may be selected from the species Plasmodium falciparum, Plasmodium vivax, Plasmodium ovale, Plasmodium malariae or Plasmodium knowlesi. Accordingly, the methods disclosed herein can be adapted for use in other methods (or in combination) with other methods that require quick identification of parasite species, monitoring the presence of parasites and parasite forms (for example corresponding to various stages of infection and parasite life-cycle, such as exo-erythrocytic cycle, erythrocytic cyle, sporpogonic cycle; parasite forms include merozoites, sporozoites, schizonts, gametocytes); detection of certain phenotypes (e.g. pathogen drug resistance), monitoring of disease progression and/or outbreak, and treatment (drug) screening. Further, in the case of malaria, a long time may elapse following the infective bite, namely a long incubation period, during which the patient does not show symptoms. Similarly, prophylactic treatments can delay the appearance of symptoms, and long asymptomatic periods can also be observed before a relapse. Such delays can easily cause misdiagnosis or delayed diagnosis, and thus impair the effectiveness of treatment.

Because of the rapid and sensitive diagnostic capabilities of the embodiments disclosed here, detection of parasite type, down to a single nucleotide difference, and the ability to be deployed as a POC device, the embodiments disclosed herein may be used guide therapeutic regimens, such as selection of the appropriate course of treatment. The embodiments disclosed herein may also be used to screen environmental samples (mosquito population, etc.) for the presence and the typing of the parasite. The embodiments may also be modified to detect mosquito-borne parasites and other mosquito-borne pathogens simultaneously. In some instances, malaria and other mosquito-borne pathogens may present initially with similar symptoms. Thus, the ability to quickly distinguish the type of infection can guide important treatment decisions. Other mosquito-born pathogens that may be detected in conjunction with malaria include dengue, West Nile virus, chikungunya, yellow fever, filariasis, Japanese encephalitis, Saint Louis encephalitis, western equine encephalitis, eastern equine encephalitis, Venezuelan equine encephalitits, La Crosse encephalitis, and zika.

In certain example embodiments, the devices, systems, and methods disclosed herein may be used to distinguish multiple mosquito-borne parasite species in a sample. In certain example embodiments, identification may be based on ribosomal RNA sequences, including the 18S, 16S, 23S, and 5S subunits. In certain example embodiments, identification may be based on sequences of genes that are present in multiple copies in the genome, such as mitochondrial genes like CYTB. In certain example embodiments, identification may be based on sequences of genes that are highly expressed and/or highly conserved such as GAPDH, Histone H2B, enolase, or LDH. Methods for identifying relevant rRNA sequences are disclosed in U.S. Patent Application Publication No. 2017/0029872. In certain example embodiments, a set of guide RNA may be designed to distinguish each species by a variable region that is unique to each species or strain. Guide RNAs may also be designed to target RNA genes that distinguish microbes at the genus, family, order, class, phylum, kingdom levels, or a combination thereof. In certain example embodiments where amplification is used, a set of amplification primers may be designed to flanking constant regions of the ribosomal RNA sequence and a guide RNA designed to distinguish each species by a variable internal region. In certain example embodiments, the primers and guide RNAs may be designed to conserved and variable regions in the 16S subunit respectfully. Other genes or genomic regions that uniquely variable across species or a subset of species such as the RecA gene family, RNA polymerase β subunit, may be used as well. Other suitable phylogenetic markers, and methods for identifying the same, are discussed for example in Wu et al. arXiv:1307.8690 [q-bio.GN].

In certain example embodiments, species identification can be performed based on genes that are present in multiple copies in the genome, such as mitochondrial genes like CYTB. In certain example embodiments, species identification can be performed based on highly expressed and/or highly conserved genes such as GAPDH, Histone H2B, enolase, or LDH.

In certain example embodiments, a method or diagnostic is designed to screen mosquito-borne parasites across multiple phylogenetic and/or phenotypic levels at the same time. For example, the method or diagnostic may comprise the use of multiple CRISPR systems with different guide RNAs. A first set of guide RNAs may distinguish, for example, between Plasmodium falciparum or Plasmodium vivax. These general classes can be even further subdivided. For example, guide RNAs could be designed and used in the method or diagnostic that distinguish drug-resistant strains, in general or with respect to a specific drug or combination of drugs. A second set of guide RNA can be designed to distinguish microbes at the species level. Thus, a matrix may be produced identifying all mosquito-borne parasites species or subspecies, further divided according to drug resistance. The foregoing is for example purposes only. Other means for classifying other types of mosquito-borne parasites are also contemplated and would follow the general structure described above.

In certain example embodiments, the devices, systems and methods disclosed herein may be used to screen for mosquito-borne parasite genes of interest, for example drug resistance genes. Guide RNAs may be designed to distinguish between known genes of interest. Samples, including clinical samples, may then be screened using the embodiments disclosed herein for detection of one or more such genes. The ability to screen for drug resistance at POC would have tremendous benefit in selecting an appropriate treatment regime. In certain example embodiments, the drug resistance genes are genes encoding proteins such as transporter proteins, such as protein from drug/metabolite transporter family, the ATP-binding cassette (ABC) protein involved in substrate translocation, such as the ABC transporter C subfamily or the Na+/H+ exchanger; proteins involved in the folate pathway, such as the dihydropteroate synthase, the dihydrofolate reductase activity or the dihydrofolate reductase-thymidylate synthase; and proteins involved in the translocation of protons across the inner mitochondrial membrane and notably the cytochrome b complex. Additional targets may also include the gene(s) coding for the heme polymerase. In certain example embodiments, the drug resistance genes are selected from the P. falciparum chloroquine resistance transporter gene (pfcrt), the P. falciparum multidrug resistance transporter 1 (pfmdr1), the P. falciparum multidrug resistance-associated protein gene (Pfmrp), the P. falciparum Na+/H+ exchanger gene (pfnhe), the P. falciparum Ca2+ transporting ATPase 6 (pfatp6), the P. falciparum dihydropteroate synthase (pfdhps), dihydrofolate reductase activity (pfdhpr) and dihydrofolate reductase-thymidylate synthase (pfdhfr) genes, the cytochrome b gene, gtp cyclohydrolase and the Kelch13 (K13) gene as well as their functional heterologous genes in other Plasmodium species. Other identified drug-resistance markers are known in the art, for example as described in “Susceptibility of Plasmodium falciparum to antimalarial drugs (1996-2004)”; WHO; Artemisinin and artemisinin-based combination therapy resistance (April 2016 WHO/HTM/GMP/2016.5); “Drug-resistant malaria: molecular mechanisms and implications for public health” FEBS Lett. 2011 Jun. 6; 585(11):1551-62. doi:10.1016/j.febslet.2011.04.042. Epub 2011 Apr. 23. Review. PubMed PMID: 21530510; the contents of which are herewith incorporated by reference.

In some embodiments, a CRISPR system, detection system or methods of use thereof as described herein may be used to determine the evolution of a mosquito-borne parasite outbreak. The method may comprise detecting one or more target sequences from a plurality of samples from one or more subjects, wherein the target sequence is a sequence from a mosquito-borne parasite spreading or causing the outbreaks. Such a method may further comprise determining a pattern of mosquito-borne parasite transmission, or a mechanism involved in a disease outbreak caused by a mosquito-borne parasite. The samples may be derived from one or more humans, and/or be derived from one or more mosquitoes.

The pattern of pathogen transmission may comprise continued new transmissions from the natural reservoir of the mosquito-borne parasite or other transmissions (e.g. across mosquitoes) following a single transmission from the natural reservoir or a mixture of both. In one embodiment, the target sequence is preferably a sequence within the mosquito-borne parasite genome or fragments thereof. In one embodiment, the pattern of the mosquito-borne parasite transmission is the early pattern of the mosquito-borne parasite transmission, i.e. at the beginning of the mosquito-borne parasite outbreak. Determining the pattern of the mosquito-borne parasite transmission at the beginning of the outbreak increases likelihood of stopping the outbreak at the earliest possible time thereby reducing the possibility of local and international dissemination.

Determining the pattern of the mosquito-borne parasite transmission may comprise detecting a mosquito-borne parasite sequence according to the methods described herein. Determining the pattern of the pathogen transmission may further comprise detecting shared intra-host variations of the mosquito-borne parasite sequence between the subjects and determining whether the shared intra-host variations show temporal patterns. Patterns in observed intrahost and interhost variation provide important insight about transmission and epidemiology (Gire, et al., 2014).

In addition to other sample types disclosed herein, the sample may be derived from one or more mosquitoes, for example the sample may comprise mosquito saliva.

### Biomarker Detection and Applications

In certain example embodiments, the systems, devices, and methods disclosed herein may be used for biomarker detection. For example, the systems, devices and method disclosed herein may be used for SNP detection and/or genotyping. The systems, devices and methods disclosed herein may be also used for the detection of any disease state or disorder characterized by aberrant gene expression. Aberrant gene expression includes aberration in the gene expressed, location of expression and level of expression. Multiple transcripts or protein markers related to cardiovascular, immune disorders, and cancer among other diseases may be detected. In certain example embodiments, the embodiments disclosed herein may be used for cell free DNA detection of diseases that involve lysis, such as liver fibrosis and restrictive/obstructive lung disease. In certain example embodiments, the embodiments could be utilized for faster and more portable detection for pre-natal testing of cell-free DNA. The embodiments disclosed herein may be used for screening panels of different SNPs associated with, among others, cardiovascular health, lipid/metabolic signatures, ethnicity identification, paternity matching, human ID (e.g., matching suspect to a criminal database of SNP signatures). The embodiments disclosed herein may also be used for cell free DNA detection of mutations related to and released from cancer tumors. The embodiments disclosed herein may also be used for detection of meat quality, for example, by providing rapid detection of different animal sources in a given meat product. Embodiments disclosed herein may also be used for the detection of GMOs or gene editing related to DNA. As described herein elsewhere, closely related genotypes/alleles or biomarkers (e.g. having only a single nucleotide difference in a given target sequence) may be distinguished by introduction of a synthetic mismatch in the gRNA.

In an aspect, the invention relates to a method for detecting target nucleic acids in samples, comprising:
distributing a sample or set of samples into one or more individual discrete volumes, the individual discrete volumes comprising a CRISPR system according to the invention as described herein;
incubating the sample or set of samples under conditions sufficient to allow binding of the one or more guide RNAs to one or more target molecules;
activating the CRISPR effector protein via binding of the one or more guide RNAs to the one or more target molecules, wherein activating the CRISPR effector protein results in modification of the RNA-based detection construct such that a detectable positive signal is generated; and
detecting the detectable positive signal, wherein detection of the detectable positive signal indicates a presence of one or more target molecules in the sample.

### Circulating Tumor Cells

In one embodiment, circulating cells (e.g., circulating tumor cells (CTC)) can be assayed with the present invention. Isolation of circulating tumor cells (CTC) for use in any of the methods described herein may be performed. Exemplary technologies that achieve specific and sensitive detection and capture of circulating cells that may be used in the present invention have been described (Mostert B, et al., Circulating tumor cells (CTCs): detection methods and their clinical relevance in breast cancer. Cancer Treat Rev. 2009; 35:463-474; and Talasaz A H, et al., Isolating highly enriched populations of circulating epithelial cells and other rare cells from blood using a magnetic sweeper device. Proc Natl Acad Sci USA. 2009; 106:3970-3975). As few as one CTC may be found in the background of 105-106 peripheral blood mononuclear cells (Ross A A, et al., Detection and viability of tumor cells in peripheral blood stem cell collections from breast cancer patients using immunocytochemical and clonogenic assay techniques. Blood. 1993, 82:2605-2610). The CellSearch® platform uses immunomagnetic beads coated with antibodies to Epithelial Cell Adhesion Molecule (EpCAM) to enrich for EPCAM-expressing epithelial cells, followed by immunostaining to confirm the presence of cytokeratin staining and absence of the leukocyte marker CD45 to confirm that captured cells are epithelial tumor cells (Momburg F, et al., Immunohistochemical study of the expression of a Mr 34,000 human epithelium-specific surface glycoprotein in normal and malignant tissues. Cancer Res. 1987; 47:2883-2891; and Allard W J, et al., Tumor cells circulate in the peripheral blood of all major carcinomas but not in healthy subjects or patients with nonmalignant diseases. Clin Cancer Res. 2004; 10:6897-6904). The number of cells captured have been prospectively demonstrated to have prognostic significance for breast, colorectal and prostate cancer patients with advanced disease (Cohen S J, et al., J Clin Oncol. 2008; 26:3213-3221; Cristofanilli M, et al. N Engl J Med. 2004; 351:781-791; Cristofanilli M, et al., J Clin Oncol. 2005; 23: 1420-1430; and de Bono J S, et al. Clin Cancer Res. 2008; 14:6302-6309).

The present invention also provides for isolating CTCs with CTC-Chip Technology. CTC-Chip is a microfluidic based CTC capture device where blood flows through a chamber containing thousands of microposts coated with anti-EpCAM antibodies to which the CTCs bind (Nagrath S, et al. Isolation of rare circulating tumour cells in cancer patients by microchip technology. Nature. 2007; 450: 1235-1239). CTC-Chip provides a significant increase in CTC counts and purity in comparison to the CellSearch® system (Maheswaran S, et al. Detection of mutations in EGFR in circulating lung-cancer cells, N Engl J Med. 2008; 359:366-377), both platforms may be used for downstream molecular analysis.

### Cell-Free Chromatin

In certain embodiments, cell free chromatin fragments are isolated and analyzed according to the present invention. Nucleosomes can be detected in the serum of healthy individuals (Stroun et al., Annals of the New York Academy of Sciences 906: 161-168 (2000)) as well as individuals afflicted with a disease state. Moreover, the serum concentration of nucleosomes is considerably higher in patients suffering from benign and malignant diseases, such as cancer and autoimmune disease (Holdenrieder et al (2001) Int J Cancer 95, 1 14-120, Trejo-Becerril et al (2003) Int J Cancer 104, 663-668; Kuroi et al 1999 Breast Cancer 6, 361-364; Kuroi et al (2001) Int j Oncology 19, 143-148; Amoura et al (1997) Arth Rheum 40, 2217-2225; Williams et al (2001) J Rheumatol 28, 81-94). Not being bound by a theory, the high concentration of nucleosomes in tumor bearing patients derives from apoptosis, which occurs spontaneously in proliferating tumors. Nucleosomes circulating in the blood contain uniquely modified histones. For example, U.S. Patent Publication No. 2005/0069931 (Mar. 31, 2005) relates to the use of antibodies directed against specific histone N-terminus modifications as diagnostic indicators of disease, employing such histone-specific antibodies to isolate nucleosomes from a blood or serum sample of a patient to facilitate purification and analysis of the accompanying DNA for diagnostic/screening purposes. Accordingly, the present invention may use chromatin bound DNA to detect and monitor, for example, tumor mutations. The identification of the DNA associated with modified histones can serve as diagnostic markers of disease and congenital defects.

Thus, in another embodiment, isolated chromatin fragments are derived from circulating chromatin, preferably circulating mono and oligonucleosomes. Isolated chromatin fragments may be derived from a biological sample. The biological sample may be from a subject or a patient in need thereof. The biological sample may be sera, plasma, lymph, blood, blood fractions, urine, synovial fluid, spinal fluid, saliva, circulating tumor cells or mucous.

Cell-Free DNA (cfDNA)

In certain embodiments, the present invention may be used to detect cell free DNA (cfDNA). Cell free DNA in plasma or serum may be used as a non-invasive diagnostic tool. For example, cell free fetal DNA has been studied and optimized for testing on-compatible RhD factors, sex determination for X-linked genetic disorders, testing for single gene disorders, indentificaiton of preeclampsia. For example, sequencing the fetal cell fraction of cfDNA in maternal plasma is a reliable approach for detecting copy number changes associated with fetal chromosome aneuploidy. For another example, cfDNA isolated from cancer patients has been used to detect mutations in key genes relevant for treatment decisions.

In certain example embodiments, the present disclosure provides detecting cfDNA directly from a patient sample. In certain other example embodiment, the present disclosure provides enriching cfDNA using the enrichment embodiments disclosed above and prior to detecting the target cfDNA.

### Exosomes

In one embodiment, exosomes can be assayed with the present invention. Exosomes are small extracellular vesicles that have been shown to contain RNA. Isolation of exosomes by ultracentrifugation, filtration, chemical precipitation, size exclusion chromatography, and microfluidics are known in the art. In one embodiment exosomes are purified using an exosome biomarker. Isolation and purification of exosomes from biological samples may be performed by any known methods (see e.g., WO2016172598A1).

### SNP Detection and Genotyping

In certain embodiments, the present invention may be used to detect the presence of single nucleotide polymorphisms (SNP) in a biological sample. The SNPs may be related to maternity testing (e.g., sex determination, fetal defects). They may be related to a criminal investigation. In one embodiment, a suspect in a criminal investigation may be identified by the present invention. Not being bound by a theory nucleic acid based forensic evidence may require the most sensitive assay available to detect a suspect or victim's genetic material because the samples tested may be limiting.

In other embodiments, SNPs associated with a disease are encompassed by the present invention. SNPs associated with diseases are well known in the art and one skilled in the art can apply the methods of the present invention to design suitable guide RNAs (see e.g., www.ncbi.nlm.nih.gov/clinvar?term=human %/5Borgn%5D).

In an aspect, the invention relates to a method for genotyping, such as SNP genotyping, comprising:
distributing a sample or set of samples into one or more individual discrete volumes, the individual discrete volumes comprising a CRISPR system according to the invention as described herein;
incubating the sample or set of samples under conditions sufficient to allow binding of the one or more guide RNAs to one or more target molecules;
activating the CRISPR effector protein via binding of the one or more guide RNAs to the one or more target molecules, wherein activating the CRISPR effector protein results in modification of the RNA-based detection construct such that a detectable positive signal is generated; and
detecting the detectable positive signal, wherein detection of the detectable positive signal indicates a presence of one or more target molecules characteristic for a particular genotype in the sample.

In certain embodiments, the detectable signal is compared to (e.g., by comparison of signal intensity) one or more standard signal, preferably a synthetic standard signal, such as for instance illustrated in an embodiment in FIG. 60. In certain embodiments, the standard is or corresponds to a particular genotype. In certain embodiments, the standard comprises a particular SNP or other (single) nucleotide variation. In certain embodiments, the standard is a (PCR-amplified) genotype standard. In certain embodiments, the standard is or comprises DNA. In certain embodiments, the standard is or comprises RNA. In certain embodiments, the standard is or comprised RNA which is transcribed from DNA. In certain embodiments, the standard is or comprises DNA which is reverse transcribed from RNA. In certain embodiments, the detectable signal is compared to one or more standard, each of which corresponds to a known genotype, such as a SNP or other (single) nucleotide variation. In certain embodiments, the detectable signal is compared to one or more standard signal and the comparison comprises statistical analysis, such as by parametric or non-parametric statistical analysis, such as by one- or two-way ANOVA, etc. In certain embodiments, the detectable signal is compared to one or more standard signal and when the detectable signal does not (statistically) significantly deviate from the standard, the genotype is determined as the genotype corresponding to said standard.

In other embodiments, the present invention allows rapid genotyping for emergency pharmacogenomics. In one embodiment, a single point of care assay may be used to genotype a patient brought in to the emergency room. The patient may be suspected of having a blood clot and an emergency physician needs to decide a dosage of blood thinner to administer. In exemplary embodiments, the present invention may provide guidance for administration of blood thinners during myocardial infarction or stroke treatment based on genotyping of markers such as VKORC1, CYP2C9, and CYP2C19. In one embodiment, the blood thinner is the anticoagulant warfarin (Holford, NH (December 1986). “Clinical Pharmacokinetics and Pharmacodynamics of Warfarin Understanding the Dose-Effect Relationship”. Clinical Pharmacokinetics. Springer International Publishing. 11 (6): 483-504). Genes associated with blood clotting are known in the art (see e.g., US20060166239A1; Litin S C, Gastineau D A (1995) “Current concepts in anticoagulant therapy”. Mayo Clin. Proc. 70 (3): 266-72; and Rusdiana et al., Responsiveness to low-dose warfarin associated with genetic variants of VKORC1, CYP2C9, CYP2C19, and CYP4F2 in an Indonesian population. Eur J Clin Pharmacol. 2013 March; 69(3):395-405). Specifically, in the VKORC1 1639 (or 3673) single-nucleotide polymorphism, the common (“wild-type”) G allele is replaced by the A allele. People with an A allele (or the “A haplotype”) produce less VKORC1 than do those with the G allele (or the “non-A haplotype”). The prevalence of these variants also varies by race, with 37% of Caucasians and 14% of Africans carrying the A allele. The end result is a decreased number of clotting factors and therefore, a decreased ability to clot.

In certain example embodiments, the availability of genetic material for detecting a SNP in a patient allows for detecting SNPs without amplification of a DNA or RNA sample. In the case of genotyping, the biological sample tested is easily obtained. In certain example embodiments, the incubation time of the present invention may be shortened. The assay may be performed in a period of time required for an enzymatic reaction to occur. One skilled in the art can perform biochemical reactions in 5 minutes (e.g., 5 minute ligation). The present invention may use an automated DNA extraction device to obtain DNA from blood. The DNA can then be added to a reaction that generates a target molecule for the effector protein. Immediately upon generating the target molecule the masking agent can be cut and a signal detected. In exemplary embodiments, the present invention allows a POC rapid diagnostic for determining a genotype before administering a drug (e.g., blood thinner). In the case where an amplification step is used, all of the reactions occur in the same reaction in a one step process. In preferred embodiments, the POC assay may be performed in less than an hour, preferably 10 minutes, 20 minutes, 30 minutes, 40 minutes, or 50 minutes.

In certain embodiments, the systems, devices, and methods disclosed herein may be used for detecting the presence or expression level of long non-coding RNAs (lncRNAs). Expression of certain lncRNAs are associated with disease state and/or drug resistance. In particular, certain lncRNAs (e.g., TCONS_00011252, NR_034078, TCONS_00010506, TCONS_00026344, TCONS_00015940, TCONS_00028298, TCONS_00026380, TCONS_0009861, TCONS_00026521, TCONS_00016127, NR_125939, NR_033834, TCONS_00021026, TCONS_00006579, NR_109890, and NR_026873) are associated with resistance to cancer treatment, such as resistance to one or more BRAF inhibitors (e.g., Vemurafenib, Dabrafenib, Sorafenib, GDC-0879, PLX-4720, and LGX818) for treating melanoma (e.g., nodular melanoma, lentigo maligna, lentigo maligna melanoma, acral lentiginous melanoma, superficial spreading melanoma, mucosal melanoma, polypoid melanoma, desmoplastic melanoma, amelanotic melanoma, and soft-tissue melanoma). The detection of lncRNAs using the various embodiments described herein can facilitate disease diagnosis and/or selection of treatment options.

In one embodiment, the present invention can guide DNA- or RNA-targeted therapies (e.g., CRISPR, TALE, Zinc finger proteins, RNAi), particularly in settings where rapid administration of therapy is important to treatment outcomes.

### LOH Detection

Cancer cells undergo a loss of genetic material (DNA) when compared to normal cells. This deletion of genetic material which almost all, if not all, cancers undergo is referred to as “loss of heterozygosity” (LOH). Loss of heterozygosity (LOH) is a gross chromosomal event that results in loss of the entire gene and the surrounding chromosomal region. The loss of heterozygosity is a common occurrence in cancer, where it can indicate the absence of a functional tumor suppressor gene in the lost region. However, a loss may be silent because there still is one functional gene left on the other chromosome of the chromosome pair. The remaining copy of the tumor suppressor gene can be inactivated by a point mutation, leading to loss of a tumor suppressor gene. The loss of genetic material from cancer cells can result in the selective loss of one of two or more alleles of a gene vital for cell viability or cell growth at a particular locus on the chromosome.

An “LOH marker” is DNA from a microsatellite locus, a deletion, alteration, or amplification in which, when compared to normal cells, is associated with cancer or other diseases. An LOH marker often is associated with loss of a tumor suppressor gene or another, usually tumor related, gene.

The term “microsatellites” refers to short repetitive sequences of DNA that are widely distributed in the human genome. A microsatellite is a tract of tandemly repeated (i.e. adjacent) DNA motifs that range in length from two to five nucleotides, and are typically repeated 5-50 times. For example, the sequence TATATATATA (SEQ. ID. No. 13) is a dinucleotide microsatellite, and GTCGTCGTCGTCGTC (SEQ. ID. No. 14) is a trinucleotide microsatellite (with A being Adenine, G Guanine, C Cytosine, and T Thymine). Somatic alterations in the repeat length of such microsatellites have been shown to represent a characteristic feature of tumors. Guide RNAs may be designed to detect such microsatellites. Furthermore, the present invention may be used to detect alterations in repeat length, as well as amplifications and deletions based upon quantitation of the detectable signal. Certain microsatellites are located in regulatory flanking or intronic regions of genes, or directly in codons of genes. Microsatellite mutations in such cases can lead to phenotypic changes and diseases, notably in triplet expansion diseases such as fragile X syndrome and Huntington's disease.

Frequent loss of heterozygosity (LOH) on specific chromosomal regions has been reported in many kinds of malignancies. Allelic losses on specific chromosomal regions are the most common genetic alterations observed in a variety of malignancies, thus microsatellite analysis has been applied to detect DNA of cancer cells in specimens from body fluids, such as sputum for lung cancer and urine for bladder cancer. (Rouleau, et al. Nature 363, 515-521 (1993); and Latif, et al. Science 260, 1317-1320 (1993)). Moreover, it has been established that markedly increased concentrations of soluble DNA are present in plasma of individuals with cancer and some other diseases, indicating that cell free serum or plasma can be used for detecting cancer DNA with microsatellite abnormalities. (Kamp, et al. Science 264, 436-440 (1994); and Steck, et al. Nat Genet. 15(4), 356-362 (1997)). Two groups have reported microsatellite alterations in plasma or serum of a limited number of patients with small cell lung cancer or head and neck cancer. (Hahn, et al. Science 271, 350-353 (1996); and Miozzo, et al. Cancer Res. 56, 2285-2288 (1996)). Detection of loss of heterozygosity in tumors and serum of melanoma patients has also been previously shown (see, e.g., United States patent number U.S. Pat. No. 6,465,177B1).

Thus, it is advantageous to detect of LOH markers in a subject suffering from or at risk of cancer. The present invention may be used to detect LOH in tumor cells. In one embodiment, circulating tumor cells may be used as a biological sample. In preferred embodiments, cell free DNA obtained from serum or plasma is used to noninvasively detect and/or monitor LOH. In other embodiments, the biological sample may be any sample described herein (e.g., a urine sample for bladder cancer). Not being bound by a theory, the present invention may be used to detect LOH markers with improved sensitivity as compared to any prior method, thus providing early detection of mutational events. In one embodiment, LOH is detected in biological fluids, wherein the presence of LOH is associated with the occurrence of cancer. The method and systems described herein represents a significant advance over prior techniques, such as PCR or tissue biopsy by providing a non-invasive, rapid, and accurate method for detecting LOH of specific alleles associated with cancer. Thus, the present invention provides a methods and systems which can be used to screen high-risk populations and to monitor high risk patients undergoing chemoprevention, chemotherapy, immunotherapy or other treatments.

Because the method of the present invention requires only DNA extraction from bodily fluid such as blood, it can be performed at any time and repeatedly on a single patient. Blood can be taken and monitored for LOH before or after surgery; before, during, and after treatment, such as chemotherapy, radiation therapy, gene therapy or immunotherapy; or during follow-up examination after treatment for disease progression, stability, or recurrence. Not being bound by a theory, the method of the present invention also may be used to detect subclinical disease presence or recurrence with an LOH marker specific for that patient since LOH markers are specific to an individual patient's tumor. The method also can detect if multiple metastases may be present using tumor specific LOH markers.

### Detection of Epigenetic Modifications

Histone variants, DNA modifications, and histone modifications indicative of cancer or cancer progression may be used in the present invention. For example, U.S. patent publication 20140206014 describes that cancer samples had elevated nucleosome H2AZ, macroH2A1.1, 5-methylcytosine, P-H2AX(Ser139) levels as compared to healthy subjects. The presence of cancer cells in an individual may generate a higher level of cell free nucleosomes in the blood as a result of the increased apoptosis of the cancer cells. In one embodiment, an antibody directed against marks associated with apoptosis, such as H2B Ser 14(P), may be used to identify single nucleosomes that have been released from apoptotic neoplastic cells. Thus, DNA arising from tumor cells may be advantageously analyzed according to the present invention with high sensitivity and accuracy.

### Pre-Natal Screening

In certain embodiments, the method and systems of the present invention may be used in prenatal screening. In certain embodiments, cell-free DNA is used in a method of prenatal screening. In certain embodiments, DNA associated with single nucleosomes or oligonucleosomes may be detected with the present invention. In preferred embodiments, detection of DNA associated with single nucleosomes or oligonucleosomes is used for prenatal screening. In certain embodiments, cell-free chromatin fragments are used in a method of prenatal screening.

Prenatal diagnosis or prenatal screening refers to testing for diseases or conditions in a fetus or embryo before it is born. The aim is to detect birth defects such as neural tube defects, Down syndrome, chromosome abnormalities, genetic disorders and other conditions, such as spina bifida, cleft palate, Tay Sachs disease, sickle cell anemia, thalassemia, cystic fibrosis, Muscular dystrophy, and fragile X syndrome. Screening can also be used for prenatal sex discernment. Common testing procedures include amniocentesis, ultrasonography including nuchal translucency ultrasound, serum marker testing, or genetic screening. In some cases, the tests are administered to determine if the fetus will be aborted, though physicians and patients also find it useful to diagnose high-risk pregnancies early so that delivery can be scheduled in a tertian,’ care hospital where the baby can receive appropriate care.

It has been realized that there are fetal cells which are present in the mother's blood, and that these cells present a potential source of fetal chromosomes for prenatal DNA-based diagnostics. Additionally, fetal DNA ranges from about 2-10% of the total DNA in maternal blood. Currently available prenatal genetic tests usually involve invasive procedures. For example, chorionic villus sampling (CVS) performed on a pregnant woman around 10-12 weeks into the pregnancy and amniocentesis performed at around 14-16 weeks all contain invasive procedures to obtain the sample for testing chromosomal abnormalities in a fetus. Fetal cells obtained via these sampling procedures are usually tested for chromosomal abnormalities using cytogenetic or fluorescent in situ hybridization (FISH) analyses. Cell-free fetal DNA has been shown to exist in plasma and serum of pregnant women as early as the sixth week of gestation, with concentrations rising during pregnancy and peaking prior to parturition. Because these cells appear very early in the pregnancy, they could form the basis of an accurate, noninvasive, first trimester test. Not being bound by a theory, the present invention provides unprecedented sensitivity in detecting low amounts of fetal DNA. Not being bound by a theory, abundant amounts of maternal DNA is generally concomitantly recovered along with the fetal DNA of interest, thus decreasing sensitivity in fetal DNA quantification and mutation detection. The present invention overcomes such problems by the unexpectedly high sensitivity of the assay.

The H3 class of histones consists of four different protein types: the main types, H3.1 and H3.2; the replacement type, H3.3; and the testis specific variant, H3t. Although H3.1 and H3.2 are closely related, only differing at Ser96, H3.1 differs from H3.3 in at least 5 amino acid positions. Further, H3.1 is highly enriched in fetal liver, in comparison to its presence in adult tissues including liver, kidney and heart. In adult human tissue, the H3.3 variant is more abundant than the H3.1 variant, whereas the converse is true for fetal liver. The present invention may use these differences to detect fetal nucleosomes and fetal nucleic acid in a maternal biological sample that comprises both fetal and maternal cells and/or fetal nucleic acid.

In one embodiment, fetal nucleosomes may be obtained from blood. In other embodiments, fetal nucleosomes are obtained from a cervical mucus sample. In certain embodiments, a cervical mucus sample is obtained by swabbing or lavage from a pregnant woman early in the second trimester or late in the first trimester of pregnancy. The sample may be placed in an incubator to release DNA trapped in mucus. The incubator may be set at 37° C. The sample may be rocked for approximately 15 to 30 minutes. Mucus may be further dissolved with a mucinase for the purpose of releasing DNA. The sample may also be subjected to conditions, such as chemical treatment and the like, as well known in the art, to induce apoptosis to release fetal nucleosomes. Thus, a cervical mucus sample may be treated with an agent that induces apoptosis, whereby fetal nucleosomes are released. Regarding enrichment of circulating fetal DNA, reference is made to U.S. patent publication Nos. 20070243549 and 20100240054. The present invention is especially advantageous when applying the methods and systems to prenatal screening where only a small fraction of nucleosomes or DNA may be fetal in origin.

Prenatal screening according to the present invention may be for a disease including, but not limited to Trisomy 13, Trisomy 16, Trisomy 18, Klinefelter syndrome (47, XXY), (47, XYY) and (47, XXX), Turner syndrome, Down syndrome (Trisomy 21), Cystic Fibrosis, Huntington's Disease, Beta Thalassaemia, Myotonic Dystrophy, Sickle Cell Anemia, Porphyria, Fragile-X-Syndrome, Robertsonian translocation, Angelman syndrome, DiGeorge syndrome and Wolf-Hirschhorn Syndrome.

Several further aspects of the invention relate to diagnosing, prognosing and/or treating defects associated with a wide range of genetic diseases which are further described on the website of the National Institutes of Health under the topic subsection Genetic Disorders (website at health.nih.gov/topic/Genetic Disorders).

### Cancer and Cancer Drug Resistance Detection

In certain embodiments, the present invention may be used to detect genes and mutations associated with cancer. In certain embodiments, mutations associated with resistance are detected. The amplification of resistant tumor cells or appearance of resistant mutations in clonal populations of tumor cells may arise during treatment (see, e.g., Burger J A, et al., Clonal evolution in patients with chronic lymphocytic leukaemia developing resistance to BTK inhibition. Nat Commun. 2016 May 20; 7:11589; Landau D A, et al., Mutations driving CLL and their evolution in progression and relapse. Nature. 2015 Oct. 22; 526(7574):525-30; Landau D A, et al., Clonal evolution in hematological malignancies and therapeutic implications. Leukemia. 2014 January; 28(1):34-43; and Landau D A, et al., Evolution and impact of subclonal mutations in chronic lymphocytic leukemia. Cell. 2013 Feb. 14; 152(4):714-26). Accordingly, detecting such mutations requires highly sensitive assays and monitoring requires repeated biopsy. Repeated biopsies are inconvenient, invasive and costly. Resistant mutations can be difficult to detect in a blood sample or other noninvasively collected biological sample (e.g., blood, saliva, urine) using the prior methods known in the art. Resistant mutations may refer to mutations associated with resistance to a chemotherapy, targeted therapy, or immunotherapy.

In certain embodiments, mutations occur in individual cancers that may be used to detect cancer progression. In one embodiment, mutations related to T cell cytolytic activity against tumors have been characterized and may be detected by the present invention (see e.g., Rooney et al., Molecular and genetic properties of tumors associated with local immune cytolytic activity, Cell. 2015 Jan. 15; 160(1-2): 48-61). Personalized therapies may be developed for a patient based on detection of these mutations (see e.g., WO2016100975A1). In certain embodiments, cancer specific mutations associated with cytolytic activity may be a mutation in a gene selected from the group consisting of CASP8, B2M, PIK3CA, SMC1A, ARID5B, TET2, ALPK2, COL5A1, TP53, DNER, NCOR1, MORC4, CIC, IRF6, MYOCD, ANKLE1, CNKSR1, NF1, SOS1, ARID2, CUL4B, DDX3X, FUBP1, TCP11L2, HLA-A, B or C, CSNK2A1, MET, ASXL1, PD-L1, PD-L2, IDO1, ID02, ALOX12B and ALOX15B, or copy number gain, excluding whole-chromosome events, impacting any of the following chromosomal bands: 6q16.1-q21, 6q22.31-q24.1, 6q25.1-q26, 7p11.2-q11.1, 8p23.1, 8p11.23-p11.21 (containing IDO1, IDO2), 9p24.2-p23 (containing PDL1, PDL2), 10p15.3, 10p15.1-p13, 11p14.1, 12p13.32-p13.2, 17p13.1 (containing ALOX12B, ALOX15B), and 22q11.1-q11.21.

In certain embodiments, the present invention is used to detect a cancer mutation (e.g., resistance mutation) during the course of a treatment and after treatment is completed. The sensitivity of the present invention may allow for noninvasive detection of clonal mutations arising during treatment and can be used to detect a recurrence in the disease.

In certain example embodiments, detection of microRNAs (miRNA) and/or miRNA signatures of differentially expressed miRNA, may used to detect or monitor progression of a cancer and/or detect drug resistance to a cancer therapy. As an example, Nadal et al. (Nature Scientific Reports, (2015) doi:10.1038/srep12464) describe mRNA signatures that may be used to detect non-small cell lung cancer (NSCLC).

In certain example embodiments, the presence of resistance mutations in clonal subpopulations of cells may be used in determining a treatment regimen. In other embodiments, personalized therapies for treating a patient may be administered based on common tumor mutations. In certain embodiments, common mutations arise in response to treatment and lead to drug resistance. In certain embodiments, the present invention may be used in monitoring patients for cells acquiring a mutation or amplification of cells harboring such drug resistant mutations.

Treatment with various chemotherapeutic agents, particularly with targeted therapies such as tyrosine kinase inhibitors, frequently leads to new mutations in the target molecules that resist the activity of the therapeutic. Multiple strategies to overcome this resistance are being evaluated, including development of second generation therapies that are not affected by these mutations and treatment with multiple agents including those that act downstream of the resistance mutation. In an exemplary embodiment, a common mutation to ibrutinib, a molecule targeting Bruton's Tyrosine Kinase (BTK) and used for CLL and certain lymphomas, is a Cysteine to Serine change at position 481 (BTK/C481S). Erlotinib, which targets the tyrosine kinase domain of the Epidermal Growth Factor Receptor (EGFR), is commonly used in the treatment of lung cancer and resistant tumors invariably develop following therapy. A common mutation found in resistant clones is a threonine to methionine mutation at position 790.

Non-silent mutations shared between populations of cancer patients and common resistant mutations that may be detected with the present invention are known in the art (see e.g., WO/2016/187508). In certain embodiments, drug resistance mutations may be induced by treatment with ibrutinib, erlotinib, imatinib, gefitinib, crizotinib, trastuzumab, vemurafenib, RAF/MEK, check point blockade therapy, or antiestrogen therapy. In certain embodiments, the cancer specific mutations are present in one or more genes encoding a protein selected from the group consisting of Programmed Death-Ligand 1 (PD-L1), androgen receptor (AR), Bruton's Tyrosine Kinase (BTK), Epidermal Growth Factor Receptor (EGFR), BCR-Abl, c-kit, PIK3CA, HER2, EML4-ALK, KRAS, ALK, ROS1, AKT1, BRAF, MEK1, MEK2, NRAS, RAC1, and ESR1.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Recently, gene expression in tumors and their microenvironments have been characterized at the single cell level (see e.g., Tirosh, et al. Dissecting the multicellular ecosystem of metastatic melanoma by single cell RNA-seq. Science 352, 189-196, doi:10.1126/science.aad0501 (2016)); Tirosh et al., Single-cell RNA-seq supports a developmental hierarchy in human oligodendroglioma. Nature. 2016 Nov. 10; 539(7628):309-313. doi: 10.1038/nature20123. Epub 2016 Nov. 2; and International patent publication serial number WO 2017004153 A1). In certain embodiments, gene signatures may be detected using the present invention. In one embodiment complement genes are monitored or detected in a tumor microenvironment. In one embodiment MITF and AXL programs are monitored or detected. In one embodiment, a tumor specific stem cell or progenitor cell signature is detected. Such signatures indicate the state of an immune response and state of a tumor. In certain embodiments, the state of a tumor in terms of proliferation, resistance to treatment and abundance of immune cells may be detected.

Thus, in certain embodiments, the invention provides low-cost, rapid, multiplexed cancer detection panels for circulating DNA, such as tumor DNA, particularly for monitoring disease recurrence or the development of common resistance mutations.

### Immunotherapy Applications

The embodiments disclosed herein can also be useful in further immunotherapy contexts. For instance, in some embodiments methods of diagnosing, prognosing and/or staging an immune response in a subject comprise detecting a first level of expression, activity and/or function of one or more biomarker and comparing the detected level to a control level wherein a difference in the detected level and the control level indicates that the presence of an immune response in the subject.

In certain embodiments, the present invention may be used to determine dysfunction or activation of tumor infiltrating lymphocytes (TIL). TILs may be isolated from a tumor using known methods. The TILs may be analyzed to determine whether they should be used in adoptive cell transfer therapies. Additionally, chimeric antigen receptor T cells (CAR T cells) may be analyzed for a signature of dysfunction or activation before administering them to a subject. Exemplary signatures for dysfunctional and activated T cell have been described (see e.g., Singer M, et al., A Distinct Gene Module for Dysfunction Uncoupled from Activation in Tumor-Infiltrating T Cells. Cell. 2016 Sep. 8; 166(6):1500-1511.e9. doi: 10.1016/j.cell.2016.08.052).

In some embodiments, C2c2 is used to evaluate that state of immune cells, such as T cells (e.g., CD8+ and/or CD4+ T cells). In particular, T cell activation and/or dysfunction can be determined, e.g., based on genes or gene signatures associated with one or more of the T cell states. In this way, c2c2 can be used to determine the presence of one or more subpopulations of T cells.

In some embodiments, C2c2 can be used in a diagnostic assay or may be used as a method of determining whether a patient is suitable for administering an immunotherapy or another type of therapy. For example, detection of gene or biomarker signatures may be performed via c2c2 to determine whether a patient is responding to a given treatment or, if the patient is not responding, if this may be due to T cell dysfunction. Such detection is informative regarding the types of therapy the patient is best suited to receive. For example, whether the patient should receive immunotherapy.

In some embodiments, the systems and assays disclosed herein may allow clinicians to identify whether a patient's response to a therapy (e.g., an adoptive cell transfer (ACT) therapy) is due to cell dysfunction, and if it is, levels of up-regulation and down-regulation across the biomarker signature will allow problems to be addressed. For example, if a patient receiving ACT is non-responsive, the cells administered as part of the ACT may be assayed by an assay disclosed herein to determine the relative level of expression of a biomarker signature known to be associated with cell activation and/or dysfunction states. If a particular inhibitory receptor or molecule is up-regulated in the ACT cells, the patient may be treated with an inhibitor of that receptor or molecule. If a particular stimulatory receptor or molecule is down-regulated in the ACT cells, the patient may be treated with an agonist of that receptor or molecule.

In certain example embodiments, the systems, methods, and devices described herein may be used to screen gene signatures that identify a particular cell type, cell phenotype, or cell state. Likewise, through the use of such methods as compressed sensing, the embodiments disclosed herein may be used to detect transcriptomes. Gene expression data are highly structured, such that the expression level of some genes is predictive of the expression level of others. Knowledge that gene expression data are highly structured allows for the assumption that the number of degrees of freedom in the system are small, which allows for assuming that the basis for computation of the relative gene abundances is sparse. It is possible to make several biologically motivated assumptions that allow Applicants to recover the nonlinear interaction terms while under-sampling without having any specific knowledge of which genes are likely to interact. In particular, if Applicants assume that genetic interactions are low rank, sparse, or a combination of these, then the true number of degrees of freedom is small relative to the complete combinatorial expansion, which enables Applicants to infer the full nonlinear landscape with a relatively small number of perturbations. Working around these assumptions, analytical theories of matrix completion and compressed sensing may be used to design under-sampled combinatorial perturbation experiments. In addition, a kernel-learning framework may be used to employ under-sampling by building predictive functions of combinatorial perturbations without directly learning any individual interaction coefficient Compresses sensing provides a way to identify the minimal number of target transcripts to be detected in order obtain a comprehensive gene-expression profile. Methods for compressed sensing are disclosed in PCT/US2016/059230 “Systems and Methods for Determining Relative Abundances of Biomolecules” filed Oct. 27, 2016, which is incorporated herein by reference. Having used methods like compressed sensing to identify a minimal transcript target set, a set of corresponding guide RNAs may then be designed to detect said transcripts. Accordingly, in certain example embodiments, a method for obtaining a gene-expression profile of cell comprises detecting, using the embodiments disclosed, herein a minimal transcript set that provides a gene-expression profile of a cell or population of cells.

### Detecting Nucleic Acid Tagged Molecules

Alternatively, the embodiments described herein may be used to detect nucleic acid identifiers. Nucleic acid identifiers are non-coding nucleic acids that may be used to identify a particular article. Example nucleic acid identifiers, such as DNA watermarks, are described in Heider and Barnekow. “DNA watermarks: A proof of concept” BMC Molecular Biology 9:40 (2008). The nucleic acid identifiers may also be a nucleic acid barcode. A nucleic-acid based barcode is a short sequence of nucleotides (for example, DNA, RNA, or combinations thereof) that is used as an identifier for an associated molecule, such as a target molecule and/or target nucleic acid. A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes can be attached, or “tagged,” to a target molecule and/or target nucleic acid. This attachment can be direct (for example, covalent or non-covalent binding of the barcode to the target molecule) or indirect (for example, via an additional molecule, for example, a specific binding agent, such as an antibody (or other protein) or a barcode receiving adaptor (or other nucleic acid molecule). Target molecule and/or target nucleic acids can be labeled with multiple nucleic acid barcodes in combinatorial fashion, such as a nucleic acid barcode concatemer. Typically, a nucleic acid barcode is used to identify target molecules and/or target nucleic acids as being from a particular compartment (for example a discrete volume), having a particular physical property (for example, affinity, length, sequence, etc.), or having been subject to certain treatment conditions. Target molecule and/or target nucleic acid can be associated with multiple nucleic acid barcodes to provide information about all of these features (and more). Methods of generating nucleic acid-barcodes are disclosed, for example, in International Patent Application Publication No. WO/2014/047561.

### Sample Types

Appropriate samples for use in the methods disclosed herein include any conventional biological sample obtained from an organism or a part thereof, such as a plant, animal, bacteria, and the like. In particular embodiments, the biological sample is obtained from an animal subject, such as a human subject. A biological sample is any solid or fluid sample obtained from, excreted by or secreted by any living organism, including, without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as an infection with a pathogenic microorganism, such as a pathogenic bacteria or virus). For example, a biological sample can be a biological fluid obtained from, for example, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

A sample can also be a sample obtained from any organ or tissue (including a biopsy or autopsy specimen, such as a tumor biopsy) or can include a cell (whether a primary cell or cultured cell) or medium conditioned by any cell, tissue or organ. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, etc.), tissue biopsies (e.g., tumor biopsies), fine-needle aspirates, and/or tissue sections (e.g., cryostat tissue sections and/or paraffin-embedded tissue sections). In other examples, the sample includes circulating tumor cells (which can be identified by cell surface markers). In particular examples, samples are used directly (e.g., fresh or frozen), or can be manipulated prior to use, for example, by fixation (e.g., using formalin) and/or embedding in wax (such as formalin-fixed paraffin-embedded (FFPE) tissue samples). It will be appreciated that any method of obtaining tissue from a subject can be utilized, and that the selection of the method used will depend upon various factors such as the type of tissue, age of the subject, or procedures available to the practitioner. Standard techniques for acquisition of such samples are available in the art. See, for example Schluger et al., J. Exp. Med. 176:1327-33 (1992); Bigby et al., Am. Rev. Respir. Dis. 133:515-18 (1986); Kovacs et al., NEJM 318:589-93 (1988); and Ognibene et al., Am. Rev. Respir. Dis. 129:929-32 (1984).

In other embodiments, a sample may be an environmental sample, such as water, soil, or a surface such as industrial or medical surface. In some embodiments, methods such as disclosed in US patent publication No. 2013/0190196 may be applied for detection of nucleic acid signatures, specifically RNA levels, directly from crude cellular samples with a high degree of sensitivity and specificity. Sequences specific to each pathogen of interest may be identified or selected by comparing the coding sequences from the pathogen of interest to all coding sequences in other organisms by BLAST software.

Several embodiments of the present disclosure involve the use of procedures and approaches known in the art to successfully fractionate clinical blood samples. See, e.g. the procedure described in Han Wei Hou et al., Microfluidic Devices for Blood Fractionation, Micromachines 2011, 2, 319-343; Ali Asgar S. Bhagat et al., Dean Flow Fractionation (DFF) Isolation of Circulating Tumor Cells (CTCs) from Blood, 15^{th} International Conference on Miniaturized Systems for Chemistry and Life Sciences, Oct. 2-6, 2011, Seattle, WA; and International Patent Publication No. WO2011109762, the disclosures of which are herein incorporated by reference in their entirety. Blood samples are commonly expanded in culture to increase sample size for testing purposes. In some embodiments of the present invention, blood or other biological samples may be used in methods as described herein without the need for expansion in culture.

Further, several embodiments of the present disclosure involve the use of procedures and approaches known in the art to successfully isolate pathogens from whole blood using spiral microchannel, as described in Han Wei Hou et al., Pathogen Isolation from Whole Blood Using Spiral Microchannel, Case No. 15995JR, Massachusetts Institute of Technology, manuscript in preparation, the disclosure of which is herein incorporated by reference in its entirety.

Owing to the increased sensitivity of the embodiments disclosed herein, in certain example embodiments, the assays and methods may be run on crude samples or samples where the target molecules to be detected are not further fractionated or purified from the sample.

### Biomarker Sample Types

The sensitivity of the assays described herein are well suited for detection of target nucleic acids in a wide variety of biological sample types, including sample types in which the target nucleic acid is dilute or for which sample material is limited. Biomarker screening may be carried out on a number of sample types including, but not limited to, saliva, urine, blood, feces, sputum, and cerebrospinal fluid. The embodiments disclosed herein may also be used to detect up- and/or down-regulation of genes. For example, a s sample may be serially diluted such that only over-expressed genes remain above the detection limit threshold of the assay.

In certain embodiments, the present invention provides steps of obtaining a sample of biological fluid (e.g., urine, blood plasma or serum, sputum, cerebral spinal fluid), and extracting the DNA. The mutant nucleotide sequence to be detected, may be a fraction of a larger molecule or can be present initially as a discrete molecule.

In certain embodiments, DNA is isolated from plasma/serum of a cancer patient. For comparison, DNA samples isolated from neoplastic tissue and a second sample may be isolated from non-neoplastic tissue from the same patient (control), for example, lymphocytes. The non-neoplastic tissue can be of the same type as the neoplastic tissue or from a different organ source. In certain embodiments, blood samples are collected, and plasma immediately separated from the blood cells by centrifugation. Serum may be filtered and stored frozen until DNA extraction.

In certain example embodiments, target nucleic acids are detected directly from a crude or unprocessed sample, such as blood, serum, saliva, cerebrospinal fluid, sputum, or urine. In certain example embodiments, the target nucleic acid is cell free DNA.

Further embodiments are illustrated in the following Examples which are given for illustrative purposes only and are not intended to limit the scope of the invention.

### EXAMPLES

### Example 1—Design and Testing of a Two-Step SARS-CoV-2 SHERLOCK Assay

A two-step SHERLOCK assay which sensitively detected SARS-CoV-2 RNA at 10 copies per microliter (cp/μL). Using ADAPT, a computational design tool for nucleic acid diagnostics (Metsky et al. 2020. bioRxiv preprint doi: https://doi.org/10.1101/2020.02.26.967026), primers and a crRNA within open reading frame 1a (ORF1a) of SARS-CoV-2 that comprehensively captures known sequence diversity, with high predicted Cas13 targeting activity and SARS-CoV-2 specificity (FIG. 1B)¹⁹ Using both colorimetric and fluorescent readouts, 10 cp/μL of synthetic RNA was detected after incubating samples for 1 h or less. However, preparing the separate amplification and Cas13 detection reaction mixtures and combining each reaction mixture with each sample tested required at least 45 minutes for a small number (<10) of samples (FIGS. 1C and 1D and FIG. 4A). This assay was tested on HUDSON-treated SARS-CoV-2 viral seedstocks, detecting down to 1.31e5 plaque forming units (PFU) per ml via colorimetric readout (FIG. 4B). Finally, the two-step SHERLOCK assay was compared to a RT-qPCR assay, which demonstrated similar limits of detection using fluorescent and lateral-flow based readouts in two laboratories on different continents (FIGS. 4C-4D).

### Example 2—Development of a Single-Step SHERLOCK Assay

It was sought to develop an integrated, streamlined assay that was significantly less time- and labor-intensive than two-step SHERLOCK. However, when RT-RPA (step 1), T7 transcription, and Cas13-based detection (step 2) was combined into a single step (i.e., single-step SHERLOCK), the sensitivity of the assay decreased dramatically. This decrease was specific for RNA input, and, without being bound by theory, likely due to incompatibility of enzymatic reactions with the given conditions (limit of detection (LOD) 10⁶ cp/μL; FIG. 1D and FIG. 5A). As a result, it was evaluated whether additional reaction components and optimized reaction conditions could increase the sensitivity and speed of the assay. Addition of RNase H, in the presence of reverse transcriptase, improved the sensitivity of Cas13-based detection of RNA 10-fold (LOD 105 cp/μL; FIG. 2A and FIGS. 5B and 5C). Without being bound by theory, RNase H likely enhanced the sensitivity by increasing the efficiency of RT through degradation of DNA:RNA hybrid intermediates¹⁵.

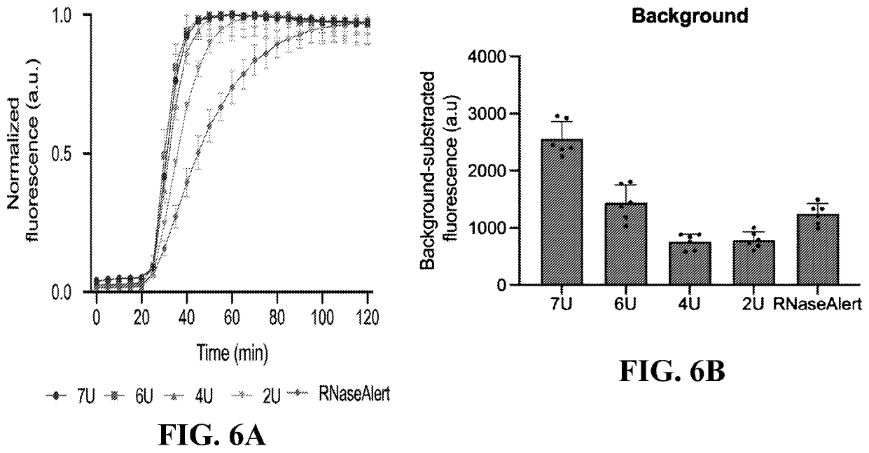

Given that each enzyme involved has optimal activity at distinct reaction conditions, the role of different pHs, monovalent salt, magnesium, and primer concentrations on assay sensitivity was evaluated. Optimized buffer, magnesium, and primer conditions resulted in an LOD of 1,000 cp/μL (FIGS. 2B and 2C and FIGS. 5D and 5E). The speed of Cas13 cleavage and RT was improved to reduce the sample-to-answer time. Given the uracil-cleavage preference of Cas13a^{25,28,29}, detection of RNA in the single-step SHERLOCK assay reached half-maximal fluorescence in ˜67% of the time when RNaseAlert was substituted for a polyU reporter (FIG. 2D, left and FIGS. 6A-6B). In addition, reactions containing SuperScript IV reverse transcriptase reached half-maximal fluorescence two times faster than RevertAid reverse transcriptase (FIG. 2D, right).

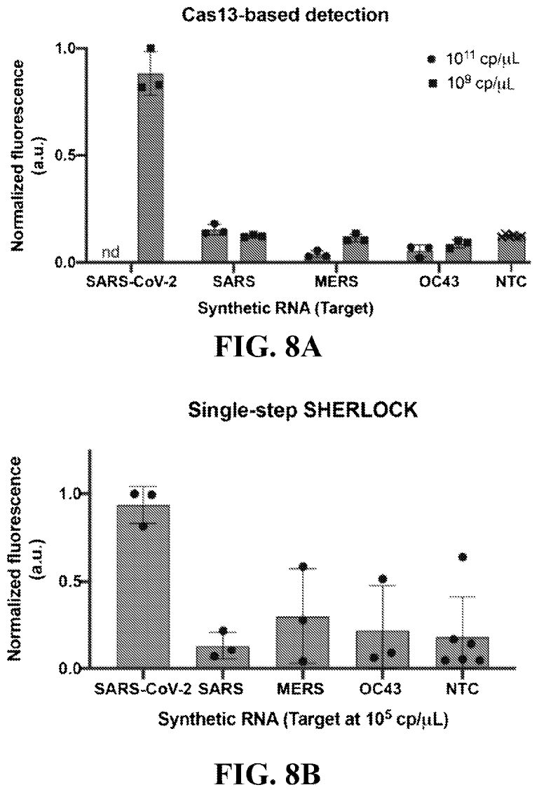

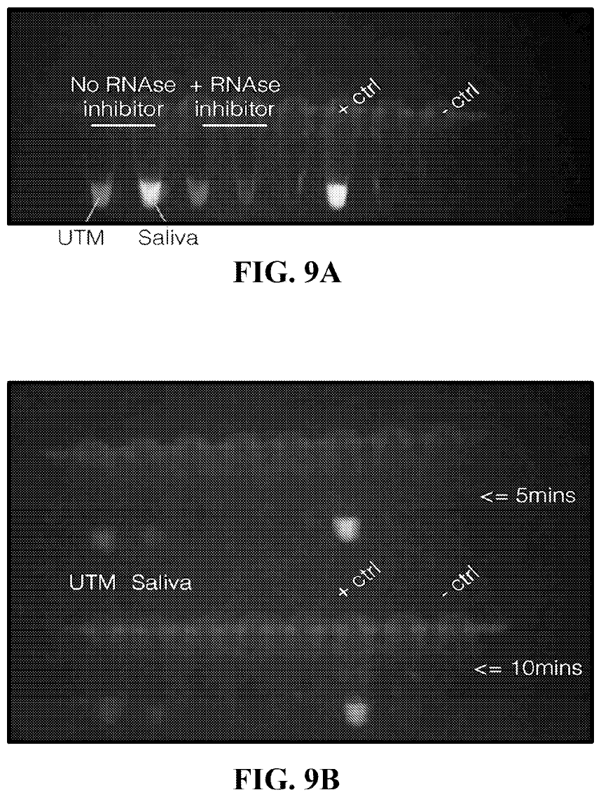

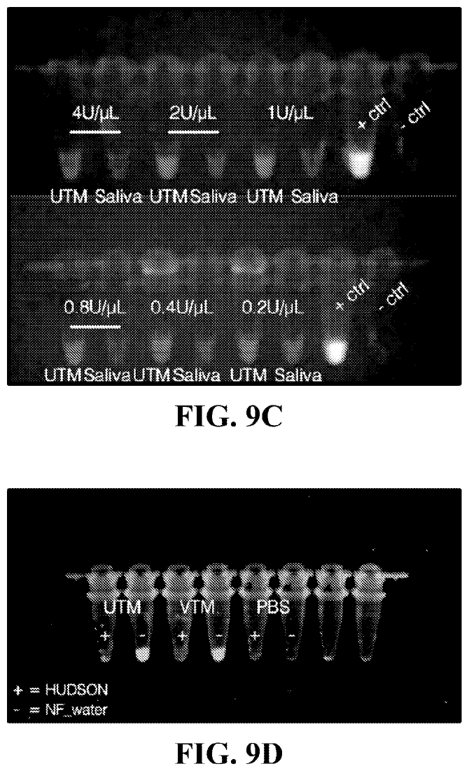

Together, these improvements resulted in an optimized single-step SHERLOCK assay that could specifically detect SARS-CoV-2 RNA with reduced sample-to-answer time and comparable sensitivity compared to the two-step assay. The specificity was tested and the LOD of the optimized single-step SHERLOCK assay was quantified on synthetic SARS-CoV-2 and other human coronavirus RNA targets. This assay detected as few as 10 cp/μL with 100 percent specificity using a fluorescent readout 100,000 times more sensitive than the initial assay—and 100 cp/μL using the lateral-flow-based colorimetric readout (FIGS. 2E and 2F and FIGS. 7 and 8A-8B).

The assay's performance on SARS-CoV-2 RNA extracted from patient nasopharyngeal (NP) swabs was evaluated. The fluorescent single-step SHERLOCK assay was then compared to previously-performed RT-qPCR diagnostic using a pilot set of 9 samples. SARS-CoV-2 was detected from 5 of 5 SARS-CoV-2-positive patient samples tested, demonstrating 100% concordance with RT-qPCR, with no false positives for 4 SARS-CoV-2-negative extracted samples nor 2 non-template controls (FIGS. 2G and 2H and Table 11).

TABLE 11
Patient Sample Information
For samples E42-E47 and P1-P30, the viral quantity listed below is adjusted as sample
was diluted 1:3 for input into in-house RT-qPCR assay. For sample type “S & NP in
VTM”, saliva and nasal specimens were combined during nucleic acid extraction. NP,
nasopharyngeal swab; S, saliva; UTM, universal viral transport medium; VTM, viral
transport medium.

| Sample | | | | Viral quantity | |
|---|---|---|---|---|---|
| name | Sample ID | Sample type | Viral Ct | (cp/μL) | Figures |
| E1 | ON507 | S & NP in VTM | 12.94 | — | 4D |
| E2 | KW326 | S & NP in VTM | 19.81 | — | 4D |
| E3 | KW298 | S & NP in VTM | 17.63 | — | 4D |
| E4 | KW151 | NP in VTM | 29.06 | — | 4D |
| E5 | EK220 | NP in VTM | 19.75 | — | 4D |
| E6 | OS555 | S & NP in VTM | 19.70 | — | 4D |
| E7 | EK272 | NP in VTM | 31.43 | — | 4D |
| E8 | EK271 | NP in VTM | 33.01 | — | 4D |
| E9 | EK239 | NP in VTM | 38.67 | — | 4D |
| E10 | EK219 | NP in VTM | 37.77 | — | 4D |
| E11 | EK204 | NP in VTM | 37.87 | — | 4D |
| E12 | KW311 | S & NP in VTM | 30.18 | — | 4D |
| E13 | OY213 | NP in VTM | 31.73 | — | 4D |
| E14 | OS533 | S & NP in VTM | 36.33 | — | 4D |
| E15 | ON444 | S & NP in VTM | 38.56 | — | 4D |
| E16 | ON356 | S & NP in VTM | 32.36 | — | 4D |
| E17 | ON1010 | S & NP in VTM | 15.11 | — | 4D |
| E18 | ON969 | S & NP in VTM | 18.19 | — | 4D |
| E19 | ON752 | S & NP in VTM | 20.46 | — | 4D |
| E20 | ON964 | S & NP in VTM | 20.43 | — | 4D |
| E21 | ON997 | NP in VTM | 21.21 | — | 4D |
| E22 | ON802 | S & NP in VTM | 22.23 | — | 4D |
| E23 | ON915 | NP in VTM | 23.81 | — | 4D |
| E24 | ON829 | S & NP in VTM | 24.17 | — | 4D |
| E25 | ON718 | S & NP in VTM | 25.52 | — | 4D |
| E26 | ON686 | S & NP in VTM | 26.55 | — | 4D |
| E27 | ON848 | S & NP in VTM | 27.89 | — | 4D |
| E28 | ON1012 | S & NP in VTM | 28.91 | — | 4D |
| E29 | ON1014 | S & NP in VTM | 29.4 | — | 4D |
| E30 | ON900 | S & NP in VTM | 30.55 | — | 4D |
| E31 | ON700 | S & NP in VTM | 31.27 | — | 4D |
| E32 | ON973 | NP in VTM | 32.71 | — | 4D |
| E33 | OS597 | S & NP in VTM | 33.05 | — | 4D |
| E34 | OS634 | S & NP in VTM | 34.03 | — | 4D |
| E35 | OY358 | NP in VTM | 35.42 | — | 4D |
| E36 | OY396 | NP in VTM | 36.12 | — | 4D |
| E37 | OY351 | NP in VTM | 37.25 | — | 4D |
| E38 | OY349 | NP in VTM | 38.58 | — | 4D |
| E39 | OY975 | S & NP in VTM | 39.05 | — | 4D |
| E40 | ON646 | NP in VTM | 40.6 | — | 4D |
| E41 | ON986 | NP in VTM | 41.33 | — | 4D |
| E42 | MA_MGH_00155 | NP in UTM | 25.989 | 892.22 | 2g, h |
| E43 | MA_MGH_00156 | NP in UTM | 17.753 | 85,470.64 | 2g, h |
| E44 | MA_MGH_00157 | NP in UTM | 17.235 | 113,800.532 | 2g, h |
| E45 | MA_MGH_00159 | NP in UTM | 20.861 | 15,298.96 | 2g, h |
| E46 | MA_MGH_00160 | NP in UTM | 20.006 | 24,515.988 | 2g, h |
| E47 | MA_MGH_00166 | NP in UTM | 25.548 | 1,136.696 | 2g, h |
| P1 | MA_MGH_00441 | NP in UTM | 22.109 | 52787.064 | 3g, h |
| P2 | MA_MGH_00442 | NP in UTM | 25.764 | 4386.186 | 3g, h |
| P3 | MA_MGH_00443 | NP in UTM | 26.809 | 2154.246 | 3g, h |
| P4 | MA_MGH_00445 | NP in UTM | 16.429 | 3390658.2 | 3g, h |
| P5 | MA_MGH_00446 | NP in UTM | 17.136 | 2022960.6 | 3g, h |
| P6 | MA_MGH_00447 | NP in UTM | 16.39 | 3426433.56 | 3g, h |
| P7 | MA_MGH_00453 | NP in UTM | 21.935 | 68312.187 | 3g, h |
| P8 | MA_MGH_00454 | NP in UTM | 21.936 | 68293.593 | 3g, h |
| P9 | MA_MGH_00456 | NP in UTM | 15.678 | 5660253.54 | 3g, h |
| P10 | MA_MGH_00458 | NP in UTM | 23.331 | 25530.948 | 3g, h |
| P11 | MA_MGH_00459 | NP in UTM | 15.313 | 7329902.04 | 3g, h |
| P12 | MA_MGH_00460 | NP in UTM | 24.979 | 7977.141 | 3g, h |
| P13 | MA_MGH_00461 | NP in UTM | 16.2 | 3914305.92 | 3g, h |
| P14 | MA_MGH_00463 | NP in UTM | 17.05 | 2157841.26 | 3g, h |
| P15 | MA_MGH_00464 | NP in UTM | 20.234 | 227657.529 | 3g, h |
| P16 | MA_MGH_00465 | NP in UTM | 16.102 | 4201061.4 | 3g, h |
| P17 | MA_MGH_00466 | NP in UTM | 17.933 | 1151605.12 | 3g, h |
| P18 | MA_MGH_00467 | NP in UTM | 19.023 | 541607.094 | 3g, h |
| P19 | MA_MGH_00468 | NP in UTM | 18.661 | 689269.86 | 3g, h |
| P20 | MA_MGH_00469 | NP in UTM | 14.983 | 9240984 | 3g, h |
| P21 | MA_MGH_00471 | NP in UTM | 22.963 | 33130.494 | 3g, h |
| P22 | MA_MGH_00472 | NP in UTM | 13.271 | 30975183 | 3g, h |
| P23 | MA_MGH_00473 | NP in UTM | 18.757 | 645122.16 | 3f, g, h |
| P24 | MA_MGH_00474 | NP in UTM | 17.397 | 1682047.8 | 3f, g, h |
| P25 | MA_MGH_00475 | NP in UTM | 19.896 | 289419.75 | 3f, g, h |
| P26 | MA_MGH_00476 | NP in UTM | 16.181 | 3970140.3 | 3f, g, h |
| P27 | MA_MGH_00477 | NP in UTM | 19.812 | 305659.512 | 3f, g, h |
| P28 | MA_MGH_00479 | NP in UTM | 13.868 | 20324826 | 3f, g, h |
| P29 | MA_MGH_00480 | NP in UTM | 21.362 | 102417.507 | 3g, h |
| P30 | MA_MGH_00481 | NP in UTM | 22.187 | 57196.188 | 3g, h |
| P31 | BocaBio_58 | NP in VTM | 26.778 | 378.502 | 15 |
| P32 | BocaBio_59 | NP in VTM | 23.752 | 3107.53 | 15 |
| P33 | BocaBio_61 | NP in VTM | 27.356 | 253.299 | 15 |
| P34 | BocaBio_66 | NP in VTM | 25.549 | 605.047 | 15 |
| P35 | BocaBio_67 | NP in VTM | 30.27 | 20.938 | 15 |
| P36 | BocaBio_69 | NP in VTM | 22.577 | 5073.833 | 15 |
| P37 | BocaBio_70 | NP in VTM | 31.299 | 9.99 | 15 |
| P38 | BocaBio_73 | NP in VTM | 19.235 | 55318.152 | 15 |
| P39 | BocaBio_78 | NP in VTM | 16.798 | 316178.75 | 15 |
| P40 | BocaBio_80 | NP in VTM | 32.006 | 6.924 | 15 |
| P41 | BocaBio_82 | NP in VTM | 28.433 | 77.379 | 15 |
| P42 | BocaBio_83 | NP in VTM | 26.303 | 353.353 | 15 |

### Example 3—App-Enabled, Extraction-Free Detection of SARS-CoV-2 Using SHINE

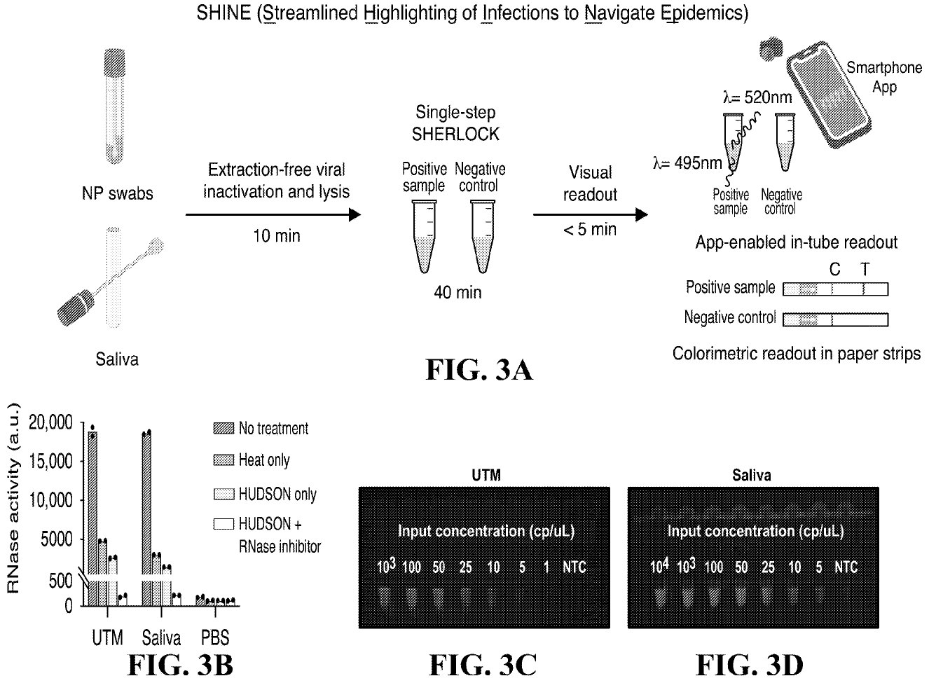

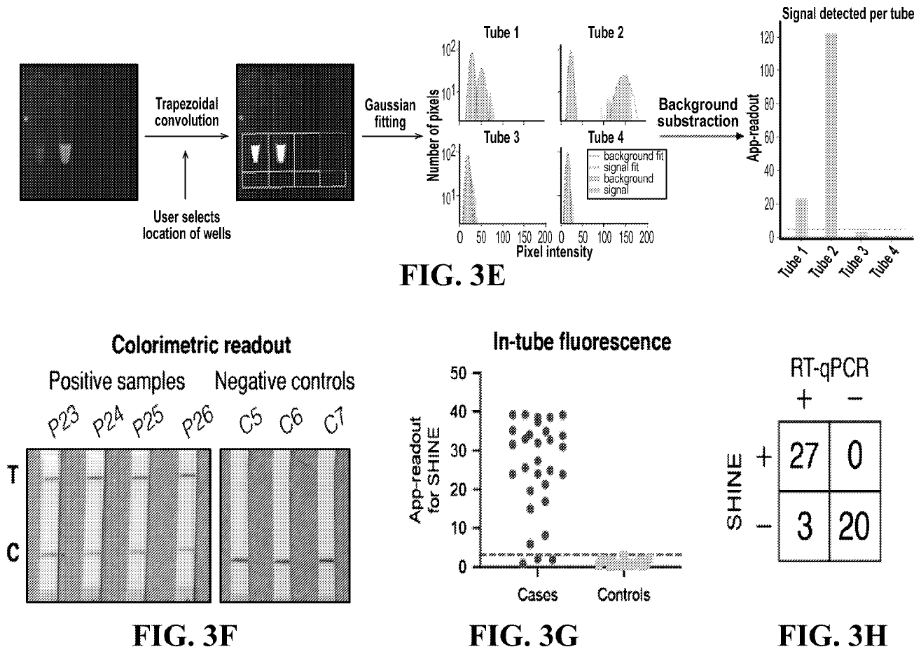

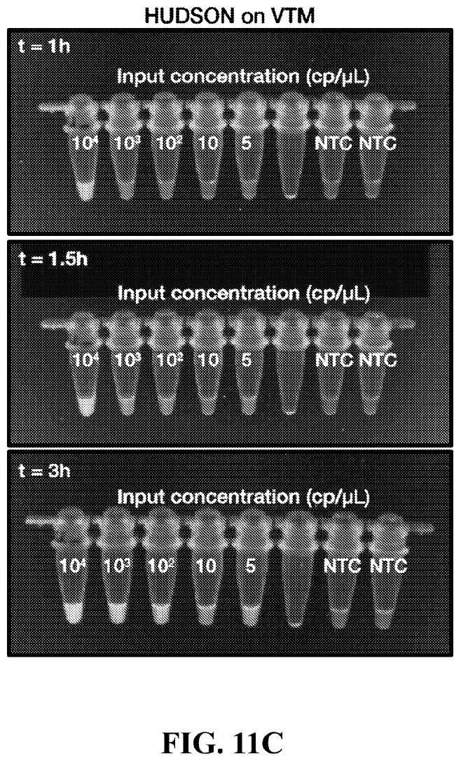

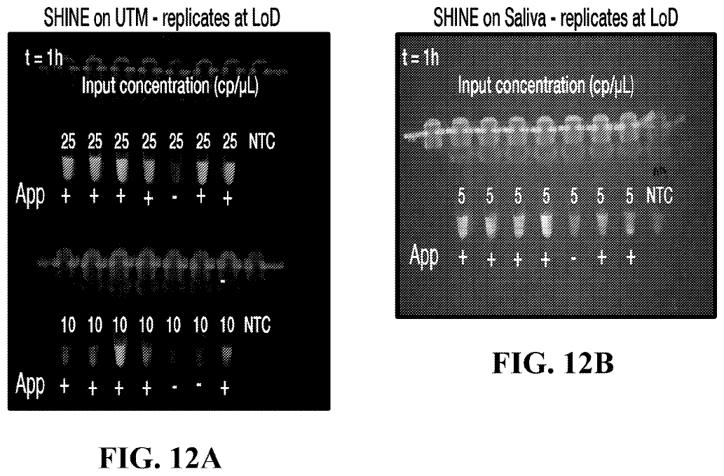

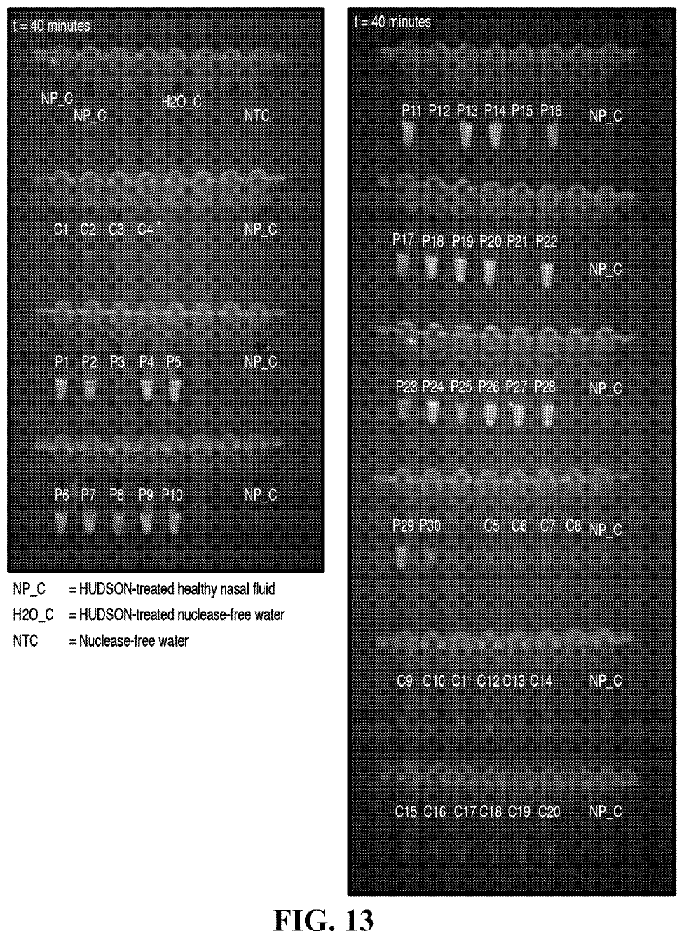

To simplify sample processing, assay output, and data interpretation, we created SHINE, a SHERLOCK-based diagnostic platform for extraction-free viral RNA detection with results interpreted by a companion smartphone application (FIG. 3A). In order to eliminate the need for purified nucleic acids and to reduce total run time, efforts were made to improve HUDSON²⁶ and test its compatibility with COVID-19 collection matrices. During optimization, HUDSON's ability to inactivate RNases by adding RNaseAlert to samples following treatment was assessed, with higher fluorescence corresponding to decreased nuclease inactivation. Through the addition of RNase inhibitors, we reduced the incubation time of HUDSON from 30 minutes to 10 minutes for universal viral transport medium (UTM) and viral transport media (VTM), both used for NP swab samples, and for saliva (FIGS. 3B and 10). With this faster HUDSON protocol, 50 cp/μL of synthetic RNA was detected when spiked into UTM and 100 cp/μL when spiked into saliva, using a colorimetric readout (FIGS. 11A-11C). However, the lateral flow readout requires opening of tubes containing amplified products, which introduce risks of sample contamination. Thus, an in-tube fluorescent readout was incorporated with SHINE. Within 1 hour, as few as 10 cp/μL of SARS-CoV-2 synthetic RNA in HUDSON-treated UTM, 5 cp/μL in HUDSON-treated VTM, and 5 cp/μL in HUDSON-treated saliva was detected with the in-tube fluorescent readout (FIGS. 3C-3D, 12A-12B and 13). To reduce user bias in interpreting results of this in-tube readout, a companion smartphone app was developed which uses the built-in smartphone camera to image the illuminated reaction tubes. The application then calculates the distance of the experimental tube's pixel intensity distribution from that of a user-selected negative control tube, and returns a binary result indicating the presence or absence of viral RNA in the sample (FIG. 3a, e; see Methods in Working Examples for details). Thus, SHINE both minimizes equipment requirements and user interpretation bias when implemented with this in-tube readout and the smartphone application.

### Example 4—Assessment of SHINE's Performance on Patient Samples

SHINE was used to test a set of 50 unextracted NP samples from 30 SARS-CoV-2-positive patients with samples previously tested and confirmed by RT-qPCR and 20 SARS-CoV-2-negative patients. First, SHINE was used with the paper-based colorimetric readout on a subset of 6 SARS-CoV-2-positive samples and detected SARS-CoV-2 RNA in all 6 positive samples, and in none of the negative controls (100% concordance, FIG. 3F). Subsequently, for all 50 samples, SHINE was used with the in-tube fluorescence readout and companion smartphone application.

SARS-CoV-2 RNA was detected in 27 of 30 COVID-19-positive samples (90% sensitivity) and none of the COVID-19-negative samples (100% specificity) after a 10-minute HUDSON and a 40-minute single-step SHERLOCK incubation (FIGS. 3G-3H and 13 and Tables 11 and 12) Thus, SHINE demonstrated 94% concordance using the in-tube readout with a total run time of 50 minutes. Notably, the RT-qPCR-positive patient NP swabs that SHINE failed to detect have higher Ct values than those that SHINE detected as positive (p=0.0017 via one-sided Wilcoxon rank sum test; FIG. 14).

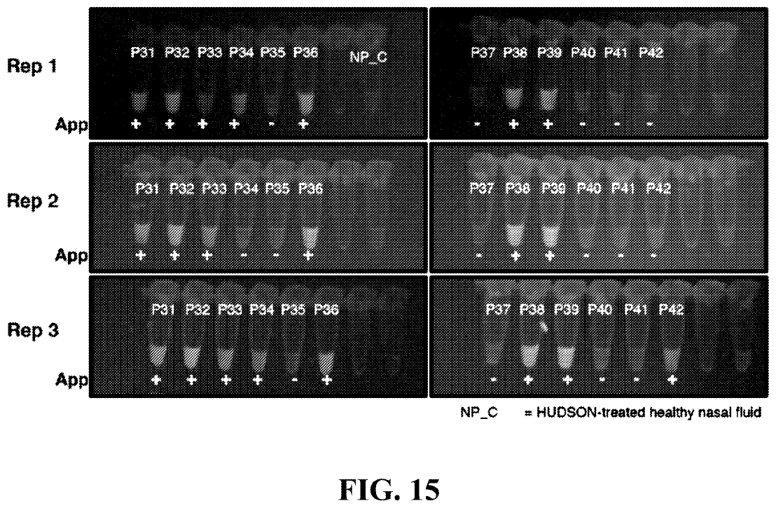

To assess our limit of detection and assay variability across replicates with patient samples, SHINE was tested on a set of 12 independent, unextracted NP samples of varying viral titer as determined by RT-qPCR. For these 12 samples, SHINE was performed and the CDC RT-qPCR N1 assay³⁰ on identical sample aliquots to eliminate potential differences in titer due to uneven numbers of freeze-thaw cycles. We found that samples with titers of less than 100 cp/μL were not detected (4 of 12 samples), those with titers ranging from 100-1,000 cp/μL were detected in one or more technical replicates (4 of 12 samples), and those with titers above 1,000 cp/μL were detected in all technical replicates (4 of 12 samples) (FIG. 15). Therefore, SHINE's performance was equivalent to the CDC assay for RT-qPCR-imputed titers above >1,000 cp/μL, but multiple replicates are needed for samples with lower titers. Importantly, SHINE's sensitivity on patient samples falls well within the range suggested for screening in reopening settings, while offering the rapid turnaround time necessary for testing at a frequency as high as daily³¹.

TABLE 12
COVID-19 negative patient sample information
C1-C9 samples were collected from symptomatic, suspected COVID-19-
positive patients but tested negative for SARS-CoV-2 RNA by RT-qPCR.
C10-C29 samples were collected prior to the start of the coronavirus
pandemic, and therefore are assumed not to contain SARS-CoV-2 RNA.
NP, nasopharyngeal swab; S, saliva; UTM, universal viral transport
medium

| | | | | Viral | |
|---|---|---|---|---|---|
| Sample | | Sample | Viral | quantity | |
| name | Sample ID | type | Ct | (cp/μL) | Figures |
| C1 | EK276 | NP in VTM | — | — | 4D |
| C2 | OY314 | NP in VTM | — | — | 4D |
| C3 | OS572 | S & NP in | — | — | 4D |
| | | VTM | | | |
| C4 | ON513 | S & NP in | — | — | 4D |
| | | VTM | | | |
| C5 | ON1179 | S & NP in | — | — | 4D |
| | | VTM | | | |
| C6 | ON1180 | S & NP in | — | — | 4D |
| | | VTM | | | |
| C7 | ON1181 | S & NP in | — | — | 4D |
| | | VTM | | | |
| C8 | ON1182 | S & NP in | — | — | 4D |
| | | VTM | | | |
| C9 | ON1183 | S & NP in | — | — | 4D |
| | | VTM | | | |
| C10 | MA_MGH_00411 | NP in UTM | — | — | 3g, h |
| C11 | MA_MGH_00412 | NP in UTM | — | — | 3g, h |
| C12 | MA_MGH_00413 | NP in UTM | — | — | 3g, h |
| C13 | MA_MGH_00414 | NP in UTM | — | — | 3g, h |
| C14 | MA_MGH_00320 | NP in UTM | — | — | 3f, g, h |
| C15 | MA_MGH_00321 | NP in UTM | — | — | 3f, g, h |
| C16 | MA_MGH_00322 | NP in UTM | — | — | 3f, g, h |
| C17 | MA_MGH_00323 | NP in UTM | — | — | 3f, g, h |
| C18 | MA_MGH_00324 | NP in UTM | — | — | 3g, h |
| C19 | MA_MGH_00325 | NP in UTM | — | — | 3g, h |
| C20 | MA_MGH_00326 | NP in UTM | — | — | 3g, h |
| C21 | MA_MGH_00327 | NP in UTM | — | — | 3g, h |
| C22 | MA_MGH_00328 | NP in UTM | — | — | 3g, h |
| C23 | MA_MGH_00329 | NP in UTM | — | — | 3g, h |
| C24 | MA_MGH_00338 | NP in UTM | — | — | 3g, h |
| C25 | MA_MGH_00353 | NP in UTM | — | — | 3g, h |
| C26 | MA_MGH_00354 | NP in UTM | — | — | 3g, h |
| C27 | MA_MGH_00355 | NP in UTM | — | — | 3g, h |
| C28 | MA_MGH_00356 | NP in UTM | — | — | 3g, h |
| C29 | MA_MGH_00357 | NP in UTM | — | — | 3g, h |

### Example 5—Discussion of Examples 1-4

These Examples can demonstrate SHINE, a simple method for detecting viral RNA from unextracted patient samples with minimal equipment requirements and multiple readouts. SHINE's simplicity matches that of the most streamlined nucleic acid diagnostics while other isothermal methods require nucleic acid purification or additional readout steps. The use of HUDSON for both NP swabs and less invasive sample types, like saliva, greatly simplifies sample processing. Furthermore, SHINE's performance with saliva is particularly important as it reliably contains SARS-CoV-2RNA and is ideal for routine or daily testing³²⁻³⁴. SHINE's two readouts, lateral flow and in-tube fluorescence, have tradeoffs between equipment needs and sample batch size. Specifically, the lateral flow readout reduces equipment requirements to solely a heat block, but requires longer incubation times to detect samples with lower viral titers. This readout is less amenable to testing large numbers of samples simultaneously and introduces potential risk of sample cross-contamination, as lateral flow strips must be manually inserted into an opened tube for each sample. In contrast, many samples can be imaged in parallel using the in-tube fluorescence readout, but a blue light-emitting device is required. The use of portable transilluminators (0.45 kg in weight for <$500) or small, blue LED lights (˜$15) would eliminate the need for large or expensive fluorescent readers³⁵. Furthermore, the in-tube fluorescence readout and companion smartphone application lend themselves to automated interpretation of results, which is both unbiased and fast. It is believed that SHINE is particularly well-suited for community surveillance testing, as it combines user-friendly, simple preparation methods with sufficient sensitivity and a rapid turn-around time.

With the improvements described, CRISPR-based assays have the potential to address diagnostic needs during the COVID-19 pandemic and in outbreaks to come. Previously developed CRISPR-based detection methods for COVID-19 are highly sensitive and specific, but these assays were primarily tested with purified nucleic acid and require multiple sample-manipulation steps^{19,20,24,26,28,36,37}. SHINE addresses these limitations, using solely two reaction mixtures and sample transfer steps for sample processing and viral detection. With SHINE, CRISPR-based diagnostic testing can now be high-throughput while still only requiring portable equipment, highlighting the technology's potential to disrupt the centralized testing model for diagnosis of infections.

Comparing the performance of SHINE to the gold-standard RT-qPCR methods is essential for understanding its utility for clinical testing. Notably, SHINE demonstrates perfect concordance with RT-qPCR in our samples with titers above 1,000 viral cp/μL.

### Example 6—Materials and Methods for Examples 1-5

### Reagents and Materials

Detailed information about reagents, including the commercial vendors and stock concentrations, is provided in Table 13.

TABLE 13
Reagents used in either the optimized single-step assay or in the optimization process
HUDSON = Heating Unextracted Diagnostic Samples to Obliterate Nucleases. IVT = In-vitro
Transcription. PCR = Polymerase Chain Reaction. RPA = Recombinase Polymerase
Amplification. SHERLOCK = Specific High Sensitivity Enzymatic Reporter Unlocking

| | | | Stock | | |
|---|---|---|---|---|---|
| Reagent | Reaction | Source | Concentration | | Notes |
| EDTA | HUDSON | Thermo Fisher | 0.5M | | |
| | | Scientific ™ | | | |
| TCEP-HCl | HUDSON | Thermo Fisher | 0.5M | | |
| | | Scientific ™ | | | |
| Universal Viral | HUDSON | BD | N/A | | |
| Transport | | | | | |
| Medium (UTM) | | | | | |
| Saliva, Pooled | HUDSON | Lee Biosolutions, | N/A | | |
| Human Donors | | Inc. | | | |
| RNase Inhibitor | HUDSON; | NEB ® | 40 | U/μL | Murine |
| | RPA; | | | | |
| | SHERLOCK | | | | |
| HiScribe ™ T7 | IVT | NEB ® | N/A | | |
| High Yield RNA | | | | | |
| Synthesis Kit | | | | | |
| RNAClean XP | IVT | Beckman Coulter, | N/A | | |
| | | Inc. | | | |
| RNase-Free | IVT | QIAGEN | | | |
| DNase I | | | | | |
| T7 Promoter | IVT | Integrated DNA | | | See Table 14 for |
| ssDNA Primer | | Technologies ™ | | | sequences |
| Ambion ® Linear | PCR | Thermo Fisher | 5 | mg/mL | |
| Acrylamide | | Scientific ™ | | | |
| AMPure XP | PCR | Beckman Coulter, | N/A | | |
| | | Inc. | | | |
| Inter-amplicon | PCR | Integrated DNA | 5 | μM | See Table 14 for sequences |
| Primer | | Technologies ™ | | | |
| MagMAX ™ | PCR | Thermo Fisher | N/A | | |
| mirVana ™ Total | | Scientific ™ | | | |
| RNA Isolation Kit | | | | | |
| PCR Primers | PCR | Integrated DNA | | | See Table 14 for sequences |
| (forward, reverse) | | Technologies ™ | | | |
| Power SYBR ® | PCR | Thermo Fisher | N/A | | |
| Green Master Mix | | Scientific ™ | | | |
| TaqPath ™ 1- | PCR | Thermo Fisher | 4× | | |
| Step RT-qPCR | | Scientific ™ | | | |
| Master Mix | | | | | |
| TURBO ™ DNase | PCR | Thermo Fisher | 2 | U/μL | |
| | | Scientific ™ | | | |
| Magnesium | RPA | TwistDx ™ | 280 | mM | TwistAmp ® Basic Kit |
| Acetate (MgOAc) | | | | | |
| RevertAid | RPA | Thermo Fisher | 200 | U/μL | |
| Reverse | | Scientific ™ | | | |
| Transcriptase | | | | | |
| RNase H | RPA | NEB ® | 5 | U/μL | |
| RPA Pellets | RPA | TwistDx ™ | N/A | | TwistAmp ® Basic Kit |
| (lyophilized) | | | | | |
| RPA Primers | RPA | Integrated DNA | 5 μM of each | | See Table 14 for sequences |
| (forward, reverse) | | Technologies ™ | primer | | |
| SuperScript IV | RPA | Thermo Fisher | 200 | U/μL | Invitrogen |
| Reverse | | Scientific ™ | | | |
| Transcriptase | | | | | |
| Synthetic DNA | RPA | Integrated DNA | 10¹⁰ | copies/μL | See Table 14 for sequences |
| Target | | Technologies ™ | | | |
| Synthetic RNA | RPA | N/A | 10¹⁰ | copies/μL | generated via IVT of |
| Target | | | | | synthetic DNA target |
| Nuclease-free | RPA; | Thermo Fisher | N/A | | Invitrogen |
| Water | SHERLOCK | Scientific ™ | | | |
| Reaction Buffer | RPA; | N/A | 5× | | 0.1M HEPES pH 8.0, |
| (Optimized) | SHERLOCK | | | | 300 mM KCl, 25% PEG- |
| | | | | | 8000 |
| Reaction Buffer | RPA; | N/A | 5× | | 0.1M HEPES pH 6.8, |
| (Original) | SHERLOCK | | | | 300 mM NaCl, 25% |
| | | | | | PEG-8000, 25 uM DTT |
| Cas13a crRNA | SHERLOCK | Integrated DNA | 2 | μM | See Table 14 for sequences |
| | | Technologies ™ | | | |
| Cleavage Buffer | SHERLOCK | N/A | 10× | | 400 mM Tris pH 7.5, 10 |
| (CB) | | | | | mM DTT |
| FAM Cleavage | SHERLOCK | Integrated DNA | 16 | μM | See Table 14 for |
| Reporter | | Technologies ™ | | | sequences |
| (Biotinylated) | | | | | |
| polyU FAM | SHERLOCK | Integrated DNA | 2 | μM | See Table 14 for |
| Cleavage | | Technologies ™ | | | sequences |
| Reporter | | | | | |
| (Quencher) | | | | | |
| LwaCas13a | SHERLOCK | GenScript ® | 0.5 | mg/mL | Custom protein |
| protein | | | | | purification as described |
| | | | | | previously (23) |
| Magnesium | SHERLOCK | Thermo Fisher | 1M | | Invitrogen |
| Chloride (MgCl2) | | Scientific ™ | | | |
| HybriDetect | SHERLOCK | Milenia ® Biotec | N/A | | |
| Assay Buffer | | | | | |
| HybriDetect 1 | SHERLOCK | Milenia ® Biotec | N/A | | |
| Lateral Flow | | | | | |
| Strips | | | | | |
| RNaseAlert ® | SHERLOCK | Thermo Fisher | 2 | μM | |
| Substrate v2 | | Scientific ™ | | | |
| rNTPs | SHERLOCK | NEB ® | 25 mM of each | | |
| | | | nucleotide | | |
| Storage Buffer (SB) | SHERLOCK | N/A | 1× | | 50 mM Tris pH 7.5, 600 |
| | | | | | mM NaCl, 5% glycerol, |
| | | | | | 2 mM DTT |
| T7 RNA | SHERLOCK | Lucigen ® | 50 | U/μL | NextGen ® |

| | | | | |
|---|---|---|---|---|
| Polymerase | | | | |
| Vero (CCL-81 ™) | VIRAL | ATCC ® | N/A | Grivet kidney epithelial |
| | STOCK | | | cells |
| Dulbecco's | VIRAL | Thermo Fisher | N/A | Gibco ® |
| Modified Eagle | STOCK | Scientific ™ | | |
| Medium (DMEM), | | | | |
| high glucose, | | | | |
| pyruvate | | | | |
| Fetal Bovine | VIRAL | SAFC | N/A | |
| Serum (FBS) | STOCK | Biosciences, Inc. | | |
| Vero C1008 | VIRAL | ATCC ® | N/A | Grivet kidney epithelial |
| (CRL-1586 ™) | STOCK | | | cells |
| Avicel ® PH-101 | VIRAL | Millipore Sigma | N/A | |
| | STOCK | | | |
| Crystal Violet | VIRAL | MilliporeSigma | N/A | |
| | STOCK | | | |

### Clinical Samples and Ethics Statement

Clinical samples were de-identified and acquired from clinical studies evaluated and approved by the Institutional Review Board/Ethics Review Committee of the Massachusetts General Hospital and Massachusetts Institute of Technology (MIT) or Redeemer's University Ethical Review Committee. De-identified clinical samples from Boca Biolistics were obtained commercially under their ethical approvals. The Office of Research Subject Projection at the Broad Institute of MIT and Harvard University approved use of samples for the work performed in these Examples.

### Viral and Extracted Sample Preparation and RT-qPCR Testing

For side-by-side comparisons of the two-step SHERLOCK assay and RT-qPCR on viral seedstocks, the 2019-nCoV/USA-WA1-A12/2020 isolate of SARS-CoV-2 was provided by the US Centers for Disease Control and Prevention (CDC). The virus was passaged at the Integrated Research Facility-Frederick in high containment (BSL-3) by inoculating grivet kidney epithelial Vero cells (American Type Culture Collection (ATCC) #CCL-81) at a multiplicity of infection (MOI) of 0.01. Infected cells were incubated for 48 or 72 hours in Dulbecco's Modified Eagle Medium with 4.5 g/L D-glucose, L-glutamine, and 110 mg/L sodium pyruvate (DMEM, Gibco) containing 2% heat-inactivated fetal bovine serum (SAFC Biosciences) in a humidified atmosphere at 37° C. with 5% CO₂. The resulting viral stock was harvested and quantified by plaque assay using Vero E6 cells (ATCC #CRL-1586) with a 2.5% Avicel overlay and stained after 48 hours with a 0.2% crystal violet stain.

For side-by-side comparisons of the two-step SHERLOCK assay and RT-qPCR on patient samples, nasal swab or combined nasal and saliva samples were collected from symptomatic patients in whom COVID-19 was suspected. Nasal swabs were collected and stored in viral transport medium (VTM)⁴². All nucleic acid extractions were performed using the QIAamp Viral RNA Mini Kit (Qiagen). For a subset of patients, saliva samples were combined with nasal samples during extraction. The starting volume for extraction was 70 μL and extracted nucleic acid was eluted into 60 μL of nuclease-free water. RT-qPCR was performed using either the RT-PCR Reagent Set for COVID-19 Real-time detection (DaAn-GENE) or the GeneFinder™ COVID-19 Plus RealAmp Kit (OSANG Healthcare) using the N target (primer and probe sequences not publicly available). RT-qPCR cycling conditions for DnAn-GENE kit were as follows: RT at 50° C. for 15 min, heat activation at 95° C. for 15 min and 45 cycles of a denaturing step at 94° C. for 15 s followed by annealing and elongation steps at 55° C. for 45 s. RT-qPCR cycling conditions for OSANG Healthcare's kit were as follows: RT at 50° C. for 20 min, heat activation at 95° C. for 5 min, and 45 cycles with a denaturing step at 95° C. for 15 s followed by annealing and elongation steps at 58° C. for 60 s.

Nasal swabs were collected and stored in universal viral transport medium (UTM; BD) and stored at −80° C. prior to nucleic acid extraction. For the initial set of 50 NP patient samples, nucleic acid extraction was performed using MagMAX™ mirVana™ Total RNA isolation kit. The starting volume for the extraction was 250 μl and extracted nucleic acid was eluted into 60 μl of nuclease-free water. Extracted nucleic acid was then immediately Turbo DNase-treated (Thermo Fisher Scientific), purified twice with RNACleanXP SPRI beads (Beckman Coulter), and eluted into 15 l of Ambion Linear Acrylamide (Thermo Fisher Scientific) water (0.8%).

Turbo DNase-treated extracted RNA was then tested for the presence of SARS-CoV-2 RNA using a lab-developed, probe-based RT-qPCR assay based on the N1 target of the CDC assay. RT-qPCR was performed on a 1:3 dilution of the extracted RNA using TaqPath™ 1-Step RT-qPCR Master Mix (Thermo Fisher Scientific) with the following forward and reverse primer sequences, respectively: GACCCCAAAATCAGCGAAAT (SEQ ID NO: 15), TCTGGTTACTGCCAGTTGAATCTG (SEQ ID NO: 16). The RT-PCR assay was run with a double-quenched FAM probe with the following sequence: 5′-FAM-ACCCCGCATTACGTTTGGTGGACC-BHQ1-3′ (SEQ ID NO: 17). RT-qPCR was run on a QuantStudio 6 (Applied Biosystems) with RT at 48° C. for 30 min and 45 cycles with a denaturing step at 95° C. for 10 s followed by annealing and elongation steps at 60° C. for 45 s. The data were analyzed using the Standard Curve (SC) module of the Applied Biosystems Analysis Software.

Patient samples for side-by-side SHINE and RT-qPCR testing (from Boca Biolistics) were extracted using the QIAamp Viral RNA Mini Kit (Qiagen). The starting volume for the extraction was 100 μL and extracted nucleic acid was eluted into 40 μL of nuclease-free water. Extracted RNA was then tested for the presence of SARS-CoV-2 RNA using the lab-developed, probe-based RT-qPCR assay mentioned above (based on the N1 target of the CDC assay). Primers, probes and conditions are the same as mentioned above.

### SARS-CoV-2 Assay Design and Synthetic Template Information

SARS-CoV-2-specific forward and reverse RPA primers and Cas13-crRNAs were designed as previously described¹⁹. In short, the designs were algorithmically selected, targeting 100% of 20 published SARS-CoV-2 genomes, and predicted by a machine learning model to be highly active (Metsky et al. in prep). Moreover, the crRNA was selected for its high predicted specificity towards detection of SARS-CoV-2, versus related viruses, including other bat and mammalian coronaviruses and other human respiratory viruses (https://adapt.sabetilab.org/covid-19/).

Specificity target sequences were generated using the same design software noted above by providing the amplicon coordinates of the designed assay within the viral species of interest and an alignment of the selected viral species. The specificity targets tested represent the overall medoid of sequence clusters at the provided amplicon for each selected viral species within the designed SARS-CoV-2 SHERLOCK assay.

Synthetic DNA targets with appended upstream T7 promoter sequences (5′-GAAATTAATACGACTCACTATAGGG-3′ (SEQ ID NO: 18)) were ordered as double-stranded DNA (dsDNA) gene fragments from IDT, and were in vitro transcribed to generate synthetic RNA targets. In vitro transcription was conducted using the HiScribe T7 High Yield RNA Synthesis Kit (New England Biolabs (NEB)) as previously described²⁴. In brief, a T7 promoter ssDNA primer (5′-GAAATTAATACGACTCACTATAGGG-3′ (SEQ ID NO: 19)) was annealed to the dsDNA template and the template was transcribed at 37° C. overnight. Transcribed RNA was then treated with RNase-free DNase I (QIAGEN) to remove any remaining DNA according to the manufacturer's instructions. Finally, purification occurred using RNAClean SPRI XP beads at 2× transcript volumes in 37.5% isopropanol.

Sequence information for the synthetic targets, RPA primers, and Cas13-crRNA is listed in Table 14.

TABLE 14
Oligonucleotides used in these Examples 1-6
MERS = Middle East Respiratory Syndrome. OC43 = Human coronavirus OC43.
SARS = Severe Acute Respiratory Syndrome

| | | SARS-COV-2 |
|---|---|---|
| | | Gene |
| Reagent | Sequence | Location |
| Cas13a crRNA | CUCUUCUUCAGGUUGAAGAGCAGCAGAA (SEQ ID NO: | Orf1ab |
| | 20) | |
| PCR Primer | GACCCCAAAATCAGCGAAAT (SEQ ID NO: 21) | N1 |
| (forward) | | |
| PCR Primer | TCTGGTTACTGCCAGTTGAATCTG (SEQ ID NO: 22) | N1 |
| (reverse) | | |
| PCR Probe | /FAM/ACCCCGCATTACGTTTGGTGGACC/BHQ1 (SEQ ID | |
| | NO: 23) | |
| RPA Primer | CCAAGGTAAACCTTTGGAATTTGGTGCCAC (SEQ ID NO: | Orf1ab |
| (forward) | 24) | |
| RPA Primer | ACTATCATCATCTAACCAATCTTCTTCTTG (SEQ ID NO: | Orf1ab |
| (reverse) | 25) | |
| Synthetic MERS | GCACCATGCTATTGCTTTAACGCTGAGGGTGATGCATC | |
| DNA Target | TTGGTCTTCTACTATGATCTTCTCTCTTCACCCCGTCGA | |
| | GTGTGACGAGGAGTGTTCTGAAGTAGAGGCTTCAGATT | |
| | TAGAAGAAGGTGAATCAGAGTGCATTTCTGAGACTTCAA | |
| | CTGAACAAGTTGACGTTTCTCATGAGGTTTCTGACGACG | |
| | AGTGGGCTGCTGCAGTTGATGAAGCGTTCCCCCTCGAT | |
| | GAAGCAGAAGATGTTACTGAATCTGTGCAAGAAGAAGC | |
| | ACAACCAGTAGAAGTACCTGTTGAAGATATTGCGCAGG | |
| | TTGTCATAGCTGACACCTTACAGGAAACTCCTGTTGTGT | |
| | CTGATACTGTTGAAGTCCCACCGCAAGTGGTGAAACTT | |
| | CCGTCTGAACCTCAGACTATCCAGCCCGAGGTAAAAGA | |
| | AGTTGCACCTGTCTATGAGGCTGATACCGAACAGACAC | |
| | AGAGTGTTACTGTTA (SEQ ID NO: 26) | |
| Synthetic OC43 | ATATGGAGGAATTTTATGCTGTGGTGATTGATGCCATAG | |
| DNA Target | AAGAGAAACTTTCTCCATGTAAGGAGCTTGAAGGTGTA | |
| | GGTGCTAAAGTTAGTGCCTTTTTACAGAAATTAGAGGAT | |
| | AATCCCCTATTTTTATTTGATGAGGCTGGCGAAGAAGTT | |
| | TTTGCTCCTAAATTGTATTGTGCCTTTACAGCTCCTGAA | |
| | GATGATGACTTTCTTGAGGAAAGTGATGTTGAAGAAGAT | |
| | GATGTAGAAGGTGAGGAAACTGATTTAACTATCACAAGT | |
| | GCTGGACAGCCTTGTGTTGCTAGTGAACAGGAGGAATC | |
| | TTCTGAAGTCTTAGAGGACACTTTGGATGATGGTCCAAG | |
| | TGTGGAGACATCTGATTCACAAGTTGAAGAAGATGTAGA | |
| | AATGTCGGATTTTGTTGATCTTGAATCTGTGATTCAGGA | |
| | TTATGAAAATGTTTGTTTTGAGTTTTATACTACAGAGCCA | |
| | GAATTTGT (SEQ ID NO: 27) | |
| Synthetic SARS | GAATGTGAGAATCACATTTGAGCTTGATGAACGTGTTGA | |
| DNA Target | CAAAGTGCTTAATGAAAAGTGCTCTGTCTACACTGTTGA | |
| | ATCCGGTACCGAAGTTACTGAGTTTGCATGTGTTGTAGC | |
| | AGAGGCTGTTGTGAAGACTTTACAACCAGTTTCTGATCT | |
| | CCTTACCAACATGGGTATTGATCTTGATGAGTGGAGTGT | |
| | AGCTACATTCTACTTATTTGATGATGCTGGTGAAGAAAA | |
| | CTTTTCATCACGTATGTATTGTTCCTTTTACCCTCCAGAT | |
| | GAGGAAGAAGAGGACGATGCAGAGTGTGAGGAAGAAG | |
| | AAATTGATGAAACCTGTGAACATGAGTACGGTACAGAG | |
| | GATGATTATCAAGGTCTCCCTCTGGAATTTGGTGCCTCA | |
| | GCTGAAACAGTTCGAGTTGAGGAAGAAGAAGAGGAAGA | |
| | CTGGCTGGATGATACTACTGAGCAATCAGAGATTGAGC | |
| | CAGAACCAGAA (SEQ ID NO: 28) | |
| Synthetic SARS- | GTGAGTTTAAATTGGCTTCACATATGTATTGTTCTTTCTA | Orf1ab |
| CoV-2 DNA Target | CCCTCCAGATGAGGATGAAGAAGAAGGTGATTGTGAAG | |
| | AAGAAGAGTTTGAGCCATCAACTCAATATGAGTATGGTA | |
| | CTGAAGATGATTACCAAGGTAAACCTTTGGAATTTGGTG | |
| | CCACTTCTGCTGCTCTTCAACCTGAAGAAGAGCAAGAA | |
| | GAAGATTGGTTAGATGATGATAGTCAACAAACTGTTGGT | |
| | CAACAAGACGGCAGTGAGGACAATCAGACAACTACTAT | |
| | TCAAACAATTGTTGAGGTTCAACCTCAATTAGAGATGGA | |
| | ACTTACACCAGTTGTTCAGACTATTGAAGTGAATAGTTT | |
| | TAGTGGTTATTTAAAACTTACTGACAATGTATACATTAA | |
| | A | |
| | AATGCAGACATTGTGGAAGAAGCTAAAAAGGTAAAACC | |
| | AACAGTGGTTGTTAATGCAGCCAATGTTTACCTTAAACA | |
| | TGGAGGAGG (SEQ ID NO: 29) | |
| T7 Promoter ssDNA | GAAATTAATACGACTCACTATAGGG (SEQ ID NO: 30) | dsDNA |
| Primer | | appended |
| | | upstream of |
| | | target |
| FAM Cleavage | /56-FAM/rUrUrUrUrUrUrUrUrUrUrUrUrUrU/3Bio/ | |
| Reporter | (SEQ ID NO: 31) | |
| (Biotinylated) | | |
| polyU (i.e., 6U or | /56-FAM/rUrUrUrUrUrU(rU)/3IABKFQ/ | |
| 7U) FAM Cleavage | (SEQ ID NO: 32) | |
| Reporter | | |
| (Quencher) | | |

### Two-Step SARS-CoV-2 Assay

The two-step SHERLOCK assay was performed as previously described^{19,24,26}. Briefly, the assay was performed in two steps: (1) isothermal amplification via recombinase polymerase amplification (RPA) and (2) LwaCas13a-based detection using a single-stranded RNA (ssRNA) fluorescent reporter. For RPA, the TwistAmp Basic Kit (TwistDx) was used as previously described (i. e., with RPA forward and reverse primer concentrations of 400 nM and a magnesium acetate concentration of 14 mM)²⁶ (with the following modifications: RevertAid reverse transcriptase (Thermo Fisher Scientific) and murine RNase inhibitor (NEB) were added at final concentrations of 4 U/μl each, and synthetic RNAs or viral seedstocks were added at known input concentrations making up 10% of the total reaction volume. The RPA reaction was then incubated on the thermocycler for 20 minutes at 41° C. For the detection step, 1 μl of RPA product was added to 19 μl detection master mix. The detection master mix consisted of the following reagents (final concentrations in master mix listed), with magnesium chloride added last: 45 nM LwaCas13a protein resuspended in 1× storage buffer (SB: 50 mM Tris pH 7.5, 600 mM NaCl, 5% glycerol, and 2 mM dithiothreitol (DTT); such that the resuspended protein is at 473.7 nM), 22.5 nM crRNA, 125 nM RNaseAlert substrate v2 (Thermo Fisher Scientific), 1× cleavage buffer (CB; 400 mM Tris pH 7.5 and 10 mM DTT), 2 U/μL murine RNase inhibitor (NEB), 1.5 U/μl NextGen T7 RNA polymerase (Lucigen), 1 mM of each rNTP (NEB), and 9 mM magnesium chloride. Reporter fluorescence kinetics were measured at 37° C. on a Biotek Cytation 5 plate reader using a monochromator (excitation: 485 nm, emission: 520 nm) every 5 minutes for up to 3 hours.

### Single-Step SARS-CoV-2 Assay Optimization

The starting point for optimization of the single-step SHERLOCK assay was generated by combining the essential reaction components of both the RPA and the detection steps in the two—

step assay, described above^{24,26}. Briefly, a master mix was created with final concentrations of 1× original reaction buffer (20 mM HEPES pH 6.8 with 60 mM NaCl, 5% PEG, and 5 μM DTT), 45 nM LwaCas13a protein resuspended in 1×SB (such that the resuspended protein is at 2.26 μM), 136 nM RNaseAlert substrate v2, 1 U/μl murine RNase inhibitor, 2 mM of each rNTP, 1 U/μl NextGen T7 RNA polymerase, 4 U/μl RevertAid reverse transcriptase, 0.32 μM forward and reverse RPA primers, and 22.5 nM crRNA. The TwistAmp Basic Kit lyophilized reaction components (1 lyophilized pellet per 102 μl final master mix volume) were resuspended using the master mix. After pellet resuspension, cofactors magnesium chloride and magnesium acetate were added at final concentrations of 5 mM and 17 mM, respectively, to complete the reaction.

Master mix and synthetic RNA template were mixed and aliquoted into a 384-well plate in triplicate, with 20 μl per replicate at a ratio of 19:1 master mix:sample. Fluorescence kinetics were measured at 37° C. on a Biotek Cytation 5 or Biotek Synergy H1 plate reader every 5 minutes for 3 hours, as described above. No significant difference was observed in performance between the two plate reader models.

Optimization occurred iteratively, with a single reagent modified in each experiment. The reagent condition (e.g., concentration, vendor, or sequence) that produced the most optimal results defined as either a lower limit of detection (LOD) or improved reaction kinetics (i. e., reaction saturates faster) was incorporated into the protocol. Thus, the protocol used for every future reagent optimization consisted of the most optimal reagent conditions for every reagent tested previously.

For all optimization experiments, the modulated reaction component is described in the figures, associated captions, or associated legends. Across all experiments, the following components of the master mix were held constant: 45 nM LwaCas3a protein resuspended in 1×SB (such that the resuspended protein is at 2.26 M), 1 U/μl murine RNase inhibitor, 2 mM of each rNTP, 1 U/μl NextGen T7 RNA polymerase, and 22.5 nM crRNA, and TwistDx RPA TwistAmp Basic Kit lyophilized reaction components (1 lyophilized pellet per 102 μl final master mix volume). In all experiments, the master mix components except for the magnesium cofactor(s) were used to resuspend the lyophilized reaction components, and the magnesium cofactor(s) were added last. All other experimental conditions, which differ among the experiments due to real-time optimization, are detailed in Table 15.

TABLE 15

| | | RPA | Magnesium | Reverse | Fluorescent | | |
|---|---|---|---|---|---|---|---|
| FIG. | Buffer | Primer | Cofactor | Transcriptase | Reporter | RNase H | HUDSON |
| 2A | 20 mM | 320 nM | 5 mM | 4 U/μL | 136 nM | 0 or | |
| | HEPES | | MgCl₂, | RevertAid | RNaseAlert | 0.05 U/μL | |
| | pH 6.8 | | 17 mM | | substrate v2 | | |
| | with | | MgOAc | | | | |
| | 60 mM | | | | | | |
| | NaCl, 5% | | | | | | |
| | PEG, and | | | | | | |
| | 5 μM | | | | | | |
| | DTT | | | | | | |
| 2B | 20 mM | 120 nM | 5 mM | 4 U/μL | 136 nM | 0.1 U/μL | |
| | HEPES | or 320 | MgCl₂, | RevertAid or | RNaseAlert | | |
| | pH 6.8, | nM | 17 mM | SuperScript | substrate v2 | | |
| | 7.3, or | | MgOAc | IV | or polyU | | |
| | 8.0 with | | | | FAM | | |
| | 20, 60, or | | | | | | |
| | 100 mM | | | | | | |
| | NaCl or | | | | | | |
| | KCl | | | | | | |
| 2C | 20 mM | 120 or | 5 mM | 4 U/μL | 136 nM | 0.1 U/μL | |
| | HEPES | 320 nM | MgCl₂, | RevertAid | RNaseAlert | | |
| | pH 6.8 | | 17 mM | | substrate v2 | | |
| | with | | MgOAc | | | | |
| | 60 mM | | | | | | |
| | NaCl, 5% | | | | | | |
| | PEG, and | | | | | | |
| | 5 μM | | | | | | |
| | DTT | | | | | | |
| 2D | 20 mM | 120 | 14 mM | 2 U/μL | 136 nM | 0.1 U/μL | |
| (left) | HEPES | nM | MgOAc | SuperScript | RNaseAlert | | |
| | pH 8.0 | | | IV | substrate v2 | | |
| | with 60 | | | | or polyU | | |
| | mM KCl | | | | FAM | | |
| | and 5% | | | | | | |
| | PEG | | | | | | |
| 2D | 20 mM | 120 nM | 5 mM | 4.2 U/μL | 136 nM | 0.1 U/μL | |
| (right) | HEPES | | MgCl₂, | SuperScript | RNaseAlert | | |
| | pH 8.0 | | 17 mM | IV | substrate v2 | | |
| | with | | MgOAc | | | | |
| | 60 mM | | | | | | |
| | NaCl and | | | | | | |
| | 5% PEG | | | | | | |
| 3B | UTM, | | | | 200 mM | | 0 or 8 |
| | saliva, or | | | | RNaseAlert | | U/μL |
| | PBS +/−100 | | | | substrate v2 | | RNase |
| | mM | | | | added after | | inhibitor, |
| | TCEP | | | | heating | | 5 min |
| | and 1 | | | | | | heating at |
| | mM | | | | | | 40° C. |
| | EDTA | | | | | | |
| 5A | 20 mM | 320 nM | 5 mM | 4 U/μL | 136 nM | 0.1 U/μL | |
| | HEPES | | MgCl₂, | RevertAid | RNaseAlert | | |
| | pH 8.0 | | 17 mM | | substrate v2 | | |
| | with 60 | | MgOAc | | | | |
| | mM KCl | | | | | | |
| | and 5% | | | | | | |
| | PEG | | | | | | |
| 5B | 20 mM | 320 nM | 5 mM | 4 U/μL | 136 nM | 0.1 U/μL | |
| | HEPES | | MgCl₂, | RevertAid | RNaseAlert | | |
| | pH 6.8 | | 17 mM | | substrate v2 | | |
| | with | | MgOAc | | | | |
| | 60 mM | | | | | | |
| | NaCl, 5% | | | | | | |
| | PEG, and | | | | | | |
| | 5 μM | | | | | | |
| | DTT | | | | | | |
| 5C | 20 mM | 320 nM | 5 mM | 4 U/μL | 136 nM | 0.1, 1, or | |
| | HEPES | | MgCl₂, | RevertAid | RNaseAlert | 1.5 U/μL | |
| | pH 6.8 | | 17 mM | | substrate v2 | | |
| | with | | MgOAc | | | | |
| | 60 mM | | | | | | |
| | NaCl, 5% | | | | | | |
| | PEG, and | | | | | | |
| | 5 μM | | | | | | |
| | DTT | | | | | | |
| 5D | 20 mM | 120 or | 11.7, 14, | 2 U/μL | 125 nM | 0.1 U/μL | |
| | HEPES | 150 nM | 23.4, or | SuperScript | polyU FAM | | |
| | pH 8.0 | | 35.1 mM | IV | | | |
| | with 60 | | MgOAc | | | | |
| | mM KCl | | | | | | |
| | and 5% | | | | | | |
| | PEG | | | | | | |
| 5E | 20 mM | 90, 120, | 5 mM | 4 U/μL | 136 nM | 0.1 U/μL | |
| | HEPES | 240, | MgCl₂, | RevertAid | RNaseAlert | | |
| | pH 8.0 | 320, or | 17 mM | | substrate v2 | | |
| | with 60 | 480 nM | MgOAc | | | | |
| | mM KCl | | | | | | |
| | and 5% | | | | | | |
| | PEG | | | | | | |
| 6A | 20 mM | 120 | 14 mM | 2 U/μL | 136 nM | 0.1 U/μL | |
| | HEPES | nM | MgOAc | SuperScript | RNaseAlert | | |
| | pH 8.0 | | | IV | substrate v2 | | |
| | with 60 | | | | or 7 U, 6 U, | | |
| | mM KCl | | | | 4 U, or 2 U | | |
| | and 5% | | | | FAM | | |
| | PEG | | | | | | |
| 6B | 20 mM | 120 nM | 14 mM | 2 U/μL | 136 nM | 0.1 U/μL | |
| | HEPES | | MgOAc | SuperScript | RNaseAlert | | |
| | pH 8.0 | | | IV | substrate v2 | | |
| | with 60 | | | | or 7 U, 6 U, | | |
| | mM KCl | | | | 4 U, or 2 U | | |
| | and 5% | | | | FAM | | |
| | PEG | | | | | | |
| 9A | 100 mM | | | | 200 mM | | 4 U/μL |
| | TCEP | | | | RNaseAlert | | RNase |
| | and 1 | | | | substrate v2 | | inhibitor, |
| | mM | | | | added after | | 20 min |
| | EDTA | | | | heating | | heating at |
| | | | | | | | 50° C. |
| 9B | 100 mM | | | | 200 mM | | 4 U/μL |
| | TCEP | | | | RNaseAlert | | RNase |
| | and 1 | | | | substrate v2 | | inhibitor, |
| | mM | | | | added after | | 5 min |
| | EDTA | | | | heating | | heating at |
| | | | | | | | 50° C. |
| 9C | 100 mM | | | | 200 mM | | 4, 2, 1, |
| | TCEP | | | | RNaseAlert | | 0.8, 0.4, |
| | and 1 | | | | substrate v2 | | 0.2 U/μL |
| | mM | | | | added after | | RNase |
| | EDTA | | | | heating | | inhibitor, |
| | | | | | | | 5 min |
| | | | | | | | heating at |
| | | | | | | | 50° C. |

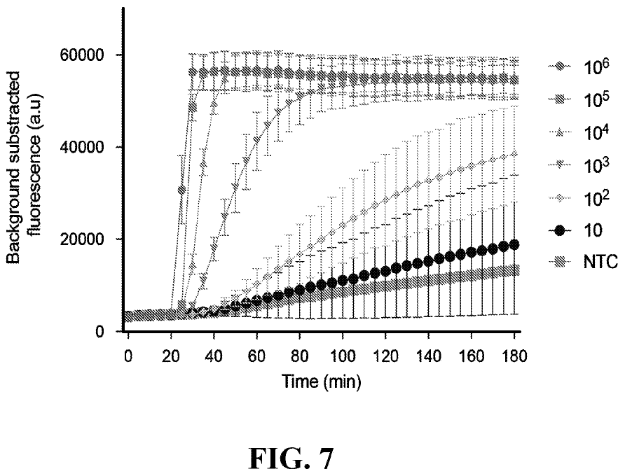

Experiments in FIGS. 1C, 1D, 2E, and 4A-4D use the two-step assay, the experiment in FIG. 1D uses the original one-step assay, experiments in FIGS. 2E, 2F, 2G, and 7 use the optimized one-step assay, and experiments in FIGS. 3C, 3D, 3F, 3G, 10, 11A-11C, 12A-12B, 13, and S14 use SHINE, all described in Methods. MgCl₂ = magnesium chloride. MgOAc = magnesium acetate.

### Single-Step SARS-CoV-2 Optimized Reaction

The optimized reaction (see Supplementary Protocol below for exemplary implementation) consists of a master mix with final concentrations of 1× optimized reaction buffer (20 mM HEPES pH 8.0 with 60 mM KCl and 5% PEG), 45 nM LwaCas13a protein resuspended in 1×SB (such that the resuspended protein is at 2.26 μM), 125 nM polyU [i.e., 6 uracils (6 U) or 7 uracils (7 U) in length, unless otherwise stated] FAM quenched reporter, 1 U/μl murine RNase inhibitor, 2 mM of each rNTP, 1 U/μl NextGen T7 RNA polymerase, 2 U/μl Invitrogen SuperScript IV (SSIV) reverse transcriptase (Thermo Fisher Scientific), 0.1 U/μl RNase H (NEB), 120 nM forward and reverse RPA primers, and 22.5 nM crRNA. Once the master mix is created, it is used to resuspend the TwistAmp Basic Kit lyophilized reaction components (1 lyophilized pellet per 102 μl final master mix volume). Finally, magnesium acetate is the sole magnesium cofactor, and is added at a final concentration of 14 mM to generate the final master mix.

The sample is added to the complete master mix at a ratio of 1:19 and the fluorescence kinetics are measured at 37° C. using a Biotek Cytation 5 or Biotek Synergy H1 plate reader as described above.

For the specificity data, fluorescence kinetics were measured at 37° C. using a Molecular Devices, SpectraMax M2 plate reader using the same excitation and emission parameters described above; notably, this plate reader model required twice the reporter concentration (250 nM polyU FAM) to achieve a comparable LOD to the Biotek models

### Detection Via In-Tube Fluorescence and Via Lateral Flow Strip

Minor modifications were made to the single-step SARS-CoV-2 optimized reaction to visualize the readout via in-tube fluorescence or lateral flow strip.

For in-tube fluorescence with the optimized single-step reaction, we generated the master mix as described above, except the 7 U FAM quenched reporter was used at a concentration of 62.5 nM. The sample was added to the complete master mix at a ratio of 1:19. Samples were incubated at 37° C. and images were collected after 30, 45, 60, 90, 120 and 180 minutes of incubation, with image collection terminating once experimental results were clear. A dark reader transilluminator (DR196 model, Clare Chemical Research) or Gel Doc™ EZ Imager (BioRad) with the blue tray was used to illuminate the tubes.

For lateral-flow readout with the two-step SHERLOCK method, the Cas13-based detection mix was generated as described above, except we used a biotinylated FAM reporter at a final concentration of 1 μM rather than RNase Alert v2. For lateral-flow readout using the optimized single-step SHERLOCK assay, we generated the single-step master mix as described above, except a biotinylated FAM reporter at a final concentration of 1 μM rather than the quenched polyU FAM reporters was used. For both two-step and single-step SHERLOCK, the sample was added to the complete master mix at a ratio of 1:19. After 1-3 hours of incubation at 37° C., the detection reaction was diluted 1:4 in Milenia HybriDetect Assay Buffer, and the Milenia HybriDetect 1 (TwistDx) lateral flow strip was added. Sample images were collected 5 minutes following incubation of the strip. Lateral flow results were assessed either by the user or in an automated fashion by measuring the pixel intensity of the test band using ImageJ.

### In-Tube Fluorescence Reader Mobile Phone Application

To enable smartphone-based fluorescence analysis, a companion application was designed. Using the application, the user captures an image of a set of strip tubes illuminated by a transilluminator. The user then identifies regions of interest in the captured image by overlaying a set of pre-drawn boxes onto experimental and control tubes. Image and sample information is then transmitted to a server for analysis. Within each of the user-selected squares, the server models the bottom of each tube as a trapezoid and uses a convolutional kernel to determine the location of maximal signal within each tube, using data from the green channel of the RGB image. The server then identifies the background signal proximal to each tube and fits a Gaussian distribution around the background signal and around the in-tube signal. The difference between the mean pixel intensity of the background signal and the mean pixel intensity of the in-tube signal is then calculated as the background-subtracted fluorescence signal for each tube. To identify experimentally significant fluorescent signals, a score is computed for each experimental tube; this score is equal to the distance between the experimental and control background-subtracted fluorescence divided by the standard deviation of pixel intensities in the control signal. Finally, positive or negative samples are determined based on whether the score is above (positive, +) or below (negative, −) 1.5, a threshold identified empirically.

### HUDSON Protocols

HUDSON nuclease and viral inactivation were performed on viral seedstock as previously described with minor modifications to the temperatures and incubation times (25). In short, 100 mM TCEP (Thermo Fisher Scientific) and 1 mM EDTA (Thermo Fisher Scientific) were added to non-extracted viral seedstock and incubated for 20 minutes at 50° C., followed by 10 minutes at 95° C. The resulting product was then used as input into the two-step SHERLOCK assay.

The improved HUDSON nuclease and viral inactivation protocol was performed as previously described, with minor modifications (26). Briefly, 100 mM TCEP, 1 mM EDTA, and 0.8 U/μl murine RNase inhibitor were added to clinical samples in universal viral transport medium or human saliva (Lee Biosolutions). These samples were incubated for 5 minutes at 40° C., followed by 5 minutes at 70° C. (or 5 minutes at 95° C., if saliva). The resulting product was used in the single-step detection assay. In cases where synthetic RNA targets were used, rather than clinical samples (e.g., during reaction optimization), targets were added after the initial heating step (40° C. at 5 minutes). This is meant to recapitulate patient samples, as RNA release occurs after the initial heating step when the temperature is increased, and viral particles lyse.

For optimization of nuclease inactivation using HUDSON, only the initial heating step was performed. The products were then mixed 1:1 with 400 mM RNaseAlert substrate v2 in nuclease-free water and incubated at room temperature for 30 minutes before imaging on a transilluminator or measuring reporter fluorescence on a Biotek Synergy H1 [at room temperature using a monochromator (excitation: 485 nm, emission: 520 nm) every 5 minutes for up to 30 minutes]. The specific HUDSON protocol parameters modified are indicated in the figure captions.

### Data Analysis and Schematic Generation

Conservation of SARS-CoV-2 sequences across the SHERLOCK assay was determined using publicly available genome sequences via GISAID. Analysis was based on an alignment of 5376 SARS-CoV-2 genomic sequences. Percent conservation was measured at each nucleotide within the RPA primer and Cas13-crRNA binding sites and represents the percentage of genomes that have the consensus base at each nucleotide position.

As described above, fluorescence values are reported as background-subtracted, with the fluorescence value collected before reaction progression (i.e., the latest time at which no change in fluorescence is observed, usually time 0, 5, or 10 minutes) subtracted from the final fluorescence value (3 hours, unless otherwise indicated).

Normalized fluorescence values are calculated using data aggregated from multiple experiments with at least one condition in common and for the specificity testing where all conditions were performed in the same experiment on a SpectraMax M2 (Molecular Devices). The maximal fluorescence value across all experiments is set to 1, with fluorescence values from the same experiment set as ratios of the maximal fluorescence value. Common conditions across experiments are set to the same normalized value, and that value is propagated to determine the normalized values within an experiment.

The Wilcoxon rank sum test was conducted in MATLAB (MathWorks). Schematics shown in FIG. 1A and FIG. 3A were created using BioRender.com. All other schematics were generated in Adobe Illustrator (v24.1.2). Data panels were primarily generated via Prism 8 (GraphPad), except FIG. 3E which was generated using Python (version 3.7.2), seaborn (version 0.10.1) and matplotlib (version 3.2.1)^{43,44}.

### Supplementary Protocol

Example protocol for in-tube fluorescent readout of N clinical samples with 20 uL reaction volumes.

Nuclease inactivation and viral particle lysis
1. Mix 2.28*N μL TCEP-HCl (0.5 M) with 0.023*N μL EDTA (0.5 M) at room temperature.
2. Add 8.8 μL of each sample [e.g., nasopharyngeal swab in universal viral transport medium (UTM) or saliva] to a strip tube. Add 2 μL TCEP/EDTA mixture and 0.2 μL RNase inhibitor (40 U/uL) to each sample.
3. Incubate samples for 5 minutes at 40° C., followed by 5 minutes at 70° C. (if UTM) or 5 minutes at 95° C. (if saliva).

Viral RNA Amplification and Detection

For N samples with 20 μL reaction volumes and a 6.5% pipetting loss, the reaction factor M is N/5, rounded up to the nearest integer.

TABLE 16

| | Amount |
|---|---|
| Reagent | (μL) |
| RPA Pellets | 1 * M |
| Reaction Buffer: 0.1M HEPES pH 8.0, 300 | 21.50 * M |
| mM KCl, 25% PEG-8000 (Optimized) (5X) | |
| Cas13a resuspended in SB (50 mM Tris pH 7.5, | 2.14 * M |
| 600 mM NaCl, 5% glycerol, 2 mM DTT) (2.26 μM) | |
| 6U FAM Reporter (2 μM) | 3.19 * M |
| RNase Inhibitor (40 U/μL) | 2.69 * M |
| rNTPs (25 mM each nucleotide) | 8.60 * M |
| SuperScript IV Reverse Transcriptase (200 U/μL) | 1.08 * M |
| RNase H (5 U/μL) | 2.15 * M |
| T7 RNA Polymerase (50 U/μL) | 2.15 * M |
| Nuclease-free H2O | 50.60 * M |
| RPA Primers (5 μM each primer) | 2.45 * M |
| Cas13a crRNA (2 μM) | 0.46 * M |
| Magnesium Acetate (280 μM) | 5.1 * M |

1. Make a master mix of all components listed above, except the RPA pellets and the magnesium acetate. Keep the master mix on ice.
2. Resuspend the RPA pellets with 65 uL of the master mix each, returning the resuspended material to the master mix and mixing well.
3. Add magnesium acetate to the reaction mixture.
4. Aliquot 19 uL master mix into wells of a strip tube that is pre-chilled on ice. Add 1 uL of sample (i.e., HUDSON product) or negative control (e.g., nuclease-free water) to each aliquot, mixing thoroughly.
5. Incubate samples at 37° C. for 30 to 90 minutes.

Download the companion smartphone application, which is available at https://github.com/sameeds/sherlock-reader-app. Follow instructions in the application to upload a picture of the results. The application will return binary outcomes (i.e., whether each sample is positive or negative relative to the negative control).

### REFERENCES RELATED TO EXAMPLES 1-6

1. Kaplan, S. & Thomas, K. Despite Promises, Testing Delays Leave Americans ‘Flying Blind’. https://www.nytimes.com/2020/04/06/health/coronavirus-testing-us.html (2020).
2. Bai, Y. et al. Presumed Asymptomatic Carrier Transmission of COVID-19. JAMA (2020) doi:10.1001/jama.2020.2565.
3. Rothe, C. et al. Transmission of 2019-nCoV Infection from an Asymptomatic Contact in Germany. N. Engl. J. Med. 382, 970-971 (2020).
4. Wang, C., Horby, P. W., Hayden, F. G. & Gao, G. F. A novel coronavirus outbreak of global health concern. Lancet 395, 470-473 (2020).
5. Zhu, N. et al. A Novel Coronavirus from Patients with Pneumonia in China, 2019. N. Engl. J. Med. 382, 727-733 (2020).
6. Zhou, P. et al. A pneumonia outbreak associated with a new coronavirus of probable bat origin. Nature 579, 270-273 (2020).
7. World Health Organization (WHO). COVID-19 Weekly Epidemiological Update, Aug. 23, 2020. https://www.who.int/docs/default-source/coronaviruse/situation-reports/20200824—weekly-epi-update.pdf?sfvrsn=806986d1_4.
8. U.S. Food and Drug Administration. Policy for COVID-19 Tests During the Public Health Emergency (Revised). https://www.fda.gov/regulatory-information/search-fda-guidance-documents/policy-coronavirus-disease-2019-tests-during-public-health-emergency-revised (2020).
9. Notomi, T. et al. Loop-mediated isothermal amplification of DNA. Nucleic Acids Res. 28, E63 (2000).
10. Park, G.-S. et al. Development of Reverse Transcription Loop-Mediated Isother Amplification Assays Targeting SARS-CoV-2. J Mol. Diagn. (2020) doi:10.1016/j.jmoldx.2020.03.006.
11. Baek, Y. H. et al. Development of a reverse transcription-loop-mediated isothermal amplification as a rapid early-detection method for novel SARS-CoV-2. Emerg. Microbes Infect. 1-31 (2020).
12. Niemz, A., Ferguson, T. M. & Boyle, D. S. Point-of-care nucleic acid testing for infectious diseases. Trends in Biotechnology vol. 29 240-250 (2011).
13. Abott ID NOW™ COVID-19—Alere is now. https://www.alere.com/en/home/product-details/id-now-covid-19.html.
14. Piepenburg, O., Williams, C. H., Stemple, D. L. & Armes, N. A. DNA detection using recombination proteins. PLoS Biol. 4, e204 (2006).
15. Qian, J. et al. An enhanced isothermal amplification assay for viral detection. Preprint at https://www.biorxiv.org/content/10.1101/2020.05.28.118059v1.full (2020)
16. Basu, A. et al. Performance of Abbott ID NOW COVID-19 rapid nucleic acid amplification test in nasopharyngeal swabs transported in viral media and dry nasal swabs, in a New York City academic institution. J. Clin. Microbiol. (2020) doi:10.1128/JCM.01136-20.
17. Svoboda, E. A sticking point for rapid flu tests? Nature 573, S56-S57 (2019).
18. Zhang F, Abudayyeh 00, Gootenberg J S. A protocol for detection of COVID-19 using CRISPR diagnostics. https://www.broadinstitute.org/files/publications/special/COVID-19%20detection%20(updated).pdf.
19. Metsky, H. C., Freije, C. A., Kosoko-Thoroddsen, T.-S. F., Sabeti, P. C. & Myhrvold, C. CRISPR-based surveillance for COVID-19 using genomically-comprehensive machine learning design. Preprint a https://www.biorxiv.org/content/10.1101/2020.02.26.967026v2 (2020)
Broughton, J. P. et al. CRISPR-Cas12-based detection of SARS-CoV-2. Nat. Biotechnol. 2020) doi:10.1038/s41587-020-0513-4.
21. Rauch, J. N. et al. A Scalable, Easy-to-Deploy, Protocol for Cas13-Based Detection of SARS-CoV-2 Genetic Material. Preprint at https://www.biorxiv.org/content/10.1101/2020.04.20.052159v1 (2020).
22. U.S. Food and Drug Administration. Sherlock CRISPR SARS-CoV-2 Kit. https://www.fda.gov/media/137747/download.
23 Guo, L. et al. SARS-CoV-2 detection with CRISPR diagnostics. Cell Discovery 6, 1-4 (2020).
24. Gootenberg, J. S. et al. Nucleic acid detection with CRISPR-Cas13a/C2c2. Science 356, 438-442 (2017).
25. Abudayyeh, O. O. et al. C2c2 is a single-component programmable RNA-guided RNA-targeting CRISPR effector. Science 353, aaf5573 (2016).
26. Myhrvold, C. et al. Field-deployable viral diagnostics using CRISPR-Cas13. Science 360, 444-448 (2018).
27. Barnes, K. G. et al. Deployable CRISPR-Cas13a diagnostic tools to detect and report Ebola and Lassa virus cases in real-time. Nat. Commun. 11, 1-10 (2020).
28. Gootenberg, J. S. et al. Multiplexed and portable nucleic acid detection platform with Cas13, Cas12a, and Csm6. Science 360, 439-444 (2018).
29. East-Seletsky, A., O'Connell, M. R., Burstein, D., Knott, G. J. & Doudna, J. A. RNA Targeting by Functionally Orthogonal Type VI-A CRISPR-Cas Enzymes. Mol. Cell 66, 373-383.e3 (2017).
30. U.S. Food and Drug Administration (FDA). CDC 2019-Novel Coronavirus (2019-nCoV) Real-Time RT-PCR Diagnostic Panel. https://www.fda.gov/media/134922/download.
31. Paltiel, A. D., David Paltiel, A., Zheng, A. & Walensky, R. P. COVID-19 screening strategies that permit the safe re-opening of college campuses. Preprint at https://www.medrxiv.org/content/10.1101/2020.07.06.20147702v1 (2020).
32. Williams, E., Bond, K., Zhang, B., Putland, M. & Williamson, D. A. Saliva as a non-invasive specimen for detection of SARS-CoV-2. J. Clin. Microbiol. (2020) doi:10.1128/JCM.00776-20.
33. To, K. K.-W. et al. Temporal profiles of viral load in posterior oropharyngeal saliva samples and serum antibody responses during infection by SARS-CoV-2: an observational cohort study. Lancet Infect. Dis. 20, 565-574 (2020).
34. Wyllie, A. L. et al. Saliva is more sensitive for SARS-CoV-2 detection in COVID-19 patients than nasopharyngeal swabs. Preprint at https://www.medrxiv.org/content/10.1101/2020.04.16.20067835v1 (2020).
35. Katzmeier, F. et al. A low-cost fluorescence reader for in vitro transcription and nucleic acid detection with Cas13a. PLoS One 14, e0220091 (2019).
36. Chen, J. S. et al. CRISPR-Cas12a target binding unleashes indiscriminate single-stranded DNase activity. Science 360, 436-439 (2018).
37. Joung, J. et al. Point-of-care testing for COVID-19 using SHERLOCK diagnostics. Preprint at https://www.medrxiv.org/content/10.1101/2020.05.04.20091231v1 (2020)
38. Lu, J. et al. Genomic Epidemiology of SARS-CoV-2 in Guangdong Province, China. Cell 181, 997-1003.e9 (2020).
39. Srivatsan, S. et al. Preliminary support for a ‘dry swab, extraction free’ protocol for SARS-CoV-2 testing via RT-qPCR. Preprint at https://www.biorxiv.org/content/10.1101/2020.04.22.056283v1 (2020)
40. Calvert, A. E., Biggerstaff, B. J., Tanner, N. A., Lauterbach, M. & Lanciotti, R. S. Rapid colorimetric detection of Zika virus from serum and urine specimens by reverse transcription loop-mediated isothermal amplification (RT-LAMP). PLoS One 12, e0185340 (2017).
41. Rabe, B. A. & Cepko, C. SARS-CoV-2 Detection Using an Isothermal Amplification Reaction and a Rapid, Inexpensive Protocol for Sample Inactivation and Purification. Preprint at https://www.medrxiv.org/content/10.1101/2020.04.23.20076877v1 (2020)
42. World Health Organization (WHO). Annex 8. Viral transport media (VTM). https://www.who.int/ihr/publications/Annex8.pdf?ua=1.
43. Waskom, M. et al. mwaskom/seaborn: v0.8.1 (September 2017). (2017) doi:10.5281/zenodo.883859.
44. Hunter, J. D. Matplotlib: A 2D Graphics Environment. Comput. Sci. Eng. 9, 90-95 (2007).

### Example 7—Optimization of SHINE

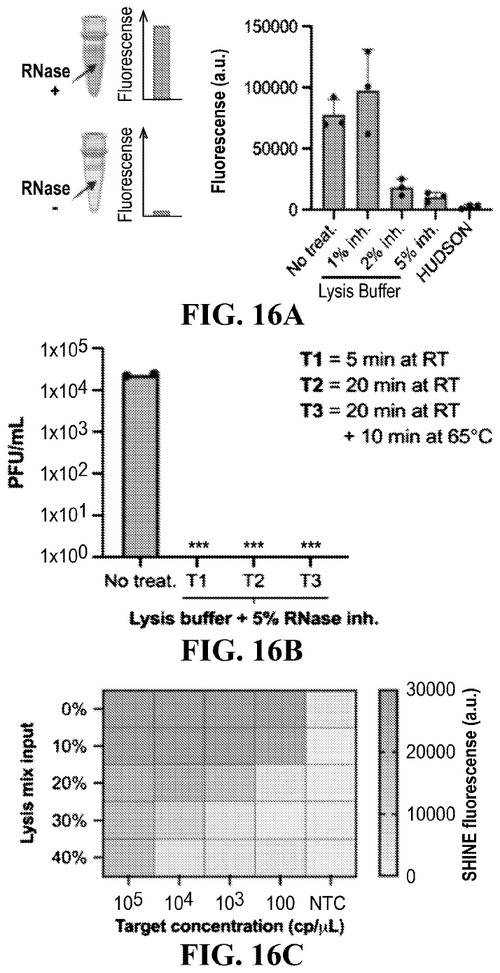

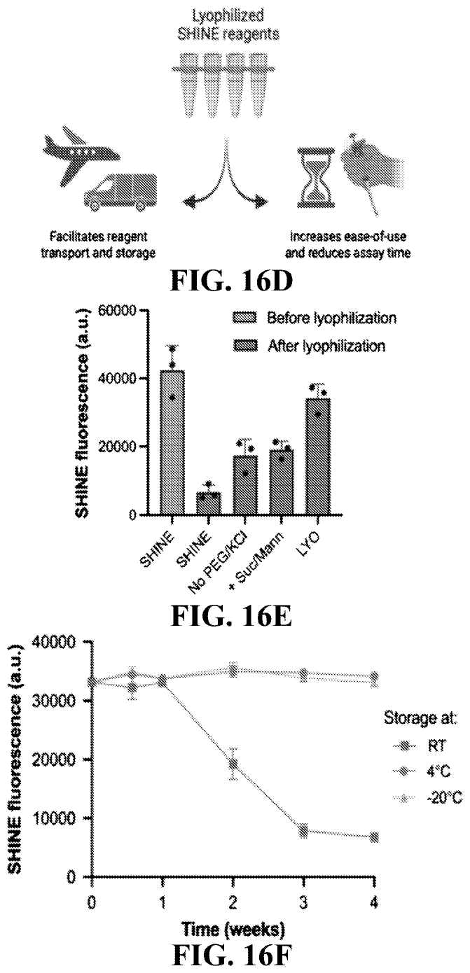

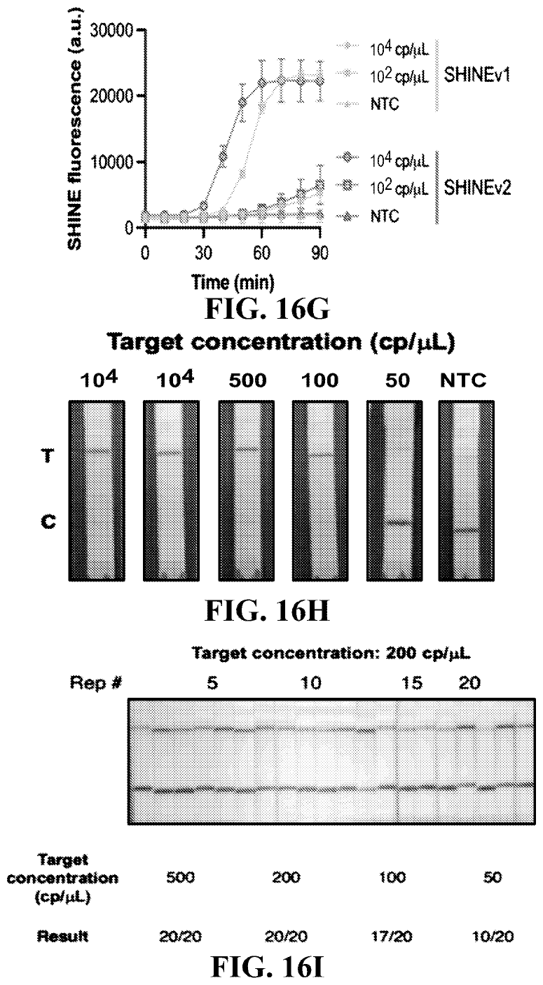

This example shows optimization of SHINE for improved assay readout and deployability of a diagnostic assay. Briefly, optimization included optimization of lysis, amplification and/or CRISPR-Cas detection reagents and compositions. See also FIGS. 16A-19C. More specifically, for lyophilization optimization, PEG (e.g., PEG-8000) and potassium chloride was removed from prior exemplary embodiments of SHINE lysis, amplification, and/or CRISPR-Cas detection reagents (see e.g., Examples 1-6 herein). See e.g., FIGS. 16A-16H, particularly at FIG. 16E. The lyophilization buffer was composed of about 20 mM HEPES (pH 8.0) with about 5% w/v sucrose and about 150 mM mannitol. This lyophilization buffer facilitated SHINE's assay activity post-lyophilization (see e.g., FIG. 16E).

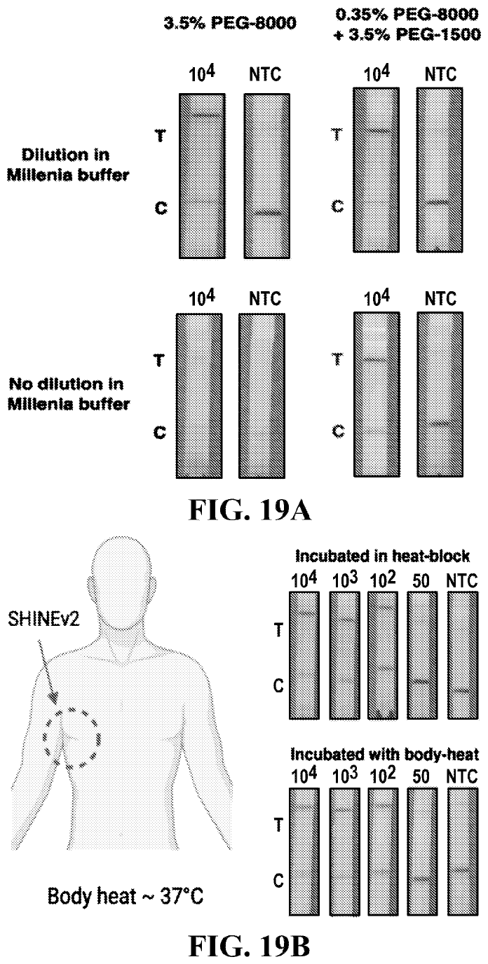

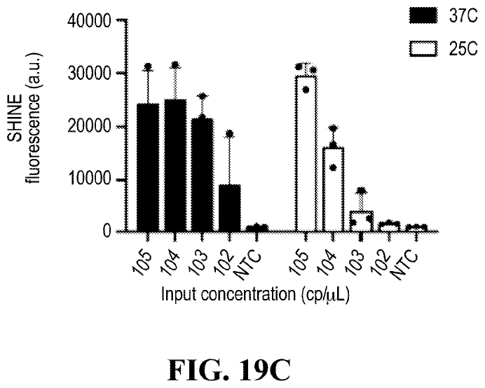

For optimizing and/or simplifying assay readout the 3.5% PEG-8000 was removed from prior exemplary embodiments of SHINE lysis, amplification, and/or CRISPR-Cas detection reagents (see e.g., Examples 1-6 herein). The 3.5% PEG-8000 was substituted in the buffers with about 0.35% PEG-8000 w/v and 3.5% PEG-1500 w/v. This modification facilitated flow through to a lateral flow detection without requiring prior dilution and simplifying the process for lateral flow read out. See e.g., FIGS. 19A-19C.

Optimization and results are described in greater detail below.

As shown and described in FIGS. 16A-16I, Applicant optimized various SHINE reagents and compositions to increase the ease-of-use and deployability of SHINE. FIG. 16A shows RNase activity in nasal fluid mixed with universal viral transport medium (UTM) untreated or treated with FastAmp Lysis buffer supplemented with RNase inhibitor or treated with HUDSON (a heat- and chemical-treatment) (For HUDSON see e.g., Barnes et al., Nat. Comm. 11:4131. 2020. https://doi.org/10.1038/s41467-020-17994-9). Activity was measured using RNaseAlert at room temperature (RT) for 30 minutes. FIG. 16B shows SARS-CoV-2 seedstock titer without treatment or after being incubated with lysis buffer (+5% RNase inhibitor) at RT for 5 minutes, 20 minutes or 20 minutes plus 10 minutes at 65° C. ***, infection not detected; PFU, plaque forming units. FIG. 16C shows SHINE fluorescence with different proportions of lysis mix (i.e., FastAmp lysis buffer, RNase inhibitor and UTM) input after a 90-minute incubation. FIG. 16D shows a schematic of the advantages of lyophilizing SHINE. FIG. 16E shows SHINE fluorescence after a 90-minute incubation on synthetic RNA target (10⁴ copies/μL) before and after lyophilization using different buffers. Fluorescence measured after 90 minutes. For buffer composition, see below. FIG. 16F shows SHINE fluorescence after a 90-minute incubation using lyophilized (LYO) reagents stored at RT, 4° C. or −20° C. over time. The Target concentration was 10⁴ copies/uL. FIG. 16G shows fluorescence kinetics for SHINEv1 (see e.g., Arizti-Sanz et al. Nat. Comm. 11:5921(2020). https://doi.org/10.1038/s41467-020-19097-x and elsewhere herein) and SHINEv2 (e.g., this example and elsewhere herein) using synthetic RNA targets; NTC, no target control. FIG. 16H shows lateral-flow detection of SARS-CoV-2 RNA in lysis buffer treated viral seedstocks using SHINE. Incubated for 90 minutes. C=control band; T=test band. FIG. 16I shows determination of an analytical limit of detection with 20 replicates of SHINE at different concentrations of SARS-CoV-2 RNA from lysis buffer treated viral seedstocks. Incubated for 90 minutes. For (FIGS. 16A,16E, and 16G), center=mean and error bars=s.d. for 3 technical replicates. In FIG. 16C, the heatmap values represent the mean for 3 technical replicates. For FIG. 16F, center=mean and error bars=s.d. for 3 biological replicates with 3 technical replicates each.

FIGS. 17A-17D demonstrate performance of SHINEv2 on clinical samples. FIG. 17A shows a schematic of side-by-side clinical sample testing using SHINEv2 and commercially available assays (e.g., BinaxNow, CareStart) and RT-qPCR. FIG. 17B shows SHINEv2, BinaxNow and CareStart test results for a subset of clinical nasopharyngeal (NP) swab samples with different Ct values (CDC EUA N1 RT-qPCR). C=control band; T=test band. FIG. 17C shows positive and negative test results for SHINEv2, BinaxNow and CareStart tests for RT-qPCR-positive clinical samples relative to viral RNA concentration and Ct value. FIG. 17D shoes side-by-side clinical performance of SHINEv2, BinaxNow and CareStart versus RT-qPCR. SHINEv2 reactions were incubated for 90 minutes.

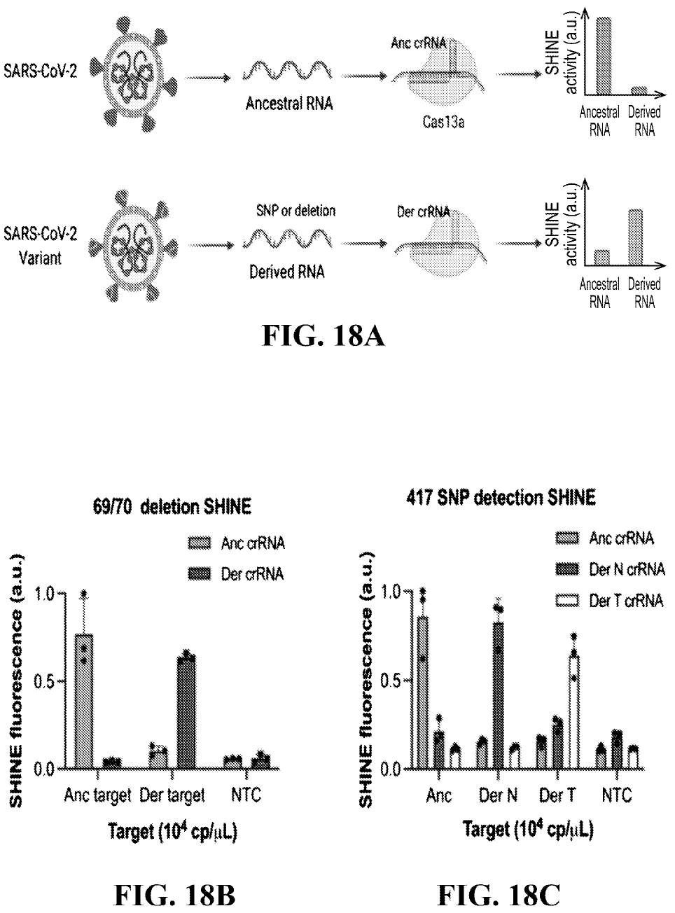

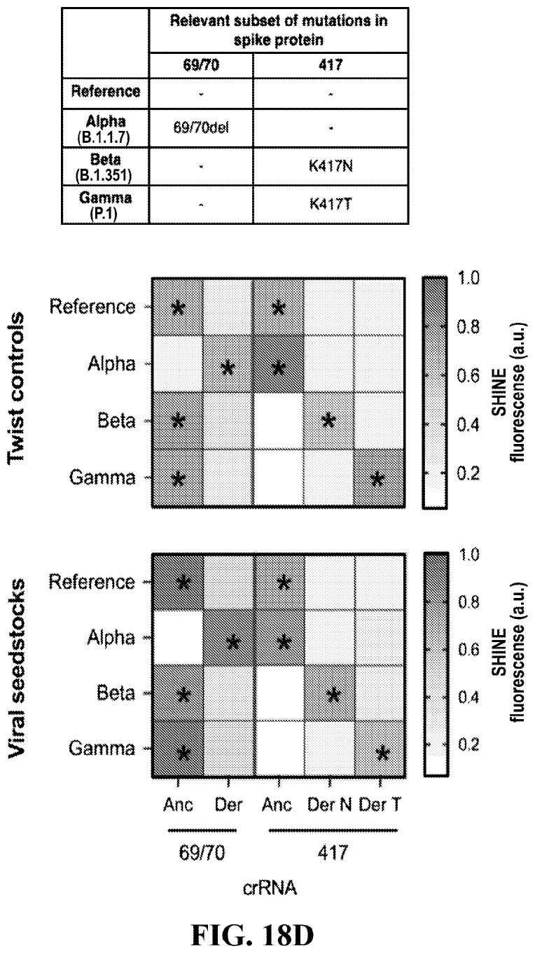

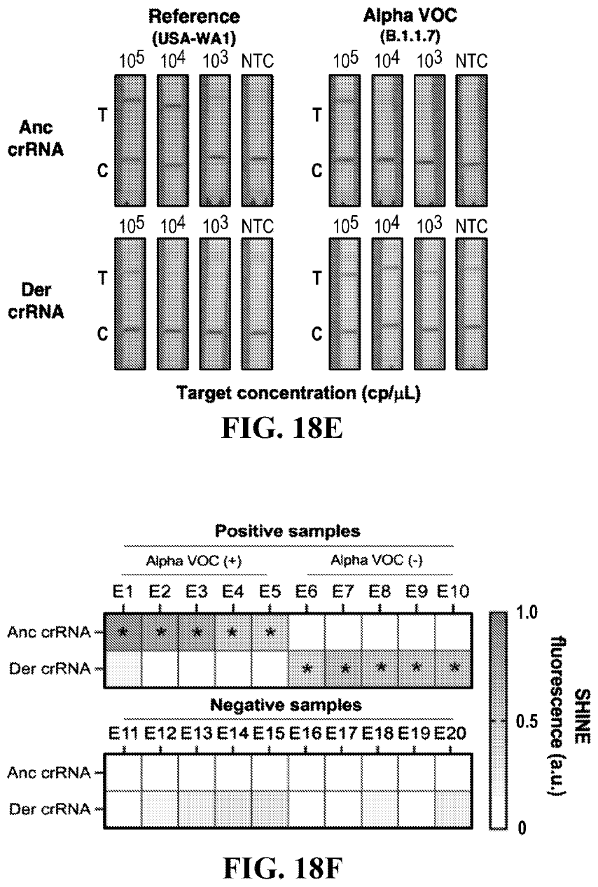

FIGS. 18A-18F demonstrates development of SHINEv2 assays for the detection of SARS-CoV-2 VOC. FIG. 18A shows a schematic of Cas13a-based detection of mutations in SARS-CoV-2 using a fluorescent readout. SNP, single nucleotide polymorphism; anc, ancestral; der, derived. FIG. 18B shows normalized SHINE fluorescence of the anc and der crRNAs for the 69/70 deletion assay against synthetic RNA targets after 90 minutes; NTC, no-target control. FIG. 18C shows normalized SHINE fluorescence of the ancestral (anc) and derived (derN/T) crRNAs for the 417 SNP detection assay against synthetic RNA targets after 90 minutes; NTC, no-target control. FIG. 18D shows identification of SARS-CoV-2 variants-of-concern (VOC) using normalized SHINE fluorescence on full-genome synthetic RNA controls (full genome synthetic RNA) and RNA extracted from viral seedstock; target RNA concentration: 10⁴ copies/μL. FIG. 18E shows colorimetric lateral flow-based detection of SARS-CoV-2 RNA in contrived clinical samples using the 69/70 SHINEv2 assay. SHINEv2 incubation time: 90 minutes. NTC, no-target control. T, test line; C, control line. FIG. 18F shows mean fluorescence of 69/70 SHINEv2 assay on SARS-CoV-2 RNA extracted from clinical samples, after 90 minutes. For FIG. 18B and FIG. 18C, center=mean and error bars=s.d. for 3 technical replicates. In FIG. 18D and FIG. 18F, the heatmap values represent the mean for 3 technical replicates. * in FIG. 18D and FIG. 18F indicate signal above threshold.

FIGS. 19A-19C demonstrates optimization of assay readout and processing for lateral-flow detection. FIG. 19A shows lateral-flow based detection of SARS-CoV-2 RNA using SHINEv2 with different polyethylene glycol (i.e., PEG) compositions. Dilution refers to lateral flow buffer being mixed in (e.g., diluted) prior to adding it to the paper strip. Incubation was for 90 minutes. NTC, no-target control. FIG. 19B shows lateral flow based SHINEv2 detection of SARS-CoV-2 RNA after a 90-minute incubation in a heat-block or using body-heat (urderarm). NTC, no-target control. FIG. 19C shows SHINE fluorescence on SARS-CoV-2 RNA after 90 minutes at 37° C. or 25° C.; NTC, no-target control. For FIG. 19C, center=mean and error bars=s.d. for 3 technical replicates.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition comprising:
a nucleic acid detection system comprising:
a CRISPR system comprising an effector protein and one or more guide RNAs, capable of specifically binding a virus-specific target molecule; and
a viral polynucleotide preparation formulation capable of releasing virus polynucleotides from viruses, inactivating nucleases, inactivating viruses, or a combination thereof at a temperature of about 15 degrees Celsius or greater.

2. The composition of claim 1, further comprising a detection construct.

3. The composition of claim 2, wherein the detection construct comprises an RNA or DNA oligonucleotide and further comprises a first molecule on a first end and a second molecule on a second end.

4. The composition of claim 1, further comprising one or more nucleic acid amplification reagents.

5. A method of detecting a virus in a sample comprising:
releasing virus polynucleotides from a virus in the sample;
inactivating nucleases present in the sample;
inactivating viruses present in the sample;
amplifying virus polynucleotides in the sample;
combining the sample with a nucleic acid detection system comprising:
a CRISPR system comprising an effector protein and one or more guide RNAs, capable of specifically binding a virus-specific target molecule; and
a detection construct;
activating the effector protein such that a detectable positive signal is produced, wherein activating the effector protein occurs via specific binding of the one or more guide RNAs to one or more virus-specific target molecules and results in modification of the detection construct such that a detectable signal is produced; and
detecting the detectable signal, wherein the detectable signal indicates a presence of one or more viruses in the sample,
wherein amplifying and activating occur in the same reaction and wherein the method does not include a step of extracting a virus polynucleotide from the sample.

6. The method of claim 5, wherein the steps of releasing, inactivating nucleases, inactivating viruses, amplifying and activating occur in the same reaction vessel.

7. The method of claim 5, wherein the steps of releasing, inactivating nucleases, inactivating viruses, amplifying, activating, and detecting occur in the same reaction vessel.

8. The method of claim 5, wherein the step of releasing, inactivating nucleases, inactivating virus, or a combination thereof occurs in a viral polynucleotide preparation formulation capable of releasing virus polynucleotides from viruses, inactivating nucleases, inactivating viruses, or a combination thereof at a temperature of about 15 degrees Celsius or greater.

10. The method of claim 8, wherein the nucleic acid detection system is contained in the viral polynucleotide preparation formulation.

11. The method of claim 10, wherein the viral polynucleotide preparation formulation comprises one or more of the following: a buffer, wherein the buffer is optionally HEPES, an amount of sucrose, an amount mannitol, a salt, PEG-8000, and PEG-1500.

12. The method of claim 10, wherein the viral polynucleotide preparation formulation does not comprise PEG.

13. The method of claim 10, wherein the viral polynucleotide preparation formulation is lyophilized.

14. The method of claim 5, wherein inactivating nucleases is carried out at a temperature ranging from about 15 degrees C. to about 50 degrees C.

15. The method of claim 5, wherein inactivating viruses occurs at a temperature ranging from about 15 degrees C. to about 95 degrees C.

16. The method of claim 5, wherein inactivating nucleases and inactivating viruses occurs at the same temperature.

17. The method of claim 5, wherein inactivating nucleases and inactivating viruses occurs at different temperatures.

18. The method of claim 5, wherein inactivating nucleases, inactivating viruses, or both together occurs for a period of time ranging from about 5 minutes to about 60 minutes.

19. The method of claim 5, further comprising distributing a sample or set of samples into one or more individual discrete volumes, wherein the individual discrete volumes comprise the nucleic acid detection.

20. The method of claim 5, further comprising incubating the sample or set of samples under conditions sufficient to allow binding of the one or more guide RNAs to one or more virus-specific target molecules.

21. The method of claim 5, wherein the one or more guide RNAs comprise one or more synthetic mismatches.

22. The method of claim 5, wherein the one or more guide RNAs comprise a pan-viral guide RNA set that is capable of detecting each virus, viral strain, or both in a set of viruses.

23. The method of claim 19, wherein the guide RNAs are derived using a set cover approach.

24. The method of claim 5, wherein the amplification step occurs for a period of time ranging from about 10 minutes to 2 hours.

25. The method of claim 5, wherein the detection step is of a period of time ranging from about 10 minutes to 3 hours.

26. The method of claim 5, wherein the sample volume ranges from about 1 microliter to about 100 microliters.

27. The method of claim 5, wherein the detection construct comprises or consists of an RNA-based detection construct comprising an RNA oligonucleotide to which a detectable molecule and masking component are attached.

28. The method of claim 5, wherein the effector protein is a Cas protein having collateral polynucleotide cleavage activity.

29. The method of claim 27, wherein the Cas protein having collateral polynucleotide cleavage activity is selected from: Cas13a (C2c2), Cas13b (Group 29/30), Cas13c, Cas13d, Cas12a (Cpf1), Cas12b (C2c1), Cas12c (C2c3), Cas12d (CasY), Cas12e (CasX), Cas14 (Cas 12f), and combinations thereof.

30. The method of claim 5, wherein the sample comprises two or more viruses and wherein the method distinguishes between the two or more viruses.

31. The method of claim 5, wherein the guide RNAs are capable of detecting single nucleotide variants of one or more viruses.

32. The method of claim 5, wherein the detectable signal is an optical signal.

33. The method of claim 25, wherein the optical signal is a fluorescent signal or a colorimetric signal.

34. The method of claim 5, wherein the nucleic acid detection system is not contained in/on a substrate.

35. The method of claim 5, wherein the nucleic acid detection system is contained in/on a substrate, and wherein the substrate is exposed to the sample.

36. The method of claim 34, wherein the same or a different nucleic acid detection system is present at multiple discrete locations on the substrate.

37. The method of claim 34, wherein the substrate is a flexible materials substrate.

38. The method of claim 37, wherein the flexible materials substrate is a paper substrate, a fabric substrate, or a flexible polymer-based substrate.

39. The method of claim 35, wherein each different nucleic acid detection system detects a different virus or viral strain at each discrete location.

40. The method of claim 35, wherein the substrate is exposed to the sample passively, by immersing the substrate in a fluid to be sampled, by applying a fluid to be tested to the substrate, or by contacting a surface to be tested with the substrate.

41. The method of claim 35, wherein the substrate is configured as a lateral flow strip.

42. The method of claim 41, wherein the detection construct comprises a first and a second molecule and wherein the method comprises detecting the first and the second molecule optionally at discrete locations on the lateral flow strip.

43. The method of claim 42, wherein the first molecule and the second molecule are detected by binding a first antibody capable of specifically binding the first molecule or the second molecule, and optionally further comprising detecting the bound first antibody, optionally with a second antibody capable of specifically binding the first antibody.

44. The method of claim 42, wherein said lateral flow strip comprises an upstream first antibody directed against the first molecule and a downstream second antibody directed against the second molecule, and wherein an uncleaved detection construct is bound by the first antibody when the target molecule is not present in said sample, and wherein a cleaved detection construct is bound both by the first antibody and the second antibody when the target nucleic acid is present in said sample.

45. The method of claim 5, wherein the sample is a biological or environmental sample.

46. The method of claim 5, wherein the biological sample is obtained from a tissue sample, saliva, blood, plasma, sera, stool, urine, sputum, mucous, lymph, synovial fluid, cerebrospinal fluid, ascites, pleural effusion, seroma, pus, or swab of skin or a mucosal membrane surface.

47. The method of claim 5, wherein the environmental sample is obtained from a food sample, a beverage sample, a paper surface, a fabric surface, a metal surface, a wood surface, a plastic surface, a soil sample, a freshwater sample, a waste water sample, a saline water sample, exposure to atmospheric air or other gas sample, or a combination thereof.

48. The method of claim 45, wherein the environmental sample or biological samples are crude samples and/or wherein the one or more target molecules are not purified or amplified from the sample prior to application of the method.

49. The method of claim 5, wherein the virus is a DNA virus.

50. The method of claim 5, wherein the virus is a double-stranded RNA virus, a positive sense RNA virus, a negative sense RNA virus, a retrovirus, or a combination thereof.

51. The method of claim 5, wherein the virus is a coronavirus, an Ebola virus, measles, SARS, Chikungunya virus, Marburg, MERS, Dengue, Lassa, influenza, rhabdovirus, HIV, a hepatitis virus (including hepatitis A, B, C, D, or E), an influenza virus (including an influenza A or influenza B), a human respiratory syncytial virus, Sudan ebola virus, Bundibugyo virus, Tai Forest ebola virus, Reston ebola virus, Achimota virus, Aedes flavivirus, Aguacate virus, Akabane virus, Alethinophid reptarenavirus, Allpahuayo mammarenavirus, Amapari mmarenavirus, Andes virus, Apoi virus, Aravan virus, Aroa virus, Arumwot virus, Atlantic salmon paramyxovirus, Australian bat lyssavirus, Avian bornavirus, Avian metapneumovirus, Avian paramyxoviruses, penguin or Falkland Islandsvirus, BK polyomavirus, Bagaza virus, Banna virus, Bat herpesvirus, Bat sapovirus, Bear Canon mammarenavirus, Beilong virus, Betacoronavirus, Betapapillomavirus 1-6, Bhanja virus, Bokeloh bat lyssavirus, Boma disease virus, Bourbon virus, Bovine hepacivirus, Bovine parainfluenza virus 3, Bovine respiratory syncytial virus, Brazoran virus, Bunyamwera virus, Caliciviridae virus. California encephalitis virus, Candiru virus, Canine distemper virus, Canine pneumovirus, Cedar virus, Cell fusing agent virus, Cetacean morbillivirus, Chandipura virus, Chaoyang virus, Chapare mammarenavirus, Chikungunya virus, Colobus monkey papillomavirus, Colorado tick fever virus, Cowpox virus, Crimean-Congo hemorrhagic fever virus, Culex flavivirus, Cupixi mammarenavirus, Dengue virus, Dobrava-Belgrade virus, Donggang virus, Dugbe virus, Duvenhage virus, Eastern equine encephalitis virus, Entebbe bat virus, Enterovirus A-D, European bat lyssavirus 1-2, Eyach virus, Feline morbillivirus, Fer-de-Lance paramyxovirus, Fitzroy River virus, Flaviviridae virus, Flexal mammarenavirus, GB virus C, Gairo virus, Gemycircularvirus, Goose paramyxovirus SF02, Great Island virus, Guanarito mammarenavirus, Hantaan virus, Hantavirus Z10, Heartland virus, Hendra virus, Hepatitis A/B/C/E, Hepatitis delta virus, Human bocavirus, Human coronavirus, Human endogenous retrovirus K, Human enteric coronavirus, Human genital-associated circular DNA virus-1, Human herpesvirus 1-8, Human mastadenovirus A-G, Human papillomavirus, Human parainfluenza virus 1-4, Human paraechovirus, Human picornavirus, Human smacovirus, Ikoma lyssavirus, Ilheus virus, Influenza A-C, Ippy mammarenavirus, Irkut virus, J-virus, JC polyomavirus, Japanese encephalitis virus, Junin mammarenavirus, KI polyomavirus, Kadipiro virus, Kamiti River virus, Kedougou virus, Khujand virus, Kokobera virus, Kyasanur forest disease virus, Lagos bat virus, Langat virus, Lassa mammarenavirus, Latino mammarenavirus, Leopards Hill virus, Liao ning virus, Ljungan virus, Lloviu virus, Louping ill virus, Lujo mammarenavirus, Luna mammarenavirus, Lunk virus, Lymphocytic choriomeningitis mammarenavirus, Lyssavirus Ozernoe, MSSI2Y225 virus, Machupo mammarenavirus, Mamastrovirus 1, Manzanilla virus, Mapuera virus, Marburg virus, Mayaro virus, Measles virus, Menangle virus, Mercadeo virus, Merkel cell polyomavirus, Middle East respiratory syndrome coronavirus, Mobala mammarenavirus, Modoc virus, Moijang virus, Mokolo virus, Monkeypox virus, Montana myotis leukoenchalitis virus, Mopeia lassa virus reassortant 29, Mopeia mammarenavirus, Morogoro virus, Mossman virus, Mumps virus, Murine pneumonia virus, Murray Valley encephalitis virus, Nariva virus, Newcastle disease virus, Nipah virus, Norwalk virus, Norway rat hepacivirus, Ntaya virus, O'nyong-nyong virus, Oliveros mammarenavirus, Omsk hemorrhagic fever virus, Oropouche virus, Parainfluenza virus 5, Parana mammarenavirus, Parramatta River virus, Peste-des-petits-ruminants virus, Pichande mammarenavirus, Picornaviridae virus, Pirital mammarenavirus, Piscihepevirus A, Porcine parainfluenza virus 1, porcine rubulavirus, Powassan virus, Primate T-lymphotropic virus 1-2, Primate erythroparvovirus 1, Punta Toro virus, Puumala virus, Quang Binh virus, Rabies virus, Razdan virus, Reptile bornavirus 1, Rhinovirus A-B, Rift Valley fever virus, Rinderpest virus, Rio Bravo virus, Rodent Torque Teno virus, Rodent hepacivirus, Ross River virus, Rotavirus A-I, Royal Farm virus, Rubella virus, Sabia mammarenavirus, Salem virus, Sandfly fever Naples virus, Sandfly fever Sicilian virus, Sapporo virus, Sathuperi virus, Seal anellovirus, Semliki Forest virus, Sendai virus, Seoul virus, Sepik virus, Severe acute respiratory syndrome-related coronavirus, Severe fever with thrombocytopenia syndrome virus, Shamonda virus, Shimoni bat virus, Shuni virus, Simbu virus, Simian torque teno virus, Simian virus 40-41, Sin Nombre virus, Sindbis virus, Small anellovirus, Sosuga virus, Spanish goat encephalitis virus, Spondweni virus, St. Louis encephalitis virus, Sunshine virus, TTV-like mini virus, Tacaribe mammarenavirus, Taila virus, Tamana bat virus, Tamiami mammarenavirus, Tembusu virus, Thogoto virus, Thottapalayam virus, Tick-borne encephalitis virus, Tioman virus, Togaviridae virus, Torque teno canis virus, Torque teno douroucouli virus, Torque teno felis virus, Torque teno midi virus, Torque teno sus virus, Torque teno tamarin virus, Torque teno virus, Torque teno zalophus virus, Tuhoko virus, Tula virus, Tupaia paramyxovirus, Usutu virus, Uukuniemi virus, Vaccinia virus, Variola virus, Venezuelan Vesicular stomatitis Indiana virus, WU Polyomavirus, Wesselsbron virus, West Caucasian bat virus, West Nile virus, Western equine encephalitis virus, Whitewater Arroyo mammarenavirus, Yellow fever virus, Yokose virus, Yug Bogdanovac virus, Zaire ebolavirus, Zika virus, or Zygosaccharomyces bailii virus Z viral sequence, or a combination thereof.

52. The method of claim 5, wherein the virus is a coronavirus.

53. The method of claim 52, wherein the coronavirus is SARS-CoV-2.

54. The method of claim 5, wherein the method is performed in one hour or less.

55. The method of claim 5, wherein the virus polynucleotide is RNA.

56. The method of any one of claims 5-55, wherein the virus polynucleotide is DNA.

57. A method of monitoring viral disease outbreaks and/or evolution, comprising performing a method as in any one of claims 5-56.

58. A kit comprising: one or more compositions of any of claims 1-4.

59. A diagnostic device comprising:
one or more individual discrete volumes, one or more of the one or more individual discrete volumes comprises:
one or more nucleic acid detection systems comprising:
a CRISPR system comprising an effector protein and one or more guide RNAs, capable of specifically binding a virus-specific target molecule; and
a viral polynucleotide preparation formulation capable of releasing virus polynucleotides from viruses, inactivating nucleases, inactivating viruses, or a combination thereof at a temperature ranging of 15 degrees Celsius or greater.

60. The diagnostic device of claim 59, wherein one or more of the one or more individual discrete volumes further comprises a detection construct, wherein the detection construction is or optionally comprises and RNA detection construct.

61. The diagnostic device of claim 59, wherein one or more of the one or more individual discrete volumes further comprises a detection construct, wherein the detection construction is or optionally comprises and RNA detection construct.

62. The diagnostic device of claim 59, wherein one or more of the one or more individual discrete volumes further comprises one or more nucleic amplification reagents.

63. The diagnostic device of claim 59, wherein the one or more individual discrete volumes are droplets.

64. The diagnostic device of claim 59, wherein the one or more individual discrete volumes are defined on a solid substrate, are spots defined on a substrate, are contained within microwells, are contained within microfluidic channels, or a combination thereof.

65. The diagnostic device of claim 59, wherein the substrate is a flexible materials substrate.

66. The diagnostic device of claim 65, wherein the flexible materials substrate is a paper substrate, a fabric substrate, or a flexible polymer-based substrate.

67. The diagnostic device of claim 59, wherein the diagnostic device forms or comprises a lab on a chip (LOC) device.

68. The diagnostic device of claim 67, wherein the LOC device is or comprises a radio frequency identification (RFID) tag system.

69. The diagnostic device of claim 68, further comprising a wireless devices configured to communicate with the RFID tag system.

70. The diagnostic device of claim 59, wherein the effector protein is a Cas protein having collateral polynucleotide cleavage activity.

71. The diagnostic device of claim 70, wherein the Cas protein having collateral polynucleotide cleavage activity is selected from: Cas13a (C2c2), Cas13b (Group 29/30), Cas13c, Cas13d, Cas12a (Cpf1), Cas12b (C2c1), Cas12c (C2c3), Cas12d (CasY), Cas12e (CasX), Cas14 (Cas12f), and combinations thereof.

72. The diagnostic device of claim 59, wherein the effector protein comprises or is linked to an affinity tag, wherein each individual discrete volume comprises a capture molecule capable of specifically binding the affinity tag.

73. The diagnostic device of claim 59, wherein the one or more guide RNAs comprise one or more synthetic mismatches.

74. The diagnostic device of claim 59, wherein the one or more guide RNAs comprise a pan-viral guide RNA set that is capable of detecting each virus, viral strain, or both in a set of viruses.

75. The diagnostic device of claim 59, wherein the guide RNAs are derived using a set cover approach.

76. The diagnostic device of claim 59, further comprising one or more of the following:
i) a heating element, wherein the heating element is configured to heat the discrete volume(s) to a predetermined temperature;
ii) a mixing element;
iii) a pipetting element;
iv) one or more reservoirs configured to contain a reagent;
v) a removable cartridge configured for adding one or more samples and/or holding reagents;
vi) a sensor capable of detecting and measuring an optical signal;
vii) a controller;
viii) a transmitter configured to transmit a signal;
ix) a receiver configured to receive a signal;
x) a processor;
xi) memory; and
xii) a user interface.

77. The diagnostic device of claim 59, wherein the diagnostic device is a lateral flow device.

78. The diagnostic device of claim 77, wherein the diagnostic device comprises a substrate comprising a first end, wherein the first end comprises a sample loading portion and a first region loaded with a detectable ligand, the nucleic acid detection system, a detection construct, a first capture region comprising a first binding agent, and a second capture region comprising a second binding agent.

79. The diagnostic device of claim 78, wherein the detection construct comprises an RNA or DNA oligonucleotide and further comprises a first molecule on a first end and a molecule on a second end.

80. The diagnostic device of claim 78, wherein the sample loading portion further comprises the viral polynucleotide preparation formulation and optionally one or more amplification reagents.

81. The diagnostic device of claim 78, wherein the first capture region is proximate to and on the same end of the lateral flow substrate as the sample loading portion.

82. The diagnostic device of claim 78, wherein the first capture region comprises a first binding agent that is capable of specifically binding the first molecule of the detection construct.

83. The diagnostic device of claim 78, wherein the first binding agent is an antibody that is fixed or otherwise immobilized to the first capture region.

84. The diagnostic device of claim 78, wherein the second capture region is located towards the opposite end of the lateral flow substrate from the first binding region.

85. The diagnostic device of claim 78, wherein the second capture region comprises a second binding agent that is capable of specifically binding the second molecule of the detection construct or the detectable ligand.

86. The diagnostic device of claim 78, wherein the second binding agent is an antibody or an antibody-binding protein that is fixed or otherwise immobilized to the second capture region.
